(19) United States
(12) Patent Application Publication
(10) Pub. No.: US 2010/0145032 A1
(43) Pub. Date: Jun. 10, 2010

(54) NOVEL CARBOHYDRATE PROFILE COMPOSITIONS FROM HUMAN CELLS AND METHODS FOR ANALYSIS AND MODIFICATION THEREOF

(75) Inventors: Jarmo Laine, Helsinki (FI); Tero Satomaa, Helsinki (FI); Jari Natunen, Vantaa (FI); Annamari Heiskanen, Helsinki (FI); Maria Blomqvist, Itasalmi (FI); Anne Olonen, Lahti (FI); Juhani Saarinin, Helsinki (FI); Sari Tiitinen, Vantaa (FI); Ulla Impola, Helsinki (FI); Milla Mikkola, Helsinki (FI); Hanna Salo, Helsinki (FI); Olli Aitio, Helsinki (FI); Leena Valmu, Helsinki (FI); Suvi Natunen, Vantaa (FI); Heidi Anderson, Helsinki (FI); Virve Pitkänen, Helsinki (FI); Jukka Partanen, Helsinki (FI); Taina Jaatinen, Helsinki (FI); Minna Tiitanen, Espoo (FI); Tia Hirvonen, Helsinki (FI)

Correspondence Address: BIRCH STEWART KOLASCH & BIRCH, PO BOX 747, FALLS CHURCH, VA, 22040-0747, US

(73) Assignees: Suomen Punainen Risti, Veripalelu, Helsinki (FI); Glykos Finland Ltd., Helsinki (FI)

(21) Appl. No.: 12/523,628

(22) PCT Filed: Jan. 18, 2008
(86) PCT No.: PCT/FI08/50018

§ 371 Date: Nov. 20, 2009

(30) Foreign Application Priority Data
Jan. 18, 2007 (FI) 20075033
Jan. 18, 2007 (FI) 20075034
Mar. 8, 2007 (FI) 20070200
Mar. 13, 2007 (FI) 20070205
May 10, 2007 (FI) 20070368
May 10, 2007 (FI) 20070369
Jun. 29, 2007 (FI) PCT/FI2007/050405
Aug. 28, 2007 (FI) 20070650

(51) Int. Cl. C12Q 1/02; C12N 5/074; C12P 21/00; C07K 14/435

(52) U.S. Cl. 530/395; 435/29; 435/366; 435/71.1

(57) ABSTRACT

The invention describes reagents and methods for specific binders to glycan structures of stem cells. Furthermore the invention is directed to screening of additional binding reagents against specific glycan epitopes on the surfaces of the stem cells. The preferred binders of the glycans structures includes proteins such as enzymes, lectins and antibodies.

### FIELD OF THE INVENTION

The invention describes reagents and methods for specific binders to glycan structures of stem cells. Furthermore the invention is directed to screening of additional binding reagents against specific glycan epitopes on the surfaces of the stem cells. The preferred binders of the glycans structures includes proteins such as enzymes, lectins and antibodies.

The invention describes novel compositions of glycans, glycomes, from stem cells in blood, especially cord blood (CB) derived stem cells, (most preferably CD133+ cells,) and especially novel subcompositions of the glycomes with specific monosaccharide compositions and glycan structures. The invention is further directed to methods for modifying the glycomes and analysis of the glycomes and the modified glycomes. Furthermore, the invention is directed to stem cells carrying the modified glycomes on their surfaces. The glycomes are preferably analysed by profiling methods able to detect reproducibly and quantitatively numerous individual glycan structures at the same time. The most preferred type of the profile is a mass spectrometric profile. The invention specifically revealed novel target structures and is especially directed to the development of reagents recognizing the structures.

### BACKGROUND OF THE INVENTION

Stem Cells

Stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

The first evidence for the existence of stem cells came from studies of embryonic carcinoma (EC) cells, the undifferentiated stem cells of teratocarcinomas, which are tumors derived from germ cells. These cells were found to be pluripotent and immortal, but possess limited developmental potential and abnormal karyotypes (Rossant and Papaioannou, Cell Differ 15,155-161, 1984). ES cells, on the other hand, are thought to retain greater developmental potential because they are derived from normal embryonic cells, without the selective pressures of the teratocarcinoma environment.

Pluripotent embryonic stem cells have traditionally been derived principally from two embryonic sources. One type can be isolated in culture from cells of the inner cell mass of a pre-implantation embryo and are termed embryonic stem (ES) cells (Evans and Kaufman, Nature 292,154-156, 1981; U.S. Pat. No. 6,200,806). A second type of pluripotent stem cell can be isolated from primordial germ cells (PGCS) in the mesenteric or genital ridges of embryos and has been termed embryonic germ cell (EG) (U.S. Pat. No. 5,453,357, U.S. Pat. No. 6,245,566). Both human ES and EG cells are pluripotent. This has been shown by differentiating cells in vitro and by injecting human cells into immunocompromised (SCUM) mice and analyzing resulting teratomas (U.S. Pat. No. 6,200,806). The term “stem cell” as used herein means stem cells including embryonic stem cells or embryonic type stem cells and stem cells diffentiated thereof to more tissue specific stem cells, adults stem cells including mesenchymal stem cells and blood stem cells such as stem cells obtained from bone marrow or cord blood.

The present invention provides novel markers and target structures and binders to these for especially embryonic and adult stem cells, when these cells are not hematopoietic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. The invention further provides novel markers for CD133+ cells and novel hematopoietic stem cell markers according to the invention, especially when the structures does not include NeuNAcα3Galβ4(Fucα3)₀₋₁GlcNAc. Preferably the hematopoietic stem cell structures are non-sialylated, fucosylated structures Galβ1-3-structures according to the invention and even more preferably type 1 N-acetyllactosamine structures Galβ3GlcNAc or separately preferred Galβ3GalNAc based structures.

Human ES, EG and EC cells, as well as primate ES cells, express alkaline phosphatase, the stage-specific embryonic antigens SSEA-3 and SSEA-4, and surface proteoglycans that are recognized by the TRA-1-60; and TRA-1-81 antibodies. All these markers typically stain these cells, but are not entirely specific to stem cells, and thus cannot be used to isolate stem cells from organs or peripheral blood.

The SSEA-3 and SSEA-4 structures are known as galactosylgloboside and sialylgalactosylgloboside, which are among the few suggested structures on embryonal stem cells, though the nature of the structures in not ambigious. An antibody called K21 has been suggested to bind a sulfated polysaccharide on embryonal carcinoma cells (Badcock G et al Cancer Res (1999) 4715-19. Due to cell type, species, tissue and other specificity aspects of glycosylation (Furukawa, K., and Kobata, A. (1992) Curr. Opin. Struct. Biol. 3, 554-559, Gagneux, and Varki, A. (1999) Glycobiology 9, 747-755;Gawlitzek, M. et al. (1995), J. Biotechnol. 42, 117-131; Goelz, S., Kumar, R., Potvin, B., Sundaram, S., Brickelmaier, M., and Stanley, P. (1994) J. Biol. Chem. 269, 1033-1040; Kobata, A (1992) Eur. J. Biochem. 209 (2) 483-501.) This result does not indicate the presence of the structure on native embryonal stem cells. The present invention is directed to human stem cells.

Some low specificity plant lectin reagents have been reported in binding of embryonal stem cell like materials. Venable et al 2005, (Dev. Biol. 5:15) measured lectins the binding of SSEA-4 antibod positive subpopulation of embryonal stem cells. This approach suffers obvious problems. It does not tell the expression of the structures in antive non-selected embryonal strem cells. The SSEA-4 was chosen select especially pluripotent stem cells. The scientists of the same Bresagen company have further revealed that actual role of SSEA-4 with the specific stem cell lines is not relevant for the pluripotency.

The work does not reveal: 1) The actual amount of molecules binding to the lectins or 2) presence of any molecules due to defects caused by the cell sorting and experimental problems such as trypsination of the cells. It is really alerting that the cells were trypsinized, which removes protein and then enriched by possible glycolipid binding SSEA4 antibody and secondary antimouse antibody, fixed with paraformaldehyde without removing the antibodies, and labelled by simultaneous with lectin and the same antibody and then the observed glycan profile is the similar as revealed by lectin analysis by same scientist for antibody glycosylation (M. Pierce US2005) or 3) the actual structures, which are bound by the lectins. To reveal the possible residual binding to the cells would require analysis of of the glycosylations of the antibodies used (sources and lots not revealed).

The purity of the SSEA-4 positive cells was reported to be 98-99%, which is unusually high. The quantitation of the binding is not clear as a figure shows about 10% binding by lectins LTL and DBA, which are not bound to hESC-cells 3^{rd} page, column 2, paragraph 2 and by immunocytochemistry 4the page last line.

It appears that skilled artisan would consider the results of Venable et al such convienent colocalization of SSEA-4 and the lectin binding by binding of the lectins to the anti-SSEA-4 antibody. It appears that the more rare binding would reflect lower proportion of the terminal epitope per antibody molecule leading to lower density of the labellable antibodies. It is also realized that the non-controlled cell culture process with animal derived material would lead to contamination of the cells by N-glycolyl-neuraminic acid, which may be recognized by anti-mouse antibodies used as secondary antibody (not defined what kind of anti-mouse) used in purification and analysis of purity, which could lead to convieniently high cell purity. The work is directed only to the “pluripotent” embryonal stem cells associated with SSEA-4 labelling and not to differentiated variants thereof as the present invention. The results indicated possible binding (likely on the antibodies) to certain potential monosaccharide epitopes (Tables) such Gal and Galactosamine for RCA (ricin, inhitable by Gal or lactose), GlcNAc for TL (tomato lectin), Man or Glc for ConA, Sialic acid/Sialic acid α6GalNAc for SNA, Manα for HHL; lectins with partial binding not correlating with SSEA-4: GalNAc/GalNAcβ4Gal (in text) WFA, Gal for PNA, and Sialic acid/Sialic acid α6GalNAc for SNA; and lectins associated by part of SSEA-4 cells were indicated to bind Gal by PHA-L and PHA-E, GalNAc by VVA and Fuc by UEA, and Gal by MAA (inhibited by lactose). UEA binding was discussed with reference as endothelial marker and O-linked fucose which is directly bound to Ser (Thr) on protein. The background has indicated a H type 2 specificity for the endothelial UEA receptor. The specifities of the lectins are somawhat unusual, but the product codes or isolectin numbers/names of the lectins were not indicated (except for PHA-E and PHA-L) and it is known that plants contain numerous isolectins with varying specificities.

The present invention revealed specifc structures by mass spectrometric profiling, NMR spectrometry and binding reagents including glycan modifying enzymes. The lectins are in general low specificity molecules. The present invention revealed binding epitiopes larger than the previously described monosaccharide epitopes. The larger epitopes allowed us to design more specific binding substances with typical binding specificities of at least disaccharides. The invention also revealed lectin reagents with speficified with useful specificities for analysis of native embryonal stem cells without selection against an uncontrolled marker and/or coating with an antibody or two from different species. Clearly the binding to native embryonal stem cells is different as the binding with MAA was clear to most of cells, there was differences between cell line so that RCA, LTA and UEA was clearly binding a HESC cell line but not another.

Methods for separation and use of stem cells are known in the art.

Characterizations and isolation of hematopoietic stem cells are reported in U.S. Pat. No. 5,061,620. The hematopoietic CD34 marker is the most common marker known to identify specifically blood stem cells, and CD34 antibodies are used to isolate stem cells from blood for transplantation purposes. However, CD34+ cells can differentiate only or mainly to blood cells and differ from embryonic stem cells which have the capability of developing into different body cells. Moreover, expansion of CD34+ cells is limited as compared to embryonic stem cells which are immortal. U.S. Pat. No. 5,677,136 discloses a method for obtaining human hematopoietic stem cells by enrichment for stem cells using an antibody which is specific for the CD59 stem cell marker. The CD59 epitope is highly accessible on stem cells and less accessible or absent on mature cells. U.S. Pat. No. 6,127,135 provides an antibody specific for a unique cell marker (EM10) that is expressed on stem cells, and methods of determining hematopoietic stem cell content in a sample of hematopoietic cells. These disclosures are specific for hematopoietic cells and the markers used for selection are not absolutely absent on more mature cells.

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. In addition, the ability to isolate stem cells may serve in the treatment of lymphomas and leukemias, as well as other neoplastic conditions where the stem cells are purified from tumor cells in the bone marrow or peripheral blood, and reinfused into a patient after myelosuppressive or myeloablative chemotherapy.

Multiple adult stem cell populations have been discovered from various adult tissues. In addition to hematopoietic stem cells, neural stem cells were identified in adult mammalian central nervous system (Ourednik et al. Clin. Genet. 56, 267, 1999). Adult stem cells have also been identified from epithelial and adipose tissues (Zuk et al. Tissue Engineering 7, 211, 2001). Mesenchymal stem cells (MSCs) have been cultured from many sources, including liver and pancreas (Hu et al. J. Lab Clin Med. 141, 342-349, 2003). Recent studies have demonstrated that certain somatic stem cells appear to have the ability to differentiate into cells of a completely different lineage (Pfendler KC and Kawase E, Obstet Gynecol Sury 58, 197-208, 2003). Monocyte derived (Zhao et al. Proc. Natl. Acad. Sci. USA 100, 2426-2431, 2003) and mesodermal derived (Schwartz et al. J. Clin. Invest 109, 1291-1301, 2002) cells that possess some multipotent characteristics were identified. The presence of multipotent “embryonic-like” progenitor cells in blood was suggested also by in-vivo experiments following bone marrow transplantations (Zhao et al. Brain Res Protoc 11, 38-45, 2003). However, such multipotent “embryonic-like” stem cells cannot be identified and isolated using the known markers.

The possibility of recovering fetal cells from the maternal circulation has generated interest as a possible means, non-invasive to the fetus, of diagnosing fetal anomalies (Simpson and Elias, J. Am. Med. Assoc. 270, 2357-2361, 1993). Prenatal diagnosis is carried out widely in hospitals throughout the world. Existing procedures such as fetal, hepatic or chorionic biopsy for diagnosis of chromosomal disorders including Down's syndrome, as well as single gene defects including cystic fibrosis are very invasive and carry a considerable risk to the fetus. Amniocentesis, for example, involves a needle being inserted into the womb to collect cells from the embryonic tissue or amniotic fluid. The test, which can detect Down's syndrome and other chromosomal abnormalities, carries a miscarriage risk estimated at 1%. Fetal therapy is in its very early stages and the possibility of early tests for a wide range of disorders would undoubtedly greatly increase the pace of research in this area. Thus, relatively non-invasive methods of prenatal diagnosis are an attractive alternative to the very invasive existing procedures. A method based on maternal blood should make earlier and easier diagnosis more widely available in the first trimester, increasing options to parents and obstetricians and allowing for the eventual development of specific fetal therapy.

The present invention provides methods of identifying, characterizing and separating stem cells having characteristics of embryonic stem (ES) cells for diagnostic, therapy and tissue engineering. In particular, the present invention provides methods of identifying, selecting and separating embryonic stem cells or fetal cells from maternal blood and to reagents for use in prenatal diagnosis and tissue engineering methods. The present invention provides for the first time a specific marker/binder/binding agent that can be used for identification, separation and characterization of valuable stem cells from tissues and organs, overcoming the ethical and logistical difficulties in the currently available methods for obtaining embryonic stem cells.

The present invention overcomes the limitations of known binders/markers for identification and separation of embryonic or fetal stem cells by disclosing a very specific type of marker/binder, which does not react with differentiated somatic maternal cell types. In other aspect of the invention, a specific binder/marker/binding agent is provided which does not react, i.e. is not expressed on feeder cells, thus enabling positive selection of feeder cells and negative selection of stem cells.

By way of exemplification, the binder to Formula (I) are now disclosed as useful for identifying, selecting and isolating pluripotent or multipotent stem cells including embryonic stem cells, which have the capability of differentiating into varied cell lineages.

According to one aspect of the present invention a novel method for identifying pluripotent or multipotent stem cells in peripheral blood and other organs is disclosed. According to this aspect an embryonic stem cell binder/marker is selected based on its selective expression in stem cells and/or germ stem cells and its absence in differentiated somatic cells and/or feeder cells. Thus, glycan structures expressed in stem cells are used according to the present invention as selective binders/markers for isolation of pluripotent or multipotent stem cells from blood, tissue and organs. Preferably the blood cells and tissue samples are of mammalian origin, more preferably human origin.

According to a specific embodiment the present invention provides a method for identifying a selective embryonic stem cell binder/marker comprising the steps of:

A method for identifying a selective stem cell binder to a glycan structure of Formula (I) which comprises:

i. selecting a glycan structure exhibiting specific expression in/on stem cells and absence of expression in/on feeder cells and/or differentiated somatic cells; ii. and confirming the binding of binder to the glycan structure in/on stem cells.

By way of a non-limiting example, adult, mesenchymal, embryonal type, or hematopoietic stem cells selected using the binder may be used in regenerating the hematopoietic or ther tissue system of a host deficient in any class of stem cells. A host that is diseased can be treated by removal of bone marrow, isolation of stem cells and treatment with drugs or irradiation prior to re-engraftment of stem cells. The novel markers of the present invention may be used for identifying and isolating various stem cells; detecting and evaluating growth factors relevant to stem cell self-regneration; the development of stem cell lineages; and assaying for factors associated with stem cell development.

### BRIEF DESCRIPTION OF THE DRAWINGS

FIG. 1. FACS analysis of seven cord blood mononuclear cell samples (parallel columns) by FITC-labelled lectins. The percentages refer to proportion of cells binding to lectin. For abbreviations of FITC-labelled lectins see text.

FIG. 2. Portrait of the hESC N-glycome. MALDI-TOF mass spectrometric profiling of the most abundant 50 neutral N-glycans (A.) and 50 sialylated N-glycans (B.) of the four hESC lines FES 21, 22, 29, and 30 (black columns), four EB samples (gray columns), and four st.3 differentiated cell samples (white columns) derived from the four hESC lines, respectively. The columns indicate the mean abundance of each glycan signal (% of the total glycan signals). The observed m/z values for either [M+Na]+ or [M−H]− ions for the neutral and sialylated N-glycan fractions, respectively, are indicated on the x-axis. Proposed monosaccharide compositions and N-glycan types are presented in Tables.

FIG. 3. Detection of hESC glycans by structure-specific reagents. To study the localization of the detected glycan components in hESC, stem cell colonies grown on mouse feeder cell layers were labeled by fluoresceinated glycan-specific reagents selected based on the analysis results. A. The hESC surfaces were stained by Maackia amurensis agglutinin (MAA), indicating that α2,3-sialylated glycans are abundant on hESC but not on feeder cells (MEF, mouse feeder cells). B. In contrast, the hESC cell surfaces were not stained by Pisum sativum agglutinin (PSA) that recognized mouse feeder cells, indicating that α-mannosylated glycans are not abundant on hESC surfaces but are present on mouse feeder cells. C. Addition of 3′-sialyllactose blocks MAA binding, and D. addition of D-mannose blocks PSA binding.

FIG. 4. Mass spectrometric profiling of human embryonic stem cell and differentiated cell N-glycans. a Neutral N-glycans and b 50 most abundant acidic N-glycans of the four hESC lines (white columns), embryoid bodies derived from FES 29 and FES 30 hESC lines (EB, light columns), and stage 3 differentiated cells derived from FES 29 (st.3, black columns). The columns indicate the mean abundance of each glycan signal (% of the total detected glycan signals). Error bars indicate the range of detected signal intensities. Proposed monosaccharide compositions are indicated on the x-axis. H: hexose, N: N-acetylhexosamine, F: deoxyhexose, S: N-acetylneuraminic acid, G: N-glycolylneuraminic acid, P: sulphate/phosphate ester.

FIG. 5. A) Baboon polyclonal anti-Galα3Gal antibody staining of mouse fibroblast feeder cells (left) showing absence of staining in hESC colony (right). B) UEA (Ulex Europaeus) lectin staining of stage 3 human embryonic stem cells. FES 30 line.

FIG. 6. A) UEA lectin staining of FES22 human embryonic stem cells (pluripotent, undifferentiated). B) UEA staining of FES30 human embryonic stem cells (pluripotent, undifferentiated).

FIG. 7. A) RCA lectin staining of FES22 human embryonic stem cells (pluripotent, undifferentiated). B) WFA lectin staining of FES30 human embryonic stem cells (pluripotent, undifferentiated).

FIG. 8. A) PWA lectin staining of FES30 human embryonic stem cells (pluripotent, undifferentiated). B) PNA lectin staining of FES30 human embryonic stem cells (pluripotent, undifferentiated).

FIG. 9. A) GF 284 immunostaining of FES30 human embryonic stem cell line Immunostaining is seen in the edges of colonies in cells of early differentiation (10× magnification). Mouse feeder cells do not stain. B) Detail of GF284 as seen in 40× magnification. This antibody is suitable for detecting a subset of hESC lineage.

FIG. 10. A) GF 287 immunostaining of FES30 human embryonic stem cell line. Immunostaining is seen throughout the colonies (10× magnification). Mouse feeder cells do not stain. B) Detail of GF287 as seen in 40× magnification. This antibody is suitable for detecting undifferentiated, pluripotent stem cells.

FIG. 11. A) GF 288 immunostaining of FES30 human embryonic stem cells Immunostaining is seen mostly in the edges of colonies in cells of early differentiation (10× magnification). Mouse feeder cells do not stain. B) Detail of GF288 as seen in 40× magnification. This antibody is suitable for detecting a subset of hESC lineage.

FIG. 12. Immunostaining of CA15-3 in MSC and osteogenically differentiated cells (sialylated carbohydrate epitope in MUC-1,=GF275). Punctate staining is seen in MSC and more cell membrane localized staining pattern in osteogenically differentiated cells (6 weeks of differentiation, confluent culture). The FACS analysis shows the percentace of MSCs expressing GF275 immunostaining. Majority (more than 80-90%) of osteogenically differentiated cells express GF275

FIG. 13. Immunostaining of MSC and osteogenically differentiated cells. Blood group H1(0) antigen, Lewis d (BG4=GF303). No clear staining is seen in MSC whereas osteogenically differentiated cells show clear immunostaining in more than 70-90% of cells.

FIG. 14. H type 2 blood group antigen (=GF302) immunostaining of MSC and osteogenically differentiated MSCs. The immunostaining in MSCs is seen in approx. 20-75% of both cell types.

FIG. 15. SSEA-3 (=GF353) and SSEA-4 (=GF354) immunostaining of MSC and osteogenically differentiated MSCs. SSEA-3 immunostaining decreases when MSC differentiate into osteogenic direction. SSEA-4 (=GF354) immunostaining decreases when MSC differentiate into osteogenic direction.

FIG. 16. Tn (CD175=GF278) immunostaining of MSC and osteogenically differentiated MSCs. Few (5-45%) MSCs express CD175 compared to MSCs differentiated into osteogenic direction.

FIG. 17. sialyl Tn (sCD175=GF277) immunostaining of MSC and osteogenically differentiated MSCs. Few MSCs express sialyl Tn, 5-45%. Osteogenically differentiated cells express more or mainly the epitope.

FIG. 18: Results of FACS analysis of bone BM-MSCs and osteogenic cells derived thereof. FACS results are shown as an average percentage of positive cells in a cell population (n=1-3 individual experiment(s)).

FIG. 19. Portrait of the hESC N-glycome. A. Mass spectrometric profiling of the most abundant 50 neutral N-glycans (A) and 50 sialylated N-glycans (B) of the four hESC lines (blue columns/left), four EB samples (middle columns), and four stage 3 differentiated cell samples (light columns/right). The columns indicate the mean abundance of each glycan signal (% of the total glycan signals). Proposed N-glycan monosaccharide compositions are indicated on the x-axis: S: NeuAc, H: Hex, N: HexNAc, F: dHex, Ac: acetyl. The mass spectrometric glycan profile was rearranged and the glycan signals grouped in the main N-glycan structure classes. Glycan signals in the group ‘Other’ are marked with m/z ratio of their [M+Na]+ (left panel) or [M−H]− ions (right panel). The isolated N-glycan fractions of hESC were structurally analyzed by proton NMR spectroscopy to characterize the major N-glycan core and backbone structures, and specific exoglycosidase digestions with α-mannosidase (Jack beans), α1,2-and α1,3/4-fucosidases (X. manihotis/recombinant), galactosidase (S. pneumoniae), and neuraminidase (A. ureafaciens) to characterize the non-reducing terminal epitopes. Structures proposed for the major N-glycan signals are indicated by schematic drawings in the bar diagram. The major sialylated N-glycan structures are based on the trimannosyl core with or without core fucosylation as demonstrated in the NMR analysis. Galactose linkages or branch specificity of the antennae are not specified in the present data. The Lewis x antigen was detected in the same cells by monoclonal antibody staining (not shown).

FIG. 20. A. Classification rules for human N-glycan biosynthetic groups. The minimal structures of each biosynthetic group (solid lines) form the basis for the classification rules. Variation of the basic structures by additional monosaccharide units (dashed lines) generates complexity to stem cell glycosylation as revealed in the present study. H: hexose, N: N-acetylhexosamine, F: deoxyhexose, S: N-acetylneuraminic acid. B. Diagram showing relative differences in N-glycan classes between hESC and stage 3 differentiated cells (st.3). Although the major N-glycan classes are expressed in both hESC and the differentiated cell types, their relative proportions are changed during hESC differentiation. Complex fucosylation (F≧2) of sialylated N-glycans as well as high-mannose type and complex-type N-glycans were identified as the major hESC-associated N-glycosylation features. In contrast, fucosylation as such (F≧1) was not similarly specific. Hybrid-type or monoantennary, low-mannose type, and terminal N-acetylhexosamine (N>H≧2 or N═H≧5) type N-glycans were associated with differentiated cells. The relative differences were calculated according to Equation 2 from the N-glycan profiles (Supplementary Tables). Schematic examples of glycan structures included in each glycan class are inserted in the diagram. Glycan symbols: ▪, N-acetyl-D-glucosamine; ∘, D-mannose; , D-galactose; ♦, N-acetylneuraminic acid; Δ, L-fucose; □, N-acetyl-D-galactosamine.

FIG. 21. The major N-glycan structures in hESC N-glycome were determined by MALDI-TOF mass spectrometry combined with exoglycosidase digestion and proton NMR spectroscopy. A, High-mannose type N-glycans with five to nine mannose residues dominated the neutral N-glycan fraction. B, In the sialylated N-glycan fraction, the most abundant components were biantennary complex-type N-glycans with either α2,3 or α2,6-sialylated type II N-acetyllactosamine antennae and with or without core α1,6-fucosylation. Monosaccharide symbols: N-acetylhexosamines (N): ▪, N-acetyl-D-glucosamine, GlcNAc; Hexoses (H): ◯, D-mannose, Man; ◯, D-galactose, Gal; , D-glucose, Glc; And deoxyhexoses (F): Δ, L-fucose, Fuc. Sialic acids (S): ♦, N-acetylneuraminic acid, Neu5Ac; and sulphate or phosphate esters (P). Glycosidic linkages are indicated by lines connecting the monosaccharides; lines indicate glycosidic linkages between monosaccharide residues; dashed lines indicate the presence of multiple structures; →Asn indicates site of linkage to glycoprotein.

FIG. 22. Statistical discrimination analysis of the four hESC lines, embryoid bodies derived from FES 29 and FES 30 hESC lines (EB), and stage 3 differentiated cells derived from FES 29 (st.3). The calculation of the glycan score is detailed in the Supplementary data.

FIG. 23. 50 most abundant signals from the neutral N-glycome of human embryonic stem cells.

FIG. 24. Hybrid and complex N-glycans picked from the 50 most abundant signals front the neutral N-glycome of human embryonic stem cells.

FIG. 25. 50 most abundant signals from the acidic N-glycome of human embryonic stem cells.

FIG. 26. (A) Hybrid N-glycans of human embryonic stem cells and changes in their relative abundance during differentiation. (B) Enlargement of the X-axis of (A).

FIG. 27. High mannose N-glycans (Man≧5) of human embryonic stem cells and changes in their relative abundance during differentiation.

FIG. 28. “Low mannose” N-glycans (Man 1-4) of human embryonic stem cells and changes in their relative abundance during differentiation.

FIG. 29. (A) Fucosylated N-glycans of human embryonic stem cells and changes in their relative abundance during differentiation. (B) Enlargement of the X-axis of (A).

FIG. 30. (A) “Complexly fucosylated” (Fuc≧2) N-glycans of human embryonic stem cells and changes in their relative abundance during differentiation. (B) Enlargement of the X-axis of (A).

FIG. 31. Sulfated N-glycans of human embryonic stem cells and changes in their relative abundance during differentiation.

FIG. 32. Large N-glycans (H≧7, N≧6) of human embryonic stem cells and changes in their relative abundance during differentiation.

FIG. 33. The canonical means of the first discriminant analysis for neutral hESC, EB and st3. Root 1 is represented on the x-axis and Root 2 on the y-axis. From the figure we can see that the means are further differentiated on the x-axis and therefore we use Root 1 to determine the function.

FIG. 34. Lectin FACS of hESCs. hESCs were detached with EDTA, washed with FCS-PBS. FES30 cells were double staining with SSEA-3+.

FIG. 35. FACS analysis using various antibodies. The cells were detached with EDTA and washed with buffer containing FCS.

FIG. 36. The N-glycome of human bone marrow MSC:s.

a) MALDI-TOF mass spectrum of the neutral N-glycan fraction from MSC:s.

b) Schematic representation of the relative signal. intensities (% of total signals) of 50 most abundant glycan signals (positive mode) from MSC:s and osteoblasts differentiated from them.

c) MALDI-TOF mass spectrum of the acidic N-glycan fraction from MSC:s.

d) Schematic representation of the relative signal intensities (% of total signals) of 50 most abundant glycan signals (negative mode) from MSC:s and osteoblasts differentiated from them.

The structures shown are based on known biosynthetic routes, NMR-analysis and exoglycosidase experiments. The columns indicate the mean abundance of each glycan signal (% of the total glycan signals). Proposed N-glycan monosaccharide compositions are indicated on the x-axis: S: NeuAc, H: Hex, N: HexNAc, F: dHex, Ac: acetyl. The mass spectrometric glycan profile was rearranged and the glycan signals grouped in the main N-glycan structure classes. The isolated N-glycan fractions of the mesenchymal stem cells were structurally analyzed by proton NMR spectroscopy to characterize the major N-glycan core and backbone structures, and specific exoglycosidase digestions with a-mannosidase (Jack beans), α1,2-and α1,3/4-fucosidases (X. manihotis/recombinant), β1,4-galactosidase (S. pneumoniae), and neuraminidase (A. ureafaciens) to characterize the non-reducing terminal epitopes. Structures proposed for the major N-glycan signals are indicated by schematic drawings in the bar diagram. The major sialylated N-glycan structures are based on the trimannosyl core with or without core fucosylation as demonstrated in the NMR analysis. Galactose linkages or branch specificity of the antennae are not specified in the present data. The Lewis x structure can be detected in the same cells by staining with specific binding reagent.

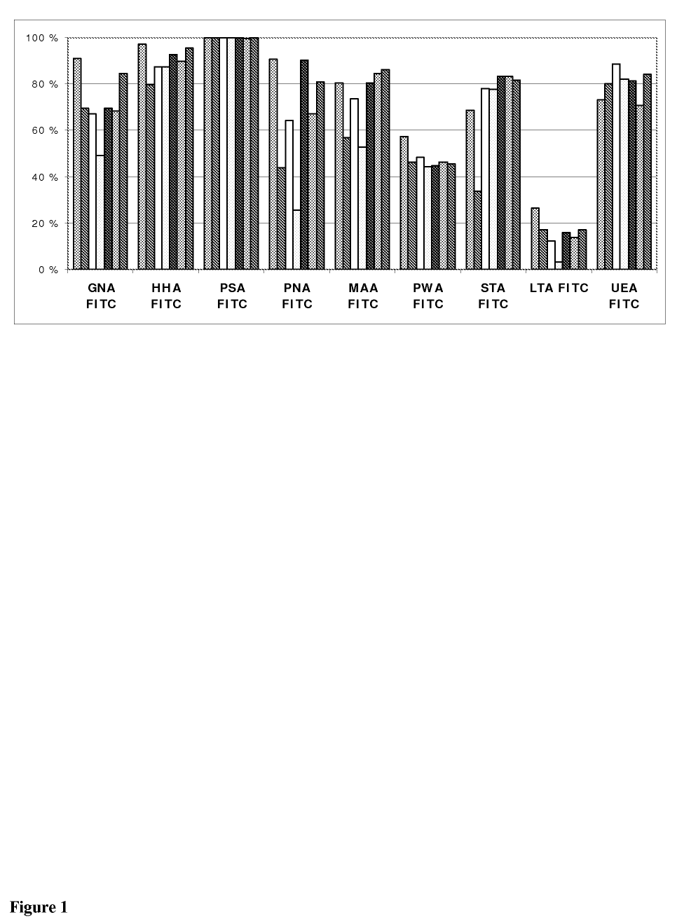

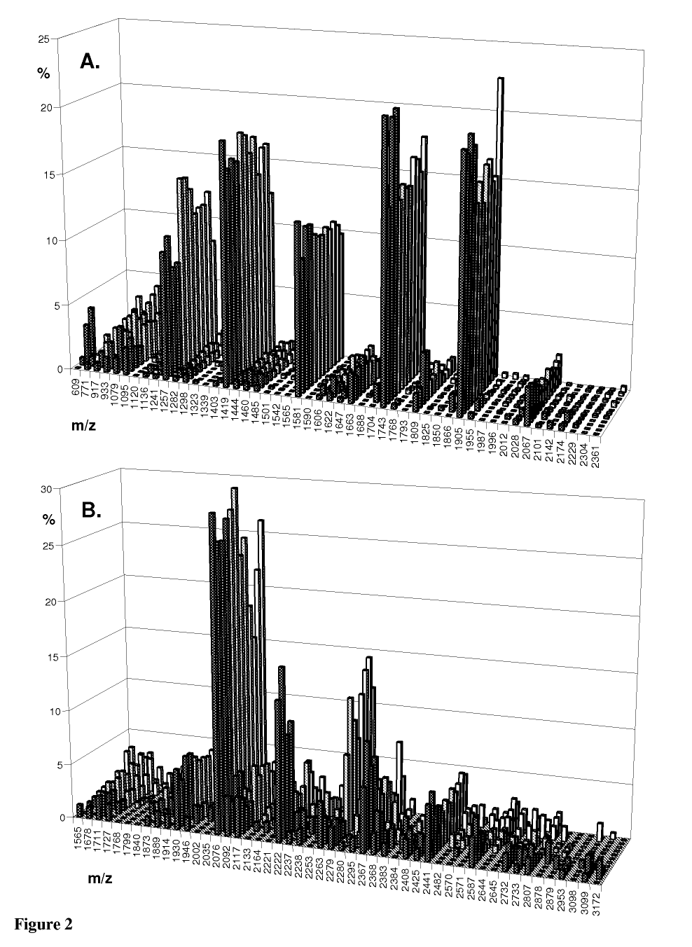

FIG. 37. α3/4-fucosidase treatment of the neutral N-glycan fraction from mesenchymal stem cells. The reaction indicates the presence of structures with Formula Galβ4/3(Fucα3/4)GlcNAc. Lewis x, Galβ4(Fucα3)GlcNAc, structures were revealed by other experiments to be major structures of this type Part of the MALDI-TOF mass spectrum a) before treatment; b) after treatment. Panel c shows the colour code of monosaccharide residues and single letter symbols of monosaccharide residues used in FIG. 1 and FIG. 2.

FIG. 38. Immunofluorescent staining with anti-sialyl Lewis x antibody reveals that the structure Neu5Acα3Galβ4(Fucα3)GlcNAc is a major mesenchymal cell marker associated with stem cell state.

a) bone marrow MSC:s

b) osteoblasts differentiated from bone marrow MSC:s

FIG. 39. Fucosylated acidic N-glycans of bone marrow mesenchymal stem cells (BM MSC) analyzed by MALDI-TOF mass spectrometric profiling. A preferred terminal structure type is sialyl-Lewis x, Neu5Acα3Galβ4(Fucα3)GlcNAc.

FIG. 40. Complex fucosylated neutral (upper panel) and acidic (lower panel) N-glycans of BM MSC analyzed by MALDI-TOF mass spectrometric profiling. The Complex fucosylated (Fuc≧2) N-glycans of human mesenchymal stem cells and changes in their relative abundance during differentiation. The group includes preferred structures Lewis x, Galβ4(Fucα3)GlcNAc, and sialyl-Lewis x, Neu5Acα3 Galβ4(Fucα3)GlcNAc.

FIG. 41. Sulfated N-glycans and phosphorylated N-glycans of BM MSC analyzed by MALDI-TOF mass spectrometric profiling. Sulfated N-glycans of human mesenchymal stem cells change in their relative abundance during differentiation.

FIG. 42. Mass spectrometric profiling analysis of neutral N-glycans. A.) Positive-ion MALDI-TOF mass spectrum of CD133+ neutral N-glycan fraction, wherein major glycan signals arise from [M+Na]⁺ sodium adduct ions. B.) Comparison of processed neutral N-glycan profiles of CD133+ and CD133− cells, wherein relative abundance of each glycan signal is expressed as % of total profile, allowing direct comparison between cell types. Known interfering signals, adduct ion signals, and effect of isotope pattern overlapping present in the original mass spectra have been removed (see Materials and methods). Each glycan signal has been assigned a proposed monosaccharide composition based on the mlz of the detected ion. C.) Rearrangement analysis of the profile data based on biosynthetic classification rules for the amounts of H and N residues in the proposed monosaccharide compositions, as indicated in the figure. Within each proposed biosynthetic class, glycan signals are arranged in the order of relative abundance in CD133+ cells. Relative abundances of the proposed glycan structure groups are indicated as % of total profile. Monosaccharide symbols as in FIG. 1. Abbreviations: F; fucose, H; Hexose and N; N-acetylhexoamine.

FIG. 43. Mass spectrometric profiling analysis of sialylated N-glycans. A.) Negative-ion MALDI-TOF mass spectrum of CD133+ acidic N-glycan fraction, wherein major glycan signals arise from [M−H]⁻ deprotonated ions. Asterisks mark known contaminating polyhexose series that has been removed from B and C. B.) Comparison of sialylated N-glycan profiles of CD133+ and CD133− cells. C.) rearrangement analysis of the profile data, performed similarly as in FIG. 2. Further monosaccharide composition features associated with either CD133+ or CD133− cells (Hex5HexNAc3 and Hex6HexNAc3) are treated as additional glycan signal structural groups and their interpretation is indicated. Monosaccharide symbols as in FIG. 1. Abbreviations: F; fucose, H; Hexose, N; N-acetylhexoamine and S; sialic acid.

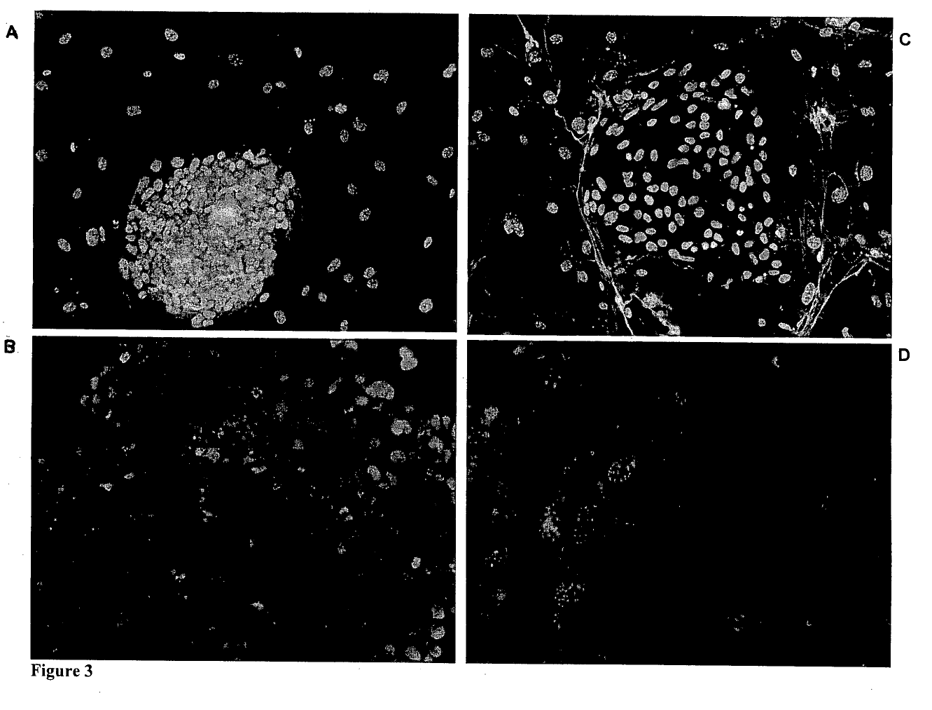

FIG. 44. Exoglycosidase digestion with α2,3-sialidase in sialylated CD133+ and CD133− cell N-glycans. Sialylated N-glycan samples were treated α2,3-sialidase, and mass spectra were recorded before (dashed bars) and after the treatment (solid bars). The data was processed into normalized glycan profiles similarly as in FIGS. 2 and 3. For clarity, only the major sialylated N-glycan signals with H5N4 core composition are presented here. Change in the relative abundances of the glycans is indicated by arrows. The sum of monosialylated (S1) relative to the corresponding disialylated (S2) glycan species was increased in CD133+ cells, whereas in CD133− cells no similar profile change was observed. Abbreviations: F; fucose, H; Hexose, N; N-acetylhexoamine and S; sialic acid.

FIG. 45. Schematic representation of N-linked glycan structures according to their biosynthetic entities. N-linked glycans consist of dinstinct regions of N-glycan core, backbone and terminal epitopes that are synthesized by different glycosyltransferase and glycosidase families. The gene familes encoding these enzymes analyzed in the present study are given in brackets. Monosaccharide symbols and schematic N-glycan structures are as presented in the legend of FIG. 1.

FIG. 46. Schematic representation of favored N-glycan structures in CD133+ cells. Favored N-glycan structures in CD133+ cells are shown in dark background. Overexpressed and underexpressed genes are marked with black arrows upwards and downwards to show the difference in gene expression compared to CD133− cells. A. N-glycan core structures in CD133+ cells are polarized into both high-mannose type N-glycans and biantennary N-glycan structures, correlating with the differential expression of N-glycan processing enzymes. B. α2,3- and α2,6-sialyltransferases compete for the same N-glycan substrates. Overexpression of ST3GAL6 is accompanied with increased α2,3-sialylation in CD133+ cells. Monosaccharide symbols and schematic N-glycan structures are as presented in the legend of FIG. 1.

FIG. 47. Results from CB-HSC FACS analysis.

### SUMMARY OF THE INVENTION

The present invention is directed to analysis of broad glycan mixtures from stem cell samples by specific binder (binding) molecules.

The present invention is specifically directed to glycomes of stem cells according to the invention comprising glycan material with monosaccharide composition for each of glycan mass components according to the Formula I:

R₁Hexβz{R₃}ₙ₁HexNAcXyR₂ (I),

wherein X is nothing or a glycosidically linked disaccharide epitope β4(Fucα6)ₙGN,

wherein n is 0 or 1;

Hex is Gal or Man or GlcA;

HexNAc is GlcNAc or GalNAc;

y is anomeric linkage structure α and/or β or a linkage from a derivatized anomeric carbon,

z is linkage position 3 or 4, with the provision that when z is 4, then HexNAc is GlcNAc and Hex is Man or Hex is Gal or Hex is GlcA, and

when z is 3, then Hex is GlcA or Gal and HexNAc is GlcNAc or GalNAc;

R₁ indicates 1-4 natural type carbohydrate substituents linked to the core structures,

R₂ is reducing end hydroxyl, a chemical reducing end derivative or a natural asparagine linked N-glycoside derivative including asparagines, N-glycoside aminoacids and/or peptides derived from proteins, or a natural serine or threonine linked O-glycoside derivative including asparagines, N-glycoside aminoacids and/or peptides derived from proteins;

R3 is nothing or a branching structure representing GlcNAcβ6 or an oligosaccharide with GlcNAcβ6 at its reducing end linked to GalNAc, when HexNAc is GalNAc, or

R3 is nothing or Fucα4, when Hex is Gal, HexNAc is GlcNAc, and z is 3, or R3 is nothing or Fucα3, when z is 4.

Typical glycomes comprise of subgroups of glycans, including N-glycans, O-glycans, glycolipid glycans, and neutral and acidic subglycomes.

The invention is directed to diagnosis of clinical state of stem cell samples, based on analysis of glycans present in the samples. The invention is especially directed to diagnosing cancer and the clinical state of cancer, preferentially to differentiation between stem cells and cancerous cells and detection of cancerous changes in stem cell lines and preparations.

The invention is further directed to structural analysis of glycan mixtures present in stem cell samples.

### DESCRIPTION OF THE INVENTION

Human Embryonic Type Stem Cells

Under broadest embodiment the present invention is directed to all types of human embryonic type stem cells, meaning fresh and cultured human embryonic type stem cells.

The stem cells according to the invention do not include traditional cancer cell lines, which may differentiate to resemble natural cells, but represent non-natural development, which is typically due to chromosomal alteration or viral transfection. It is realized that the data from embryonal carcinomas (EC) and EC cell lines is not relevant for embryonic stem cells.

The embryonic stem cells include all types of non-malignant embryonic multipotent or totipotent cells capable of differentiating to other cell types. The embryonic stem cells have special capacity stay as stem cells after cell division, the self-reneval capacity. The preferred differentiated derivatives of embryonic stem cells includes embryonic bodies, also referred as stage 2 differentiated embryonic stem cells and stage three differentiated embryonic stem cells. In a preferred embodiment the the stage 3 embryonic stem cells have at least partial characteristics of specific tissue or more preferably characteristics of a specific tissue stem cells.

Under the broadest embodiment for the human stem cells, the present invention describes novel special glycan profiles and novel analytics, reagents and other methods directed to the glycan profiles. The invention shows special differences in cell populations with regard to the novel glycan profiles of human stem cells.

The present invention is further directed to the novel structures and related inventions with regard to the preferred cell populations according to the invention. The present invention is further directed to specific glycan structures, especially terminal epitopes, with regard to specific preferred cell population for which the structures are new.

Embryonic Type Cell Populations

The present invention is specifically directed to methods directed to embryonic type or “embryonic like” cell populations, preferably when the use does not involve commercial or industrial use of human embryos and/or involve destruction of human embryos. The invention is under a specific embodiment directed to use of embryonic cells and embryo derived materials such as embryonic stem cells, whenever or wherever it is legally acceptable. It is realized that the legislation varies between countries and regions. The inventors reserve possibility to disclaim legally restricted types of embryonic stem cells.

The present invention is further directed to use of embryonic-related, discarded or spontaneously damaged material, which would not be viable as human embryo and cannot be considered as a human embryo. In yet another embodiment the present invention is directed to use of accidentally damaged embryonic material, which would not be viable as human embryo and cannot be considered as human embryo. Gene technology and embryonic biopsy based methods producing ES cells from embryos without damging the embryo to produce embryonic or embryonic type stem cells are expected to produce ethically acceptable or more cells.

In a preferred embodiment the invention is directed to embryonic type stem cells, which are produced from other cell types by programming the cells to undifferentiated status corresponding to embryonic stem cells or cells corresponding to the preferred differentiated variants of the ES cells.

The invention is further directed to cell materials equivalent to the cell materials according to the invention. It is further realized that functionally and even biologically similar cells may be obtained by artificial methods including cloning technologies.

N-Glycan Structures and Compositions Associated with Differentiation of Stem Cells

The invention revealed specific glycan monosaccharide compositions and corresponding structures, which associated with
i) non-differentiated human embryonic stem cells, hESCs (stage 1) or
ii) stage 2 (embryoid bodies) and/or
iii) stage 3 differentiated cells differentiated from the hESCs.

It is realized that the structures revealed are useful for the characterization of the cells at different stages of development. The invention is directed to the use of the structures as markers for differentiation of embryonic stem cells. The invention is further directed to the use of the specific glycans as markers enriched or increased at specific level of differentiation for the analysis of the cells at specific differentiation level.

Glycan Structures and Compositions are Associated with Individual Specific Differences Between Stem Cell Lines or Batches.

The invention further revelead that specific glycan types are presented in the embryonic stem cell preparations on a specific differentiation stage in varying manner. It is realized that such individually varying glycans are useful for characterization of individual stem cell lines and batches. The specific structures of a individual cell preparation are useful for comparison and standardization of stem cell lines and cells prepared thereof.

The specific structures of a individual cell preparation are used for characterization of usefulness of specific stem cell line or batch or preparation for stem cell therapy in a patient, who may have antibodies or cell mediated immune defence recognizing the individually varying glycans.

The invention is especially directed to analysis of glycans with large and moderate variations as described in examples.

Recognition of Multiple Structures

The invention revealed multiple glycan structures and corresponding mass spectrometric signals, which are characteristic for the stem cell populations according to the invention. In a preferred embodiment the invention is directed to recognition of specific combinations glycans such as whole glycans and/or corresponding signals, such as mass spectrometric signals and/or specific structural epitopes, preferably non-reducing end terminal glycans structures.

It is realized that certain combination of structures are useful for detection because the change of structures can be correlated with the status of the cell, in a preferred embodiment the differentiation status of the cells is correlated with the glycans. The invention specifically revealed glycans changing during the differentiation of the cells. It was revealed that certain glycan structures are increased and others decreased during differentiation of cells. The invention is directed to use of combinations of structures changing similaliry during differentiation and/or structures changing differently (at least one decreasing and at least one decreasing).

Analysis Methods by Mass Spectrometry or Specific Binding Reagents

The invention is specifically directed to the recognition of the terminal structures by either specific binder reagents and/or by mass spectrometric profiling of the glycan structures.

In a preferred embodiment the invention is directed to the recognition of the structures and/or compositions based on mass spectrometric signals corresponding to the structures.

The preferred binder reagents are directed to characteristic epitopes of the structures such as terminal epitopes and/or characteristic branching epitopes, such as monoantennary structures comprising a Manα-branch or not comprising a Manα-branch.

The preferred binder is an antibody, more preferably a monoclonal antibody.

In a preferred embodiment the invention is directed to a monoclonal antibody specifically recognizing at least one of the terminal epitope structures according to the invention.

Recognition of Preferred Terminal Epitopes

The invention is in a preferred embodiment directed to the analysis of the stem cells by specific antibodies and other binding reagents recognizing preferred structural epitopes according to the invention.

The preferred structural epitopes includes non-reducing end terminal Gal/GalNAcβ3/4-epitope comprising structures and sialyated and/or fucosylated derivatives thereof. The invention is directed to recognition of at at least one N-acetylactos

Non-Reducing End Terminal Gal(NAc)Beta Structures

Terminal Galactose epitopes including
i) terminal N-acetyllactosamines Galβ3GlcNAc and/or Galβ4GlcNAc, and fucosylated branched variants thereof such as Lewis a [Galβ3(Fucα4)GlcNAc] and Lewis x [Galβ4(Fucα3)GlcNAc]
ii) O-glycan core structures including Galβ3GalNAcα in linear core I epitope and/or branched Galβ3(R-GlcNAcβ6)GalNAcα,
iii) Glycolipid structures with terminal Galβ3GalNAcβ-structures

Terminal GalNAc epitopes including
i) terminal di-N-acetyllactosediamine GalNAcβ4GlcNAc (LacdiNAc), and α3fucosylated derivative thereof, LexNAc [GalNAcβ4(Fucα3)GlcNAc]
ii) Glycolipid structures with terminal GalNAcβ3Gal -structures

Sialylated Non-Reducing End Terminal Gal(NAc)Beta Structures

The preferred terminal sialylated Gal(NAc) epitopes including,

The preferred sialic acid is (SA) such Neu5Ac or Neu5Gc.
i) terminal sialyl-N-acetyllactosamines SAα3/6Galβ3GlcNAc and/or SAα3/6Galβ4GlcNAc, and fucosylated branched variants thereof such as sialyl-Lewis a [SAα3Galβ3(Fucα4)GlcNAc] and sialyl- Lewis x [SAα3Galβ4(Fucα3)GlcNAc]
ii) sialylated O-glycan core structures including SAα3Galβ3GalNAcα in linear core I epitope or disialyl-structures SAα3Galβ3(SAα6)GalNAcα, and/or branched SAα3Galβ3(R-GlcNAcβ6)GalNAcα,
iii) Glycolipid structures with terminal SAα3Galβ3GalNAcβ-structures and disialostructures SAα3Galβ3(SAα6)GalNAcβ, disialosyl-Tn).

Terminal sialylated GalNAc epitopes including sialylated GalNAcβ3/4-structures
i) terminal sialyl di-N-acetyllactosediamine SAαGalNAβ4GlcNAc, more preferably SAα6GalNAcβ4GlcNAc

Fucosylated Non-Reducing End Terminal Galbeta Structures

The position 2 of galctose carrying N-acetylgroup in GalNAc can be fucosylated to a preferred strcture group with similarity to the terminal GalNAc structures The preferred terminal fucosylated Gal epitopes includes,
i) terminal fucoslyl-N-acetyllactosamines Fucα2Galβ3GlcNAc and/or Fucα2Galβ4GlcNAc, and fucosylated branched variants thereof such as Lewis b [Fucα2Galβ3(Fucα4)GlcNAc] and Lewis y [Fucα2Galβ4(Fucα3)GlcNAc]
ii) fucosylated O-glycan core structures including Fucα2Galβ3GalNAcα in linear core I epitope and/or branched Fucα2Galβ3(R-GlcNAcβ6)GalNAcα,
iii) Glycolipid structures with terminal Fucα2Galβ3GalNAcβ-structures.

Glycomes—Novel Glycan Mixtures from Stem Cells

The present invention revealed novel glycans of different sizes from stem cells. The stem cells contain glycans ranging from small oligosaccharides to large complex structures. The analysis reveals compositions with substantial amounts of numerous components and structural types. Previously the total glycomes from these rare materials has not been available and nature of the releasable glycan mixtures, the glycomes, of stem cells has been unknown.

The invention revealed that the glycan structures on cell surfaces vary between the various populations of the early human cells, the preferred target cell populations according to the invention. It was revealed that the cell populations contained specifically increased “reporter structures”.

The glycan structures on cell surfaces in general have been known to have numerous biological roles. Thus the knowledge about exact glycan mixtures from cell surfaces is important for knowledge about the status of cells. The invention revealed that multiple conditions affect the cells and cause changes in their glycomes. The present invention revealed novel glycome components and structures from human stem cells. The invention revealed especially specific terminal Glycan epitopes, which can be analyzed by specific binder molecules.

Related data and specification was presented in PCT FI 2006/050336, FCT/FI2006/050483, and FCT/FI2006/050485 included fully as reference.

The present invention revealed novel stem cell specific glycans, with specific monosaccharide compositions and associated with differentiation status of stem cells and/or several types of stem cells and/or the differentiation levels of one stem cell type and/or lineage specific differences between stem cell lines.

N-Glycan Structures and Compositions Associated with Differentiation of Stem Cells

The invention revealed specific glycan monosaccharide compositions and corresponding structures, which associated with
iv) non-differentiated human mesenchymal stem cells, hMSCs or
v) differentiated cells derived from the hMSCs, preferably osteoblast type cells.

It is realized that the structures revealed are useful for the characterization of the cells at different stages of development. The invention is directed to the use of the structures as markers for differentiation of mesenchymal stem cells. The invention is further directed to the use of the specific glycans as markers enriched or increased at specific level of differentiation for the analysis of the cells at specific differentiation level.

The invention is further directed to analysis of the geneneral status of the cells as it is realized that the glycosylation is likely to change, when any condition affecting the cells is changed. The invention is further directed to the analysis of the differentiation status of the cells, when the differentiation is expected to be associated with any of the following conditions: change of cell culture conditions including nutritional conditions, growth factor types or amounts, amount of gasses available, pH of the cell culture medium; protein, lipid, or carbohydrate content of a medium; physical factors affecting the cells including pressure, shaking, temperature, storage in lowered temperature, freezing and/or thawing and conditions associated with it; contact with different cell culture container surfaces, cell culture matrixes including polymers and gels, and contact with other cell types or materials secreted by these.

N-Glycan Structures and Compositions are Associated with Individual Specific Differences Between Stem Cell Lines or Batches.

The invention further revelead that specific glycan types are presented in the mesenchymal stem cell preparations in varying manner. Most of the altering glycan types are associated on a specific differentiation stage. It is realized that such individually varying glycans are useful for characterization of individual stem cell lines and batches. The specific structures of an individual cell preparation are useful for comparison and standardization of stem cell lines and cells prepared thereof. The specific structures of an individual cell preparation are used for characterization of usefulness of specific stem cell line or batch or preparation for stem cell therapy in a patient, who may have antibodies or cell mediated immune defence recognizing the individually varying glycans.

The invention is especially directed to analysis of glycans with large and moderate individual variations in glycomes.

Analysis Methods by Mass Spectrometry or Specific Binding Reagents

The invention is specifically directed to the recognition of the terminal structures by either specific binder reagents and/or by mass spectrometric profiling of the glycan structures. The preferred methods includes recognition of N-glycans, preferably a biantennary, or triantennary N-glycan is recognized by mass spectrometry and/or binder reagent. Preferably the N-glycan is recognized by mass spectrometry and the binder reagent is preferably a glycosidase enzyme.

In a preferred embodiment the invention is directed to the recognition of the structures and/or compositions based on mass spectrometric signals corresponding to the structures.

The preferred binder reagents are directed to characteristic epitopes of the structures such as terminal epitopes and/or characteristic branching epitopes, such as fucosylated structures including sialyl-Lewis x and Lewis x structures and sulfated structures. The invention is directed to specific antibodies recognizing the preferred terminal epitopes, the invention is further directed to other binders with the same or similar specificity, preferably with the same specificity as the preferred antibodies.

The preferred binder is a protein or peptide binding to carbohydrate, preferably a lectin, enzyme or antibody or a carbohydrate binding fragment thereof. In a preferred embodiment the binder is an antibody, more preferably a monoclonal antibody.

In a preferred embodiment the invention is directed to a monoclonal antibody specifically recognizing at least one of the terminal epitope structures according to the invention.

The mass spectrometric profiling of released N-glycans revealed characteristic changes in the glycan profiles. The mass spectrometric method allows detection of multiple glycans and glycan type simultaneously. The mass profiles reveal individual glycan structures specific for specific cell types. The invention is especially directed to the recongnition of the glycan structures from very low amounts of material such as from 1000 to 5 000 000 cells, preferably between 10 0000 and million cells and most preferably between 100 000 and million cells.

Use of the Binding Reagents for the Analysis of Cellular Interactions

It is realized that the carbohydrate structures on cell surfaces are associated with contacts with other cells and surrounding cellular matrix. Therefore the identified cell surface glycan structures and especially binding reagents specifically recognizing these are useful for the analysis of the cells. The preferred analysis method includes the step of contacting the cell with a binding reagent and evaluating the effect of the binding reagent to the cell. In a preferred embodiment the cells are contacted with the binder under cell culture condition. In a preferred embodiment the binder is represented in multivalent or more preferably polyvalent form or in another preferred embodiment in surface attached form. The effect may be change in the growth characteristics or cellular signalling in the cells.

Preferred Terminal Structural Epitopes

The invention is directed to the use of type II N-acetyllactosamine type structures including closely homologous structures, such as LacdiNAc (GalNAcβ4GlcNAc) and lactosyl (Galβ4Glc) structures for the evaluation of mesenchymal stem cells and derivatives thereof.

The invention is preferably directed to evaluating the status of a human mesenchymal stem cell preparation comprising the step of detecting the presence of a glycan structure or a group of glycan structures in said preparation, wherein said glycan structure or a group of glycan structures is according to Formula LN1

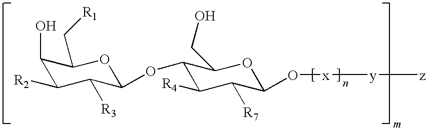

wherein

X is linkage position

R₁, and R₂, are OH or glycosidically linked monosaccharide residue Sialic acid,

preferably Neu5Acα or Neu5Gcα, most preferably Neu5Acα or sulfate ester groups or

R₃, is OH or glycosidically linked monosaccharide residue Fucα (L-fucose) or N-acetyl (N-acetamido, NCOCH₃);

R₄, is OH or glycosidically linked monosaccharide residue Fucα (L-fucose),

R7 is N-acetyl or OH

X is natural oligosaccharide backbone structure from the cells, preferably N-glycan,

O-glycan or glycolipid structure; or X is nothing, when n is 0,

Y is linker group preferably oxygen for O-glycans and O-linked terminal oligosaccharides and glycolipids and N for N-glycans or nothing when n is 0;

Z is the carrier structure, preferably natural carrier produced by the cells, such as protein or lipid, which is preferably a ceramide or branched glycan core structure on the carrier or H;

n is an integer 0 or 1, and m is an integer from 1 to 1000, preferably 1 to 100, and most preferably 1 to 10 (the number of the glycans on the carrier) and with the provision that when R7 is N-acetyl then 6 position hydroxyl of the GlcNAc residue may be substituted by sulfate ester.

The invention is further directed to the structures according to the Formula LN2

[Mα]ₘGalβ1-4[Nα]ₙGlcNAβxMan

wherein

wherein m, n and p are integers 0, or 1, independently,

x is linkage position selected from the group 2, 4 or 6

M and N are substituents or monosaccharide residues being
I. independently nothing (free hydroxyl groups at the positions) and/or
II. SA which is Sialic acid linked to 3-position or 6-position of Gal and/or
III. Fuc (L-fucose) residue linked to 2-position of Gal and/or 3 position of GlcNAc, and/or
IV. Sulfate ester on position 3 or 6-of Gal and/or position 6 of GlcNAc,

with the provision that when sialic acid is linked to position 6, then preferably n is 0,

The invention is further directed to the structures according to the Formula LN3

[Mα]ₘGalβ1-4 [Nα]ₙGlcNAcβ2Man,

wherein the variables are as described for Formula LN2 and the structure is preferably linked to N-glycan core.

The specifically preferred structure is fucosylated structures according to the Formula LN4

[Mα]ₘGalβ1-4(Fucα3)ₙGlcNAcβ2Man,

wherein M is α3-linked sialic acid (SAα3) preferably Neu5Acα3 or Fucα2.

The preferred LN4 structure is a N-glycan linked structure being:

Lewis x structure, Galβ1-4(Fucα3)GlcNAcβ2Man, or

sialyl-Lewis x structure Neu5Acα3Galβ1-4(Fucα3)GlcNAcβ2Man.

Another preferred structure group includes a fucosylated structure according to the Formula LN4a

[SAα3]ₘGalβ1-4GlcNAcβ2Man,

wherein SA is sialic acid preferably Neu5Ac and

and the structure is a N-glycan linked type II LacNAc structure, Galβ1-4GlcNAcβ2Man, or

sialyl-type II LacNAc structure Neu5Acα3Galβ1-4GlcNAcβ2Man

The invention is further directed to structures according to the Formula LN3

[SE3/6]ₘGalβ1-4[SE6]ₙGlcNAcβ2Man,

wherein SE is sulfate ester and 3/6 indicates either 3 or 6 and the structure comprises at least one sulfate residue.

The invention is further directed to structures according LN2 are selected from the group consisting of Galβ4GlcNAcβ2Man, Galβ4(Fucα3)GlcNAβ2Man, Fucα2Galβ4GlcNAβ2Man, SAα6Galβ4GlcNAcβ2Man, and SAα3Galβ4GlcNAcβ2Man.

The isomeric fucosylated and sialylated structures, H type II Fucα2Galβ4GlcNAcβ2Man, and SAα6Galβ4GlcNAcβ2Man are preferred as controls for the other structures. The structures are also associated with certain differentiated cell populations.

In a preferred embodiment the structure is H type II structure associated with differentiated cells.

The invention is directed to the method further involving the recognition of a triantennary terminal structure according to the Formula LN4b

[SAα3]ₘGalβ1-4GlcNAcβ4Man,

wherein SA is sialic acid preferably Neu5Ac and

and the structure is a N-glycan linked

type II LacNAc structure, Galβ1-4GlcNAcβ4Man, or

sialyl- type II LacNAc structure Neu5Acα3Galβ1-4GlcNAcβ4Man.

Analysis of N-Glycans of Mesenchymal Stem Cells and Differentiated Variants Thereof

MALDI-TOF mass spectrometric analysis of mesenchymal cell N-glycans is shown in Figures. In panel a) MALDI-TOF mass spectrum of the neutral N-glycan fraction from MSC:s and in panel b) Schematic representation of the relative signal intensities (% of total signals) of 50 most abundant glycan signals (positive mode) from MSC:s and osteoblasts differentiated from them.

The panel c) of Figures shows MALDI-TOF mass spectrum of the acidic N-glycan fraction from MSC:s. and panel d) Schematic representation of the relative signal intensities (% of total signals) of 50 most abundant glycan signals (negative mode) from MSC:s and osteoblasts differentiated from them. The comparision of the relative intensities in panel b) and d) allowed the determination of structures specific for non-differentiated cells and for differentiated cells.

Figures further indicates colour symbol coded structures of the N-glycans. The symbols are used essentially similarily to those used by the Consortium for Functional Glycomics.

Briefly, in Tables the reducing end of the N-glycans is on the left, β1-4 linkages (Manβ4,GlcNAcβ4,Galβ4) and GlcNAcβ2 are indicated by horizontal line −, 1-6 linkages (Manα6, NeuAc/sialic acidα6, GlcNAcβ6) are indicated by line upwards / , except Fucα6 above above reducing end GlcNAc, 1-3 linkages (Manα3,Fucα3,Neu5Ac/Neu5Gc/sialic acidα3), are indicated by \, Fucα2 is indicated by vertical line below Galβ, or in the cases where H— structures and GlcNAc fucosylation are alternative structures in the same epitope, line is drawn to both residues. SP represent a sulfate or phosphoryl ester linked to a LacNAc unit, part of the SP symbols are represented as mirror images. The Tabless include representative structures and it is realized that isomeric structures exist, for example when N-glycans carry different terminal epitopes the actual branch location of sialyl, fucosyl or sulfate moieties with regard to two or more N-acetyllactosamines is not definitely indicated, but includes isomeric variants(s). Formulas written for preferred monosaccharide compositions can be used for verification of the structures written with symbols. The same structures have been turned 90 degrees counterclockwise in Figures, the reducing end points downwards, the linkages of similar or same oligosaccharides are represented in Tabless. The glycan structures comprising multiple isomeric structures are indicated by line and separated monosaccharide or disaccharide (LacNAc) elements, the sialic acid residues (Neu5Ac and Neu5Gc) are linked preferably to terminal Gal residues, fucose to Gal or GlcNAc and LacNAc to Gal (another LacNAc unit) as described in the invention.

The structures shown are based on known biosynthetic routes, NMR-analysis and exoglycosidase experiments. The columns indicate the mean abundance of each glycan signal (% of the total glycan signals). Proposed N-glycan monosaccharide compositions are indicated on the x-axis: S: NeuAc, H: Hex, N: HexNAc, F: dHex, Ac: acetyl, SP sulfate of phosphate. The mass spectrometric glycan profile was rearranged and the glycan signals grouped in the main N-glycan structure classes. Glycan signals in the group ‘Other’ are marked with m/z ratio of their [M+Na]+ (left panel) or [M−H]− ions (right panel) and monosaccharide compositions. The isolated N-glycan fractions of the mesenchymal stem cells were structurally analyzed by proton NMR spectroscopy to characterize the major N-glycan core and backbone structures, and specific exoglycosidase digestions with α-mannosidase (Jack beans), α1,2-and α1,3/4-fucosidases (X. manihotis/recombinant), β1,4-galactosidase (S. pneumoniae), and neuraminidase (A. ureafaciens) to characterize the non-reducing terminal epitopes. Structures proposed for the major N-glycan signals are indicated by schematic drawings in the bar diagram. The major sialylated N-glycan structures are based on the trimannosyl core with or without core fucosylation as demonstrated in the NMR analysis. The Lewis x structure can be detected in the same cells by staining with a specific binding reagent.

Preferred Terminal Non-Fucosylated Structures

Type 2 N-acetyllactosamine Structures

The preferred complex type epitopes on N-glycans includes type 2 N-acetyllactosamine structure epitopes of biantennary N-glycans Galβ4GlcNAcβ2, Galβ4GlcNAcβ2Man, Galβ4GlcNAcβ2Manα, Galβ4GlcNAcβ2Manα3, Galβ4GlcNAcβ2Manα6 and Galβ4GlcNAcβ2Manα3/6. Galactosidase analysis revealed that the structures are present on both arms of biantennary N-glycans.

Sialyl-Type 2 N-acetyllactosamine Structures

The preferred complex type epitopes on N-glycans include sialyl- type 2 N-acetyllactosamine structural epitopes of biantennary N-glycans Neu5Acα3Galβ4GlcNAcβ2, Neu5Acα3Galβ4GlcNAcβ2Man, Neu5Acα3Galβ4GlcNAcβ32Manα, Neu5Acα3Galβ4GlcNAcβ2Manα3, Neu5Acα3Galβ4GlcNAcβ2Manα6 and Neu5Acα3Galβ4GlcNAcβ2Manα3/6.

Preferred Fucosylated Structure Types

The invention revealed fucosylated glycan structures in N-glycomes of the mesenchymal cells. The preferred structure types includes terminal structures comprising α3/4 linked fucoses revealed by specific fucosidase digestion. These includes especially type II structures Lewis x and sialyl Lewis x. The major linkage type of galactose as β4 and terminal α3-sialylation were revealed by specific glycosidase digestions. The terminal structure types were analyzed from various glycan types from the mesenchymal cells of the invention. The invention is directed to specific antibodies known to recognize Lewis x (e.g. Dubet et al abstract Glycobiology Society Meeting 2006, Los Angeles) and sialyl-Lewis x on specific preferred N-glycan structures according to the invention.

The invention is further directed to the use and testing/selection of antibodies specific for the structures on O-glycans or glycolipids for the analysis of mesenchymal type stem cells. The invention is further directed to lower specificity antibodies and/or other binding reagents recognizing the terminal epitopes on all or at least two glycan classes selected from the group N-glycans, O-glycans and glycolipids. The invention is further directed to the use of the antibodies and/or other corresponding binder reagents for methods including the step of binding of the reagent to the cells including cell sorting, cell manipulation or cell culture.

Fucosylated Structures on Complex Type N-Glycans

The invention is especially directed to the fucosylated structures carried on complex type N-glycans (referred also as Complex fucosylated structures). The terminal epitopes in the complex fucosylated structures are mainly linked to Manα-residues of N-glycan core structures, the linkage is β2-linkage in biantennary structures, and preferably in triantennary structures also β4-linkage, and in tetra-antennary and more branched structures further include β6-linkage. The invention further revealed unusually large N-glycans, which carry polylactosamine structures where lactosamines are linked to each other with β3 and/or β6 linkages forming epitopes like Galβ4GlcNAcβ3/6Galβ4GlcNAcβ2, which can be further sialylated and/or fucosylated.

The invention revealed especially biantennary but also triantennary and larger N-glycans and the invention is in a preferred embodiment especially directed to these N-glycans carrying fucose residues.

The preferred complex type epitopes on N-glycans includes Lewis x structure epitopes of biantennary N-glycans Galβ4(Fucα3)GlcNAcβ2, Galβ4(Fucα3)GlcNAcβ2Man, Galβ4(Fucα3)GlcNAcβ2Manα, Galβ4(Fucα3)GlcNAcβ2Manα3, Galβ4(Fucα3)GlcNAcβ2Manα6 and Galβ4(Fucα3)GlcNAcβ2Manα3/6. Fucosidase analysis revealed that Lewis x structures are present on both arms of biantennary N-glycans.

The preferred complex type epitopes on N-glycans include sialyl-Lewis x structure epitopes of biantennary N-glycans Neu5Acα3Galβ4(Fucα3)GlcNAcβ2, Neu5Acα3Galβ4(Fucα3)GlcNAcβ2Man, Neu5Acα3Galβ4(Fucα3)GlcNAcβ2Manα, Neu5Acα3Galβ4(Fucα3)GlcNAcβ2Manα3, Neu5Acα3Galβ4(Fucα3)GlcNAcβ2Manα6 and Neu5Acα3Galβ4(Fucα3)GlcNAcβ2Manα3/6.

Figures shows α3/4-fucosidase treatment of the neutral N-glycan fraction from mesenchymal stem cells. The reaction indicates the presence of structures with Formula Galβ4/3(Fucα3/4)GlcNAc. Lewis x, Galβ4(Fucα3)GlcNAc, structures were revealed by other experiments to be major structures of this type Part of the MALDI-TOF mass spectrum a) before treatment; b) after treatment. Panel c shows the colour code of monosaccharide residues and single letter symbols of monosaccharide residues used in Figs.

Figures reveals immunofluorescent staining with anti-sialyl Lewis x antibody reveals that the structure Neu5Acα3Galβ4(Fucα3)GlcNAc is a major mesenchymal cell marker associated with stem cell state. In panel a) bone marrow MSC:s are stained effectively and panel b) shows no or very little binding on the osteoblasts differentiated from bone marrow MSC:s by the specific anti-sialyl-Lewis x antibody.

Figures shows fucosylated acidic N-glycans of bone marrow mesenchymal stem cells (BM MSC) analyzed by MALDI-TOF mass spectrometric profiling. A preferred terminal structure type is sialyl-Lewis x, Neu5Acα3Gal≢24(Fucα3)GlcNAc.

Figures. shows selected complex fucosylated neutral (upper panel) and acidic (lower panel) N-glycans of BM MSC analyzed by MALDI-TOF mass spectrometric profiling. The Complex fucosylated (Fuc≧2) N-glycans of human mesenchymal stem cells and changes in their relative abundance during differentiation. The group includes preferred structures Lewis x, Galβ4(Fucα3)GlcNAc, and sialyl-Lewis x, Neu5Acα3Galβ4(Fucα3)GlcNAc. The level of fucosylation on complex type N-glycan increases during differentiation and the invention is in a preferred embodiment directed to use of the amount of fucosylated structures on N-glycans for characterization of the mesenchymal cells

Sulfated N-acetyllactosamine Structures

The invention further revealed that sulfation on complex type N-glcyans is very characteristic to differentiated osteobalst type cells as shown in Figures. Sulfated N-glycans and phosphorylated N-glycans of BM MSC analyzed by MALDI-TOF mass spectrometric profiling. Sulfated N-glycans of human mesenchymal stem cells change in their relative abundance during differentiation.

The invention is especially directed to terminal sulfated N-acetyllactosamine (LacNAc) structures comprising sulfate on 3- and/or 6-position Gal and/or 6 position of GlcNAc. The LacNAc is preferably type 2 LacNAc Galβ4GlcNAc, and even more preferably a N-glycan linked type II N-acetyllactosamine

Combination of Terminal N-Glycan Structures and Complete N-Glycans

It is realized that the terminal type 2 N-acetyllactosamines are linked to N-glycan core structures and can be recognized by high specificity reagents or by mass spectrometry or combinations thereof as part of larger N-glycan structures. The mass spectrometric analysis is also directed to recognition of specific terminal structures based on mass spectrometric signals and/or corresponding monosaccharide compositions when the connection of the structures and the signals or compositions is established as in present invention for the mesenchymal cells.

Methods and reagents and combination thereof recognizing terminal epitopes of N-glycans are also in a preferred embodiment used for recognizing specific N-glycan structures. It is realized that methods directed to the complete N-glycan structures effectively characterize the stem cells.

Structures Associated with Nondifferentiated Human Mesenchymal Stem Cells

The Tables show specific structure groups with specific monosaccharide compositions associated with the differentiation status of human mesenchymal stem cells.

For the preferred assignment of the structures corresponding to preferred monosaccharide composition of preferred altering or variable glycans see Tables. The structures correspond to the mass numbers and monosaccharide compositions of Tables, and glycosidase Table and monosaccharide; and compositions and structures described for glycans in Figures.

Analysis of Individual Specific Variation in Glycan Signal

Variation between glycan signals in the 5 measured MSC lines was measured as proportion of standard deviation to the average glycan signal. Most variation was detected (Tables):
a) in the neutral fraction in multifucosylated glycans, in glycans with terminal N-acetylhexosamine, and in glycans with terminal hexose;
b) in the acidic fraction in multifucosylated glycans, in multisialylated glycans, in glycans with terminal N-acetylhexosamine, and in glycans with sulfate esters.

In conclusion, there is most inter-cell line variation in N-glycan fucosylation, sialylation, sulphation, and glycan backbone formation with terminal N-acetylhexosamine

The Structures Present in Higher Amount in hMSCs than in Corresponding Differentiated Cells

The invention revealed novel structures present in higher amounts in hMSCs than in corresponding differentiated cells.

The preferred hMSC enriched glycan groups are represented by groups hMSC 1 to hMSC 8, corresponding to several types of N-glycans. The glycans are preferred in the order from hMSC-i to hMSC-ix, based on the relative specificity for the non-differentiated hMSCs, the differences in expression are shown in Tables. The glycans are grouped based on similar composition and similar structures present to group comprising Complex type N-glycans, or High-Mannose type N-glycans and other preferred glycan groups.

Complex Type Glycans

hMSC 1, Disialylated Biantennary-Size Complex-Type N-Glycans

Specific expression in hMSCs was revealed for a specific group of biantennary complex type N-glycan structures. This group includes disialylated glycans including S2H5N4, S2H5N4F1, and S2H5N4F2.

Preferred Structural Subgroups of the Biantennary Complex Type Glycans Include NeuAc Comprising Glycans, and Fucosylated Glycans.

NeuAc Comprising Glycans

The sialylated glycans include NeuAc comprising glycans that shares the composition:

S₂H₅N₄F_{q}

Wherein H is preferably Gal or Man and N is GlcNAc, S is Neu5Ac and F is Fuc,

q is an integer from 0 to 3.

The group comprises disialylated glycans with all levels of fucosylation. The preferred subgroups of this category include low fucosylation level glycans comprising no or one fucose residue (low fucosylation) and glycans with two or three fucose residues.

Preferred Biantennary Structures with Low Fucosylation

The preferred biantennary structures according to the invention include structures according to the Formula:

[NeuAcα]₀₋₁GalβGNβ2Manα3([NeuAcα]₀₋₁GalβGNβ2Manβ4GNβ4(Fucα6)₀₋₁GN,

The GalβGlcNAc structures are preferably Galβ4GlcNAc-structures (type II N-acetyllactosamine antennae). The presence of type 2 structures was revealed by specific β4-linkage cleaving galactosidase (D. pneumoniae).

In a preferred embodiment the sialic acid is NeuAcα3- and the glycan comprises the NeuAc linked to Manα3-arm or Manα6-arm of the molecule. The assignment is based on the presence of α3-linked sialic acid revealed by specific sialidase digestion. NeuAcα3 GalβGNβ2Manα3/6([NeuAcα]₀₋₁GalβGNβ2Manα6/3)Manβ4GNβ4(Fucα6)₀₋₁GN, more preferably type II structures: NeuAcα3 Galβ4GNβ2Manα3/6([NeuAcα]₀₋₁Galβ4GNβ2Manα6/3)Manβ4GNβ4(Fucα6)₀₋₁GN.

The invention thus revealed preferred terminal epitopes, NeuAcα3Galβ3GN, NeuAcα3GalβGNβ2Man, NeuAcα3Galβ3GNβ2Manα3/6, to be recognized by specific binder molecules. It is realized that higher specificity preferred for application in context of similar structures can be obtained by using a binder that recognizes larger epitopes and thus differentiating e.g. between N-glycans and other glycan types in the context of the terminal epitopes.

Preferred Difucosylated and Sialylated Structures

Preferred difucosylated sialylated structures include structures, wherein the one fucose is in the core of the N-glycan and

a) one fucose on one arm of the molecule, and sialic acid is on the other arm (antenna of the molecule and the fucose is in Lewis x or H-structure:

Galβ4(Fucα3)GNβ2Manα3/6(NeuNAcαGalβGNβ(2Manα6/3)Manβ4GNβ4(Fucα6)GN,

and/or

Fucα2GalβGNβ2Manα3/6(NeuNAcαGalβGNβ2Manα6/3)Manβ4GNβ4(Fucα6)GN, and when the sialic acid is α3-linked preferred antennary structures contain preferably the sialyl-lactosamine on α3-linked or α6-linked arm of the molecule according to formula:

Galβ4(Fucα3)GNβ2Manα6(NeuNAcα3Galβ4GNβ2Manα3)Manβ4GNβ4(Fucα6)GN,

and/or

Fucα2GalβGNβ2Manα6(NeuNAcα3Galβ4GNβ2Manα3)Manβ4GNβ4(Fucα6)GN.

and/or

Galβ4(Fucα3)GNβ2Manα3(NeuNAcα3Galβ4GNβ2Manα6)Manβ4GNβ4(Fucα6GN,

and/or

Fucα2GalβGNβ2Manα3(NeuNAcα3Galβ4GNβ2Manα6)Manβ4GNβ4(Fucα6)GN.

It is realized that the structures, wherein the sialic acid and fucose are on different arms of the molecules can be recognized as characteristic specific epitopes. b) Fucose and NeuAc are on the same arm in the structure: NeuNAcα3Galβ3/4(Fucα4/3)GNβ2Manα3/6(GalβGNβ2Manα6/3)Manβ4GNβ4(Fucα6)GN, more preferably the structure is a N-glycan sialyl-Lewis x structure: NeuNAcα3Galβ4(Fucα3)GNβ2Manα3/6(GalβGNβ2Manα6/3)Manβ4GNβ4(Fucα6)GN

Preferred Sialylated Trifucosylated Structures

Preferred sialylated trifucosylated structures include glycans comprising core fucose and the terminal sialyl-Lewis x or sialyl-Lewis a, preferably sialyl-Lewis x due to the relatively high abundance presence of type 2 lactosamines, or Lewis y on either arm of the biantennary N-glycan according to the formulae: NeuNAcα3Galβ4(Fucα3)GNβ2Manα3/6([Fucα]GalβGNβ2Manα6/3)Manβ4GNβ4(Fucα6)GN, and/or Fucα2Galβ4(Fucα3)GNβ2Manα3/6(NeuNAcα3/6Galβ3GNβ2Manα6/3)Manβ4GNβ4(Fucα6)GN. NeuNAc is preferably α3-linked on the same arm as fucose due to known biosynthetic preference and sialidase analysis. Preferably the structure comprises NeuNAcα3.

hMSC 5, Disialylated Hybrid-Type, Monoantennary, and Other Glycans

including S2H5N3F2P1 , S2H5N3F1, S2H5N3, S2H6N3F1P1, S2H3N3F1, S2H3N3, S2H4N3, and S2H4N3F1, which correspond to unusual amount of sialic acid on regular core structures described for other glycan groups.

further including very unusual glycan compositions also corresponding to characteristic mass spectrometric signals S2H4N2F1, S2H3N2F1, S2H2N2, and S2H1N3F1

The preferred glycans include complex fucosylated glycans that shares the composition:

S₂HₚN₃F_{q}Pₛ

Wherein H is preferably Gal or Man and N is GlcNAc, S is Neu5Ac, F is Fuc, P is sulfate residue, p is an integer from 1 to 6, r is an integer from 2 to 3, q is an integer from 0 to 2; and s is an integer 0 or 1.

The unusual sialic acid structures include numerous possible variants known in the nature.

hMSC 6, Large Monosialylated Complex-Type N-Glycans

including S1H6N5, S1H6N5F1, S1H6N5F2, S1H6N5F3, S1H6N5F4, S1H6N6F1, S1H7N6F1, S1H7N6F2, S1H7N6F3, S1H7N6F4, S1H7N6F5, S1H8N7, S1H8N7F1, S1H8N7F3, and S1H11N10

The sialylated glycans include NeuAc comprising glycans that shares the composition:

S₁HₚNᵣF_{q}

Wherein H is preferably Gal or Man and N is GlcNAc, S is Neu5Ac, F is Fuc, P is sulfate residue, p is an integer from 6 to 11, preferably 6-8 or 11, r is an integer from 5 to 10, preferably 5-7 or 10 and q is an integer from 0 to 4.

An unusual feature in this group of glycans is presence of only single sialic acid resuidue (NeuNAc/Neu5Ac) in glycans comprising multiple N-acetyllactosamine units. The monosialylation indicates branch specific sialylation of multiantennary structures and presence of repative N-acetyllactosamines (LacNAcs providing only limited amount of sialylation sites).

This group includes N-glycans comprising three LacNAc units with core composition H6N5, four LacNAc units with core composition H7N6, five LacNAc units with core composition H8N7, and eight LacNAc units with core composition H11N10. The glycans of this group includes multiantennary N-glycans and poly-N-cetyllactosamine comprising glycans. The presence of eight N-acetyllactosamien units indicates poly-N-acetyllactosamine structures.

The preferred structures in this group comprising S1H6N5F1-4 include tri-LacNac molecules triantennary N-glycans and elongated biantennary N-glycans. In a preferred embodiment the group includes

a) triantennary N-glycan comprising β1,4-linked N-acetyllactosamine branch, preferably linked to Manα6-arm of the N-glycan (mgat4 product N-glycan) Gβ4GNβ2Mα3(Gβ4GNβ2{Gβ4GNβ4}Mα6)Mβ4GNβ4(Fα6)GN, wherein G is Gal, Gn is GlcNAc, M is Man, and F is Fuc and ( ) and { } indicated branches in the structure, and one of the LacNAc units comprises terminal Neu5Acα3-unit linked to Gal and each may LacNAc unit may comprise Fucα3 residue linked to GlcNAc or Fucα2 residue linked to Gal, which is not sialylated, so that the structure may comprise 1-3 fucose residues. and/or

b) poly-N-acetyllactosamine elongated biantennary complex-type N-glycans, wherein a LacNAc unit is linked to terminal Gal of a regular binatennary structure. [Gβ4GNβ3].ₙ₁Gβ4GNβ2Mα3([Gβ4GNβ3]ₙ₂Gβ4GNβ2Mα6)Mβ4GNβ4(Fα6)GN, wherein G is Gal, Gn is GlcNAc, M is Man, and F is Fuc and ( ) indicates a branch in the structure and [ ] indicates elongating LacNAc unit either present or absent, n1 and n2 are integers being either 0 or 1 independently and and either of the non-reducing end terminal LacNAc units comprises terminal Neu5Acα3-unit linked to Gal and each LacNAc unit may comprise Fucα3 residue linked to GlcNAc units or Fucα2 residue linked to Gal, which is not sialylated, so that the structure may comprise 1-3 fucose residues.

hMSC 7, Monosialylated Hybrid-Type and Monoantennary N-Glycans

including monoantennary glycans S1H3N3, S1H4N3, G1H4N3, S1H4N3F1, S1H4N3F3, and S1H4N3F1P1;

and hybrid-type glycans S1H5N3, G1H5N3, S1H5N3F1, S1H6N3, and S1H7N3

The preferred glycans include hybrid type and monoantennary glycans that shares the composition:

S₁HₚN₃F_{q}Pₛ

Wherein H is preferably Gal or Man and N is GlcNAc, S is Neu5Ac or Neu5Gc, preferably Neu5Ac, F is Fuc, P is sulfate residue (SP in Tables), p is an integer from 3 to 7, q is an integer from 0, 1 or 3; and s is an integer 0 or 1.

The invention revealed characteristic monosialyalted structures comprising only one LacNAc, preferably type II LacNAc unit. Based on biosynthetic consideration the sialyl-lacNAc unit is preferably linked to Manα3-structure in the N-glycan core. Thus this data reveals novel preferred type II sialyl N-acetyllactosamine structure epitopes SAα3/6Galβ4GlcNAcβ2Manα3, more preferably SAα3Galβ4GlcNAcβ2Manα3, wherein SA is Neu5Ac or Neu5Gc, more preferably Neu5Ac.

The preferred core structure for H3-7N3(F) glycans is: Galβ4GlcNAcβ2Manα3({Manα}ₚManα6)Manβ4GlcNAcβ4(Fucα6)_{q}GlcNAc, Wherein p is anteger from 0 to 3 indicating presence of a3, and/or a6 and/or a2-linked Man residues as present in monoantennary (p is 0)/hybrid type (p is 1-3) N-glycans, q is an integer 0 or 1, preferably additional fucose is Fucα2 linked to Gal, and/or Fucα3 linked to GlcNAc; and sulfate is linked to Gal or GlcNAc and sialic acid to Gal on the LacNAc units as decribed by the invention more preferentially with type II N-acetyllactosamine antennae

hMSC 8, Complex-Fucosylated Sialylated Glycans

Including S1H7N6F3, S2H7N6F3, S3H7N6F3, S1H7N6F4, S2H7N6F4, S3H7N6F4, S1H7N6F5, S1H6N5F2, S1H6N5F3, S1H6N5F4, S1H5N4F2, S2H5N4F2, S1H4N3F3, S2H3N5F2, S1H5N4F4, S2H3N4F2, S1H4N4F2, S1H8N7F3, S1H7N6F2, S2H5N3F2P1, H5N3F2P 1, and H3N6F3P1

A preferred group of N-glycans includes structures comprising more than one fucose residue. The structures comprise at least one fucose residue linked to LacNAc unit as described by the invention. The core structures are described for other groups and fucose residues are linked to LacNAc units as described by the invention.

The preferred glycans include complex fucosylated glycans that shares the composition:

SₙHₚNᵣF_{q}Pₛ

Wherein H is preferably Gal or Man and N is GlcNAc, S is Neu5Ac, F is Fuc, P is sulfate residue (SP in Tables),

n is an integer from 0 to 2; p is an integer from 3 to 8, r is an integer from 3 to 7, q is an integer from 2 to 4; and s is an integer 0 or 1.

High Mannose Type Glycans

hMSC 2, Large High-Mannose Type N-Glycans

The invention is directed to the group of Large high-mannose type N-glycans including non-fucosylated structures H6N2, H7N2, H8N2, and H9N2; and a fucosylated structure including H6N2F1.

The preferred high Mannose type glycans are according to the formula LHM:

[Mα2]ₙ₁Mα3{[Mα2]ₙ₃Mα6}Mα6{[Mα2]ₙ₆[Mα2]ₙ₇Mα3}Mβ4GNβ4[Fucα6]ₙ₈GNyR₂

wherein n1, n3, n6, and n7 and n8 are either independently 0 or 1;

with the provision that when n8 is 1 then the glycan comprises 6 Mannose residues, preferably n6 and n3 are 0 and either of n1 or n7 is 0.

y is anomeric linkage structure a and/or 13 or linkage from derivatized anomeric carbon, and

R₂ is reducing end hydroxyl, chemical reducing end derivative or natural asparagine N-glycoside derivative such as asparagine N-glycosides including aminoacid and/or peptides derived from protein;

[ ] indicates determinant either being present or absent depending on the value of n1, n3, n6, n7; and

{ } indicates a branch in the structure;

M is D-Man, GN is N-acetyl-D-glucosamine, y is anomeric structure or linkage type, preferably beta to Asn.

The preferred non-fucosylated structures in this group include:

Manα2Manα6(Manα2Manα3)Manα6(Manα2Manα2Manα3)Manβ4GNβ4GN,

Manα2Manα6([Manα2]ₙ₃Manα3)Manα6([Manα2]ₙ₆Manα2Manα3)Manβ4GNβ4GN,

Manα2Manα6(Manα3)Manα6(Manα2Manα2Manα3)Manβ4GNβ4GN

Manα2Manα6(Manα2Manα3)Manα6(Manα2Manα3)Manβ4GNβ4GN

Manα2Manα6(Manα3)Manα6(Manα2Manα3)Manβ4GNβ4GN

The preferred fucosylated structures includes

[Manα2]ₙ₁Manα6(Manα3)Manα6([Manα2]ₙ₇Manα3)Manβ4GNβ4(Fucα6)GN,

Manα2Manα6(Manα3)Manα6(Manα3)Manβ4GNβ4(Fucα6)GN,

Manα6(Manα3)Manα6(Manα2Manα3)Manβ4GNβ4(Fucα6)GN,

hMSC 4, Glucosylated High-Mannose Type N-Glycans

The preferred group of glucosylated high-mannose type N-glycans includes H10N2, H11N2, and H12N2

The group of glucosylated high-mannose type glycans is continuous to high-mannose glycans. The glycans group is involved in quality control in ER of cells. The presence of glucosylated high-mannose glycans is considered to correspond to protein synthesis activity and folding efficiency in the cells. The terminal glucose residue is characteristic structure for glycans of this group and in a preferred embodiment the invention is directed to the recognition of the terminal Glc residues by specific binding agents. It is further realized that reagents recognizing high mannos glycan also recognize this structure especially when the recognition is directed to terminal Manα2-structures on non-glucosylated arms of the molecule

The invention revealed substantially more of this type of glycans in mesenchymal stem cells than differentiated cells, especially osteogenically differentiated bone marrow derived stem cells.

The preferred structures are according to the Formula:

Mα2Mα6(Mα2Mα3)Mα6([Gα2]ₙ₁[Gα3]ₙ₂[Gα3]ₙ₃Mα2Mα2Mα3)Mβ4GNβ4GN,

wherein n1, n2 and n3 are either 0 or 1, idenpendently

wherein M is mannose, G is glucose, and GN is N-acetylglucosamine residue

hMSC 3, Soluble Oligomannose Glycans

including H2N1, H3N1, H4N1, H5N1, H6N1, H7N1, H8N1, and H9N1

Structures and Compositions Associated with Differentiated Mesenchymal Cells

The invention revealed novel structures present in higher amount in differentiated mesenchymal stem cells than in corresponding non-differentiated hMSCs.

The preferred glycan groups are represented in groups Diff 1 to Diff 7, corresponding to several types of N-glycans. The glycans are preferred in the order from Diff 1 to Diff 7, based on the relative specificity for the non-differentiated hMSCs, the differences in the expression are shown in Tables.

Diff 1, Sulfated Glycans

Including biantennary-size complex-type glycans H5N4P1, H5N4F1P1, S2H5N4F 1P1, H5N4F2P1, H5N4F3P1, S1H5N4P1, S1H5N4F1P1;

Large complex-type glycans H6N5F1P1, S2H6N5F1P1, H7N6F1P1, H6N5F3P1, and S1H6N5F1P1;

Terminal Hex containing glycans H6N4F3P1, G1H6N4P1, and H7N4P1;

Terminal HexNAc containing glycans S2H4N5F2P2, H4N4F1P1, H3N6F1P1, H4N5F2P1, H3N5F1P1, H3N4P1, H3N4F1P1, and and H4N4P1;

And hybrid-type or monoantennary glycans S2H4N3F1P1, H4N3F1P1, H4N3P1, H5N3F1P1, H4N3F2P1, S1H3N3F1P2, H3N3F1P1, H3N3P1, and S2H5N3P2;

And high-mannose type glycans including H1ON2F1P2, which are preferentially phosphorylated.

The preferred sulfated glycans comprise N-glycan core and preferred type N-acetyllactosamine unit or units which are sulfated, in case or theminal HexNAc units such as GlcNAcβ or GalNAcβ4GlcNAc these may be further sulfated. The presence of sulfate residue on the lactosamine/G1cNAc comprising N-glycans was analyzed by high resolution mass spectrometry and/or specific phophatase enzyme digestion. The glycans may further comprise Neu5Ac and fucose residues.

The sulfated glycans include complex type and related glycans that shares the composition:

SₙHₚNᵣF₁Pₛ

Wherein H is preferably Gal or Man and N is GlcNAc, S is Neu5Ac, F is Fuc, P is sulfate residue (SP in Tables),

n is an integer from 0 to 2; p is an integer from 3 to 7, r is an integer from 3 to 6, q is an integer from 0, 1 or 3; and s is an integer 1 or 2.

The sulfated glycans Large complex-type glycans H6N5F1P1, S2H6N5F1P1, H7N6F1P1, H6N5F3P1, and S1H6N5F1P1

include complex type and related glycans that shares the composition:

SₙHₚNᵣF_{q}P₁

Wherein H is preferably Gal or Man and N is GlcNAc, S is Neu5Ac, F is Fuc, P is sulfate residue (SP in Tables),

n is an integer from 0 to 2; p is an integer from 6 to 7, r is an integer from 5 to 6, and q is an integer 1 or 3. The preferred core structures with core composition H6N5-comprising glycans was described for hMSC 6, glycans with composition of H7N6 comprise four LacNAc units as tetraantennary and/or poly-lacNAc comprising structure. The diasialylate structure comprises two Neu5Ac units at terminal LacNAc units and one fucose residue is in a preferred embodiment linked to the core of the N-glycan.

The preferred sulfated biantennary N-glycans include glycans that shares the composition:

SₙH₅N₄F_{q}P₁

Wherein H is preferably Gal or Man and N is GlcNAc, S is Neu5Ac and F is Fuc,

n is an integer from 0 or 2; q is an integer from 0 to 3.

The preferred structures are as described for biantennary N-glycans in hMSC groups, but the glycans further comprise a sulfate group linked to N-acetyllactosamine unit as described for preferred sulfates terminal N-glycan structure comprising terminal type 2 LacNAc units. The presence of a disialylated structure indicates that the glycans comprise at least part of the sulphate residues linked to 6-position of GlcNAc and/or Gal residue.

The preferred core structures of the glycans has been representen in Tables and in other preferred groups, the invention is further directed to following preferred core structure groups comprising sulphated LacNAc or GlcNAc:

The preferred core H4H5 structures, H4N5 and H4N5F2, include following preferred structures comprising LacdiNAc:

[Fucα]ₙ₃{Gal[NAc]ₙ₁βGNβ2Manα3(Gal[NAc]ₙ₂βGNβ2Manα6)Manβ4GNβ4(Fucα6)ₙ₂GN, wherein n1 and n2 are either 0 or 1, so that either n1 or n2 is 0 and the other is 1 and n3 is either 0 or 1. The fucose residue forms preferably Lewis x or fucosylated LacdiNAc structure GalNAcβ4(Fucα3)GlcNAc.

Preferred core structures of hybrid-type N-glycans, including H5N3, according to the Formula:

[Galβ]ₙ₁GlcNAcβ2Manα3(Manα3/6[Manα6/3]ₙ₃Manα6)Manβ4GlcNAcβ4(Fucα6)ₙ₂GlcNAc

Wherein n1 and n2 and n3 are either 0 or 1, so that there is 5 hexose (Gal/Man) units.

The preferred H5N3 comprising structures comprise core structure according to the Formula

GlcNAcβ2Manα3(Manα3[Manα6]Manα6)Manβ4GlcNAcβ4(Fucα6)ₙ₂GlcNAc

Wherein n2 is either 0 or 1.

Terminal HexNAc monoantennary N-glycans, with core structure compositions H3N3F1;

preferentially includes core structures (Galβ4)₀₋₁GlcNAcβ2Manα3([Manα6]₀₋₁)Manβ4GlcNAcβ4(Fucα6)GlcNAc, more preferentially with type II N-acetyllactosamine antennae (without Manα6 branch), wherein galactose residue is β1,4-linked.

Diff 2, Low-Mannose Type N-Glycans

Including non-fucosylated glycans H1N2, H3N2, and H4N2;

And fucosylated glycans H2N2F1, H3N2F1, and H4N2F1

Diff 3, Small High-Mannose Type (Mans) N-Glycans

comprising non-fucosylated H5N2 and fucosylated H5N2F1

Diff 4, Neutral Hybrid-Type and Monoantennary N-Glycans

Including monoantennary glycans H2N3, H2N3F1, H3N3, H3N3F1, H3N3F2;

Hybrid-type and/or monoantennary glycans H4N3 and H4N3F1;

And hybrid-type glycans H4N3F2, H5N3, H5N3F1, H5N3F2, H6N3, H6N3F1, and H7N3

Diff 5, Neutral Complex-Type N-Glycans

Including biantennary-size complex-type glycans H5N4, H5N4F1, H5N4F2, and H5N4F3;

Large complex-type glycans H6N5, H6N5F1, H6N5F2, H6N5F3, H6N5F4, H7N6, H7N6F1, and H8N7;

Terminal HexNAc containing glycans H5N5, H5N5F1, H5N5F2, H5N5F3, H6N6, H3N4, H4N4, H4N4F1, H4N4F2, H4N5, H4N5F2, and H3N6F1;

Terminal Hex containing glycans H6N4, H6N4F1, H7N4, H6N4F2, H7N4F1, and H8N4.

Preferred core structures of the glycans has been described in context of other glycan groups and

The preferred H4H5 structures, such as H4N5F2 and H4N5, include following preferred structures comprising LacdiNAc: [Fucα]ₙ₃{Gal[NAc]ₙ₁βGNβ2Manα3(Gal[NAc]ₙ₂βGNβ2Manα6)Manβ4GNβ4(Fucα6)ₙ₄GN,

wherein n1 and n2 are either 0 or 1, so that either n1 or n2 is 0 and the other is 1 and n3 and n4 are either 0 or 1, independently. The fucose residue forms preferably Lewis x or fucosylated LacdiNAc structure GalNAcβ4(Fucα3)GlcNAc.

The glycans comprising core composition H═N=5 type are preferably terminal HexNAc comprising N-glycans, including H5N5F1, H5N5, H5N5F3 Comprising the binatennary N-glycan core structure and terminal HexNAc, especially terminal GlcNAc glycans linked to the core of the N-glycan

Diff 7, Monosialylated Biantennary-Size Complex-Type N-Glycans

Including G1H5N4, S1H5N4P1, S1H5N4F1, G1H5N4F1, S1H5N4F1P1, and S1H5N4F3

S₁H₅N₄F_{q}Pₛ

Wherein H is preferably Gal or Man and N is GlcNAc, S is Neu5Ac or Neu5Gc, preferably Neu5Ac and F is Fuc and P is sulfate residue,

q is an integer from 0 to 3, preferably 0, 1 or 3, s is an integer 0 or 1.

The preferred core structures of the biantennary N-glycans are describe in other groups according of the invention. The glycans comprise one preferred sialyl-LacNAc unit and one LacNAc unit, which may be further sulphated and/or fucosylated.

Preferred N-Glycan Structure Types

The invention revealed N-glycans with common core structure of N-glycans, which change according to differentiation and/or between individual cell lines. For assignment of the structures see also TABLEs. The structures correspond also to the mass numbers and monosaccharide compositions of Tables, glycosidase Table and monosaccharide compositions and structures described of glycans changing in context of differentiation and in Figures. Monosaccharide composition corresponding to a glycan structure is obtained by indicating Gal and Man as Hex (or H in shorter presentation), the number of Hex units is sum of amount of Man and Gal residue; and GlcNAc (or GalNAc) residue as HexNAc or N and indicating the number of fucose residues (F), sialic acid residues (S/Neu5Ac or G/Neu5Gc), Ac indicates O-acetyl residues and possible sulfate or phosphoryl residues are indicated with number after SP or P sharing similar molecular weight.

The N-glycans of mesenchymal stem cells comprise the core structure comprising Manβ4GlcNAc structure in the core structure of N-linked glycan according to the

[Manα3]ₙ₁(Manα6)ₙ₂Manβ4GlcNAcβ4(Fucα6)ₙ₃GlcNAcxR, Formula CGN:

wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures (for the analysis of the status of stem cells and/or manipulation of the stem cells), wherein xR indicates reducing end structure of N-glycan linked to protein or peptide such as βAsn or βAsn-peptide or βAsn-protein, or free reducing end of N-glycan or chemical derivative of the reducing end produced for analysis.

The preferred Mannose type glycans are according to the formula:

[Mα2]ₙ₁[Mα3]ₙ₂{[Mα2]ₙ₃[Mα6)]ₙ₄}[Mα6]ₙ₅{[Mα2]ₙ₆[Mα2]ₙ₇[Mα3]ₙ₈}Mβ4GNβ4[{Fucα6}]ₘGNyR₂ Formula M2:

wherein n1, n2, n3, n4, n5, n6, n7, n8, and m are either independently 0 or 1; with the provision that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; y is anomeric linkage structure α and/or β or linkage from derivatized anomeric carbon, and

R₂ is reducing end hydroxyl, chemical reducing end derivative or natural asparagine N-glycoside derivative such as asparagine N-glycosides including asparagines N-glycoside amino acid and/or peptides derived from protein;

[ ] indicates determinant either being present or absent depending on the value of n1, n2, n3, n4, n5, n6, n7, n8, and m; and

{ } indicates a branch in the structure;

M is D-Man, GN is N-acetyl-D-glucosamine and Fuc is L-Fucose,

and the structure is optionally a high mannose structure, which is further substituted by glucose residue or residues linked to mannose residue indicated by n6.

Several preferred low Man glycans described above can be presented in a single Formula:

[Mα3]ₙ₂{[Mα6)]ₙ₄}[Mα6]ₙ₅{[Mα3]ₙ₈}Mβ4GNβ4[{Fucα6}]ₘGNyR₂

wherein n2, n4, n5, n8, and m are either independently 0 or 1; with the provision that when n5 is 0, also n2, and n4 are 0;the sum of n2, n4, n5, and n8 is less than or equal to (m+3); [ ] indicates determinant either being present or absent depending on the value of n2, n4, n5, n8, and m; and

{ } indicates a branch in the structure;

y and R2 are as indicated above.

Preferred non-fucosylated low-mannose glycans are according to the formula:

[Mα3]ₙ₂([Mα6)]ₙ₄)[Mα6]ₙ₅{[Mα3]ₙ₈}Mβ4GNβ4GNyR₂

wherein n2, n4, n5, n8, and m are either independently 0 or 1,

with the provision that when n5 is 0, also n2 and n4 are 0, and preferably either n2 or n4 is 0,

[ ] indicates determinant either being present or absent depending on the value of , n2, n4, n5, n8,

{ } and ( ) indicates a branch in the structure,

y and R2 are as indicated above.

Preferred Individual Structures of Non-Fucosylated Low-Mannose Glycans

Special Small Structures

Small non-fucosylated low-mannose structures are especially unusual among known

N-linked glycans and characteristic glycan group useful for separation of cells according to the present invention. These include: Mβ4GNβ4GNyR₂

Mα6Mβ4GNβ4GNyR₂

Mα3 Mβ4GNβ4GNyR₂ and

Mα6{Mα3}Mβ4GNβ4GNyR₂.

Mβ4GNβ4GNyR₂ trisaccharide epitope is a preferred common structure alone and together with its mono-mannose derivatives Mα6Mβ4GNβ4GNyR₂ and/or

Mα3Mβ4GNβ4GNyR₂, because these are characteristic structures commonly present in glycomes according to the invention. The invention is specifically directed to the glycomes comprising one or several of the small non-fucosylated low-mannose structures. The tetrasaccharides are in a specific embodiment preferred for specific recognition directed to α-linked, preferably α3/6-linked Mannoses as preferred terminal recognition element.

Special Large Structures

The invention further revealed large non-fucosylated low-mannose structures that are unusual among known N-linked glycans and have special characteristic expression features among the preferred cells according to the invention. The preferred large structures include

[Mα3]ₙ₂([Mα6]ₙ₄)Mα6{Mα3}Mβ4GNβ4GNyR₂

more specifically

Mα6Mα6{Mα3}Mβ4GNβ4GNyR₂

Mα3Mα6{Mα3}Mβ4GNβ4GNyR₂ and

Mα3(Mα6)Mα6{Mα3}Mβ4GNβ4GNyR₂.

The hexasaccharide epitopes are preferred in a specific embodiment as rare and characteristic structures in preferred cell types and as structures with preferred terminal epitopes. The heptasaccharide is also preferred as a structure comprising a preferred unusual terminal epitope Mα3(Mα6)Mα useful for analysis of cells according to the invention.

Preferred fucosylated low-mannose glycans are derived according to the formula:

[Mα3]ₙ₂{[Mα6]ₙ₄}[Mα6]ₙ₅{[Mα3]ₙ₈}Mβ4GNβ4(Fucα6)GNyR₂

wherein n2, n4, n5, n8, and m are either independently 0 or 1,with the provision that when n5 is 0, also n2 and n4 are 0,

[ ] indicates determinant either being present or absent depending on the value of n2, n4, n5, n8, and m;

{ } and ( ) indicate a branch in the structure.

Preferred Individual Structures of Fucosylated Low-Mannose Glycans

Small fucosylated low-mannose structures are especially unusual among known N-linked glycans and form a characteristic glycan group useful for separation of cells according to the present invention. These include:

Mβ4GNβ4(Fucα6)GNyR₂

Mα6Mβ4GNβ4(Fucα6)GNyR₂

Mα3Mβ4GNβ4(Fucα6)GNyR₂ and

Mα6{Mα3}Mβ4GNβ4(Fucα6)GNyR₂.

Mβ4GNβ4(Fucα6)GNyR₂ tetrasaccharide epitope is a preferred common structure alone and together with its monomannose derivatives Mα6Mβ4GNβ4(Fucα6)GNyR₂ and/or Mα3Mβ4GNβ4(Fucα6)GNyR₂, because these are commonly present characteristic structures in glycomes according to the invention. The invention is specifically directed to the glycomes comprising one or several of the small fucosylated low-mannose structures. The tetrasaccharides are in a specific embodiment preferred for specific recognition directed to α-linked, preferably β3/6-linked Mannoses as preferred terminal recognition element.

Special Large Structures

The invention further revealed large fucosylated low-mannose structures that are unusual among known N-linked glycans and have special characteristic expression features among the preferred cells according to the invention. The preferred large structures include

[Mα3]ₙ₂([Mα6]ₙ₄)Mα6{Mα3}Mβ4GNβ4(Fucα6)GNyR₂

more specifically

Mα6Mα6{Mα3}Mβ4GNβ4(Fucα6)GNyR₂

Mα3Mα6{Mα3}Mβ4GNβ4(Fucα6)GNyR₂ and

Mα3(Mα6)Mα6{Mα3}Mβ4GNβ4(Fucα6)GNyR₂.

The heptasaccharide epitopes are preferred in a specific embodiment as rare and characteristic structures in preferred cell types and as structures with preferred terminal epitopes. The octasaccharide is also preferred as structure comprising a preferred unusual terminal epitope Mα3(Mα6)Mα useful for analysis of cells according to the invention.

Preferred Non-Reducing End Terminal Mannose-Epitopes

The inventors revealed that mannose-structures can be labeled and/or otherwise specifically recognized on cell surfaces or cell derived fractions/materials of specific cell types. The present invention is directed to the recognition of specific mannose epitopes on cell surfaces by reagents binding to specific mannose structures on cell surfaces.

The preferred reagents for recognition of any structures according to the invention include specific antibodies and other carbohydrate recognizing binding molecules. It is known that antibodies can be produced for the specific structures by various immunization and/or library technologies such as phage display methods representing variable domains of antibodies. Similarly with antibody library technologies, including aptamer technologies and including phage display for peptides, exist for synthesis of library molecules such as polyamide molecules including peptides, especially cyclic peptides, or nucleotide type molecules such as aptamer molecules.

The invention is specifically directed to specific recognition of high-mannose and low-mannose structures according to the invention. The invention is specifically directed to recognition of non-reducing end terminal Manα-epitopes, preferably at least disaccharide epitopes, according to the formula:

[Mα2]ₘ₁[Mαx]ₘ₂[Mα6]ₘ₃{{[Mα2]ₘ₉[Mα2]ₘ₈[Mα3]ₘ₇}ₘ₁₀(Mβ4[GN]ₘ₄)ₘ₅}ₘ₆yR₂

wherein m1, m2, m3, m4, m5, m6, m7, m8, m9 and m10 are independently either 0 or 1; with the provision that when m3 is 0, then ml is 0, and when m7 is 0 then either m1-5 are 0 and m8 and m9 are 1 forming a Mα2Mα2-disaccharide, or both m8 and m9 are 0;

y is anomeric linkage structure a and/or 0 or linkage from derivatized anomeric carbon, and

R₂ is reducing end hydroxyl or chemical reducing end derivative and x is linkage position 3 or 6 or both 3 and 6 forming branched structure,

{ } indicates a branch in the structure.

The invention is further directed to terminal Mα2-containing glycans containg at least one Mα2-group and preferably Mα2-group on each branch so that m1 and at least one of m8 or m9 is 1. The invention is further directed to terminal Mα3 and/or Mα6-epitopes without terminal Mα2-groups, when all m1, m8 and m9 are 1.

The invention is further directed in a preferred embodiment to the terminal epitopes linked to a Mβ-residue and for application directed to larger epitopes. The invention is especially directed to Mβ4GN-comprising reducing end terminal epitopes.

The preferred terminal epitopes comprise typically 2-5 monosaccharide residues in a linear chain. According to the invention short epitopes comprising at least 2 monosaccharide residues can be recognized under suitable background conditions and the invention is specifically directed to epitopes comprising 2 to 4 monosaccharide units and more preferably 2-3 monosaccharide units, even more preferred epitopes include linear disaccharide units and/or branched trisaccharide non-reducing residue with natural anomeric linkage structures at reducing end. The shorter epitopes may be preferred for specific applications due to practical reasons including effective production of control molecules for potential binding reagents aimed for recognition of the structures.

The shorter epitopes such as Mα2M is often more abundant on target cell surface as it is present on multiple arms of several common structures according to the invention.

Preferred Disaccharide Epitopes Include

Manα2Man, Manα3Man, Manα6Man, and more preferred anomeric forms Manα2Manα, Manα3Manβ, Manα6Manβ, Manα3Manα and Manα6Manα.

Preferred branched trisaccharides include Manα3(Manα6)Man, Manα3(Manα6)Manβ, and Manα3(Manα6)Manα.

The invention is specifically directed to the specific recognition of non-reducing terminal Manα2-structures especially in context of high-mannose structures.

The invention is specifically directed to following linear terminal mannose epitopes:

a) preferred terminal Manα2-epitopes including following oligosaccharide sequences:

Manα2Man,

Manα2Manα,

Manα2Manα2Man, Manα2Manα3Man, Manα2Manα6Man,

Manα2Manα2Manα, Manα2Manα3Manβ, Manα2Manα6Manα,

Manα2Manα2Manα3Man, Manα2Manα3Manα6Man, Manα2Manα6Manα6Man

Manα2Manα2Manα3Manβ, Manα2Manα3Manα6Manβ,

Manα2Manα6Manα6Manβ;

The invention is further directed to recognition of and methods directed to non-reducing end terminal Manα3- and/or Manα6-comprising target structures, which are characteristic features of specifically important low-mannose glycans according to the invention. The preferred structural groups include linear epitopes according to b) and branched epitopes according to the c3) especially depending on the status of the target material.

b) preferred terminal Manα3- and/or Manα6-epitopes including following oligosaccharide sequences:

Manα3Man, Manα6Man, Manα3Manβ, Manα6Manβ, Manα3Manα, Manα6Manα, Manα3 Manα6Man, Manα6Manα6Man, Manα3Manα6Manβ, Manα6Manα6Manβ and to following:

c) branched terminal mannose epitopes are preferred as characteristic structures of especially high-mannose structures (c1 and c2) and low-mannose structures (c3), the preferred branched epitopes including:

c1) branched terminal Manα2-epitopes

Manα2Manα3(Manα2Manα6)Man, Manα2Manα3(Manα2Manα6)Manα,

Manα2Manα3(Manα2Manα6)Manα6Man,

Manα2Manα3(Manα2Manα6)Manα6Manβ,

Manα2Manα3(Manα2Manα6)Manα6(Manα2Manα3)Man,

Manα2Manα3(Manα2Manα6)Manα6(Manα2Manα2Manα3)Man,

Manα2Manα3(Manα2Manα6)Manα6(Manα2Manα3)Manβ

Manα2Manα3(Manα2Manα6)Manα6(ManαManα2Manα3)Manβ

c2) branched terminal Manα2- and Manα3 or Manα6-epitopes according to formula when m1 and/or m8 and/m9 is 1 and the molecule comprise at least one nonreducing end terminal Manα3 or Manα6-epitope

c3) branched terminal Manα3 or Manα6-epitopes

Manα3(Manα6)Man, Manα3(Manα6)Manβ, Manα3(Manα6)Manα,

Manα3(Manα6)Manα6Man, Manα3(Manα6)Manα6Manβ,

Manα3(Manα6)Manα6(Manα3)Man, Manα3(Manα6)Manα6(Manα3)Manβ

The present invention is further directed to increase the selectivity and sensitivity in recognition of target glycans by combining recognition methods for terminal Manα2 and Manα3 and/or Manα6-comprising structures. Such methods would be especially useful in the context of cell material according to the invention comprising both high-mannose and low-mannose glycans.

Complex Type N-Glycans

According to the present invention, complex-type structures are preferentially identified by mass spectrometry, preferentially based on characteristic monosaccharide compositions, wherein HexNAc≧4 and Hex≧3. In a more preferred embodiment of the present invention, 4≦HexNAc≦20 and 3≦Hex≦21, and in an even more preferred embodiment of the present invention, 4≦HexNAc≦10 and 3≦Hex≦11. The complex-type structures are further preferentially identified by sensitivity to endoglycosidase digestion, preferentially N-glycosidase F detachment from glycoproteins. The complex-type structures are further preferentially identified in NMR spectroscopy based on characteristic resonances of the Manα3(Manα6)Manβ4GlcNAcβ4GlcNAc N-glycan core structure and GlcNAc residues attached to the Manα3 and/or Manα6 residues.

Beside Mannose-type glycans the preferred N-linked glycomes include GlcNAcβ2-type glycans including Complex type glycans comprising only GlcNAcβ2-branches and Hydrid type glycan comprising both Mannose-type branch and GlcNAcβ2-branch.

GlcNAcβ2-Type Glycans

The invention revealed GlcNAcβ2Man structures in the glycomes according to the invention. Preferably GlcNAcβ2Man-structures comprise one or several of GlcNAcβ2Manα-structures, more preferably GlcNAcβ2Manα3- or GlcNAcβ2Manα6-structure.

The Complex type glycans of the invention comprise preferably two GlcNAcβ2Manα structures, which are preferably GlcNAcβ2Manα3 and GlcNAcβ2Manα6. The Hybrid type glycans comprise preferably GlcNAcβ2Manα3-structure.

The present invention is directed to at least one of natural oligosaccharide sequence structures and structures truncated from the reducing end of the N-glycan according to the Formul CO1 (also referred as GNβ2):

[R₁GNβ2]ₙ₁[Mα3]ₙ₂{[R₃]ₙ₃[GNβ2]ₙ₄Mα6}ₙ₅Mβ4GNXyR₂,

with optionally one or two or three additional branches according to formula [RₓGNβz]ₙₓ linked to Mα6-, Mα3-, or Mβ4, and Rₓ may be different in each branch

wherein n1, n2, n3, n4, n5 and nx, are either 0 or 1, independently,

with the provision that when n2 is 0 then n1 is 0 and when n3 is 1 and/or n4 is 1 then n5 is also 1, and at least n1 or n4 is 1, or n3 is 1;

when n4 is 0 and n3 is 1 then R₃ is a mannose type substituent or nothing and wherein X is a glycosidically linked disaccharide epitope β4(Fucα6)ₙGN, wherein n is 0 or 1, or X is nothing and

y is anomeric linkage structure α and/or β or linkage from derivatized anomeric carbon, and

R₁, Rₓ and R₃ indicate independently one, two or three natural substituents linked to the core structure,

R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

Elongation of GlcNAcβ2-Type Structures Forming Complex/Hydrid Type Structures

The substituents R₁, Rₓ and R₃ may form elongated structures. In the elongated structures R₁, and Rₓ represent substituents of GlcNAc (GN) and R₃ is either substituent of GlcNAc or when n4 is 0 and n3 is 1 then R3 is a mannose type substituent linked to Manα6-branch forming a Hybrid type structure. The substituents of GN are monosaccharide Gal, GalNAc, or Fuc and/or acidic residue such as sialic acid or sulfate or phosphate ester.

GlcNAc or GN may be elongated to N-acetyllactosaminyl also marked as GalβGN or di-N-acetyllactosdiaminyl GalNAcβGlcNAc, preferably GalNAcβ4GlcNAc. LNβ2M can be further elongated and/or branched with one or several other monosaccharide residues such as galactose, fucose, SA or LN-unit(s) which may be further substituted by SAα-strutures,

and/or Mα6 residue and/or Mα3 residue can be further substituted by one or two β6-, and/or β4-linked additional branches according to the formula;

and/or either of Mα6 residue or Mα3 residue may be absent;

and/or Mα6-residue can be additionally substituted by other Manα units to form a hybrid type structures;

and/or Manβ4 can be further substituted by GNβ4,

and/or SA may include natural substituents of sialic acid and/or it may be substituted by other SA-residues preferably by α8- or α9-linkages.

The SAα-groups are linked to either 3- or 6-position of neighboring Gal residue or on 6-position of GlcNAc, preferably 3- or 6-position of neighboring Gal residue. In separately preferred embodiments the invention is directed to structures comprising solely 3-linked SA or 6-linked SA, or mixtures thereof.

Preferred Complex Type Structures

Incomplete Monoantennary N-Glycans

The present invention revealed incomplete Complex monoantennary N-glycans, which are unusual and useful for characterization of glycomes according to the invention. The most of the incomplete monoantennary structures indicate potential degradation of biantennary N-glycan structures and are thus preferred as indicators of cellular status. The incomplete Complex type monoantennary glycans comprise only one GNβ2-structure.

The invention is specifically directed to structures according to the Formula CO1 or Formula GNb2 above when only n1 is 1 or n4 is 1 and mixtures of such structures.

The preferred mixtures comprise at least one monoantennary complex type glycans

A) with a single branch likely from a degradative biosynthetic process:

R₁GNβ2Mα3β4GNXyR₂

R₃GNβ2Mα6Mβ4GNXyR₂ and

B) with two branches comprising mannose branches
B1) R₁GNβ2Mα3{Mα6}ₙ₅Mβ4GNXyR₂
B2) Mα3{R₃GNβ2Mα6}ₙ₅Mβ4GNXyR₂

The structure B2 is preferred over A structures as product of degradative biosynthesis, it is especially preferred in context of lower degradation of Manα3 -structures. The structure B1 is useful for indication of either degradative biosynthesis or delay of biosynthetic process.

Biantennary and Multiantennary Structures

The inventors revealed a major group of biantennary and multiantennary N-glycans from cells according to the invention. The preferred biantennary and multiantennary structures comprise two GNβ2 structures. These are preferred as an additional characteristic group of glycomes according to the invention and are represented according to the Formula CO2:

R₁GNβ2Mα3{R₃GNβ2Mα6}Mβ4GNXyR₂

with optionally one or two or three additional branches according to formula [RₓGNβz]ₙₓ linked to Mα6-, Mα3-, or Mβ4 and Rₓ may be different in each branch

wherein nx is either 0 or 1,

and other variables are according to the Formula C01.

Preferred Biantennary Structure

A biantennary structure comprising two terminal GNβ-epitopes is preferred as a potential indicator of degradative biosynthesis and/or delay of biosynthetic process. The more preferred structures are according to the Formula CO2 when R₁ and R₃ are nothing.

Elongated Structures

The invention revealed specific elongated complex type glycans comprising Gal and/or GalNAc-structures and elongated variants thereof. Such structures are especially preferred as informative structures because the terminal epitopes include multiple informative modifications of lactosamine type, which characterize cell types according to the invention.

The present invention is directed to at least one of natural oligosaccharide sequence structure or group of structures and corresponding structure(s) truncated from the reducing end of the N-glycan according to the Formula CO3:

[R₁Gal[NAc]ₒ₂βz2]ₒ₁GNβ2Mα3{[R₁Gal[NAc]ₒ₄βz2]ₒ₃GNβ2Mα6}Mβ4GNXyR₂,

with optionally one or two or three additional branches according to formula [RₓGNβz1]ₘₓ linked to Mα6-, Mα3-, or M134 and Rₓ may be different in each branch

wherein nx, o1, o2, o3, and o4 are either 0 or 1, independently,

with the provision that at least ol or o3 is 1, in a preferred embodiment both are 1;

z2 is linkage position to GN being 3 or 4, in a preferred embodiment 4;

z1 is linkage position of the additional branches;

R₁, Rx and R₃ indicate one or two a N-acetyllactosamine type elongation groups or nothing,

{ } and ( ) indicates branching which may be also present or absent, other variables are as described in Formula GNb2.

Galactosylated Structures

The inventors characterized useful structures especially directed to digalactosylated structure

GalβzGNβ2Mα3{GalβzGNβ2Mα6}Mβ4GNXyR₂,

and monogalactosylated structures:

GalβzGNβ2Mα3{GNβ2Mα6}Mβ4GNXyR₂,

GNβ2Mα3{GalβzGNβ2Mα6}Mβ4GNXyR₂,

and/or elongated variants thereof preferred for carrying additional characteristic terminal structures useful for characterization of glycan materials

R₁GalβzGNβ2Mα3{R₃GalβzGNβ2Mα6}Mβ4GNXyR₂

R₁GalβzGNβ2Mα3{GNβ2Mα6}Mβ4GNXyR₂, and

GNβ2Mα3{R₃GalβzGNβ2Mα6}Mβ4GNXyR₂.

Preferred elongated materials include structures wherein R₁ is a sialic acid, more preferably NeuNAc or NeuGc.

LacdiNAc-Structure Comprising N-Glycans

The present invention revealed for the first time LacdiNAc, GalNAcβGlcNAc structures from the cell according to the invention. Preferred N-glycan lacdiNAc structures are included in structures according to the Formula CO1, when at least one the variable o2 and o4 is 1.

The Major Acidic Glycan Types

The acidic glycomes mean glycomes comprising at least one acidic monosaccharide residue such as sialic acids (especially NeuNAc and NeuGc) forming sialylated glycome, HexA (especially GlcA, glucuronic acid) and/or acid modification groups such as phosphate and/or sulfate esters.

According to the present invention, presence of sulfate and/or phosphate ester (SP) groups in acidic glycan structures is preferentially indicated by characteristic monosaccharide compositions containing one or more SP groups. The preferred compositions containing SP groups include those formed by adding one or more SP groups into non-SP group containing glycan compositions, while the most preferential compositions containing SP groups according to the present invention are selected from the compositions described in the acidic N-glycan fraction glycan group Tables of the present invention. The presence of phosphate and/or sulfate ester groups in acidic glycan structures is preferentially further indicated by the characteristic fragments observed in fragmentation mass spectrometry corresponding to loss of one or more SP groups, the insensitivity of the glycans carrying SP groups to sialidase digestion. The presence of phosphate and/or sulfate ester groups in acidic glycan structures is preferentially also indicated in positive ion mode mass spectrometry by the tendency of such glycans to form salts such as sodium salts as described in the Examples of the present invention. Sulfate and phosphate ester groups are further preferentially identified based on their sensitivity to specific sulphatase and phosphatase enzyme treatments, respectively, and/or specific complexes they form with cationic probes in analytical techniques such as mass spectrometry.

Sialylated Complex N-Glycan Glycomes

The present invention is directed to at least one of natural oligosaccharide sequence structures and structures truncated from the reducing end of the N-glycan according to the Formula

[{SAα3/6}ₛ₁LNβ2]ᵣ₁Mα3{({SAα3/6}ₛ₂LNβ2)ᵣ₂Mα6}ᵣ₈{M[β4GN[β4{Fucα6}ᵣ₃GN]ᵣ₄]ᵣ₅}ᵣ₆ (1)

with optionally one or two or three additional branches according to formula

{SAα3/6}ₛ₃LNβ, (IIb)

wherein r1, r2, r3, r4, r5, r6, r7 and r8 are either 0 or 1, independently,

wherein s1, s2 and s3 are either 0 or 1, independently,

with the provision that at least r1 is 1 or r2 is 1, and at least one of s1, s2 or s3 is 1.

LN is N-acetyllactosaminyl also marked as GalβGN or di-N-acetyllactosdiaminyl GalNAcβGlcNAc preferably GalNAcβ4GlcNAc, GN is GlcNAc, M is mannosyl-, with the provision that LNβ2M or GNβ2M can be further elongated and/or branched with one or several other monosaccharide residues such as galactose, fucose, SA or LN-unit(s) which may be further substituted by SAα-strutures,

and/or one LNβ can be truncated to GNP

and/or Mα6 residue and/or Mα3 residue can be further substituted by one or two β6-, and/or β4-linked additional branches according to the formula,

and/or either of Mα6 residue or Mα3 residue may be absent;

and/or Mα6-residue can be additionally substituted by other Manα units to form a hybrid type structures

and/or Manβ4 can be further substituted by GNβ4,

and/or SA may include natural substituents of sialic acid and/or it may be substituted by other SA-residues preferably by α8- or α9-linkages.

( ), { }, [ ] and [ ] indicate groups either present or absent in a linear sequence. { } indicates branching which may be also present or absent.

The SAα-groups are linked to either 3- or 6-position of neighboring Gal residue or on 6-position of GlcNAc, preferably 3- or 6-position of neighboring Gal residue. In separately preferred embodiments the invention is directed structures comprising solely 3-linked SA or 6-linked SA, or mixtures thereof In a preferred embodiment the invention is directed to glycans wherein r6 is 1 and r5 is 0, corresponding to N-glycans lacking the reducing end GlcNAc structure.

The LN unit with its various substituents can be represented in a preferred general embodiment by the formula:

[Gal(NAc)ₙ₁α3]ₙ₂{Fucα2}ₙ₃Gal(NAc)ₙ₄β3/4{Fucα4/3}ₙ₅GlcNAcβ

wherein n1, n2, n3, n4, and n5 are independently either 1 or 0,

with the provision that the substituents defined by n2 and n3 are alternative to the presence of SA at the non-reducing end terminal structure;

the reducing end GlcNAc-unit can be further β3- and/or β6-linked to another similar LN-structure forming a poly-N-acetyllactosamine structure with the provision that for this LN-unit n2, n3 and n4 are 0,

the Gal(NAc)β and GlcNAcβ units can be ester linked a sulfate ester group; ( ) and [ ] indicate groups either present or absent in a linear sequence; { } indicates branching which may be also present or absent.

LN unit is preferably Galβ4GN and/or Galβ3GN. The inventors revealed that hMSCs can express both types of N-acetyllactosamine, and therefore the invention is especially directed to mixtures of both structures. Furthermore, the invention is directed to special relatively rare type 1 N-acetyllactosamines, Galβ3GN, without any non-reducing end/site modification, also called lewis c-structures, and substituted derivatives thereof, as novel markers of hMSCs.

Hybrid Type Structures

According to the present invention, hybrid-type or monoantennary structures are preferentially identified by mass spectrometry, preferentially based on characteristic monosaccharide compositions, wherein HexNAc=3 and Hex≧2. In a more preferred embodiment of the present invention 2≦Hex≦11, and in an even more preferred embodiment of the present invention 2≦Hex≦9. The hybrid-type structures are further preferentially identified by sensitivity to exoglycosidase digestion, preferentially α-mannosidase digestion when the structures contain non-reducing terminal α-mannose residues and Hex≧3, or even more preferably when Hex≧4, and to endoglycosidase digestion, preferentially N-glycosidase F detachment from glycoproteins. The hybrid-type structures are further preferentially identified in NMR spectroscopy based on characteristic resonances of the Manα3(Manα6)Manβ4GlcNAcβ4GlcNAc N-glycan core structure, a GlcNAcrβ residue attached to a Manα residue in the N-glycan core, and the presence of characteristic resonances of non-reducing terminal α-mannose residue or residues.

The monoantennary structures are further preferentially identified by insensitivity to a-mannosidase digestion and by sensitivity to endoglycosidase digestion, preferentially N-glycosidase F detachment from glycoproteins. The monoantennary structures are further preferentially identified in NMR spectroscopy based on characteristic resonances of the Manα3Manβ4GlcNAcβ4GlcNAc N-glycan core structure, a GlcNAcβ residue attached to a Manα residue in the N-glycan core, and the absence of characteristic resonances of further non-reducing terminal α-mannose residues apart from those arising from a terminal α-mannose residue present in a ManαManβ sequence of the N-glycan core.

The invention is further directed to the N-glycans when these comprise hybrid type structures according to the Formula HY1:

R₁GNβ2Mα3{[R₃]ₙ₃Mα6}Mβ4GNXyR₂,

wherein n3, is either 0 or 1, independently,

and wherein X is glycosidically linked disaccharide epitope β4(Fucα6)ₙGN, wherein

n is 0 or 1, or X is nothing and

y is anomeric linkage structure a and/or 13 or linkage from derivatized anomeric carbon, and

R₁ indicate nothing or substituent or substituents linked to GlcNAc,

R₃ indicates nothing or Mannose-substituent(s) linked to mannose residue, so that each of R₁, and R₃ may correspond to one, two or three, more preferably one or two, and most preferably at least one natural substituents linked to the core structure,

R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

Preferred Hybrid Type Structures

The preferred hydrid type structures include one or two additional mannose residues on the preferred core stucture.

R₁GNβ2Mα3{[Mα3]ₘ₁([Mα6])ₘ₂Mα6}Mβ4GNXyR₂, Formula HY2

wherein and m1 and m2 are either 0 or 1, independently,

{ } and ( ) indicates branching which may be also present or absent, other variables are as described in Formula HY1.

Furthermore the invention is directed to structures comprising additional lactosamine type structures on GN132-branch. The preferred lactosamine type elongation structures includes N-acetyllactosamines and derivatives, galactose, GalNAc, GlcNAc, sialic acid and fucose.

Preferred structures according to the formula HY2 include:

Structures containing non-reducing end terminal GlcNAc as a specific preferred group of glycans

GNβ2Mα3{Mα3Mα6}Mβ4GNXyR₂,

GNβ2Mα3{Mα6Mα6}Mβ4GNXyR₂,

GNβ2Mα3{Mα3(Mα6)Mα6}Mβ4GNXyR₂,

and/or elongated variants thereof

R₁GNβ2Mα3{Mα3Mα6}Mβ4GNXyR₂,

R₁GNβ2Mα3{Mα6Mα6}Mβ4GNXyR₂,

R₁GNβ2Mα3{Mα3(Mα6)Mα6}Mβ4GNXyR₂,

[R₁Gal[NAc]ₒ₂βz]ₒ₁GNβ2Mα3{[Mα3]ₘ₁[(Mα6)]ₘ₂Mα6}ₙ₅Mβ4GNXyR₂, Formula HY3

wherein n5, m1, m2, of and o2 are either 0 or 1, independently,

z is linkage position to GN being 3 or 4, in a preferred embodiment 4,

R₁ indicates one or two a N-acetyllactosamine type elongation groups or nothing,

{ } and ( ) indicates branching which may be also present or absent,

other variables are as described in Formula HY1.

Preferred structures according to the formula HY3 include especially structures containing non-reducing end terminal Galβ, preferably Galβ3/4 forming a terminal N-acetyllactosamine structure. These are preferred as a special group of

Hybrid type structures, preferred as a group of specific value in characterization of balance of Complex N-glycan glycome and High mannose glycome:

GalβzGNβ2Mα3{Mα3Mα6}Mβ4GNXyR₂,

GalβzGNβ2Mα3{Mα6Mα6}Mβ4GNXyR₂,

GalβzGNβ2Mα3{Mα3(Mα6)Mα6}Mβ4GNXyR₂,

and/or elongated variants thereof preferred for carrying additional characteristic terminal structures useful for characterization of glycan materials

R₁GalβzGNβ2Mα3{Mα3Mα6}Mβ4GNXyR₂,

R₁GalβzGNβ2Mα3{Mα6Mα6}Mβ4GNXyR₂,

R₁GalβzGNβ2Mα3{Mα3(Mα6)Mα6}Mβ4GNXyR₂. Preferred elongated materials include structures wherein R₁ is a sialic acid, more preferably NeuNAc or NeuGc.

HSC

The present invention revealed novel stem cell specific glycans, with specific monosaccharide compositions and associated with differentiation status of stem cells and/or several types of stem cells and/or the differentiation levels of one stem cell type and/or lineage specific differences between stem cell lines.

N-Glycan Structures and Compositions Associated with Differentiation of Stem Cells

The invention revealed specific glycan monosaccharide compositions and corresponding structures, which associated with
vi) Blood derived stem cells especially cord blood derived stem cells
vii) Differentiated mononuclear blood cells

The preferred blood stem cells are hematopoietic stem cells more preferably CD133 or CD34 positive stem cells, most preferably cord blood derived CD133 or CD34 positive stem cells. Differentiated mononuclear blood cells are preferably CD133 or CD34 negative stem cells, most preferably cord blood derived CD133 or CD34 negative stem cells.

It is realized that the CD34+ cells resemble CD133+ cells, the invention also revealed that transferase expression of CD34+ cells was similar to the transferase expression of CD133+ cells. The invention is in a preferred embodiment directed to the use of the preferred mRNA markers according to the invention for the analysis of CD34+ cells.

It is realized that the structures revealed are useful for the characterization of the cells at different stages of development. The invention is directed to the use of the structures as markers for differentiation of blood derived stem cells.

The invention is further directed to the use of the specific glycans as markers enriched or increased at specific level of differentiation for the analysis of the cells at specific differentiation level.

N-Glycan Structures and Compositions are Associated with Individual Specific Differences Between Stem Cell Lines or Batches

The invention further revelead that specific glycan types are presented in the blood derived stem cell preparations on a specific differentiation stage in varying manner. It is realized that such individually varying glycans are useful for characterization of individual stem cell lines/preparations and batches. The specific structures of a individual cell preparation are useful for comparison and standardization of stem cell lines and cells prepared thereof.

The specific structures of a individual cell preparation are used for characterization of usefulness of specific stem cell line or batch or preparation for stem cell therapy in a patient, who may have antibodies or cell mediated immune defence recognizing the individually varying glycans.

The invention is especially directed to analysis of glycans with large and moderate variations as described in examples. The invention is especially directed to the analysis of individual specific differences, when there is a difference in the level of fucosylation and/or sialylation or in the level of mannosylation.

Analysis Methods by Mass Spectrometry or Specific Binding Reagents

The invention is specifically directed to the recognition of the terminal structures by either specific binder reagents and/or by mass spectrometric profiling of the glycan structures.

In a preferred embodiment the invention is directed to the recognition of the structures and/or compositions based on mass spectrometric signals corresponding to the structures.

The preferred binder reagents are directed to characteristic epitopes of the structures such as terminal epitopes and/or characteristic branching epitopes, such as monoantennary structures comprising a Manα-branch or not comprising a Manα-branch.

The preferred binder is an antibody, more preferably a monoclonal antibody.

In a preferred embodiment the invention is directed to a monoclonal antibody specifically recognizing at least one of the terminal epitope structures according to the invention.

Structures Associated with Blood Derived Stem Cells

The Tables show specific structure groups with specific monosaccharide compositions associated with the differentiation status of human blood derived stem cells in comparison to the mononuclear cells from blood.

The Structures Present and Enriched in Blood Stem Cell Cells

The invention revealed novel structures present in higher amounts in blood stem cell than in corresponding differentiated cells.

Structures in Specific CD133 Selected Blood Stem Cell Populations

CD133 is a commonly used marker for hematopoietic and other stem cells. The invention revealed especially variation CD133+ cells in comparison to CD133− cells.

Major N-glycans in CD133+ and CD133− cells were high-mannose and biantennary complex-type structures. CD133+ and CD133− cells also had monoantennary, hybrid, low-mannose and large complex-type N-glycans (Figures), for details see examples, showed polarization towards high-mannose type N-glycans (Figures), biantennary complex-type N-glycans with core composition 5-hexose 4-N-acetyhexosamine and sialylated monoantennary N-glycans (Figures). In contrast, CD133− cells had increased amounts of large complex-type N-glycans with core composition 6-hexose 5-N-acetylhexosamine or larger, sialylated hybrid-type N-glycans and low-mannose type N-glycans.

CD133+ Associated N-Glycan Groups CD133+ i)-CD133+ iii):

The invention revealed 3 groups of glycan compositions and glycan, named CD133+ i)—CD133+ iii, which are especially characteristic for the CD133 positive cells. All the groups share common N-glycan core structure according to Formula CCN and the glycan groups are further devided to specific Complex type and Mannose type structures. The differences in the expression are shown in Tables.

Complex Type Glycans Compositions and Structures Associated with CD133+ Cells

N-Glycan Group CD133+ i),

Biantennary-Size Complex-Type Sialylated N-Glycans with Core H5N4

A preferred group of specific expression blood derived stem cells, especially CD133+ cells, was revealed to be a specific group of Biantennary-size complex-type sialylated N-glycans with composition feature H5N4,

preferably including S1H5N4F1, S1H5N4, S2H5N4F1, S1H5N4F2, S2H5N4, and S1H5N4F3. Preferred subgroups of sialylated structures include mono-and disialyl-structures with low fucosylation (none or one) S1H5N4F1, S1H5N4, S2H5N4F1, S2H5N4, and monosialylated structures with high fucosylation S1H5N4F2, and S1H5N4F3.

The preferred structures are according to the formula:

SₖH₅N₄F_{q}

wherein

k is an integer being 1 or 2, preferably 1 for high fucosylation group and

q is an integer being 0-3, preferably 0 or 1 for low fucosylation group, and 2 or 3 for high fucosylation group.

Preferred Biantennary Structures with Low Fucosylation

The preferred biantennary structures according to the invention include structures according to the Formula:

[NeuAcα]₀₋₁GalβGNβ2Manα3([NeuAcα]₀₋₁GalβGNβ2Manα6)Manβ4GNβ4(Fucα6)₀₋₁GN,

The GalβGlcNAc structures are preferably Galβ4GlcNAc-structures (type II N-acetyllactosamine antennae). The presence of type 2 structures was revealed by specific β4-linkage cleaving galactosidase (D. pneumoniae).

In a preferred embodiment the sialic acid is NeuAcα6- and the glycan comprises the NeuAc linked to Manα3-arm of the molecule. The assignment is based on the presence of α6-linked sialic acid revealed by specific sialidase digestion and the known branch specificity of the α6-sialyltransferase (ST6GalI).

NeuAcα6GalβGNβ2Manα3([NeuAcα]₀₋₁GalβGNβ2Manα6)Manβ4GNβ4(Fucα6)₀₋₁GN, more preferably type II structures:

NeuAcα6Galβ4GNβ2Manα3([NeuAcα]₀₋₁Galβ4GNβ2Manα6)Manβ4GNβ4(Fucα6)₀₋₁GN.

The invention thus revealed preferred terminal epitopes, NeuAcα6GalβGN, NeuAcα6GalβGNβ2Man, NeuAcα6GalβGNβ2Manα3, to be recognized by specific binder molecules. It is realized that higher specificity preferred for application in context of similar structures can be obtained by using binder recognizing longer epitopes and thus differentiating e.g. between N-glycans and other glycan types in context of the terminal epitopes.

Preferred Biantennary Structures with High Fucosylation

The invention is preferably directed to biantennary structures with high fucosylation, preferably with two (difucosylated) or three fucose (trifucosylated) structures.

Preferred Difucosylated and Sialylated Structures

Preferred difucosylated sialylated structures include structures, wherein one fucose is in the core of the N-glycan and

a) one fucose on one arm of the molecule, and sialic acid is on the other arm (antenna of the molecule and the fucose is in Lewis x or H-structure:

Galβ4(Fucα3)GNβ2Manα3/6(NeuNAcαGalβGNβ2Manα6/3)Manβ4GNβ4(Fucα6)GN, and/or

Fucα2GalβGNβ2Manα3/6(NeuNAcαGalβGNβ2Manα6/3)Manβ4GNβ4(Fucα6)GN,

and when the sialic acid is α6-linked preferred antennary structures contain preferably the sialyl-lactosamine on α3-linked arm of the molecule according to formula:

Galβ4(Fucα3)GNβ2Manα6(NeuNAcα6Galβ4GNβ2Manα3)Manβ4GNβ4(Fucα6)GN,

and/or

Fucα2GalβGNβ2Manα6(NeuNAcα6Galβ4GNβ2Manα3)Manβ4GNβ4(Fucα6)GN.

It is realized that the structures, wherein the sialic acid and fucose are on different arms of the molecules can be recognized as characteristic specific epitopes.

b) Fucose and NeuAc are on the same arm in a structure:

NeuNAcα3Galβ3/4(Fucα4/3)GNβ2Manα3/6(GalβGNβ2Manα6/3)Manβ4GNβ4(Fucα6)GN,

and more preferably sialylated and fucosylated sialyl-Lewis x structures are preferred as a characteristic and bioactive structures: NeuNAcα3Galβ4(Fucα3)GNβ2Manα3/6(Galβ4GNβ2Manα6/3)Manβ4GNβ4(Fucα6)GN.

Preferred Sialylated Trifucosylated Structures

Preferred sialylated trifucosylated structures include glycans comprising core fucose and the terminal sialyl-Lewis x or sialyl-Lewis a, preferably sialyl-Lewis x due to relatively large presence of type 2 lactosamines, or Lewis y on either arm of the biantennary N-glycan according to the formulae:

NeuNAcα3Galβ4(Fucα3)GNβ2Manα3/6([Fucα]GalβGNβ2Manα6/3)Manβ4GNβ4(Fucα6)GN,

and/or

Fucα2Galβ4(Fucα3)GNβ2Manα3/6(NeuNAcα3/6GalβGNβ2Manα6/3)Manβ4GNβ4(Fucα6)GN. NeuNAc is preferably a-linked on the same arm as fucose due to known biosynthetic preferance. When the structure comprises NeuNAcα6, this is preferably linked to form NeuNAcα6Galβ4GlcNAcβ2Manα3-arm of the molecule. Galβ groups are preferably type II N-acetyllactosamine structures Galβ4-groups for blood stem cells.

N-Glycan Group CD 133 + ii)

Monoantennary-Size Sialylated N-Glycans

The invention further revealed characteristic unusual glycans with monoantennary type glycan compositions.

This preferred group includes of CD133+ cell associated structures includes: Monoantennary-size sialylated N-glycans with composition feature 3≦H≦4, preferably including S1H3N3F1, S1H3N3, S3H4N3F1, S1H4N3F1SP, S2H4N3, and optionally also S1H4N3F1 and/or S1H4N3.

Including linear monoantennary glycans S1H3N3F1, and S1H3N3 and branched monoantennary/hybrid type preferably with multiple charges S3H4N3F1, S1H4N3F1SP, S2H4N3,

and optionally also S1H4N3F1 and/or S1H4N3.

The preferred structures have monosacharide composition to the formula:

SₖHₘN₄F_{q}

wherein

k is an integer being 1, 2, or 3,

m is an integer being 3 or 4,

q is an integer being 0 or 1.

The preferred structures are according to the formula:

(NeuAc)ₙNeuAcα3/6GalβGlcNAcβ2Manα3(Manα6)₀₋₁Manβ4GlcNAcβ4(Fucα6)₀₋₁GlcNAc,

where in is 1 or 2, and the terminal sialic acids are preferably α8- or α9-linked, more preferably α8-linked more preferentially with type II N-acetyllactosamine antennae, wherein galactose residues are β1,4-linked

(NeuAc)ₙNeuAcα3/6Galβ4GlcNAcβ2Manα3(Manα6)₀₋₁Manβ4GlcNAcβ4(Fucα6)₀₋₁GlcNAc.

The preferred branched structures are according to the formula

(NeuAc)ₙNeuAcα3/6Galβ4GlcNAcβ2Manα3(Manα6)Manβ4GlcNAcβ4(Fucα6)₀₋₁GlcNAc and

preferred linear structures are according to the formula

(SP)₀₋₁(NeuAc)ₙNeuAcα3/6Galβ4GlcNAcβ2Manα3Manβ4GlcNAcβ4(Fucα6)₀₋₁GlcNAc,

optionally including in a specific embodiment a SP-structure (sulfate or fosfate structure).

Mannose Type Glycans Compositions and Structures Associated with CD133+ Cells

N-Glycan Group CD133 + iii)

High-Mannose Type Neutral N-Glycans

The preferred high-mannose type neutral N-glycans with composition feature N=2 and 5≦H≦9,

preferably including H5N2, H9N2, and H8N2.

The preferred structures are according to the formula:

[Mα2]ₙ₁Mα3{[Mα2]ₙ₃Mα6}Mα6{[Mα2]ₙ₆[Mα2]ₙ₇Mα3}Mβ4GNβ4GNyR₂

wherein n1, n3, n6, and n7are either independently 0 or 1;

y is anomeric linkage structure α and/or β or linkage from derivatized anomeric carbon, and

R₂ is reducing end hydroxyl, chemical reducing end derivative or natural asparagine

N-glycoside derivative such as asparagine N-glycosides including aminoacid and/or peptides derived from protein;

[ ] indicates determinant either being present or absent depending on the value of n1, n3, n6, n7; and

{ } indicates a branch in the structure;

M is D-Man, GN is N-acetyl-D-glucosamine , y is anomeric structure or linkage type, preferably beta to Asn.

y is anomeric linkage structure α and/or β or linkage from derivatized anomeric carbon, and

R₂ is reducing end hydroxyl, chemical reducing end derivative or natural asparagine N-glycoside derivative such as asparagine N-glycosides including aminoacid and/or peptides derived from protein;

Preferably the invention is directed to the High mannose type neutral glycans according to the formula ,with the provision that

all n1, n3, n6, and n7 are 1 (composition is H9N2) or

all n1, n3, n6, and n7 are 0 (composition is H5N2) or

one of n1, n3, n6 is 0, and others are 1, and n7 is 1, more preferably n3 is 0 (composition is H8N5).

The preferred structures in this group include:

Manα2Manα6(Manα2Manα3)Manα6(Manα2Manα2Manα3)Manβ4GlcNAcβ4GlcNAc, or

Manα2Manα6(Manα3)Manα6(Manα2Manα2Manα3)Manβ4GlcNAcβ4GlcNAc,

Manα6(Manα3)Manα6(Manα3)Manβ4GlcNAcβ4GlcNAc.

Structures and Compositions Associated with Differentiated Mononuclear Cells Cell Types from Blood

The invention revealed novel structures present in higher amount in differentiated mononuclear cells cells than in corresponding blood derived stem cells.

CD133− Associated N-Glycan Groups CD133− i)-CD133− iii):

The invention revealed 3 groups of glycan compositions and glycan, named CD133− i)-CD133− iii, which are especially characteristic for the CD133 negative cells. All the groups share common N-glycan core structure according to Formula CCN and the glycan groups are further devided to specific Complex type and Mannose type structures. The differences in the expression are shown in Tables.

Complex Type Glycans Compositions and Structures Associated with CD133− Cells

N-Glycan Group CD133− i)

Large Complex-Type Sialylated N-Glycans

The compositions indicate additional N-acetyllactosamine units in comparision to the biantennary N-glycans enriched in CD133+ cells.

The invention is especially directed to large complex-type sialylated N-glycans with composition feature N≧5 and H≧6,

preferably including S1H6N5F1, S2H6N5F1, S1H7N6F3, S1H7N6F1, S1H6N5, S3H6N5F1, S2H7N6F3, S1H6N5F3, S2H6N5F2, and S2H7N6F1. The glycans are further divided to groups of tri-LacNAc-glycans, comprising triantennary glycans, with core composition H6N5 and larger tetra-LacNAc glycans optionally including tetra-antennary glycans with core composition H7N6.

Preferred monosaccharide compositions are

the Formula

SₖHₙNₚF_{q}

wherein

k is integer from 1 to 3,

n is integer from 6 to 7,

p is integer from 5 to 6, and

q is integer being 0-3,

S is NeuSAc, G is NeuSGc, H is hexose selected from group D-Man or D-Gal, N is N-D-acetylhexosamine, preferably GlcNAc or GalNAc, more preferably GlcNAc, and F is L-fucose. The invention is directed compositions with n is 6 and p is 5 for triLacNAc-structures, and with n is 7 and p is 6 for tetra-LacNAc-structures.

The preferred tri- or tetraantennary structures are according to the formula:

{SAα3/6}ₛ₁LNβ2Mα3{{SAα3/6}ₛ₂LNβ2Mα6}Mβ4GNβ4{Fucα6}GN (I)

with one or two additional branch according to formula

{SAα3/6}ₛ₃LNβ, (IIb)

wherein s1, s2 and s3 are either 0 or 1, independently, with the provision at least one of s1, s2 or s3 is 1.

LN is N-acetyllactosaminyl also marked as GalβGN, GN is GlcNAc, M is mannosyl-, with the provision that LNβ2M can be further elongated and/or branched with one or several other monosaccharide residues such as galactose, fucose, SA or LN-unit(s) which may be further substituted by SAα-strutures,

is further substituted by one or two β6-, and/or β4-linked additional branches according to the formula IIb,

{ }, indicate groups present in a linear sequence, and { } indicates branching.

The SAα-groups are linked to either 3- or 6-position of neighboring Gal residue or on 6-position of GlcNAc, preferably 3- or 6-position of neighboring Gal residue.

Preferred Tri-LacNAc and Triantennary Glycans

The invention is especially directed to tri-LacNAc, preferably triantennary N-glycans having compositions S1H6N5F1, S2H6N5F1, S1H6N5, S3H6N5F1, S1H6N5F3, and S2H6N5F2. Presence of triantennary structures was revealed by specific galactosidase digestions. A preferred type of triantennary N-glycans includes one synthesized by MGAT4. The triantennary N-glycan comprises in a preferred embodiment a core fucose residue. The preferred terminal epitopes include Lewis x, sialyl-Lewis x, H- and Lewis y antigens.

The preferred triantennary structures are according to the Formula Tri1

{SAα3/6}ₛ₁LNβ2Mα3{{SAα3/6}ₛ₂LNβ2({SAα3/6}ₛ₃LNβ4)Mα6}Mβ4GN62 4{Fucα6}

### GN,

wherein ( ) indicates branch and other variables are as described above for Formula I.

The invention especially revealed triantennary structures, which are specific for CD133 negative cells.

Preferred Tetra-LacNAc and Tetraantennary Glycans

The invention is especially directed to tri-LacNAc, preferably triantennary N-glycans having compositions S1H7N6F3, S1H7N6F1, S2H7N6F3, and S2H7N6F1.

Preferred Tetra-LacNac Including Tetraantennary and/or Polylactosamine Structures

The invention is further directed to monosaccharide compositions and glycan corresponding to monosaccharide compositions S1H7N6F2, and S1H7N6F3, which were assigned to correspond to tetra-antennary and/or poly-N-acetyllactosamine epitope comprising N-glycans such as ones with terminal GalβGlcNAcβ3GalβGlcNAcβ-, more preferably type 2 structures Galβ4GlcNAcβ3Galβ4GlcNAcβ-.

The preferred tetra-antennary structures are according to the Formula Tet1

{SAα3/6}ₛ₁LNβ2({SAα3/6}ₛ₄LNβ4/6)Mα3{{SAα3/6}ₛ₂LNβ({SAα3/6}ₛ₃LNβ4)Mα6}Mβ4GNβ4{Fucα6}GN,

wherein ( ) indicates branch, s4 is 0 or 1 and other variables are as described above for Formula I.

N-Glycan Group CD133− ii)

Hybrid-Type Sialylated N-Glycans

The invention is especially directed to hybrid-type sialylated N-glycans with composition feature 5≦H≦6, preferably including S1H6N3, S1H5N3, and S1H6N3F1.

Preferred monosaccharide compositions are

the Formula

S₁HₙN₃F_{q}

wherein

n is integer being 5 or 6, and

q is integer being 0 or 1.

The preferred structures are according to the formula:

NeuNAcα3/6Galβ4GNβ2Mα3{[Mα3]ₘ₁[(Mα6)]ₘ₂Mα6}Mβ4GNXyR₂,

wherein m1, m2, are either 0 or 1, independently,

z is linkage position to GN being 3 or 4, in a preferred embodiment 4,

R₁ indicates one or two N-acetyllactosamine type elongation groups; NeuAcα3/6 or nothing,

{ } and ( ) indicates branching which may be also present or absent, other variables are as described in Formula HY1.

More preferably the structures are

NeuNAcα3/6Gal⊕4GNβ2Mα3{Mα3(Mα6)Mα6}Mβ4GNXyR₂,

And hex5 structures

NeuNAcα3/6Galβ4GNβ2Mα3{Mα3Mα6}Mβ4GNXyR₂, and

NeuNAcα3/6Galβ4GNβ2Mα3{Mα6Mα6}Mβ4GNXyR₂.

N-Glycan Group CD133− iv)

The Tables and Figures indicate that terminal HexNAc group structures with compositions SH5N5 and SH5N5F are especially specific for the differentiated blood cells, preferably CD133− cells. The invention is directed to the corresponding biantennary N-glycans with two lactosamines and terminal GlcNAc structures comprising GlcNAc substitutions such as bisecting GlcNAc in the N-glycan core Manβ4GlcNAc epitope.

Mannose Type Glycans Compositions and Structures Associated with CD133− Cells

N-Glycan Group CD133− iii)

Low-Mannose Type Neutral N-Glycans

The invention is especially directed to low-mannose type neutral N-glycans with composition feature N=2 and 1≦H≦4,

preferably including H3N2F1, H3N2, H2N2F1, H2N2, H1N2, and H4N2.

Preferred monosaccharide compositions are

the Formula

HₙN₂F_{q}

wherein

n is integer from 1 to 3,

q is integer being 0 or 1.

The preferred structures are according to the Formula:

[Mα3]ₙ₂{[Mα6)]ₙ₄}[Mα6]ₙ₅{[Mα3]ₙ₈}Mβ4GNβ4[{Fucα5}]ₘGNyR₂

wherein n2, n4, n5, n8, and m are either independently 0 or 1; [ ] indicates determinant being either present or absent depending on the value of n2, n4, n5, n8 and m, { } indicates a branch in the structure;

y and R2 are as indicated for Formula M2.

and with the provision that at least one of n2, n4 and n8 is 0.

Preferred non-fucosylated Low mannose N-glycans are according to the Formula:

Mα6Mβ4GNβ4GNyR₂

Mα3Mβ4GNβ4GNyR₂ and

Mα6{Mα3}Mβ4GNβ4GNyR₂.

Mα6Mα6{Mα3}Mβ4GNβ4GNyR₂

Mα3Mα6{Mα3}Mβ4GNβ4GNyR₂

Preferred Individual Structures of Fucosylated Low-Mannose Glycans

Small fucosylated low-mannose structures are especially unusual among known N-linked glycans and form a characteristic glycan group useful for the methods according to the invention, especially analysis and/or separation of cells according to the present invention. These include:

Mβ4GNβ4(Fucα6)GNyR₂

Mα6Mβ4GNβ4(Fucα6)GNyR₂,

Mα3Mβ4GNβ4(Fucα6)GNyR₂,

Mα6Mα6{Mα3}Mβ4GNβ4(Fucα6)GNyR₂, and

Mα3Mα6{Mα3}Mβ4GNβ4(Fucα6)GNyR₂.

In a specific embodiment the low mannose glycans include rare structures based on unusual mannosidase degradation

Manα2Manα2Manα3Manβ4GNβ4(Fucα6)₀₋₁GN, and

Manα2Manα3Manβ4GNβ4(Fucα6)₀₋₁GN.

Novel Terminal HexNAc N-Glycan Compositions from Stem Cells

The inventors studied human stem cells. The data revealed a specific group of altering glycan structures referred as terminal HexNAc. The data reveals changes of preferred signals in context of differentiation. The terminal HexNAc structures were assigned to include terminal N-acetylglucosamine structures by cleavage with N-acetylglucosamidase enzymes.

Preferred N-Glucans According to Structural Subgroups with Terminal HexNAc

The inventors found that there are differentiation stage specific differences with regard to terminal HexNAc containing N-glycans characterized by the formulae: n_{HexNAc}=n_{Hex}≧5 and n_{dHex}≧1 (group I), or: n_{HexNAc}=n_{Hex}≧5 and n_{dHex}=0 (group II). The present data demonstrated that these glycans were 1) detected in various N-glycan samples isolated from both stem cells, including, cord blood and bone marrow hematopoietic stem cells (CB and BM HSC), and CB HSC further including CD34+, CD133+, and lin− (lineage negative) cells, and cells directly or indirectly differentiated from these cell types; and 2) overexpressed in the analyzed differentiated cells when compared to the corresponding stem cells. There was independent expression between groups I and group II and therefore, the N-glycan structure group determined by the formula n_{HexNAc}=n_{Hex}≧5 is divided into two independently expressed subgroups I and II as described above.

The inventors also found differential expression of glycan signals corresponding to N-glycans Hex₃HexNAc₅ and Hex₃HexNAc₅dHex₁ that have the same compositional feature that the groups II and I above, respectively. Specifically, in analysis of HSC isolated from different sources it was found that Hex₃HexNAc₅dHex₁ was highly expressed in CD133+ and lin− cells, moderately expressed in all other CB MNC fractions including CD34+ and CD34− cells, and no expression was detected in CD34+ cells isolated from adult peripheral blood.

Based on the known specificities of the biosynthetic enzymes synthesizing N-glycan core α1,6-linked fucose and β1,4-linked bisecting GlcNAc, group II preferably corresponds to bisecting GlcNAc type N-glycans while group I preferentially corresponds to other terminal HexNAc containing N-glycans, preferentially with a branching HexNAc in the N-glycan core structure, more preferentially including structures with a branching GlcNAc in the N-glycan core structure. In a specific embodiment the glycan structures of this group includes core fucosylated bisecting GlcNAc comprising N-glycan, wherein the additional GlcNAc is GlcNAcβ4 linked to Manβ4GlcNAc epitope forming epitope structure GlcNAcβ4Manβ4GlcNAc preferably between the complex type N-glycan branches.

In a preferred embodiment of the present invention, such structures include GlcNAc linked to the 2-position of the β1,4-linked mannose. In a further preferred embodiment of the present invention, such structures include GlcNAc linked to the 2-position of the β1,4-linked mannose as described for LEC14 structure (Raju and Stanley J. Biol Chem (1996) 271, 7484-93), this is specifically preferred embodiment, supported by analysis of gene expression data and glycosyltransferase specificities. In a further preferred embodiment of the present invention, such structures include GlcNAc linked to the 6-position of the β1,4-linked GlcNAc of the N-glycan core as described for LEC14 structure (Raju, Ray and Stanley J. Biol Chem (1995) 270, 30294-302).

The invention is specifically directed to further analysis of the subtypes of the group I glycans comprising structures according to the group I. The invention is further directed to production of specific binding reagents against the N-glycan core marker structures and use of these for analysis of the preferred cancer marker structures. The invention is further directed to the analysis of LEC14 and/or 18 structures by negative recognition by lectins PSA (pisum sativum) or lntil (Lens culinaris) lectin or core Fuc specific monoclonal antibodies, which binding is prevented by the GlcNAcs.

Invention is specifically directed to N-glycan core marker structure, wherein the disaccharide epitope is Manβ4GlcNAc structure in the core structure of N-linked glycan according to the Formula CGN.

The invention is further directed to the N-glycan core marker structure and marker glycan compositions comprising structures of Formula CGN, wherein Manα3/Manα6− residues are elongated to the complex type, especially biantennary structures and n3 is 1 and wherein the Manβ4GlcNAc-epitope comprises the GlcNAc substitutions.

The invention is further directed to the N-glycan core marker structure and marker glycan compositions comprising structures of Formula CGN, wherein Manα3/Manα6− residues are elongated to the complex type, especially biantennary structures and n3 is 1 and wherein the Manβ4GlcNAc-epitope comprises between 1-8% of the GlcNAc substitutions.

The invention is further directed to the N-glycan core marker structure and marker glycan compositions comprising structures of Formula CGN, wherein the structure is selected from the group:

[GlcNAcβ2Manα3](GlcNAcβ2Manα6)Manβ4GlcNAcβ4(Fucα6)ₙ₃GlcNAcxR,

[Galβ4GlcNAcβ2Manα3](Galβ4GlcNAcβ2Manα6)

Manβ4GlcNAcβ4(Fucα6)ₙ₃GlcNAcxR,

and sialylated variants thereof when SA is α3 and or α6-linked to one or two Gal residues and Manβ4 or GlcNAcβ4 is substituted by GlcNAc.

The invention is further directed to the N-glycan core marker structure and marker glycan compositions comprising of Formula CGN, wherein the Manβ4GlcNAc-epitope comprises and the GlcNAc residue is β2-linked to Manβ4 forming epitope GlcNAcβ2Manβ4.

The invention is further directed to the N-glycan core marker structure and marker glycan compositions comprising of Formula CGN, wherein the Manβ4GlcNAc-epitope comprises and the GlcNAc residue is 6-linked to GlcNAc of the epitope forming epitope Manβ4(GlcNAc6)GlcNAc.

The invention is further directed to the N-glycan core marker structure and marker glycan compositions comprising of Formula CGN, wherein the Manβ4GlcNAc-epitope comprises and the GlcNAc residue is 4-linked to GlcNAc of the epitope forming epitope GlcNAcβ4Manβ4GlcNAc.

Recognition of Structures from Glycome Materials and on Cell Surfaces by Binding Methods

The present invention revealed that beside the physicochemical analysis by NMR and/or mass spectrometry several methods are useful for the analysis of the structures. The invention is especially directed to a method:
i) Recognition by molecules binding glycans referred as the binders These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived thereof, when the proteins are selected from the group of monoclonal antibody, glycosidase, glycosyl transferring enzyme, plant lectin, animal lectin or a peptide mimetic thereof, and wherein the binder may include a detectable label structure.

The genus of enzymes in carbohydrate recognition is continuous to the genus of lectins (carbohydrate binding proteins without enzymatic activity).

a) Native glycosyltransferases (Rauvala et al.(1983) PNAS (USA) 3991-3995) and glycosidases (Rauvala and Hakomori (1981) J. Cell Biol. 88, 149-159) have lectin activities.

b) The carbohydrate binding enzymes can be modified to lectins by mutating the catalytic amino acid residues (see WO9842864; Aalto J. et al. Glycoconjugate J. (2001, 18(10); 751-8; Mega and Hase (1994) BBA 1200 (3) 331-3).

c) Natural lectins, which are structurally homologous to glycosidases are also known indicating the continuity of the genus enzymes and lectins (Sun, Y-J. et al. J. Biol. Chem. (2001) 276 (20) 17507-14).

The genus of the antibodies as carbohydrate binding proteins without enzymatic activity is also very close to the concept of lectins, but antibodies are usually not classified as lectins.

Obviousness of the Peptide Concept and Continuity with the Carbohydrate Binding Protein Concept

It is further realized that proteins consist of peptide chains and thus the recognition of carbohydrates by peptides is obvious. E.g. it is known in the art that peptides derived from active sites of carbohydrate binding proteins can recognize carbohydrates (e.g. Geng J-G. et al (1992) J. Biol. Chem. 19846-53).

As described above antibody fragment are included in description and genetically engineed variants of the binding proteins. The obvious genetically engineered variants would included truncated or fragment peptides of the enzymes, antibodies and lectins.

Revealing Cell or Differantation and Individual Specific Terminal Variants of Structures

The invention is directed use the glycomics profiling methods for the revealing structural features with on-off changes as markers of specific differentiation stage or quantitative difference based on quantitative comparison of glycomes. The individual specific variants are based on genetic variations of glycosyltransferases and/or other components of the glycosylation machinery preventing or causing synthesis of individual specific structure.

Terminal Structural Epitopes

We have previously revealed glycome compositions of human glycomes, here we provide structural terminal epitopes useful for the characterization of stem cell glycomes, especially by specific binders.

The examples of characteristic altering terminal structures includes expression of competing terminal epitopes created as modification of key homologous core Galβ-epitopes, with either the same monosaccharides with difference in linkage position Galβ3GlcNAc, and analogue with either the same monosaccharides with difference in linkage position Galβ4GlcNAc; or the with the same linkage but 4-position epimeric backbone Galβ3GalNAc. These can be presented by specific core structures modifying the biological recognition and function of the structures. Another common feature is that the similar Galβ-structures are expressed both as protein linked (O- and N-glycan) and lipid linked (glycolipid structures). As an alternative for α2-fucosylation the terminal Gal may comprise NAc group on the same 2 position as the fucose. This leads to homologous epitopes GalNAcβ4GlcNAc and yet related GalNAcβ3Gal-structure on characteristic special glycolipid according to the invention.

The invention is directed to novel terminal disaccharide and derivative epitopes from human stem cells, preferably from human embryonal stem cells or adult stem cells, when these are not hematopoietic stem cells, which are preferably mesenchymal stem cells. It should realized that glycosylations are species, cell and tissue specific and results from cancer cells usually differ dramatically from normal cells, thus the vast and varying glycosylation data obtained from human embryonal carcinomas are not actually relevant or obvious to human embryonal stem cells (unless accidentally appeared similar). Additionally the exact differentiation level of teratocarcinomas cannot be known, so comparison of terminal epitope under specific modification machinery cannot be known. The terminal structures by specific binding molecules including glycosidases and antibodies and chemical analysis of the structures.

The present invention reveals group of terminal Gal(NAc)β1-3/4Hex(NAc) structures, which carry similar modifications by specific fucosylation/NAc-modification, and sialylation on corresponding positions of the terminal disaccharide epitopes. It is realized that the terminal structures are regulated by genetically controlled homologous family of fucosyltransferases and sialyltransferases. The regulation creates a characteristic structural patterns for communication between cells and recognition by other specific binder to be used for analysis of the cells. The key epitopes are presented in the TABLE. The data reveals characteristic patterns of the terminal epitopes for each types of cells, such as for example expression on hESC-cells generally much Fucα-structures such as Fucα2-structures on type 1 lactosamine (Galβ3GlcNAc), similarily β3-linked core I Galβ3GlcNAcα, and type 4 structure which is present on specific type of glycolipids and expression of α3-fucosylated structures, while α6-sialic on type II N-acetylalactosamine appear on N-glycans of embryoid bodies and st3 embryonal stem cells. E.g. terminal type lactosamine and poly-lactosamines differentiate mesenchymal stem cells from other types. The terminal Galb-information is preferably combined with information about

The invention is directed especially to high specificity binding molecules such as monoclonal antibodies for the recognition of the structures.

The structures can be presented by Formula T1. the formula describes first monosaccharide residue on left, which is a β-D-galactopyranosyl structure linked to either 3 or 4-position of

the α- or β-D-(2-deoxy-2-acetamido)galactopyranosyl structure, when R₅ is OH, or β-D-(2-deoxy-2-acetamido)glucopyranosyl, when R₄ comprises O—. The unspecified stereochemistry of the reducing end in formulas T1 and T2 is indicated additionally (in claims) with curved line. The sialic acid residues can be linked to 3 or 6-position of Gal or 6-position of GlcNAc and fucose residues to position 2 of Gal or 3- or 4-position of GlcNAc or position 3 of Glc.

The invention is directed to Galactosyl-globoside type structures comprising terminal Fucα2-revealed as novel terminal epitope Fucα2Galβ3GalNAcβ or Galβ3GalNAcβGalα3 -comprising isoglobotructures revealed from the embryonal type cells.

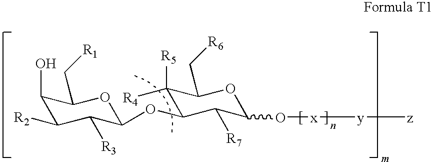

wherein

X is linkage position

R₁, R₂, and R₆ are OH or glycosidically linked monosaccharide residue Sialic acid, preferably Neu5Acα2 or Neu5Gc α2, most preferably Neu5Acα2 or

R₃, is OH or glycosidically linked monosaccharide residue Fucα1 (L-fucose) or N-acetyl (N-acetamido, NCOCH₃);

R₄, is H, OH or glycosidically linked monosaccharide residue Fucα1 (L-fucose),

R₅ is OH, when R₄ is H, and R₅ is H, when R₄ is not H;

R7 is N-acetyl or OH

X is natural oligosaccharide backbone structure from the cells, preferably N-glycan,

O-glycan or glycolipid structure; or X is nothing, when n is 0,

Y is linker group preferably oxygen for O-glycans and O-linked terminal oligosaccharides and glycolipids and N for N-glycans or nothing when n is 0;

Z is the carrier structure, preferably natural carrier produced by the cells, such as protein or lipid, which is preferably a ceramide or branched glycan core structure on the carrier or H;

The arch indicates that the linkage from the galactopyranosyl is either to position 3 or to position 4 of the residue on the left and that the R4 structure is in the other position 4 or 3;

n is an integer 0 or 1, and m is an integer from 1 to 1000, preferably 1 to 100, and most preferably 1 to 10 (the number of the glycans on the carrier),

With the provisions that one of R2 and R3 is OH or R3 is N-acetyl,

R6 is OH, when the first residue on left is linked to position 4 of the residue on right:

X is not Galα4Galβ4Glc, (the core structure of SSEA-3 or 4) or R3 is Fucosyl

R7 is preferably N-acetyl, when the first residue on left is linked to position 3 of the residue on right:

Preferred terminal β3-linked subgroup is represented

by Formula T2 indicating the situation, when the first residue on the left is linked to the 3 position with backbone structures Gal(NAc)β3Gal/GlcNAc.

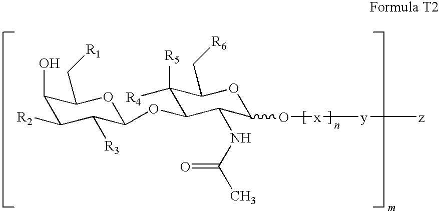

Wherein the variables including R₁ to R₇

are as described for T1

Preferred terminal β4-linked subgroup is represented by the Formula 3

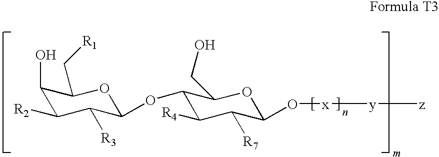

Wherein the variables including R₁ to R₄ and R7

are as described for T1 with the provision that

R₄, is OH or glycosidically linked monosaccharide residue Fucα1 (L-fucose),

Alternatively the epitope of the terminal structure can be represented by Formulas T4 and T5

Core Galβ-epitopes formula T4:

Galβ1-xHex(NAc)ₚ,

x is linkage position 3 or 4,

and Hex is Gal or Glc

with provision

p is 0 or 1

when x is linkage position 3, p is 1 and HexNAc is GlcNAc or GalNAc,

and when x is linkage position 4, Hex is Glc.

The core Galβ1-3/4 epitope is optionally substituted to hydroxyl

by one or two structures SAα or Fucα, preferably selected from the group

Gal linked SAα3 or SAα6 or Fucα2, and

Glc linked Fucα3 or GlcNAc linked Fucα3/4.

[Mα]ₘGalβ1-x[Nα]ₙHex(NAc)ₚ, Formula T5

wherein m, n and p are integers 0, or 1, independently

Hex is Gal or Glc,

X is linkage position

M and N are monosaccharide residues being

independently nothing (free hydroxyl groups at the positions)

and/or

SA which is Sialic acid linked to 3-position of Gal or/and 6-position of HexNAc

and/or

Fuc (L-fucose) residue linked to 2-position of Gal

and/or 3 or 4 position of HexNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently.

The exact structural details are essential for optimal recognition by specific binding molecules designed for the analysis and/or manipulation of the cells.

The terminal key Galβ-epitopes are modified by the same modification monosaccharides NeuX (X is 5 position modification Ac or Gc of sialic acid) or Fuc, with the same linkage type alfa(modifying the same hydroxyl-positions in both structures.

NeuXα3, Fucα2 on the terminal Galβ of all the epitopes and NeuXα6 modifying the terminal Galβ of Galβ4GlcNAc, or HexNAc, when linkage is 6 competing

or Fucα modifying the free axial primary hydroxyl left in GlcNAc (there is no free axial hydroxyl in GalNAc-residue).

The preferred structures can be divided to preferred Galβ1-3 structures analogously to T2,

[Mα]ₘGalβ1-3[Nα]ₙHexNAc, Formula T6:

Wherein the variables are as described for T5.

The preferred structures can be divided to preferred Galβ1-4 structures analogously to T4,

[Mα]ₘGalβ1-4[Nα]ₙGlc(NAc)ₚ, Formula T7:

Wherein the variables are as described for T5.

These are preferred type II N-acetyllactosamine structures and related lactosylderivatives, in a preferred embodiment p is 1 and the structures includes only type 2 N-acetyllactosamines. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells). It is notable that various fucosyl- and or sialic acid modification created characteristic pattern for the stem cell type.

Preferred Type I and Type II N-Acetyllactosamine Structures

The preferred structures can be divided to preferred type one (I) and type two (II) N-acetyllactosamine structures comprising oligosaccharide core sequence Galβ1-3/4 GlcNAc structures analogously to T4,

[Mα]ₘGalβ1-3/4[Nα]ₙGlcNAc, Formula T8:

Wherein the variables are as described for T5.

The preferred structures can be divided to preferred Galβ1-3 structures analogously to T8,

[Mα]ₘGalβ1-3[Nα]ₙGlcNAc Formula T9:

Wherein the variables are as described for T5.

These are preferred type I N-acetyllactosamine structures. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells). It is notable that various fucosyl- and or sialic acid modification created characteristic pattern for the stem cell type.

The preferred structures can be divided to preferred Galβ1-4GlcNAc core sequence comprising structures analogously to T8,

[Mα]ₘGalβ1-4 [Nα]ₙGlcNAc Formula T10:

Wherein the variables are as described for T5.

These are preferred type II N-acetyllactosamine structures. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells).

It is notable that various fucosyl- and or sialic acid modificationally N-acetyllactosamine structures create especially characteristic pattern for the stem cell type. The invention is further directed to use of combinations binder reagents recognizing at least two different type I and type II acetyllactosamines including at least one fucosylated or sialylated varient and more preferably at least two fucosylated variants or two sialylated variants

Preferred structures comprising terminal Fucα2/3/4-structures

The invention is further directed to use of combinations binder reagents recognizing:
a) type I and type II acetyllactosamines and their fucosylated variants, and in a preferred embodiment
b) non-sialylated fucosylated and even more preferably
c) fucosylated type I and type II N-acetyllactosamine structures preferably comprising Fucα2-terminal and/or Fucα3/4-branch structure and even more preferably
d) fucosylated type I and type II N-acetyllactosamine structures preferably comprising Fucα2-terminal
for the methods according to the invention of various stem cells especially embryonal type and mesenchymal stem cells and differentiated variants thereof.

Preferred subgroups of Fucα2-structures includes monofucosylated H type and H type II structures, and difucosylated Lewis b and Lewis y structures.

Preferred subgroups of Fucα3/4-structures includes monofucosylated Lewis a and Lewis x structures, sialylated sialyl-Lewis a and sialyl-Lewis x- structures and difucosylated Lewis b and Lewis y structures.

Preferred type II N-acetyllactosamine subgroups of Fucα3-structures includes monofucosylated Lewis x structures, and sialyl-Lewis x− structures and Lewis y structures.

Preferred type I N-acetyllactosamine subgroups of Fucα4-structures includes monofucosylated Lewis a sialyl-Lewis a and difucosylated Lewis b structures.

The invention is further directed to use of at least two differently fucosylated type one and or and two N-acetyllactosamine structures preferably selected from the group monofucosylated or at least two difucosylated, or at least one monofucosylated and one difucosylated structures.

The invention is further directed to use of combinations binder reagents recognizing fucosylated type I and type II N-acetyllactosamine structures together with binders recognizing other terminal structures comprising Fucα2/3/4-comprising structures, preferably Fucα2-terminal structures, preferably comprising Fucα2Galβ3GalNAc-terminal, more preferably Fucα2Galβ3GalNAcα/β and in especially preferred embodiment antibodies recognizing Fucα2Galβ3GalNAcβ− preferably in terminal structure of Globo- or isoglobotype structures.

Preferred Globo- and ganglio core type-structures

The invention is further directed to general formula comprising globo and gangliotype Glycan core structures according to formula

[M]ₘGalβ1-x[Nα]ₙHex(NAc)ₚ, wherein m, n and p are integers 0, or 1, independently Formula T11

Hex is Gal or Glc, X is linkage position;

M and N are monosaccharide residues being

independently nothing (free hydroxyl groups at the positions)

and/or

SAα which is Sialic acid linked to 3-position of Gal or/and 6-position of HexNAc

Galα linked to 3 or 4-position of Gal, or

GalNAcβ linked to 4-position of Gal and/or

Fuc (L-fucose) residue linked to 2-position of Gal

and/or 3 or 4 position of HexNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently, and

with the provision that when M is Gala then there is no sialic acid linked to Galβ1,

and

n is 0 and preferably x is 4.

with the provision that when M is GalNAcβ, then there is no sialic acid α6-linked to Galβ1, and n is 0 and x is 4.

The invention is further directed to general formula comprising globo and gangliotype Glycan core structures according to formula

[M][SAα3]ₙGalβ1-4Glc(NAc)ₚ, Formula T12

wherein n and p are integers 0, or 1, independently

M is Galα linked to 3 or 4-position of Gal, or GalNAcβ linked to 4-position of Gal and/or SAα is Sialic acid branch linked to 3-position of Gal

with the provision that when M is Galα then there is no sialic acid linked to Galβ1 (n is 0).

The invention is further directed to general formula comprising globo and gangliotype Glycan core structures according to formula

[M][SAα]ₙGalβ1-4Glc, Formula T13

wherein n and p are integer 0, or 1, independently

M is Galα linked to 3 or 4-position of Gal, or

GalNAcβ linked to 4-position of Gal

and/or

SAα which is Sialic acid linked to 3-position of Gal

with the provision that when M is Galα then there is no sialic acid linked to Galβ1 (n is 0).

The invention is further directed to general formula comprising globo type Glycan core structures according to formula

Galα3/4Galβ1-4Glc. Formula T14

The preferred Globo-type structures includes Galα3/4Galβ1-4Glc, GalNAcβ3Galα3/4Galβ4Glc, Galα4Galβ4Glc (globotriose, Gb3), Galα3Galβ4Glc (isoglobotriose), GalNAcβ3Galα4Galβ4Glc (globotetraose, Gb4 (or G14)), and Fucα2Galβ3GalNAcβ3Galα3/4Galβ4Glc. or

when the binder is not used in context of non-differentiated embryonal or mesenchymal stem cells or the binder is used together with another preferred binder according to the invention, preferably an other globo-type binder the preferred binder targets further includes

Galβ3GalNAcβ3Galα4Galβ4Glc (SSEA-3 antigen) and/or

NeuAcα3Galβ3GalNAcβ3Galα4Galβ4Glc (SSEA-4 antigen) or terminal non-reducing end di or trisaccharide epitopes thereof.

The preferred globotetraosylceramide antibodies does not recognize non-reducing end elongated variants of GalNAcβ3Galα4Galβ4Glc. The antibody in the examples has such specificity as

The invention is further directed to binders for specific epitopes of the longer oligosaccharide sequences including preferably NeuAcα3Galβ3GalNAc, NeuAcα3Galβ3GalNAcβ, NeuAcα3Galβ3GalNAcβ3Galα4Gal when these are not linked to glycolipids and novel fucosylated target structures:

Fucα2Galβ3GalNAcβ3Galα3/4Gal, Fucα2Galβ3GalNAcβ3Galα, Fucα2Galβ3GalN Acβ3Gal, Fucα2Galβ3GalNAcβ3, and Fucα2Galβ3GalNAc.

The invention is further directed to general formula comprising globo and gangliotype Glycan core structures according to formula

[GalNAcβ4][SAα]ₙGalβ1-4Glc, wherein n and p are integer 0, or 1, independently Formula T15

GalNAcβ linked to 4-position of Gal and/or SAα which is Sialic acid branch linked to 3-position of Gal.

The preferred Ganglio-type structures includes GalNAcβ4Galβ1-4Glc, GalNAcβ4[SAα3]Galβ1-4Glc, and Galβ3GalNAcβ4[SAα3]Galβ1-4Glc.

The preferred binder target structures further include glycolipid and possible glycoprotein conjugates of of the preferred oligosaccharide sequences. The preferred binders preferably specifically recognizes at least di- or trisaccharide epitope

GalNAcα-Structures

The invention is further directed to recognition of peptide/protein linked GalNAcα-structures according to the Formula T16:[SAα6]ₘGalNAcα[Ser/Thr]ₙ-[Peptide]ₚ, wherein m, n and p are integers 0 or 1, independently,

wherein SA is sialic acid preferably NeuAc,Ser/Thr indicates linking serine or threonine residues, Peptide indicates part of peptide sequence close to linking residue,

with the provisio that either m or n is 1.

Ser/Thr and/or Peptide are optionally at least partially necessary for recognition for the binding by the binder. It is realized that when Peptide is included in the specificity, the antibody have high specificity involving part of a protein structure. The preferred antigen sequences of sialyl-Tn: SAα6GalNAcα, SAα6GalNAcαSer/Thr, and SAα6GalNAcαSer/Thr-Peptide and Tn-antigen: GalNAcαSer/Thr, and GalNAcαSer/Thr-Peptide. The invention is further directed to the use of combinations of the GalNAcα-structures and combination of at least one GalNAcα-structure with other preferred structures.

Combinations of Preferred Binder Groups

The present invention is especially directed to combined use of at least a) fucosylated, preferably α2/3/4-fucosylated structures and/or b) globo-type structures and/or c) GalNAcα-type structures. It is realized that using a combination of binders recognizing structures involving different biosynthesis and thus having characteristic binding profile with a stem cell population. More preferably at least one binder for a fucosylated structure and and globostructures, or fucosylated structure and GalNAcα-type structure is used, most preferably fucosylated structure and globostructure are used.

Fucosylated and Non-Modified Structures

The invention is further directed to the core disaccharide epitope structures when the structures are not modified by sialic acid (none of the R-groups according to the Formulas T1-T3 or M or N in formulas T4-T7 is not sialic acid.

The invention is in a preferred embodiment directed to structures, which comprise at least one fucose residue according to the invention. These structures are novel specific fucosylated terminal epitopes, useful for the analysis of stem cells according to the invention. Preferably native stem cells are analyzed.

The preferred fucosylated structures include novel α3/4fucosylated markers of human stem cells such as (SAα3)₀ₒᵣ₁Galβ3/4(Fucα4/3)GlcNAc including Lewis x and and sialylated variants thereof.

Among the structures comprising terminal Fucα1-2 the invention revealed especially useful novel marker structures comprising Fucα2Galβ3GalNAcα/β and Fucα2Galβ3(Fucα4)₀ₒᵣ₁GlcNAcβ, these were found useful studying embryonal stem cells. A especially preferred antibody/binder group among this group is antibodies specific for Fucα2Galβ3GlcNAcβ, preferred for high stem cell specificity. Another preferred structural group includes Fucα2Gal comprising glycolipids revealed to form specific structural group, especially interesting structure is globo-H-type structure and glycolipids with terminal Fucα2Galβ3GalNAcβ, preferred with interesting biosynthetic context to earlier speculated stem cell markers.

Among the antibodies recognizing Fucα2Galβ4GlcNAcβ substantial variation in binding was revealed likely based on the carrier structures, the invention is especially directed to antibodies recognizing this type of structures, when the specificity of the antibody is similar to the ones binding to the embryonal stem cells as shown in Examples with fucose recognizing antibodies. The invention is preferably directed to antibodies recognizing Fucα2Galβ4GlcNAcβ on N-glycans, revealed as common structural type in terminal epitope Tables. In a separate embodiment the antibody of the non-binding clone is directed to the recognition of the feeder cells.

The preferred non-modified structures includes Galβ4Glc, Galβ3GlcNAc, Galβ3GalNAc, Galβ4GlcNAc, Galβ3GlcNAcβ, Galβ3GalNAcβ/α, and Galβ4GlcNAcβ. These are preferred novel core markers characteristics for the various stem cells. The structure Galβ3GlcNAc is especially preferred as novel marker observable in hESC cells. Preferably the structure is carried by a glycolipid core structure according to the invention or it is present on an O-glycan. The non-modified markers are preferred for the use in combination with at least one fucosylated or/and sialylated structure for analysis of cell status.

Additional preferred non-modified structures includes GalNAcβ-structures includes terminal LacdiNAc, GalNAcβ4GlcNAc, preferred on N-glycans and GalNAcβ3Gal GalNAcβ3Gal present in globoseries glycolipids as terminal of globotetraose structures.

Among these characteristic subgroup of Gal(NAc)β3-comprising Galβ3GlcNAc, Galβ3GalNAc, Galβ3GlcNAcβ, Galβ3GalNAcβ/α, and GalNAcβ3Gal GalNAcβ3Gal and

the characteristic subgroup of Gal(NAc)β4-comprising Galβ4Glc, Galβ4GlcNAc, and Galβ4GlcNAc are separately preferred.

Preferred Sialylated Structures

The preferred sialylated structures includes characteristic SAα3Galβ-structures SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, and SAα3Galβ4GlcNAcβ; and biosynthetically partially competing SAα6Galβ-structures SAα6Galβ4Glc, SAα6Galβ4Glc13; SAα6Galβ4GlcNAc and SAα6Galβ4GlcNAcβ; and disialo structures SAα3Galβ3(SAα6)GalNAcβ/α,

The invention is preferably directed to specific subgroup of Gal(NAc)β3-comprising

SAα3Galβ3GlcNAc, SAα3Galβ3GalNAc, SAα3Galβ4GlcNAc,

SAα3Galβ3GlcNAcβ, SAα3Galβ3GalNAcβ/α and

SAα3Galβ3(SAα6)GalNAcβ/α, and

Gal(NAc)β4-comprising sialylated structures. SAα3Galβ4Glc, and SAα3Galβ4GlcNAcβ; and SAα6Galβ4Glc, SAα6Galβ4Glcβ; SAα6Galβ4GlcNAc and SAα6Galβ4GlcNAcβ

These are preferred novel regulated markers characteristics for the various stem cells.

Use Together with a Terminal ManαMan-Structure

The terminal non-modified or modified epitopes are in preferred embodiment used together with at least one ManαMan-structure. This is preferred because the structure is in different N-glycan or glycan subgroup than the other epitopes.

Preferred Structural Groups for Hematopoietic Stem Cells.

The present invention provides novel markers and target structures and binders to these for especially embryonic and adult stem cells, when these cells are not heamtopoietic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures

NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. The invention further provides novel markers for CD133+ cells and novel hematopoietic stem cell markers according to the invention, especially when the structures does not include NeuNAcα3Galβ4(Fucα3)₀₋₁GlcNAc. Preferably the hematopoietic stem cell structures are non-sialylated, fucosylated structures Galβ1-3-structures according to the invention and even more preferably type 1 N-acetyllactosamine structures Galβ3GlcNAc or separately preferred Galβ3GalNAc based structures.

Core Structures of the Terminal Epitopes

It is realized that the target epitope structures are most effectively recognized on specific N-glycans, O-glycan, or on glycolipid core structures.

Elongated Epitopes—Next Monosaccharide/Structure on the Reducing End of the Epitope

The invention is especially directed to optimized binders and production thereof, when the binding epitope of the binder includes the next linkage structure and even more preferably at least part of the next structure (monosaccharide or aminoacid for O-glycans or ceramide for glycaolipid) on the reducing side of the target epitope. The invention has revealed the core structures for the terminal epitopes as shown in the Examples and ones summarized in Tables.

It is realized that antibodies with longer binding epitopes have higher specificity and thus will recognize that desired cells or cell derived components more effectively. In a preferred embodiment the antibodies for elongated epitopes are selected for effective analysis of embryonal type stem cells.

The invention is especially directed to the methods of antibody selection and optionally further purification of novel antibodies or other binders using the elongated epitopes according to the invention. The preferred selection is performed by contacting the glycan structure (synthetic or isolated natural glycan with the specific sequence) with a serum or an antibody or an antibody library, such as a phage display library. Data about these methods are well known in the art and available from internet for example by searching pubmed-medical literature database (www.ncbi.nlm.nih.gov/entrez) or patents e.g. in espacenet (fi.espacenet.com) . The specific antibodies are especially preferred for the use of the optimized recognition of the glycan type specific terminal structures as shown in the Examples and ones summarized in the Table.

It is further realized that part of the antibodies according to the invention and shown in the examples have specificity for the elongated epitopes. The inventors found out that for example Lewis x epitope can be recognized on N-glycan by certain terminal Lewis x specific antibodies, but not so effectively or at all by antibodies recognizing Lewis xβ1-3Gal present on poly-N-acetyllactosamines or neolactoseries glycolipids.

N-Glycans

The invention is especially directed to recognition of terminal N-glycan epitopes on biantennary N-glycans. The preferred non-reducing end monosaccharide epitope for N-glycans comprise β2Man

and its reducing end further elongated variants

β2Man, β2Manα, β2Manα3, and β2Manα6

The invention is especially directed to recognition of lewis x on N-glycan by N-glycan Lewis x specific antibody described by Ajit Varki and colleagues Glycobiology (2006) Abstracts of Glycobiology society meeting 2006 Los Angeles, with possible implication for neuronal cells, which are not directed (but disclaimed) with this type of antibody by the present invention.

Invention is further directed to antibodies with specificity of type 2 N-acetyllactosamineβ2Man recognizing biantennary N-glycan directed antibody as described in Ozawa H et al (1997) Arch Biochem Biophys 342, 48-57.

O-Glycans, Reducing End Elongated Epitopes

The invention is especially directed to recognition of terminal O-glycan epitopes as terminal core I epitopes and as elongated variants of core I and core II O-glycans. The preferred non-reducing end monosaccharide epitope for O-glycans comprise:

a) Core I epitopes linked to αSer/Thr-[Peptide]₀₋₁,

wherein Peptide indicates peptide which is either present or absent. The invention is preferably

b) Preferred core II-type epitopes

R1β6[R2β3Galβ3]ₙGalNAcαSer/Thr, wherein n is = or 1 indicating possible branch in the structure and R1 and R2 are preferred positions of the terminal epitopes, R1 is more preferred

c) Elongated Core I epitope

β3 Gal and its reducing end further elongated variants β3Galβ3GalNAcα,

β3Galβ3GalNAcαSer/Thr

O-glycan core I specific and ganglio/globotype core reducing end epitopes have been described in (Saito S et al. J Biol Chem (1994) 269, 5644-52), the invention is preferably directed to similar specific recognition of the epitopes according to the invention.

O-glycan core II sialyl-Lewis x specific antibody has been described in Walcheck B et al. Blood (2002) 99, 4063-69.

Peptide specificity including antibodies for recognition of O-glycans includes mucin specific antibodies further recognizing GalNAcalfa (Tn) or Galb3GalNAcalfa (T/TF) structures (Hanisch F-G et al (1995) cancer Res. 55, 4036-40; Karsten U et al. Glycobiology (2004) 14, 681-92;

Glycolipid Core Structures

The invention is furthermore directed to the recognition of the structures on lipid structures. The preferred lipid corestructures include:
a) βCer (ceramide) for Galβ4Glc and its fucosyl or sialyl derivatives
b) β3/6Gal for type I and type II N-acetyllactosamines on lactosyl Cer-glycolipids, preferred elongated variants includes β3/6[Rβ6/3]ₙGalβ, β3/6[Rβ6/3]ₙGalβ4 and β3/6[R136/3]ₙGalβ4Glc, which may be further banched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ3, β3Galβ4 and β3Galβ4Glc
c) α3/4Gal, for globoseries epitopes, and elongated variants α3/4Galβ, α3/4Galβ4Glc preferred globoepitopes have elongated epitopes α4Gal, α4Galβ, α4Galβ4Glc, and
   preferred isogloboepitopes have elongated epitopes α3Gal, α3Galβ, α3Galβ4Glc
d) β4Gal for ganglio-series epitopes comprising, and preferred elongated variants include β4Galβ, and β4Galβ4Glc

O-glycan core specific and ganglio/globotype core reducing end epitopes have been described in (Saito S et al. J Biol Chem (1994) 269, 5644-52), the invention is preferably directed to similar specific recognition of the epitopes according to the invention.

Poly-N-Acetyllactosamines

Poly-N-acetyllactosamine backbone structures on O-glycans, N-glycans, or glycolipids comprise characteristic structures similar to lactosyl(cer) core structures on type I (lactoseries) and type II (neolacto) glycolipids, but terminal epitopes are linked to another type I or type II N-acetyllactosamine, which may from a branched structure. Preferred elongated epitopes include:

β3/6Gal for type I and type II N-acetyllactosamines epitope, preferred elongated variants includes R1β3/6[R2β6/3]ₙGalβ, R1β3/6[R2β6/3]ₙGalβ3/4 and R1β3/6[R2β6/3]ₙGa1β3/4GlcNAc, which may be further banched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4GlcNAc.

Numerous antibodies are known for linear (i-antigen) and branched poly-N-acetyllactosamines (I-antigen), the invention is further directed to the use of the lectin PWA for recognition of I-antigens. The inventors revelealed that poly-N-acetyllactosamines are characteristic structures for specific types of human stem cells. Another preferred binding regent, enzyme endo-beta-galactosidase was used for characterization poly-N-acetyllactosamines on glycolipids and on glycoprotein of the stem cells. The enzyme revealed characteristic expression of both linear and branched poly-N-acetyllactosamine, which further comprised specific terminal modifications such as fucosylation and/or sialylation according to the invention on specific types of stem cells.

Combinations of Elongated Core Epitopes

It is realized that stronger labeling may be obtained if the same terminal epitope is recognized by antibody binding to target structure present on two or three of the major carrier types O-glycans, N-glycans and glycolipids. It is further realized that in context of such use the terminal epitope maust be specific enough in comparison to the epitopes present on possible contaminating cells or cell materials. It is further realized that there is highly terminally specific antibodies, which allow binding to on several elongation structures.

The invention revealed each elongated binder type useful in context of stem cells. Thus the invention is directed to the binders recognizing the terminal structure on one or several of the elongating structures according to the invention

Preferred Group of Monosaccharide Elongation Structures

The invention is directed to the preferred terminal epitopes according to the invention comprising the preferred reducing end elongation of the N-acetyllactosamine epitomes described in Formulas T1-T11, referred as T1E-T11E in elongated form

A preferred example is

[Mα]ₘGalβ1-3/4[Nα]ₙGlcNAcAxHex(NAc)ₙ Formula T8E:

wherein

wherein m, n and p are integers 0, or 1, independently

Hex is Gal or Glc,

X is linkage position

M and N are monosaccharide residues being

independently nothing (free hydroxyl groups at the positions)

and/or

SA which is Sialic acid linked to 3-position of Gal or/and 6-position of HexNAc

and/or

Fuc (L-fucose) residue linked to 2-position of Gal

and/or 3 or 4 position of GlcNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently.

A is anomeric structure alfa or beta, X is linkage position 2, 3,or 6

And Hex is hexopyranosyl residue Gal, or Man, and n is integer being 0 or 1, with the provisions that when n is 1 then AxHexNAc is β6GalNAc, when Hex is Man, then AxHex is β2Man, and when Hex is Gal, then AxHex is β3Gal or β6Gal.

The most preferred structures are according to the formula

Formula T8Ebeta, wherein the anomeric structure is beta:

[Mα]ₘGalβ1-3/4 [Nα]ₙGlcNAcβxHex(NAc)ₙ

A preferred group of type II Lactosmines are β2-linked on Man or N-glycans or β6-linked on Gal(NAc) in O-glycan/poly-LacNac structures according to the

[Mα]ₘGalβ1-4[Nα]ₙGlcNAcAxHex(NAc)ₙ Formula T10E

[Mα]ₘGalβ1-4[Nα]ₙGlcNAcβ2Man Formula T10EMan:

and

[Mα]ₘGalβ1-4[Nα]ₙGlcNAcβ6Gal(NAc) Formula T10EGal(NAc):

and further elongated structures according to the invention.

A preferred group of type I Lactosmines are β3- on Gal

According to the Formula T9E

[Mα]ₘGalβ1-3[Nα]ₙGlcNAcβ3Gal

Combination of the Preferred Elongated Epitopes

The invention is directed in a preferred embodiment combined use of the preferred structures and elongated structures for recognition of stem cells. In a preferred embodiment at least one type I LacNAc or type II lacNAc structure are used, in another preferred embodiment a non-reducing end non-modified LacNAc is used with α2Fucosylated LacNAc, Lewis x or sialylated LacNAc, in a preferred embodiment α2Fucosylated type I and type II LacNAc are used. The inventors used factor analysis to produce more preferred combinations according to the invention including use of complex type glycans together with high mannose or Low mannose glycan. In a preferred embodiment a LacNAc structure is used togerher with a preferred glycolipid structure, preferably globotriose type. The invention is preferably directed to recognition of differentiation and/or cell culture condition assosiceted changes in the stem cells.

Preferred Elongated Epitopes

It is realized that elongated glycan epitopes are useful for recognition of the embryonic type stem cells according to the invention. The invention is directed to the use of some of the structures for characterizing all the cell types, while certain structural motifs are more common at a specific differentiation stage.

It is further realized that some of the terminal structures are expressed at especially high levels and thus especially useful for the recognition of one or several types of cells.

The terminal epitopes and the glycan types are listed in Tables, based on the structural analysis of the glycan types following preferred elongated structural epitopes that are preferred as novel markers for embryonal type stem cells and for the uses according to the invention.

Preferred Terminal Galβ3/4 Structures

Type II N-Acetyllactosamine Based Structures

Terminal Type II N-Acetyllactosamine Structures

The invention revealed preferred type II N-acetyllactosamines including specific O-glycan, N-glycan and glycolipid epitopes. The invention is in a preferred embodiment especially directed to abundant O-glycan and N-glycan epitopes. The invention is further directed to the recognition of a characteristic glycolipid type II LacNAc terminal. The invention is especially directed to the use of the Type II LacNAc for recognition of non-differentiated embryonal type stem cells (stage I) and similar cells or for the analysis of the differentiation stage. It is however realized that substantial amounts of the structures are present in the more differentiated cells as well.

Elongated type II LacNAc structures are especially expressed on N-glycans. Preferred type II LacNAc structures are β2-linked to the biantennary N-glycan core structure, including the preferred epitopes Galβ4GlcNAcβ2Man, Galβ4GlcNAcβ2Manα, Galβ4GlcNAcβ2Manα3/6Man and Galβ4GlcNAcβ2Manα3/6Manβ4

The invention further revealed novel O-glycan epitopes with terminal type II N-acetyllactosamine structures expressed effectively on the embryonal type cells. The analysis of the O-glycan structures revealed especially core II N-acetyllactosamines with the terminal structure. The preferred elongated type II N-acetyllactosamines thus includes Galβ4GlcNAcβ6GalNAc, Galβ4GlcNAcβ6GalNAcα, Galβ4GlcNAcβ6(Galβ3)GalNAc, and Galβ4GlcNAcβ6(Galβ3)GalNAcα.

The invention further revealed the presence of type II LacNAc on glycolipids. The present invention reveals for the first time terminal type II N-acetyllactosamine on glycolipids of stem cells. The neolacto glycolipid family is an important glycolipid family characteristically expressed on certain tissues but not on others.

The preferred glycolipid structures include epitopes, preferably non-reducing end terminal epitopes of linear neolactotetraosyl ceramide and elongated variants thereof Galβ4GlcNAcβ3Gal, Galβ4GlcNAcβ3Galβ4, Galβ4GlcNAcβ3Galβ4Glc(NAc), Galβ4GlcNAcβ3Galβ4Glc, and Galβ4GlcNAcβ3Galβ4GlcNAc. It is further that specific reagents recognizing the linear polylactosamines can be used for the recognition of the structures, when these are linked to protein linked glycans. In a preferred embodiment the invention is directed to the poly-N-acetyllactosamines linked to N-glycans, preferably β2-linked structures such as Galβ4GlcNAcβ3Galβ4GlcNAcβ2Man on N-glycans. The invention is further directed to the characterization of the poly-N-acetyllactosamine structures of the preferred cells and their modification by SAα3, SAα6, Fucα2 to non-reducing end Gal and by Fucα3 to GlcNAc residues.

The invention is preferably directed to recognition of tetrasaccharides, hexasaccharides, and octasaccharides. The invention further revealed branched glycolipid polylactosamines including terminal type II LacNAc epitopes, preferably these include Galβ4GlcNAcβ6Gal, Galβ4GlcNAcβ6Galβ, Galβ4GlcNAcβ6(Galβ4GlcNAcβ3)Gal, and Galβ4GlcNAcβ6(Galβ4GlcNAcβ3)Galβ3, Galβ4GlcNAcβ6(Galβ4GlcNAcβ3)Galβ4Glc(NAc), Galβ4GlcNAcβ6(Galβ4GlcNAcβ3)Galβ4Glc, and Galβ4GlcNAcβ6(Galβ4GlcNAcβ3)Galβ4GlcNAc.

It is realized that antibodies specifically binding to the linear or branched poly-N-acetyllactosamines are well known in the art. The invention is further directed to reagents recognizing both branched polyLacNAcs and core II O-glycans with similar β6Gal(NAc) epitopes.

Lewis x Structures

Elongated Lewis x structures are especially expressed on N-glycans. Preferred Lewis x structures are β2-linked to the biantennary N-glycan core structure, including the preferred structures Galβ4(Fucα3)GlcNAcβ2Man, Galβ4(Fucα3)GlcNAcβ2Manα, Galβ4(Fucα3)GlcNAcβ2Manα3/6Man, Galβ4(Fucα3)GlcNAcβ2Manα3/6Manβ4

The invention further revealed the presence of Lewis x on glycolipids. The preferred glycolipid structures include Gal(Fucα3)β4GlcNAcβ3Gal, Galβ4(Fucα3)GlcNAcβ3Gal, Galβ4(Fucα3)GlcNAcβ3Galβ4, Galβ4(Fucα3)GlcNAcβ3Galβ4Glc(NAc), Galβ4(Fucα3)GlcNAcβ3Galβ4Glc, and Galβ4(Fucα3)GlcNAcβ3Galβ4GlcNAc.

The invention further revealed the presence of Lewis x on O-glycans. The preferred O-glycan structures include preferably the core II structures Galβ4(Fucα3)GlcNAcβ6GalNAc, Galβ4(Fucα3)GlcNAcβ6GalNAcα, Galβ4(Fucα3)GlcNAcβ6(Galβ3)GalNAc, and Galβ4(Fucα3)GlcNAcβ6(Galβ3)GalNAcα.

H Type II Structures

Specific elongated H type II structural epitopes are especially expressed on N-glycans. Preferred H type II structures are β2-linked to the biantennary N-glycan core structure, Fucα2Galβ4GlcNAcβ2Manα3/6Manβ4

The invention further revealed the presence of H type II on glycolipids. The preferred glycolipid structures includes Fucα2Galβ4GlcNAcβ3Gal, Fucα2Galβ4GlcNAcβ3Gal, Fucα2Galβ4GlcNAcβ3Galβ4, Fucα2Galβ4GlcNAcβ3Galβ4Glc(NAc), Fucα2Galβ4GlcNAcβ3Galβ4Glc, and Fucα2Galβ4GlcNAcβ3Galβ4GlcNAc.

The invention further revealed the presence of H type II on O-glycans. The preferred O-glycan structures include preferably core II structures Fucα2Galβ4GlcNAcβ6GalNAc, Fucα2Galβ4GlcNAcβ6GalNAcα, Fucα2Galβ4GlcNAcβ6(Galβ3)GalNAc, and Fucα2Galβ4GlcNAcβ6(Galβ3)GalNAcα.

Sialylated Type II N-Acetyllactosamine Structures

The invention revealed preferred sialylated type II N-acetyllactosamines including specific O-glycan, N-glycan and glycolipid epitopes. The invention is in a preferred embodiment especially directed to abundant O-glycan and N-glycan epitopes. SA refers here to sialic acid, preferably Neu5Ac or Neu5Gc, more preferably Neu5Ac. The sialic acid residues are SAα3Gal or SAα6Gal, it is realized that these structures when presented as specific elongated epitopes form characteristic terminal structures on glycans.

Sialylated type II LacNAc structural epitopes are especially expressed on N-glycans. Preferred type II LacNAc structures are β2-linked to biantennary N-glycan core structure, including the preferred terminal epitopes SAα3/6Galβ4GlcNAcβ2Man, SAα3/6Galβ4GlcNAcβ2Manα, and SAα3/6Galβ4GlcNAcβ2Manα3/6Manβ4. The invention is directed to both SAα3-structures (SAα3Galβ4GlcNAcβ2Man, SAα3Galβ4GlcNAcβ2Manα, and SAα3Galβ4GlcNAcβ2Manα3/6Manβ4) and SAα6-epitopes (SAα6Galβ4GlcNAcβ2Man, SAα6Galβ4GlcNAcβ2Manα, and SAα6Galβ4GlcNAcβ2Manα3/6Manβ4) on N-glycans.

The SAα3-N-glycan epitopes are preferred for the analysis of the non-differentiated stage I embryonic type cells and other stem cells. The SAα6-N-glycan epitopes are preferred for analysis of the differentiated, for differentiating embryonic type cells, such as embryoid bodies and stage III differentiated embryonic type cells. It is realized that the combined analysis of both types of N-glycans is useful for the characterization of the embryonic type stem cells.

The invention further revealed novel O-glycan epitopes with terminal sialylated type II N-acetyllactosamine structures expressed effectively on the embryonal type cells. The analysis of O-glycan structures revealed especially core II N-acetyllactosamines with the terminal structure. The preferred elongated type II sialylated N-acetyllactosamines thus include SAα3/6Galβ4GlcNAcβ6GalNAc, SAα3/6Galβ4GlcNAcβ6GalNAcα, SAα3/6Galβ4GlcNAcβ6(Galβ3)GalNAc, and SAα3/6Galβ4GlcNAcβ6(Galβ3)GalNAcα. The SAα3-structures were revealed as preferred structures in context of the O-glycans including SAα3Galβ4GlcNAcβ6GalNAc, SAα3Galβ4GlcNAcβ6GalNAcα, SAα3Galβ4GlcNAcβ6(Galβ3)GalNAc, and SAα3Galβ4GlcNAcβ6(Galβ3)GalNAcα.

Specific Preferred Tetrasaccharide Type II Lactosamine Epitopes

It is realized that highly effective reagents can in a preferred embodiment recognize epitopes which are larger than a trisaccharide. Therefore the invention is further directed to the branched terminal type II lactosamine derivatives Lewis y Fucα2Galβ4(Fucα3)GlcNAc and sialyl-Lewis x SAα3Galβ4(Fucα3)GlcNAc as preferred elongated or large glycan structural epitopes. It is realized that the structures are combinations of preferred termina trisaccharide sialyl-lactosamine, H-type II and Lewis x epitopes. The analysis of the epitopes is preferred as additionally useful method in the context of analysis of other terminal type II epitopes. The invention is especially directed to -further defining the core structures carrying the Lewis y and sialyl-Lewis x epitopes on various types of glycans and optimizing the recognition of the structures by including the recognition of the preferred glycan core structures.

Structures Analogous to the Type II Lactosamines

The invention is further directed to the recognition of elongated epitopes analogous to the type II N-acetyllactosamines including LacdiNAc especially on N-glycans and lactosylceramide (Galβ4GlcβCer) glycolipid structure. These share similarity with LacNAc the only difference being the number of NAc residues on the monosaccharide residues.

LacdiNAc Structures

It is realized that LacdiNac is relatively rare and characteristic glycan structure and it is therefore especially preferred for the characterization of the embryonic type cells. The invention revealed the presence of LacdiNAc on N-glycans at least as β2-linked terminal epitope. The structures were characterized by specific glycosidase cleavages. The LacdiNAc structures have same mass as structures with two terminal GlcNAc containing structures in structural Tables, Tables includes representative structures indicating only single isomeric structures for a specific mass number. The preferred elongated LacdiNAc epitopes thus includes GalNAcβ4GlcNAcβ2Man, GalNAcβ4GlcNAcβ2Manα, and GalNAcβ4GlcNAcβ2Manα3/6Manβ4. The invention further revealed fucosylation of LacdiNAc containing glycan structures and the preferred epitopes thus further include GalNAcβ4(Fucα3)GlcNAcβ2Man, GalNAcβ4(Fucα3)GlcNAcβ2Manα, GalNAcβ4(Fucα3)GlcNAcβ2Manα3/6Manβ4 GalNAc(Fucα3)β4GlcNAcβ2Manα3/6Manβ4. It is realized that presence of β6-linked sialic acid of LacNac of structure with mass number 2263, tables indicates that at least part of the fucose is present on the LacdiNAc arm of the molecule based on the competing nature of α6-sialylation and α3-fucosylation on enzyme specificity level (alternative assignment presented in the Tables).

Type I N-Acetyllactosamine Based Structures

Terminal Type I N-Acetyllactosamine Structures

The invention revealed preferred type I N-acetyllactosamines including specific O-glycan, N-glycan and glycolipid epitopes. The invention is in a preferred embodiment especially directed to abundant glycolipid epitopes. The invention is further preferably directed to the recognition of characteristic O-glycan type I LacNAc terminals.

The invention is especially directed to the use of the Type I LacNAc for the recognition of non-differentiated embryonal type stem cells (stage I) and similar cells and other stem cells or for the analysis of the differentiation stage. It is however realized that substantial amount of the structures are present in the more differentiated cells as well.

The invention further revealed novel O-glycan epitopes with terminal type I N-acetyllactosamine structures expressed effectively on the embryonal type cells and certain mesenchymal cells. The analysis of O-glycan structures revealed especially core II N-acetyllactosamines with the terminal structure on type II lactosamine. The preferred elongated type I N-acetyllactosamines thus includes Galβ3GlcNAcβ3Galβ4GlcNAcβ6GalNAc, Galβ3GlcNAcβ3Galβ4GlcNAcβ6GalNAcα, Galβ3GlcNAcβ3GalGlcNAcβ6(Galβ3)GalNAc, and Galβ3GlcNAcβ3Galβ4GlcNAcβ6(Galβ3)GalNAcα.

The invention further revealed the presence of type I LacNAc on glycolipids. The present invention reveals for the first time terminal type I N-acetyllactosamine on glycolipids. The Lacto glycolipid family is an important glycolipid family characteristically expressed on certain tissue but not on others.

The preferred glycolipid structures include-epitopes, preferably non-reducing end terminal epitopes, of linear lactoteraosyl ceramide and elongated variants thereof Galβ3GlcNAcβ3Gal, Galβ3GlcNAcβ3Galβ4, Galβ3GlcNAcβ3Galβ4Glc(NAc), Galβ3GlcNAcβ3Galβ4Glc, and Galβ3GlcNAcβ3Galβ4GlcNAc. It is further realized that specific reagents recognizing the linear polylactosamines can be used for the recognition of the structures, when these are linked to protein linked glycans. It is especially realized that the terminal tri-and tetrasaccharide epitopes on the preferred O-glycans and glycolipids are essentially the same. The invention is in a preferred embodiment directed to the recognition of the both structures by the same binding reagent such as a monoclonal antibody

The invention is further directed to the characterization of the terminal type I poly-N-acetyllactosamine structures of the preferred cells and their modification by SAα3, Fucα2 to non-reducing end Gal and by SAα6 or Fucα3 to GlcNAc residues and other core glycan structures of the derivatized type I N-acetyllactosamines.

A preferred elongated type I LacNAc structure is expressed on N-glycans. Preferred type I LacNAc structures are β2-linked to the biantennary N-glycan core structure, the preferred epitopes being Galβ3GlcNAcβ2Man, Galβ3GlcNAcβ2Manα and Galβ3GlcNAcβ2Manα3/6Manβ4.

Fucosylated Type I LacNAcs

Lewis a Structures

The invention revealed the presence of Lewis a structures on glycolipids. The invention is further directed to related poly-N-acetyllactosamine structures with similar terminal epitopes. The preferred glycolipid structures includes Galβ3(Fucα4)βGlcNAcβ3Gal, Galβ3(Fucα4)βGlcNAcβ3Gal, Galβ3(Fucα4)βGlcNAcβ3Galβ4, Galβ3(Fucα4)βGlcNAcβ3Galβ4Glc(NAc), Galβ3(Fucα4)βGlcNAcβ3Galβ4Glc, and Galβ3(Fucα4)βGlcNAcβ3Galβ4GlcNAc.

The invention is further directed to the presence of Lewis a on elongated O-glycans. The preferred O-glycan polylactosamine type structures include preferably the core II structures Galβ3(Fucα4)GlcNAcβ3Galβ4GlcNAcβ6GalNAc, Galβ3(Fucα4)GlcNAcβ3Galβ4GlcNAcβ6GalNAcα, Galβ3(Fucα4)GlcNAcβ3Galβ4GlcNAcβ6(Galβ3)GalNAc, and Galβ3(Fucα4)GlcNAcβ3Galβ4GlcNAcβ6(Galβ3)GalNAcα.

H Type I Structures

A Preferred elongated H type I structure is on lacto series glycolipids or related poly-N-acetyllactosamine structures. The preferred glycolipid/polylactosamine structures includes Fucα2Galβ3GlcNAcβ3Gal, Fucα2Galβ3GlcNAcβ3Gal, Fucα2Galβ3GlcNAcβ3Galβ4, Fucα2Galβ3GlcNAcβ3Galβ4Glc(NAc), Fucα2Galβ3GlcNAcβ3Galβ4Glc, and Fucα2Galβ3GlcNAcβ3Galβ4GlcNAc.

The invention is further directed to the presence of H type I on elongated O-glycans. The preferred O-glycan polylactosamine type structures include preferably the core II structures Fucα2Galβ3GlcNAcβ3Galβ4GlcNAcβ6GalNAc, Fucα2Galβ3GlcNAcβ3Galβ4GlcNAcβ6GalNAcα, Fucα2Galβ3GlcNAcβ3Galβ4GlcNAcβ6(Galβ3)GalNAc, and Fucα2Galβ3GlcNAcβ3Galβ4GlcNAcβ6(Galβ3)GalNAcα.

Specific Preferred Tetrasaccharide Type I Lactosamine Epitopes

It is realized that highly effective reagents can in a preferred embodiment recognize epitopes which are larger than a trisaccharide. Therefore the invention is further directed to the branched terminal type I lactosamine derivatives Lewis b Fucα2Galβ3(Fucα4)GlcNAc and sialyl-Lewis a SAα3Galβ3(Fucα4)GlcNAc as preferred elongated or large glycan structural epitopes. It realized that the structures are combinations of preferred terminal trisaccharide sialyl-lactosamine, H-type I and Lewis a epitopes. The analysis of the epitopes is preferred as additionally useful method in the context of analysis of other terminal type I epitopes. The invention is especially directed to-further defining the core structures carrying the type Lewis b and sialyl-Lewis a epitopes on various types of glycans and optimizing the recognition of the structures by including the recognition of preferred glycan core structures. The invention revealed that at least some of the sialyl-Lewis a epitopes are scarce on stage I cells and the structure is associated more with differentiated cell types.

As used herein, “binder”, “binding agent” and “marker” are used interchangeably.

Antibodies

Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corγnebacterium parvum.

A monoclonal antibody to a peptide or glycan motif(s) may be prepared by using any technique which provides for the production of antibody molecules by continuous cell lines in culture. These include but are not limited to the hybridoma technique originally described by Kohler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-I1, MPC11-X45-GTG 1.7 and 5194/5XXO BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81 : 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above.

An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

Preferably, the antibody is any antibody specific for a glycan structure of Formula (I) or a fragment thereof. The antibody used in the present invention encompasses any antibody or fragment thereof, either native or recombinant, synthetic or naturally-derived, monoclonal or polyclonal which retains sufficient specificity to bind specifically to the glycan structure according to Formula (I) which is indicative of stem cells. As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′).sub.2 fragments, and Fv fragments.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and .beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, .sup.125 I and amino acids comprising any radionuclides, including, but not limited to .sup.14 C, .sup.3 H and .sup.35 S.

Antibodies to glycan structure(s) of Formula (I) may be obtained from any source. They may be commercially available. Effectively, any means which detects the presence of glycan structure(s) on the stem cells is with the scope of the present invention. An example of such an antibody is a H type 1 (clone 17-206; GF 287) antibody from Abcam.

Structures Associated with Nondifferentiated hESC

The Tables show specific structure groups with specific monosaccharide compositions associated with the differentiation status of human embryonic stem cells.

The Structures Present in Higher Amount in hESCs than in Corresponding Differentiated Cells

The invention revealed novel structures present in higher amounts in hESCs than in corresponding differentiated cells.

The preferred hESC enriched glycan groups are represented by groups hESC-i to hESC-ix, corresponding to several types of N-glycans. The glycans are preferred in the order from hESC-i to hESC-ix, based on the relative specificity for the non-differentiated hESCs, the differences in expression are shown in Tables. The glycans are grouped based on similar composition and similar structures present to group comprising Complex type N-glycans other preferred glycan groups,

Complex Type Glycans

hESC-i, Biantennary-Size Complex-Type N-Glycans

The highest specific expression in hESCs was revealed for a specific group of biantennary complex type N-glycan structures. This group includes neutral glycans including H5N4F1, H5N4F2, H5N4F3; and sialylated glycans G2H5N4, G1H5N4, S1H5N4F2, G1H5N4F1, S1G1H5N4, S1H5N4F3, S2H5N4F1, S1H5N4, and S1H5N4F1.

Preferred Structural Subgroups of the Biantennary Complex Type Glycans Include Neutral Fucosylated Glycans and NeuAc Comprising Fucosylated Glycans and Glycans Comprising NeuGc.

Neutral Fucosylated Glycans

The group of neutral glycans forms a homogenous group with typical composition of biantennary N-glycans and one, two or three fucose residues. This group shares a common composition:

H₅N₄F_{q}

Wherein

q is an integer being 1, 2 or 3.

The preferred structures in this group include

[Fucα]ₘGalβGNβ2Manα3([Fucα]ₙGalβGNβ2Manα6)Manβ4GNβ4(Fucα6)GN, wherein m and n are 0 or 1, GN is GlcNAc. The structures are preferably core fucosylated, when there is only one fucose. (The core fucosylation was revealed by NMR-analysis of the hESC glycans.) The fucose residues at the antennae (branches) are preferably either Fucα2-structures linked to Gal or Fucα3/4-structures, preferably Fucα3, linked to GlcNAc of the terminal N-acetyllactosamines

Preferred fucosylated terminal epitopes [Fucα]GalβGlcNAcβ2Manα

Preferred Lewis x Epitopes

The preferred terminal epitopes, which can be recognized from hESCs by specific binder molecules, include Lewis x, Galβ4(Fucα3)GlcNAcβ, more preferably Galβ4(Fucα3)GlcNAcβ2Manα, based on binding of specific Lewis x recognizing monoclonal antibody.

The invention is further directed to the recognition of the Lewis x structure as a specific preferred arm of N-glycan selected from the group Galβ4(Fucα3)GlcNAcβ2Manα3Manβ (Lexβ2Manα3-arm) and/or Galβ4(Fucα3)GlcNAcβ2Manα6Manβ (Lexβ2Manα6-arm). The invention is directed to selection and development of reagents for the specific fucosylated N-glycan arms for recognition of N-glycans on the human embryonic stem cells and derivatives.

The H-antigens on N-glycans includes preferably the epitope Fucα2GalβGlcNAcβ, preferably H type I Fucα2Galβ3GlcNAcβ or H type II structure Fucα2Galβ4GlcNAcβ, more preferably Fucα2Galβ4GlcNAcβ, and most preferably Fucα2Galβ4GlcNAcβ2Manα.

The invention is further directed to the recognition of the H type II structure as a specific preferred arm of N-glycan selected from the group Fucα2Galβ4GlcNAcβ2Manα3Manβ (HLacNAcβ2Manα3-arm) and/or Fucα2Galβ4GlcNAcβ2Manα6Manβ (HLacNAcβ2Manα6-arm). The invention is directed to selection and development of reagents for the specific fucosylated N-glycan arms for recognition of N-glycans on the human embryonic stem cells and derivatives.

Preferred neutral difucosylated structures include glycans comprising core fucose and the terminal Lewis x or H-antigen on either arm of the biantennary N-glycan according to the formulae:

Galβ4(Fucα3)GNβ2Manα3/6(GalβGNβ2Manα6/3)Manβ4GNβ4(Fucα6)GN, and/or Fucα2GalβGNβ2Manα3/6(GalβGNβ2Manα6/3)Manβ4GNβ4(Fucα6)GN.

Preferred neutral trifucosylated structures includes glycans comprising core fucose and the terminal Lewis x or H-antigen on either arm of the biantennary N-glycan according to the formulae:

Galβ4(Fucα3)GNβ2Manα3/6([Fucα]GalβGNβ2Manα6/3)Manβ4GNβ4(Fucα6)GN, and/or

Fucα2GalβGNβ2Manα3/6([Fucα]GalβGNβ2Manα6/3)Manβ4GNβ4(Fucα6)GN,

Wherein the molecules comprise two H-structures, Lewis x in one arm and H-structure in the the other arm or two Lewis x structures:

Fucα2GalβGNβ2Manα3(Fucα2GalβGNβ2Manα6)Manβ4GNβ4(Fucα6)GN, Galβ4(Fucα3)GNβ2Manα3/6(Fucα2GalβGNβ2Manα6/3)Manβ4GNβ4(Fucα6)GN Galβ4(Fucα3)GNβ2Manα3(Galβ4(Fucα3)GNβ2Manα6)Manβ4GNβ4(Fucα6)GN,

Or molecules comprising Lewis y on one arm:

Fucα2Galβ4(Fucα3)GNβ2Manα3/6(GalβGNβ2Manα6/3)Manβ4GNβ4(Fucα6)GN

NeuAc Comprising Fucosylated Glycans

The sialylated glycans include NeuAc comprising fucosylated glycans with formulae: S1H5N4F2, S1H5N4F3, S2H5N4F1, S1H5N4, and S1H5N4F1. This group shares composition:

SₖH₅N₄F_{q}

Wherein

k is an integer being 1 or 2

q is an integer from 0 to 3.

The group comprises monosialylated glycans with all levels of fucosylation and disialylated glycan with single fucose. The preferred subgroups of this category include low fucosylation level glycans comprising no or one fucose residue (low fucosylation) and glycans with two or three fucose residues.

Preferred Biantennary Structures with Low Fucosylation

The preferred biantennary structures according to the invention include structures according to the Formula:

[NeuAcα]₀₋₁GalβGNβ2Manα3([NeuAcα]₀₋₁GalβGNβ2Manα6)Manβ4GNβ4(Fucα6)₀₋₁GN,

The GalβGlcNAc structures are preferably Galβ4GlcNAc-structures (type II N-acetyllactosamine antennae). The presence of type 2 structures was revealed by specific β4-linkage cleaving galactosidase (D. pneumoniae).

In a preferred embodiment the sialic acid is NeuAcα6− and the glycan comprises the NeuAc linked to Manα3-arm of the molecule. The assignment is based on the presence of α6-linked sialic acid revealed by specific sialidase digestion and the known branch specificity of the α6-sialyltransferase (ST6GalI).

NeuAcα6GalβGNβ2Manα3([NeuAcα]₀₋₁GalβGNβ2Manα6)Manβ4GNβ4(Fucα6)₀₋₁GN, more preferably type II structures:

NeuAcα6Galβ2GNβ2Manα3([NeuAcα]₀₋₁Galβ2GNβ2Manα6)Manβ4GNβ4(Fucα6)₀₋₁GN.

The invention thus revealed preferred terminal epitopes, NeuAcα6GalβGN, NeuAcα6GalβGNβ2Man, NeuAcα6GalβGNβ2Manα3, to be recognized by specific binder molecules. It is realized that higher specificity preferred for application in context of similar structures can be obtained by using binder recognizing longer epitopes and thus differentiating e.g. between N-glycans and other glycan types in context of the terminal epitopes.

Preferred Difucosylated and Sialylated Structures

Preferred difucosylated sialylated structures include structures, wherein the one fucose is in the core of the N-glycan and

a) one fucose on one arm of the molecule, and sialic acid is on the other arm (antenna of the molecule and the fucose is in Lewis x or H-structure:

Galβ4(Fucα3)GNβ2Manα3/6(NeuNAcαGalβGNβ2Manα6/3)Manβ4GNβ4(Fucα6)GN,

and/or

Fucα2GalβGNβ2Manα3/6(NeuNAcαGalβGNβ2Manα6/3)Manβ4GNβ4(Fucα6)GN, and when the sialic acid is α6-linked preferred antennary structures contain preferably the sialyl-lactosamine on α3-linked arm of the molecule according to formula: Galβ4(Fucα3)GNβ2Manα6(NeuNAcα6Galβ4GNβ2Manα3)Manβ4GNβ4(Fucα6)GN, and/or

Fucα2GalβGNβ2Manα6(NeuNAcα6Galβ4GNβ2Manα3)Manβ4GNβ4(Fucα6)GN.

It is realized that the structures, wherein the sialic acid and fucose are on different arms of the molecules can be recognized as characteristic specific epitopes.

b) Fucose and NeuAc are on the same arm in a structure:

NeuNAcα3Galβ3/4(Fucα4/3)GNβ2Manα3/6(GalβGNβ2Manα6/3)Manβ4GNβ4(Fucα6)GN,

and more preferably sialylated and fucosylated sialyl-Lewis x structures are preferred as a characteristic and bioactive structures:

NeuNAcα3Galβ4(Fucα3)GNβ2Manα3/6(Galβ4GNβ2Manα6/3)Manβ4GNβ4(Fucα6) GN.

Preferred Sialylated Trifucosylated Structures

Preferred sialylated trifucosylated structures include glycans comprising core fucose and the terminal sialyl-Lewis x or sialyl-Lewis a, preferably sialyl-Lewis x due to relatively large presence of type 2 lactosamines, or Lewis y on either arm of the biantennary N-glycan according to the formulae:

NeuNAcα3Galβ4(Fucα3)GNβ2Manα3/6([Fucα]GalβGNβ2Manα6/3)Manβ4GNβ4(Fucα6)GN, and/or

Fucα2Galβ4(Fucα3)GNβ2Manα3/6(NeuNAcα3/6GalβGNβ2Manα6/3)Manβ4GNβ4(Fucα6)GN. NeuNAc is preferably α-linked on the same arm as fucose due to known biosynthetic preference. When the structure comprises NeuNAcα6, this is preferably linked to form NeuNAcα6Galβ4GlcNAcβ2Manα3-arm of the molecule.

Glycans Comprising N-Glycolylneuraminic Acid

The invention is directed to glycans comprising N-glycolylneuraminic acid with following compositions G2H5N4, G1H5N4, G1H5N4F1, and S1G1H5N4. The compositions form a group of compositions with composition:

GₘSₖH₅N₄F_{q}

wherein

m is an integer being 1 or 2,

k is an integer being 0 or 1, and

q is an integer being 0 or 1.

The invention is further directed to the structures according to the formula: [NeuXα]₀₋₁GalβGNβ2Manα3/6([NeuXα]₀₋₁GalβGNβ2Manα6/3)Manβ4GNβ4(Fucα6)₀₋₁GN,

wherein X is Gc or Ac, and the sialic acids are linked by α3- and/or α6-linkages.

It is further realized that it is useful to analyze the NeuGc comprising structures in context of contamination by animal protein and or animal derived NeuGc-monosaccharide or glycoconjugate comprising material.

hESC-ii, Complex-Fucosylated N-Glycans

The invention is further directed to following neutral glycans including H5N4F2, H5N4F3, H4N5F3; and sialylated glycans including S1H7N6F2, S1H7N6F3, S1H5N4F2, S1H6N5F2, S1H6N4F2, S1H5N4F3, S1H4N4F2, S2H6N5F2, S1H6N5F3;

preferentially with α1,2-, α1,3-, and/or α1,4-linked fucose residues within the N-acetyllactosamine antenna sequence Galβ3/4GlcNAc forming H and/or Lewis antigens, more preferentially type II N-acetyllactosamine (Galβ4GlcNAc) forming H type 2, Lewis x, sialyl Lewis x, and/or Lewis y antigens.

LacdiNAc Comprising S1/0H4N5F2/3-Structures

In a preferred embodiment, the invention is directed to analysis of structure of preferred N-glycans with S1/0H4N5F2/3 structures, when the composition comprises biantennary N-glycan type structures with terminal LacdiNAc structure. The LacdiNAc epitope has structure GalNAcβGlcNAc, preferably GalNAcβ4GlcNAc and preferred sialylated LacdiNAc epitope has the structure NeuAcα6GalNAcβ4GlcNAc, based on the known mammalian glycan structure information. Based on biosynthetic knowledge the α6-sialylated structure likely not comprises fucose. The preferred sialyl-lactosamine structures includes NeuAcα3/6Galβ4GlcNAc. The presence of lacdinac structures was revealed by N-acetylhexosaminidase and N-acetylglucosaminidase digestions.

The invention is especially directed to the composition with terminal Lewis x epitope and a sialylated LacdiNAc epitope according to the Formula:

Galβ4(Fucα3)GNβ2Manα3/6(NeuAcα6GalNAcβ4GNβ2Manα6/3)Manβ4GlcNAcβ4(Fucα6)GN.

The invention is especially directed to the composition with terminal Lewis x epitope and a fucosylated LacdiNAc epitope according to the Formula:

Galβ4(Fucα3)GNβ2Manα3/6(GalNAcβ4(Fucα3)GNβ2Manα6/3)Manβ4GlcNAcβ4(Fucα6)GN, and/or structure with Lewis y and LacdiNAc:

Fucα2Galβ4(Fucα3)GNβ2Manα3/6(GalNAcβ4GNβ2Manα6/3)Manβ4GlcNAcβ4(Fucα6)GN.

Multiple N-Acetyllactosamine Comprising Structures

The invention is further directed to multiple (more than 2) N-acetyllactosamine comprising N-glycan structures according to the formulae: S1H7N6F2, S1H7N6F3, S1H6N5F2, S2H6N5F2, and S1H6N5F3.

Preferred Triantennary Glycans

The invention is especially directed to triantennary N-glycans having compositions S1H6N5F2, S2H6N5F2, and S1H6N5F3. Presence of triantennary structures was revealed by specific galactosidase digestions. A preferred type of triantennary N-glycans includes one synthesized by Mgat3. The triantennary N-glycan comprises in a preferred embodiment a core fucose residue. The preferred terminal epitopes include Lewis x, sialyl-Lewis x, H- and Lewis y antigens as described above for biantennary N-glycans.

Preferred Tetraantennary and/or Polylactosamine Structures

The invention is further directed to monosaccharide compositions and glycan corresponding to monosaccharide compositions S1H7N6F2, and S1H7N6F3, which were assigned to correspond to tetra-antennary and/or poly-N-acetyllactosamine epitope comprising N-glycans such as ones with terminal GalβGlcNAcβ3GalβGlcNAcβ−, more preferably type 2 structures Galβ4GlcNAcβ3Galβ4GlcNAcβ−.

hESC-vi, Large Complex-Type N-Glycans

The preferred group includes neutral glycans with compositions H6N5, and H6N5F1. The preferred structures in this group include:

triantennary N-glycans, in a preferred embodiment the triantennary N-glycan comprises β1,4-linked N-acetyllactosamine, preferably linked to Manα6-arm of the N-glycan (mgat4 product N-glycan) and poly-N-acetyllactosamine elongated biantennary complex-type N-glycans.

hESC-vii, Monoantennary Type N-Glycans

The preferred group includes neutral glycans with compositions including H4N3, and H4N3F1;

And preferentially corresponding to structures:

GalβGlcNAcβ2Manα3(Manα6)Manβ4GlcNAcβ4(Fucα6)₀₋₁GlcNAc, more preferentially with type II N-acetyllactosamine antennae, wherein galactose residues are β1,4-linked

Galβ4GlcNAcβ2Manα3(Manα6)Manβ4GlcNAcβ4(Fucα6)₀₋₁GlcNAc.

hESC-viii, Terminal HexNAc Complex-Type N-Glycans

The preferred group includes neutral glycans having composition H4N5F3; and sialylated glycans including S2H4N5F1, and S1H4N5F2.

hESC-ix, Elongated Large Complex-Type N-Glycans

The preferred group includes glycans having composition S1H8N7F1, S1H7N6F2, S1H7N6F3, and S1H7N6F1;

preferentially including poly-N-acetyllactosamine sequences.

Terminal Mannose N-Glycans

High Mannose Type Glycans

hESC-iii, High-mannose type N-glycans, including H6N2, H7N2, H8N2, and H9N2.The preferred high Mannose type glycans are according to the formula:

[Mα2]ₙ₁Mα3{[Mα2]ₙ₃Mα6}Mα6{[Mα2]ₙ₆[Mα2]ₙ₇Mα3}Mβ4GNβ4GNyR₂

wherein n1, n3, n6, and n7are either independently 0 or 1;

y is anomeric linkage structure α and/or β or linkage from derivatized anomeric carbon, and

R₂ is reducing end hydroxyl, chemical reducing end derivative or natural asparagine

N-glycoside derivative such as asparagine N-glycosides including aminoacid and/or peptides derived from protein;

[ ] indicates determinant either being present or absent depending on the value of n1, n3, n6, n7; and

{ } indicates a branch in the structure;

M is D-Man, GN is N-acetyl-D-glucosamine, y is anomeric structure or linkage type, preferably beta to Asn.

The preferred structures in this group include:

Manα2Manα6(Manα2Manα3)Manα6(Manα2Manα2Manα3)Manβ4GlcNAcβ4GlcNAc

Manα2Manα6([Manα2]₀₋₁Manα3)Manα6([Manα2]₀₋₁Manα2Manα3)Manβ4GlcNAcβ4GlcNAc

hESC-v, Glucosylated high-mannose type N-glycans, including H10N2, H11N2;

preferentially including:

Manα2Manα6(Manα2Manα3)Manα6([Glcα]₀₋₁GlcαManα2Manα2Manα3)Manβ4GlcNAcβ4GlcNAc

Specific Low Mannose Type Glycan

hESC-iv, Monomannose N-glycan H1N2;

preferentially including the structure Manβ4GlcNAcβ4GlcNAc.

Structures and Compositions Associated with Differentiated Cell Types (EB and St.3)

The invention revealed novel structures present in higher amount in differentiated embryonic stem cells than in corresponding non-differentiated hESCs. The preferred glycan groups are represented in groups Diff-i to Diff-ix, corresponding to several types of N-glycans. The glycans are preferred in the order from Diff-i to Diff-ix, based on the relative specificity for the non-differentiated hESCs, the differences in the expression are shown in Tables.

Analysis of Specific Glycan Groups in hESC Glycomes

The analysis of N-glycome revealed signals and monosaccharide compositions specific for embryonic stem cells at various differentiation levels. Some preferred structures are assigned in Tables. The terminal structures were assigned based on specific binding molecules NMR and glycosidase digestions. The binding molecules for terminal epitopes including structures present also in glycolipids or on proteins and lipids are indicated in Tables. The invention is directed to specific reagents recognizing the preferred terminal epitopes on N-glycans.

Over view of 50 most common structures

Neutral Glycans

Figures shows neutral glycans at three differentiation stages. The structures of glycans are indicated by symbols based on the recommendations of Consortium for Functional Glycomics. The glycans include terminal mannose comprising structures with regular high-mannose structures and low mannose structures, with characteristic changes during differentiation.

The mannose glycans further includes single HexNAc comprising structures H₄₋₁₀N₁, which also change during differentiation. A specifically characteric glycans have compositions H4N1 and H5N1,which increase during differentiation from stage 1 (ES cells) to stage 2 (EB) and further to stage 3. The other signal in this group (H6N1, H7N1, H8N1, H9N1 and H10N1 increase to stage 2 but the decrease.

The glycans are assigned as degradation products of High/Low mannose or even hybrid type structures. A preferred structural assignment is directed to glycans with High/Low mannose structures comprising single GlcNAc unit at the reducing end. This type of glycans have been known from free cytosolic glycans as degradation products of N-glycans. The glycans are produced by endo-beta-N-acetylglucosaminidase (chitobiosidase) cleaving the glycan between the GlcNAc residues. It is realized that the glycan pool may also comprise hybrid type glycans released by endo-beta-mannosidase. The product would comprise N-acetyllactosamine on one branch and mannose residues on the other branch (lower variant of H4N1).

A selection of hybrid and complex type glycans are showns in Figures. The glycans includes hybrid type (and(or monoantennary glycans). In this first group (left) signal H3N3 shows major change from stage 2 to stage 3, and H2N4F1 from stage 1 to stage 3. The glycans classified as complex type structures in the middle also change during differentiation. The major signals corresponding to biantennary N glycans H5N4 and H5N4F1 decrease during the differentiation similarily as difucosylated structure H5N4F2 and multilactosaminylated H6N5 and H6N5F 1 structures preferably corresponding to triantennary glycans. The structures increasing during the differentiation includes H4N4, H3N5F1, H4N5F3, and H5N5 (structural scheme is lacking terminal Gal or hexose units).

Acidic Glycans

The figures indicates 50 most abundant acidic glycans. The major complex type N-glycan signals with sialic acids S1H5N4F1 and S1H5N4F2 decrease during differentiation, while the amounts of sulfated structures H5N4F1P, and S1H5N4F1P (P indicates sulfate or fosfate,) similarily as a structure comprising additional HexNAc (S1H5N5F1) increases.

The figures shows approximated relative amounts of hydrid type glycans indicating quite similar amounts of acidic and neutral hydrid/monoantenanry glycans. The relative amounts of both glycan types increases during differentiation. Sulfated (or fosforylated) glycans are increased among the hybrid type glycans.

The glycans changing during differentiation with composition S1H6N4F1Ac, S1H6N4F2, and H6N4 in a specific embodiment include biantennary structures with additional terminal hexose, which may be derived from exogenous proteins, in a specific embodiment the hexose is Galα3-structure.

Figures includes high and Low mannose structures. The changes of the low mannose structures during the differentiation are characteristic for the stem cells. The smallest low mannose structure (H1N2) decreases while larger ones increase.

Neutral and acidic fucosylated glycans are presented in Figs Among the entral fucosylated glycans the amounts of apparently degraded low mannose group structures are increased (H2N2F1, H3N2F1 and H3N3F1), while the complex type structures decrease similarily in acidic and neutral glycans except the structure with additional HexNAc, S1H5N5F1.

Figures shows the neutral and acidic glycans comprising at least two fucose residues. These are considered as comprising fucosylated lactosamine and referred as complex/complexly fucosylated structures. In general decrease of the complexly fucosylated structures is observed except the structures with additional HexNAc residues, H4N4F2 (potential degradation product), H5N5F3, H5N6F3.

Preferred Sulfated Marker Structures in N-Glycome of Embryonic Stem Cells

Figures represents sulfated N-glycans of human embryonic stem cells and changes in their relative abundance during differentiation. There is major changes during differentiation. The invention is directed to use of the signals, monosaccharide compositions and structures indicated as increasing in Figures for markers of differentiating embryonic stem cells. Experiments by cleavage by specific fosfatase enzyme and high resolution mass spectrometry indicate that the structures with complex type N-glycans with N-acetyllactosamine residues preferably carry sulfate residues (sulfate ester structures) and the Mannose type N-glycans such as high Mannose N-glycans preferably carries fosfate residue(s). It is realised that the sulphated and/or fosforylated glycomes from stem cells are new inventive markers.

The invention is especially directed to the recognition of sulphated N-acetyllactosamines as differentiation markers of stem cells, embryonic stem cells. The invention is directed to testing and selectin optimal stem cell recognizing binder molecule, preferably antibodies such as monoclonal antibodies, recognizing preferred sulphated lactosamines including type I (Galβ3GlcNAc) and type II lactosamines (Galβ4GlcNAc) comprising sulfate residue(ester) at either position 3 or 6 of Gal and/or on position 6 of GlcNAc. The invention is especially directed to the recognition of the sulphated lactosamines from an N-glycan composition as shown by the invention.

Large N-Glycan Structure

Figures. shows large N-glycans (H≧7, N≧6) of human embryonic stem cells and changes in their relative abundance during differentiation. Figures represents large N-glycans of human embryonic stem cells and changes in their relative abundance during differentiation. There is major changes during differentiation. The invention is directed to use of the signals, monosaccharide compositions and structures indicated as increasing in Figures for markers of differentiating embryonic stem cells.

The invention reveals that the N-glycans of embryonic stem cells comprise multiantennary N-aglycans with at least three antennae with characteristic differentiation associated cahges. The invention reveals even much larger N-glycans containing poly-N-acetyllctosamine glycans. The invention is especially directed to use of reagents recognizing linear (example of preferred regent potato lectin, Solanum tuberosum agglutinin, STA) or branced poly-N-acetyllactosamine The results revealed that recognition of branched N-acetyllactosamines is especially useful for characterization or separation or manipulation of embyronal stem cells. Preferred reagents includes PWA, pokeweed agglutinin and/or antibody recognizing brancehed poly-N-acetyllactosamines such as I-blood group antibodies.

Cell Types

In the present text, cell types refer to stem cells, especially human embryonic stem cells (hESC) and cells differentiated from them, preferentially embryoid bodies (EB) and stage 3 (st.3) and further differentiated cells and other stem cells including hematopoietic stem cells.

Glycan Dataset and Glycan Profile Analysis

The present invention is directed to analysing glycan profiles to enable uses including the following:
1. comparison between stem cell and differentiated samples,
2. comparison between different samples of the same cell type,
3. identification of differentiation stage,
4. identification of glycan signals and glycan structures associated with different cell types or differentiation stages,
5. identification of glycan signal groups and glycan structure groups associated with different cell types or differentiation stages,
6. identification of biosynthetic glycan groups associated with different cell types or differentiation stages,
7. identification of glycan fingerprints and glycan signatures, i.e. glycan profiles or subprofiles therefrom, respectively, which are associated with different cell types or differentiation stages, and
8. evaluating glycans or glycan groups with respect to their degree of association with given cell type.

As described in the present invention, analysis of multiple samples from the same cell type reveals that some glycans or glycan groups are constantly associated with given cell type, whereas other glycans or glycan groups vary individually or between different samples within the same cell type. The present invention is especially directed to analyzing multiple samples of a given cell type to reach a point of statistical confidence, preferentially over 95% confidence level and even more preferentially over 96% confidence level, where given cell type or the glycan types associated with it can be reliably identified.

The present invention is specifically directed to comparison of multiple glycan profile data to find out which glycan signals are consistently associated with given cell type or not present in it, which are constant in all cell types, which are subject to individual or cell line specific variation, and which are indicative for the absence or presence of certain differentiation stages or lineages, more preferentially pluripotency (stem cell) or neuroectodermal differentation. The inventors found that the N-glycan profiles of human embryonic stem cells and cell derived from them contain glycan signals and glycan signal groups with the properties described above.

The present invention is further directed to establishing reference datasets from single glycan signals or glycan fingerprints or signatures (profiles or subprofiles), which can be reliably used for quality control, estimation of differential properties of new samples, control of variation between samples, or estimation of the effects of external factors or culture conditions on cell status. In this aspect of the invention, data acquired from new sample are compared to reference dataset with a predetermined equation to evaluate the status of the sample.

Structure Specific Glycan Binding Reagents

The present invention is further directed to using knowledge of glycan features associated with different cell types or differentiation stages to design glycan-binding reagents, more preferably glycan-binding proteins, for specific identification of stem cells or differentiated cells. The present invention is further directed to using such structure specific reagents to specifically recognize, label, or tag either specific stem cell or specific differentiated cell types, more preferentially animal feeder cells and more preferably mouse feeder cells. Such labels or tags can then be used to isolate and/or remove such cells by methods known in the art.

The Binding Methods for Recognition of Structures from Cell Surfaces Recognition of Structures from Glycome Materials and on Cell Surfaces by Binding Methods

The present invention revealed that beside the physicochemical analysis by NMR and/or mass spectrometry several methods are useful for the analysis of the structures. The invention is especially directed to two methods:

ii) Recognition by enzymes involving binding and alteration of structures. This method alters specific glycan structures by enzymes cabable of altering the glycan structures. The preferred enzymes includes
a) glycosidase-type enzymes capable of releasing monosaccharide units from glycans
b) glycosyltransferring enzymes, including transglycosylating enzymes and glycosyltransferases
c) glycan modifying enzymes including sulfate and or fosfate modifying enzymes

iii) Recognition by molecules binding glycans referred as the binders These molecules bind glycans and include property allowing observation of the binding such as a label linked to the binder. The preferred binders include
a) Proteins such as antibodies, lectins and enzymes
b) Peptides such as binding domains and sites of proteins, and synthetic library derived analogs such as phage display peptides
c) Other polymers or organic scaffold molecules mimicking the peptide materials

The peptides and proteins are preferably recombinant proteins or corresponding carbohydrate recognition domains derived thereof, when the proteins are selected from the group monoclonal antibody, glycosidase, glycosyl transferring enzyme, plant lectin, animal lectin or a peptide mimetic thereof, and wherein the binder includes a detectable label structure..

Binder-Label Conjugates

The present invention is specifically directed to the binding of the structures according to the present invention, when the binder is conjugated with “a label structure”. The label structure means a molecule observable in a assay such as for example a fluorescent molecule, a radioactive molecule, a detectable enzyme such as horse radish peroxidase or biotin/streptavidin/avidin. When the labelled binding molecule is contacted with the cells according to the invention, the cells can be monitored, observed and/or sorted based on the presence of the label on the cell surface. Monitoring and observation may occur by regular methods for observing labels such as fluorescence measuring devices, microscopes, scintillation counters and other devices for measuring radioactivity.

Use of Binder and Labelled Binder-Conjugates for Cell Sorting

The invention is specifically directed to use of the binders and their labelled cojugates for sorting or selecting cells from biological materials or samples including cell materials comprising other cell types. The preferred cell types includes cultivated cells and associated cells such as feeder cells. The labels can be used for sorting cell types according to invention from other similar cells. In another embodiment the cells are sorted from different cell types such as blood cells or in context of cultured cells preferably feeder cells, for example in context of complex cell cultures corresponding feeder cells such as human or mouse feeder cells. A preferred cell sorting method is FACS sorting. Another sorting methods utilized immobilized binder structures and removal of unbound cells for separation of bound and unbound cells.

Use of Immobilized Binder Structures

In a preferred embodiment the binder structure is conjugated to a solid phase. The cells are contacted with the solid phase, and part of the material is bound to surface. This method may be used to separation of cells and analysis of cell surface structures, or study cell biological changes of cells due to immobilization. In the analytics involving method the cells are preferably tagged with or labelled with a reagent for the detection of the cells bound to the solid phase through a binder structure on the solid phase. The methods preferably further include one or more steps of washing to remove unbound cells.

Preferred solid phases include cell suitable plastic materials used in contacting cells such as cell cultivation bottles, petri dishes and microtiter wells; fermentor surface materials

Specific Recognition Between Preferred Stem Cells and Contaminating Cells

The invention is further directed to methods of recognizing stem cells from differentiated cells such as feeder cells, preferably animal feeder cells and more preferably mouse feeder cells. It is further realized, that the present reagents can be used for purification of stem cells by any fractionation method using the specific binding reagents.

Preferred fractionation methods includes fluorecense activated cell sorting (FACS), affinity chromatography methods, and bead methods such as magnetic bead methods.

Preferred reagents for recognition between preferred cells, preferably embryonic type cells, and and contaminating cells, such as feeder cells most preferably mouse feeder cells, includes reagents according to the Tables, more preferably proteins with similar specificity with lectins PSA, MAA, and PNA.

The invention is further directed to positive selection methods including specific binding to the stem cell population but not to contaminating cell population. The invention is further directed to negative selection methods including specific binding to the contaminating cell population but not to the stem cell population. In yet another embodiment of recognition of stem cells the stem cell population is recognized together with a homogenous cell population such as a feeder cell population, preferably when separation of other materials is needed. It is realized that a reagent for positive selection can be selected so that it binds stem cells as in present invention and not to the contaminating cell population and a regent for negative selection by selecting opposite specificity. In case of one population of cells according to the invention is to be selected from a novel cell population not studied in the present invention, the binding molecules according to the invention maybe used when verified to have suitable specificity with regard to the novel cell population (binding or not binding). The invention is specifically directed to analysis of such binding specificity for development of a new binding or selection method according to the invention.

The preferred specificities according to the invention includes recognition of :
i) mannose type structures, especially alpha-Man structures like lectin PSA, preferably on the surface of contaminating cells
ii) α3-sialylated structures similarily as by MAA-lectin, preferably for recognition of embryonic type stem cells
iii) Gal/GalNAc binding specificity, preferably Gal1-3/GalNAc1-3 binding specificity, more preferably Galβ1-3/GalNAcβ1-3 binding specificity similar to PNA, preferably for recognition of embryonic type stem cells

Low Amounts of Cells for Glycome Analysis from Stem Cells

The invention revealed that its possible to produce glycome from very low amount of cells. The preferred embodiments amount of cells is between 1000 and 10 000 000 cells, more preferably between 10 000 and 1 000 000 cells. The invention is further directed to analysis of released glycomes of amount of at least 0.1 pmol, more preferably of at least to 1 pmol, more preferably at least of 10 pmol.

(a) Total asparagine-linked glycan (N-glycan) pool was enzymatically isolated from about 100 000 cells. (b) The total N-glycan pool (picomole quantities) was purified with microscale solid-phase extraction and divided into neutral and sialylated N-glycan fractions. The N-glycan fractions were analyzed by MALDI-TOF mass spectrometry either in positive ion mode for neutral N-glycans (c) or in negative ion mode for sialylated glycans (d). Over one hundred N-glycan signals were detected from each cell type revealing the surprising complexity of hESC glycosylation. The relative abundances of the observed glycan signals were determined based on relative signal intensities (Saarinen et al., 1999, Eur. J. Biochem. 259, 829-840).

Preferred Structures of O-Glycan Glycomes of Stem Cells

The present invention is especially directed to following O-glycan marker structures of stem cells:

Core 1 type O-glycan structures following the marker composition NeuAc₂Hex₁HexNAc₁, preferably including structures SAα3Galβ3GalNAc and/or SAα3Galβ3(Saα6)GalNAc;

and Core 2 type O-glycan structures following the marker composition NeuAc₀₋₂Hex2HexNAc₂dHex₀₋₁, more preferentially further including the glycan series NeuAc₀₋₂Hex₂₊ₙHexNAc₂₊ₙdHex₀₋₁, wherein n is either 1, 2, or 3 and more preferentially n is 1 or 2, and even more preferentially n is 1; more specifically preferably including R₁Galβ4(R₃)GlcNAcβ6(R₂Galβ3)GalNAc, wherein R₁ and R₂ are independently either nothing or sialic acid residue, preferably α2,3-linked sialic acid residue, or an elongation with HexₙHexNAcₙ, wherein n is independently an integer at least 1, preferably between 1-3, most preferably between 1-2, and most preferably 1, and the elongation may terminate in sialic acid residue, preferably α2,3-linked sialic acid residue; and

R₃ is independently either nothing or fucose residue, preferably α1,3-linked fucose residue.

It is realized that these structures correlate with expression of β6GleNAc-transferases synthesizing core 2 structures.

Preferred Branched N-Acetyllactosamine Type Glycosphingolipids

The invention further revealed branched, I-type, poly-N-acetyllactosamines with two terminal Galβ4-residues from glycolipids of human stem cells. The structures correlate with expression of β6GlcNAc-transferases capable of branching poly-N-acetyllactosamines and further to binding of lectins specific for branched poly-N-acetylalctosamines. It was further noticed that PWA-lectin had an activity in manipulation of stem cells, especially the growth rate thereof.

Analysis and Utilization of Poly-N-Acetyllactosamine Sequences and Non-Reducing Terminal Epitopes Associated with Different Glycan Types

The present invention is directed to poly-N-acetyllactosamine sequences (poly-LacNAc) associated with cell types according to the present invention. The inventors found that different types of poly-LacNAc are characteristic to different cell types, as described in the Examples of the present invention. hESC are characterized by type 1 terminating poly-LacNAc, especially on O-glycans and glycolipids. The present invention is especially directed to the analysis and utilization of these glycan characteristics according to the present invention. The present invention is further directed to the analysis and utilization of the specific cell-type accociated glycan sequences revealed in the present Examples according to the present invention.

The present invention is directed to non-reducing terminal epitopes in different glycan classes including N- and O-glycans, glycosphingolipid glycans, and poly-LacNAc. The inventors found that especially the relative amounts of β1,4-linked Gal, β1,3-linked Gal, α1,2-linked Fuc, α1,3/4-linked Fuc, α-linked sialic acid, and α2,3-linked sialic acid are characteristically different between the studied cell types; and the invention is especially directed to the analysis and utilization of these glycan characteristics according to the present invention.

The present invention is further directed to analyzing fucosylation degree in O-glycans by comparing indicative glycan signals such as neutral O-glycan signals at m/z 771 and 917 as described in the Examples. The inventors found that compared to other cell types analyzed in the present invention, hESC had low relative abundance of neutral O-glycan signal at m/z 917 compared to 771, indicating low fucosylation degree of the O-glycan sequences corresponding to the signal at m/z 771 and containing terminal β1,4-linked Gal. Another difference was the occurrence of abundant signal at m/z 552 in hESC, corresponding to Hex₁HexNAc₁dHex₁, including α1,2-fucosylated Core 1 O-glycan sequence. In contrast, in CB MNC the glycan signal at m/z 917 is relatively abundant, indicating high fucosylation degree of the O-glycan sequences corresponding to the signal at m/z 771 and containing terminal β1,4-linked Gal. The other cell types analyzed in the present invention also had characteristic fucosylation degree between these two cell types.

Especially, the present invention is directed to analyzing terminal epitopes associated with poly-LacNAc in stem cells, more preferably when these epitopes are presented in the context of a poly-LacNAc chain, most preferably in O-glycans or glycosphingolipids. The present invention is further directed to analyzing such characteristic poly-LacNAc, terminal epitope, and fucosylation profiles according to the methods of the present invention, in glycan structural characterization and specific glycosylation type identification, and other uses of the present invention; especially when this analysis is done based on endo-3-galactosidase digestion, by studying the non-reducing terminal fragments and their profile, and/or by studying the reducing terminal fragments and their profile, as described in the Examples of the present invention. The inventors found that cell-type specific glycosylation features are efficiently reflected in the endo-3-galactosidase reaction products and their profiles.

The present invention is further directed to such reaction product profiles and their analysis according to the present invention.

Especially in hESC, the inventors found that characteristic non-reducing poly-LacNAc associated sequences include Fucα2Gal, Galβ3GlcNAc, Fucα2Galβ3GlcNAc, and α3′-sialylated Galβ3GlcNAc. The present invention is especially directed to analysis of such glycan structures according to the present methods, in context of stem cells and differentiation of stem cells, preferably in context of human embryonic stem cells and their differentiation.

The inventors further found that all three most thoroughly analyzed cellular glycan classes, N-glycans, O-glycans, and glycosphingolipid glycans, were differently regulated compared to each other, especially with regard to non-reducing terminal glycan epitopes and poly-LacNAc sequences as described in the Examples and Tables of the present invention. Therefore, combining quantitative glycan profile analysis data from more than one glycan class will yield significantly more information. The present invention is especially directed to combining glycan data obtained by the methods of the present invention, from more than one glycan class selected from the group of N-glycans, O-glycans, and glycosphingolipid glycans; more preferably, all three classes are analyzed; and use of this information according to the present invention. In a preferred embodiment, N-glycan data is combined with O-glycan data; and in a further preferred embodiment, N-glycan data is combined with glycosphingolipid glycan data.

The FACS data in Tables and Figures indicates some antibodies recognizing the major elongated glycan structure epitopes according to the invention on cell surfaces. The invention is especially directed to the use of the H type II, H type I, type I LacNAc (Lewis c) and globotriose specific antibodies for the recognition of the embryonic stem cells, GF286, GF287, GF 279 and GF367. The invention is further directed to the major cell populations isolatable by the antibodies. The invention is further directed to the antibodies with similar specificties as the antibodies recognizing the major cell population of the embryonal stem cells. The invention is preferably directed to recognition of the elongated epitopes of H type II and H type I and type I LacNAc structures according to the invention by specific binder regents, preferably by antibodies. The invention is further directed to the recognition of the novel stem cell marker globotriose from the embryonal type stem cells and isolation of the cell population by the by using the specific binder for the glycan structure.

The invention is in a preferred embodiment directed to the short globoseries structures such as globotriose non-reducing end globotriose (Gb3) epitopes: Galα4Gal, Galα4Galβ and Galα4Galβ4Glc for the methods according to the invention. In a preferred embodiment the invention is directed to the recognition of the ceramide linked globotriose epitope. It is realized that though larger globoseries structures SSEA-3 and SSEA-4 has been indicated from embryonic stem cells, this structure has not been known from embryonic type stem cells and their amounts have been unpredictable.

The invention is directed to use of binders with elongated specificity, when the binders recognize or is able to bind at least one reducing end elongation monosaccharide epitope according to the formula

AxHex(NAc)ₙ, wherein A is anomeric structure alfa or beta, X is linkage position 2, 3, 4, or 6

And Hex is hexopyranosyl residue Gal, or Man, and n is integer being 0 or 1, with the provisions that when n is 1 then AxHexNAc is β4GalNAc or β6GalNAc, when Hex is Man, then AxHex is β2Man, and when Hex is Gal, then AxHex is β3Gal or β6Gal. Beside the monosaccharide elongation structures αSer/Thr are preferred reducing end elongation structures for reducing end GalNAc-comprising O-glycans and βCer is preferred for lactosyl comprising glycolipid epitopes.

The preferred subgroups of the elongation structures includes i) similar structural epitopes present on O-glycans, polylactosamine and glycolipid cores: β3/6Gal or β6GalNAc; with preferred further subgroups ia) β6GalNAc/β6Gal and ib) β3Gal; ii) N-glycan type epitope β2Man; and iii) globoseries epitopes α3Gal or α4Gal. The groups are preferred for structural similarity on possible cross reactivity within the groups, which can be used for increasing labeling intensity when background materials are controlled to be devoid of the elongated structure types.

Useful binder specifities including lectin and elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritstmci.ac.jp/epitope/, which list monoclonal antibody glycan specificities).

Preferred Binder Molecules

The present invention revealed various types of binder molecules useful for characterization of cells according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

Preferred Combinations of the Binders

The invention revealed useful combination of specific terminal structures for the analysis of status of a cells. In a preferred embodiment the invention is directed to measuring the level of two different terminal structures according to the invention, preferably by specific binding molecules, preferably at least by two different binders. In a preferred embodiment the binder molecules are directed to structures indicating modification of a terminal receptor glycan structures, preferably the structures represent sequential (substrate structure and modification thereof, such as terminal Gal-structure and corresponding sialylated structure) or competing biosynthetic steps (such as fucosylation and sialylation of terminal Galβ or terminal Galβ3GlcNAc and Galβ4GlcNAc). In another embodiment the binders are directed to three different structures representing sequential and competing steps such as such as terminal Gal-structure and corresponding sialylated structure and corresponding sialylated structure.

The invention is further directed to recognition of at least two different structures according to the invention selected from the groups of non-modified (non-sialylated or non-fucosylated) Gal(NAc)β3/4− core structures according to the invention, preferred fucosylated structures and preferred sialylated structures according to the invention. It is realized that it is useful to recognize even 3, and more preferably 4 and even more preferably five different structures, preferably within a preferred structure group.

Target Structures for Specific Binders and Examples of the Binding Molecules

Combination of Terminal Structures with Specific Glycan Core Structures

It is realized that part of the structural elements are specifically associated with specific glycan core structure. The recognition of terminal structures linked to specific core structures are especially preferred, such high specificity reagents have capacity of recognition almost complete individual glycans to the level of physicochemical characterization according to the invention. For example many specific mannose structures according to the invention are in general quite characteristic for N-glycan glycomes according to the invention. The present invention is especially directed to recognition terminal epitopes.

Common Terminal Structures on Several Glycan Core Structures

The present invention revealed that there are certain common structural features on several glycan types and that it is possible to recognize certain common epitopes on different glycan structures by specific reagents when specificity of the reagent is limited to the terminal without specificity for the core structure. The invention especially revealed characteristic terminal features for specific cell types according to the invention. The invention realized that the common epitopes increase the effect of the recognition. The common terminal structures are especially useful for recognition in the context with possible other cell types or material, which do not contain the common terminal structure in substantial amount.

The invention revealed the presence of the terminal structures on specific core structures such as N-glycan, O-glycan and/or glycolipids. The invention is preferably directed to the selection of specific binders for the structures including recognition of specific glycan core types.

The invention is further directed to glycome compositions of protein linked glycomes such as N-glycans and O-glycans and glycolipids each composition comprising specific amounts of glycan subgroups. The invention is further directed to the compositions when these comprise specific amount of Defined terminal structures.

Specific Preferred Structural Groups

The present invention is directed to recognition of oligosaccharide sequences comprising specific terminal monosaccharide types, optionally further including a specific core structure. The preferred oligosaccharide sequences are in a preferred embodiment classified based on the terminal monosaccharide structures.

The invention further revealed a family of terminal (non-reducing end terminal) disaccharide epitopes based on β-linked galactopyranosylstructures, which may be further modified by fucose and/or sialic acid residues or by N-acetylgroup, changing the terminal Gal residue to GalNAc. Such structures are present in N-glycan, O-glycan and glycolipid subglycomes. Furthermore the invention is directed to terminal disaccharide epitopes of N-glycans comprising terminal ManαMan.

The structures were derived by mass spectrometric and optionally NMR analysis and by high specificity binders according to the invention, for the analysis of glycolipid structures permethylation and fragmentation mass spectrometry was used. Biosynthetic analysis including known biosynthetic routes to N-glycans, O-glycans and glycolipids was additionally used for the analysis of the glycan compositions and additional support, though not direct evidence due to various regulation levels after mRNA, for it was obtained from gene expression profiling data of Skottman, H. et al. (2005) Stem cells and similar data obtained from the mRNA profiling for cord blood cells and used to support the biosynthetic analysis using the data of Jaatinen T et al. Stem Cells (2006) 24 (3) 631-41.

Structures with Terminal Mannose Monosaccharide

Preferred mannose-type target structures have been specifically classified by the invention. These include various types of high and low-mannose structures and hybrid type structures according to the invention.

The Preferred Terminal Manα-Target Structure Epitopes

The invention revealed the presence of Manα on low mannose N-glycans and high mannose N-glycans. Based on the biosynthetic knowledge and supporting this view by analysis of mRNAs of biosynthetic enzymes and by NMR-analysis the structures and terminal epitopes could be revealed:

Manα2Man, Manα3Man, Manα6Man and Manα3(Manα6)Man, wherein the reducing end Man is preferably either α- or β-linked glycoside and α-linked glycoside in case of Manα2Man:

The general structure of terminal Manα-structures is

Manαx(Manαy)_{z}Manα/β

Wherein x is linkage position 2, 3 or 6, and y is linkage position 3 or 6,

z is integer 0 or 1, indicating the presence or the absence of the branch,

with the provision that x and y are not the same position and

when x is 2, the z is 0 and reducing end Man is preferably α-linked;

The low_mannose structures includes preferably non-reducing end terminal epitopes with structures with α3- and/or α6-mannose linked to another mannose residue Manαx(Manαy)_{z}Manα/β

wherein x and y are linkage positions being either 3 or 6,

z is integer 0 or 1, indicating the presence or the absence of the branch,

The high mannose structure includes terminal α2-linked Mannose:

Manα2Man(α) and optionally on or several of the terminal α3- and/or α6-mannose-structures as above.

The presence of terminal Manα-structures is regulated in stem cells and the proportion of the high-Man-structures with terminal Manα2-structures in relation to the low Man structures with Manα3/6- and/or to complex type N-glycans with Gal-backbone epitopes varies cell type specifically.

The data indicated that binder revealing specific terminal Manα2Man and/or Manα3/6Man is very useful in characterization of stem cells. The prior science has not characterized the epitopes as specific signals of cell types or status.

The invention is especially directed to the measuring the levels of both low-Man and high-Man structures, preferably by quantifying two structure type the Manα2Man-structures and the Manα3/6Man-structures from the same sample.

The invention is especially directed to high specificity binders such as enzymes or monoclonal antibodies for the recognition of the terminal Manα-structures from the preferred stem cells according to the invention, more preferably from differentiated embryonal type cells, more preferably differentiated beyond embryoid bodies such as stage 3 differentiated cells, most preferably the structures are recognized from stage 3 differentiated cells. The invention is especially preferably directed to detection of the structures from adult stem cells more preferably mesenchymal stem cells, especially from the surface of mesenchymal stem cells and in separate embodiment from blood derived stem cells, with separately preferred groups of cord blood and bone marrow stem cells. In a preferred embodiment the cord blood and/or peripheral blood stem cell is not hematopoietic stem cell.

Low or Uncharacterised Specificity Binders

preferred for recognition of terminal mannose structures includes mannose-monosaccharide binding plant lectins. The invention is in preferred embodiment directed to the recognition of stem cells such as embryonal type stem cells by a Manα-recognizing lectin such as lectin PSA. In a preferred embodiment the recognition is directed to the intracellular glycans in permebilized cells. In another embodiment the Manα-binding lectin is used for intact non-permeabilized cells to recognize terminal Manα-from contaminating cell population such as fibroblast type cells or feeder cells as shown in corresponding Examples.

Preferred High Specific High Specificity Binders

include

i) Specific mannose residue releasing enzymes such as linkage specific mannosidases, more preferably an α-mannosidase or β-mannosidase.

Preferred α-mannosidases includes linkage specific α-mannosidases such as α-Mannosidases cleaving preferably non-reducing end terminal, an example of preferred mannosidases is jack bean α-mannosidase (Canavalia ensiformis; Sigma, USA) and homologous α-mannosidases

α2-linked mannose residues specifically or more effectively than other linkages, more preferably cleaving specifically Manα2-structures; or

α3-linked mannose residues specifically or more effectively than other linkages, more preferably cleaving specifically Manα3-structures; or

α6-linked mannose residues specifically or more effectively than other linkages, more preferably cleaving specifically Manα6-structures;

Preferred β-mannosidases includes β-mannosidases capable of cleaving β4-linked mannose from non-reducing end terminal of N-glycan core Manβ4GlcNAc-structure without cleaving other β-linked monosaccharides in the glycomes.

ii) Specific binding proteins recognizing preferred mannose structures according to the invention. The preferred reagents include antibodies and binding domains of antibodies (Fab-fragments and like), and other engineered carbohydrate binding proteins. The invention is directed to antibodies recognizing MS2B1 and more preferably MS3B2-structures.

Lectin Binding

α-linked mannose was demonstrated in Examples for human mesenchymal cell by lectins Hippeastrum hybrid (HHA) and Pisum sativum (PSA) lectins suggests that they express mannose, more specifically α-linked mannose residues on their surface glycoconjugates such as N-glycans. Possible α-mannose linkages include α1→2, α1→3, and α1→6. The lower binding of Galanthus nivalis (GNA) lectin suggests that some α-mannose linkages on the cell surface are more prevalent than others. The combination of the terminal Manα-recognizing low affinity reagents appears to be useful and correspond to results obtained by mannosidase screening; NMR and mass spectrometric results. Lectin binding of cord blood cells is in examples. PSA has specificity for complex type N-glycans with core Fucα6-eptopes.

Mannose-binding lectin labelling. Labelling of the mesenchymal cells in Examples was also detected with human serum mannose-binding lectin (MBL) coupled to fluorescein label. This indicate that ligands for this innate immunity system component may be expressed on in vitro cultured BM MSC cell surface.

The present invention is especially directed to analysis of terminal Manα-on cell surfaces as the structure is ligand for MBL and other lectins of innate immunity. It is further realized that terminal Manα-structures would direct cells in blood circulation to mannose receptor comprising tissues such as Kupfer cells of liver. The invention is especially directed to control of the amount of the structure by binding with a binder recognizing terminal Manα-structure.

In a preferred embodiment the present invention is directed to the testing of presence of ligands of lectins present in human, such as lectins of innate immunity and/or lectins of tissues or leukocytes, on stem cells by testing of the binding of the lectin (purified or preferably a recombinant form of the lectin, preferably in lableed form) to the stem cells. It is realized that such lectins includes especially lectins binding Manα and Galβ/GalNAcβ-structures (terminal non-reducing end or even α6-sialylated forms according to the invention.

Mannose Binding Antibodies

A high-mannose binding antibody has been described for example in Wang L X et al (2004) 11 (1) 127-34. Specific antibodies for short mannosylated structures such as the trimannosyl core structure have been also published.

Structures with Terminal Gal-Monosaccharide

Preferred galactose-type target structures have been specifically classified by the invention. These include various types of N-acetyllactosamine structures according to the invention.

Low or Uncharacterised Specificity Binders for Terminal Gal

Preferred for recognition of terminal galactose structures includes plant lectins such as ricin lectin (ricinus communis agglutinin RCA), and peanut lectin(/agglutinin PNA). The low resolution binders have different and broad specificities.

Preferred High Specific High Specificity Binders Include

i) Specific galactose residue releasing enzymes such as linkage specific galactosidases, more preferably α-galactosidase or β-galactosidase. Preferred α-galactosidases include linkage galactosidases capable of cleaving Galα3Gal-structures revealed from specific cell preparations

Preferred β-galactosidases includes β-galactosidases capable of cleaving

β4-linked galactose from non-reducing end terminal Galβ4GlcNAc-structure without cleaving other β-linked monosaccharides in the glycomes and

β3-linked galactose from non-reducing end terminal Galβ3GlcNAc-structure without cleaving other β-linked monosaccharides in the glycomes

ii) Specific binding proteins recognizing preferred galactose structures according to the invention. The preferred reagents include antibodies and binding domains of antibodies (Fab-fragments and like), and other engineered carbohydrate binding proteins and animal lectins such as galectins.

Specific Binder Experiments and Examples for Galβ-Structures

Specific exoglycosidase and glycosyltransferase analysis for the structures are included in Examples for embryonal stem cells and differentiated cells; Examples mesenchymal cells, for cord blood cells in examples and in examples on cell surface and including glycosyltransferases, for glycolipids in Examples. Sialylation level analysis related to terminal Galβ and Sialic acid expression is in Examples.

Preferred enzyme binders for the binding of the Galβ-epitopes according to the invention includes β1,4-galactosidase e.g from S. pneumoniae (rec. in E. coli, Calbiochem, USA), β1,3-galactosidase (e.g rec. in E. coli, Calbiochem); glycosyltransferases: α2,3-(N)-sialyltransferase (rat, recombinant in S. frugiperda, Calbiochem), α1,3-fucosyltransferase VI (human, recombinant in S. frugiperda, Calbiochem), which are known to recognize specific N-acetyllactosamine epitopes, Fuc-TVI especially Galβ4GlcNAc.

Plant low specificity lectin, such as RCA, PNA, ECA, STA, and

PWA, data is in Examples or hESC, Examples for MSCs, Examples for cord blood, effects of the lectin binders for the cell proliferation is in Examples, cord blood cell selection is in Examples.

Human lectin analysis by various galectin expression is Examples from cord blood and embryonal cells,

In examples there is antibody labeling of especially fucosylated and galactosylated structures.

Poly-N-acetyllactosamine sequences. Labelling of the cells by pokeweed (PWA) and less intense labelling by Solanum tuberosum (STA) lectins suggests that the cells express poly-N-acetyllactosamine sequences on their surface glycoconjugates such as N- and/or O-glycans and/or glycolipids. The results further suggest that cell surface poly-N-acetyllactosamine chains contain both linear and branched sequences.

Structures with Terminal GalNAc-Monosaccharide

Preferred GalNAc-type target structures have been specifically revealed by the invention. These include especially LacdiNAc, GalNAcβGlcNAc-type structures according to the invention.

Low or Uncharacterised Specificity Binders for Terminal GalNAc

Several plant lectins has been reported for recognition of terminal GalNAc. It is realized that some GalNAc-recognizing lectins may be selected for low specificity reconition of the preferred LacdiNAc-structures.

β-linked N-acetylgalactosamine. Abundant labelling of hESC by Wisteria floribunda lectin (WFA) suggests that hESC express β-linked non-reducing terminal N-acetylgalactosamine residues on their surface glycoconjugates such as N- and/or O-glycans. The absence of specific binding of WFA to mEF suggests that the lectin ligand epitopes are less abundant in mEF.

The low specificity binder plant lectins such as Wisteria floribunda agglutinin and Lotus tetragonolobus agglutinin bind to oligosaccharide sequences Srivatsan J. et al. Glycobiology (1992) 2 (5) 445-52: Do, K Y et al. Glycobiology (1997) 7 (2) 183-94; Yan, L., et al (1997) Glycoconjugate J. 14 (1) 45-55. The article also shows that the lectins are useful for recognition of the structures, when the cells are verified not to contain other structures recognized by the lectins.

In a preferred embodiment a low specificity leactin reagent is used in combination with another reagent verifying the binding.

Preferred High Specificity Binders Include

i) The invention revealed that β-linked GalNAc can be recognized by specific β-N-acetylhexosaminidase enzyme in combination with β-N-acetylhexosaminidase enzyme.

This combination indicates the terminal monosaccharide and at least part of the linkage structure.

Preferred β-N-acetylehexosaminidase, includes enzyme capable of cleaving β-linked GalNAc from non-reducing end terminal GalNAcβ4/3-structures without cleaving α-linked HexNAc in the glycomes; preferred N-acetylglucosaminidases include enzyme capable of cleaving β-linked GlcNAc but not GalNAc.

Specific binding proteins recognizing preferred GalNAcβ4, more preferably GalNAcβ4GlcNAc, structures according to the invention. The preferred reagents include antibodies and binding domains of antibodies (Fab-fragments and like), and other engineered carbohydrate binding proteins.

Examples antibodies recognizing LacdiNAc-structures includes publications of Nyame A. K. et al. (1999) Glycobiology 9 (10) 1029-35; van Remoortere A. et al (2000) Glycobiology 10 (6) 601-609; and van Remoortere A. et al (2001) Infect. Immun 69 (4) 2396-2401. The antibodies were characterized in context of parasite (Schistosoma) infection of mice and humans, but according to the present invention these antibodies can also be used in screening stem cells. The present invention is especially directed to selection of specific clones of LacdiNac recognizing antibodies specific for the subglycomes and glycan structures present in N-glycomes of the invention.

The articles disclose antibody binding specificities similar to the invention and methods for producing such antibodies, therefore the antibody binders are obvious for person skilled in the art. The immunogenicity of certain LacdiNAc-structures are demonstrated in human and mice.

The use of glycosidase in recognition of the structures in known in the prior art similarily as in the present invention for example in Srivatsan J. et al. (1992) 2 (5) 445-52.

Structures with Terminal GlcNAc-Monosaccharide

Preferred GlcNAc-type target structures have been specifically revealed by the invention. These include especially GlcNAcβ-type structures according to the invention.

Low or Uncharacterised Specificity Binders for Terminal GlcNAc

Several plant lectins has been reported for recognition of terminal GlcNAc. It is realized that some GlcNAc-recognizing lectins may be selected for low specificity reconition of the preferred GlcNAc-structures.

Preferred High Specific High Specificity Binders Include
i) The invention revealed that β-linked GlcNAc can be recognized by specific β-N-acetylglucosaminidase enzyme.

Preferred β-N-acetylglucosaminidase includes enzyme capable of cleaving β-linked GlcNAc from non-reducing end terminal GlcNAcβ2/3/6-structures without cleaving β-linked GalNAc or α-linked HexNAc in the glycomes;

ii) Specific binding proteins recognizing preferred GlcNAcβ2/3/6, more preferably GlcNAcβ2Manα, structures according to the invention. The preferred reagents include antibodies and binding domains of antibodies (Fab-fragments and like), and other engineered carbohydrate binding proteins.

Specific Binder Experiments and Examples for Terminal HexNAc(GalNAc/GlcNAc and GlcNAc Structures

Specific exoglycosidase analysis for the structures are included in Examples for embryonal stem cells and differentiated cells; Examples for mesenchymal cells, for cord blood cells in examples and for glycolipids in Example.

Plant low specificity lectin, such as WFA and GNAII, and data is in Examples for hESC, Examples for MSCs, Examples for cord blood, effects of the lectin binders for the cell proliferation is in Examples, cord blood cell selection is in Examples.

Preferred enzymes for the recognition of the structures includes general hexosaminidase β-hexosaminidase from Jack beans (C. ensiformis, Sigma, USA) and and specific N-acetylglucosaminidases or N-acetylgalactosaminidases such as β-glucosaminidase from S. pneumoniae (rec. in E. coli, Calbiochem, USA).

Combination of these allows determination of LacdiNAc.

The invention is further directed to analysis of the structures by specific monoclonal antibodies recognizing terminal GlcNAcβ-structures such as described in Holmes and Greene (1991) 288 (1) 87-96, with specificity for several terminal GlcNAc structures. The invention is specifically directed to the use of the terminal structures according to the invention for selection and production of antibodies for the structures.

Verification of the target structures includes mass spectrometry and permethylation/fragmentation analysis for glycolipid structures

Structures with Terminal Fucose-Monosaccharide

Preferred fucose-type target structures have been specifically classified by the invention. These include various types of N-acetyllactosamine structures according to the invention. The invention is further more directed to recognition and other methods according to the invention for lactosamine similar α6-fucosylated epitope of N-glycan core, GlcNAcβ4(Fucα6)GlcNAc. The invention revealed such structures recognizeable by the lectin PSA (Kornfeld (1981) J Biol Chem 256, 6633-6640; Cummings and Kornfeld (1982) J Biol Chem 257, 11235-40) are present e.g. in embryonal stem cells and mesenchymal stem cells.

Low or Uncharacterised Specificity Binders for Terminal Fuc

Preferred for recognition of terminal fucose structures includes fucose monosaccharide binding plant lectins. Lectins of Ulex europeaus and Lotus tetragonolobus has been reported to recognize for example terminal Fucoses with some specificity binding for α2-linked structures, and branching α3-fucose, respectively. Data is in Example for hESC, Examples for MSCs, Examples for cord blood, effects of the lectin binders for the cell proliferation is in Examples, cord blood cell selection is in Examples.

Preferred High Specific High Specificity Binders Include

i) Specific fucose residue releasing enzymes such as linkage fucosidases, more preferably α-fucosidase.

Preferred α-fucosidases include linkage fucosidases capable of cleaving Fucα2Gal−, and Galβ4/3(Fucα3/4)GlcNAc-structures revealed from specific cell preparations.

Specific exoglycosidase and for the structures are included in Examples for embryonal stem cells and differentiated cells; Examples for mesenchymal cells, for cord blood cells in examples and in examples on cell surface for glycolipids in Examples. Preferred fucosidases includes α1,3/4-fucosidase e.g. α1,3/4-fucosidase from Xanthomonas sp. (Calbiochem, USA), and α1,2-fucosidase e.g α1,2-fucosidase from X. manihotis (Glyko),

ii) Specific binding proteins recognizing preferred fucose structures according to the invention. The preferred reagents include antibodies and binding domains of antibodies (Fab-fragments and like), and other engineered carbohydrate binding proteins and animal lectins such as selectins recognizing especially Lewis type structures such as Lewis x, Galβ4(Fucα3)GlcNAc, and sialyl-Lewis x, SAα3Galβ4(Fucα3)GlcNAc.

The preferred antibodies includes antibodies recognizing specifically Lewis type structures such as Lewis x, and sialyl-Lewis x. More preferably the Lewis x-antibody is not classic SSEA-1 antibody, but the antibody recognizes specific protein linked Lewis x structures such as Galβ4(Fucα3)GlcNAcβ2Manα-linked to N-glycan core.

iii) the invention is further directed to reconition of α6-fucosylated epitope of N-glycan core, GlcNAcβ4(Fucα6)GlcNAc. The invention directed to recognition of such structures by structures by the lectin PSA or lentil lectin (Kornfeld (1981) J Biol Chem 256, 6633-6640) or by specific monoclonal antibodies (e.g. Srikrishna G. et al (1997) J Biol Chem 272, 25743-52). The invention is further directed to methods of isolation of cellular glycan components comprising the glycan epitope and isolation stem cell N-glycans, which are not bound to the lectin as control fraction for further characterization.

Structures with Terminal Sialic Acid-Monosaccharide

Preferred sialic acid-type target structures have been specifically classified by the invention.

Low or Uncharacterised Specificity Binders for Terminal Sialic Acid

Preferred for recognition of terminal sialic acid structures includes sialic acid monosaccharide binding plant lectins.

Preferred High Specific High Specificity Binders Include

i) Specific sialic acid residue releasing enzymes such as linkage sialidases, more preferably α-sialidases.

Preferred α-sialidases include linkage sialidases capable of cleaving SAα3Gal- and SAα6Gal-structures revealed from specific cell preparations by the invention. Preferred low specificity lectins, with linkage specificity include the lectins, that are specific for SAα3Gal-structures, preferably being Maackia amurensis lectin and/or lectins specific for SAα6Gal-structures, preferably being Sambucus nigra agglutinin.

ii) Specific binding proteins recognizing preferred sialic acid oligosaccharide sequence structures according to the invention. The preferred reagents include antibodies and binding domains of antibodies (Fab-fragments and like), and other engineered carbohydrate binding proteins and animal lectins such as selectins recognizing especially Lewis type structures such as sialyl-Lewis x, SAα3Galβ4(Fucα3)GlcNAc or sialic acid recognizing Siglec-proteins.

The preferred antibodies includes antibodies recognizing specifically sialyl-N-acetyllactosamines, and sialyl-Lewis x.

Preferred antibodies for NeuGc-structures includes antibodies recognizes a structure NeuGcα3Galβ4Glc(NAc)_{0 or 1} and/or GalNAcβ4[NeuGcα3]Galβ4Glc(NAc)_{0 or 1}, wherein [ ] indicates branch in the structure and ( )_{0 or 1} a structure being either present or absent. In a preferred embodiment the invention is directed recognition of the N-glycolyl-Neuraminic acid structures by antibody, preferably by a monoclonal antibody or human/humanized monoclonal antibody. A preferred antibody contains the variable domains of P3-antibody.

Specific Binder Experiments and Examples for α3/6 Sialylated Structures

Specific exoglycosidase analysis for the structures are included in Examples for embryonal stem cells and differentiated cells; Examples for mesenchymal cells, for cord blood cells in examples and in examples on cell surface and including glycosyltransferases, for glycolipids in Examples. Sialylation level analysis related to terminal Galβ and Sialic acid expression is in Examples.

Preferred enzyme binders for the binding of the Sialic acid epitopes according to the invention includes: sialidases such as general sialidase α2,3/6/8/9-sialidase from A. ureafaciens (Glyko), and α2,3-Sialidases such as: α2,3-sialidase from S. pneumoniae (Calbiochem, USA). Other useful sialidases are known from E. coli, and Vibrio cholerae.

α1,3-fucosyltransferase VI (human, recombinant in S. frugiperda, Calbiochem), which are known to recognize specific N-acetyllactosamine epitopes, Fuc-TVI especially including SAα3Galβ4GlcNAc.

Plant low specificity lectin, such as MAA and SNA, and data is in Examples for hESC, Examples for MSCs, Examples for cord blood, effects of the lectin binders for the cell proliferation is in Examples, cord blood cell selection is in Examples. In examples here is antibody labeling of sialylstructures.

Preferred Target Cell Populations and Types for Analysis According to the Invention

Early Human Cell Populations

Human Stem Cells and Multipotent Cells

Under broadest embodiment the present invention is directed to all types of human stem cells, meaning fresh and cultured human stem cells. The stem cells according to the invention do not include traditional cancer cell lines, which may differentiate to resemble natural cells, but represent non-natural development, which is typically due to chromosomal alteration or viral transfection. Stem cells include all types of non-malignant multipotent cells capable of differentiating to other cell types. The stem cells have special capacity stay as stem cells after cell division, the self-reneval capacity.

Under the broadest embodiment for the human stem cells, the present invention describes novel special glycan profiles and novel analytics, reagents and other methods directed to the glycan profiles. The invention shows special differences in cell populations with regard to the novel glycan profiles of human stem cells.

The present invention is further directed to the novel structures and related inventions with regard to the preferred cell populations according to the invention. The present invention is further directed to specific glycan structures, especially terminal epitopes, with regard to specific preferred cell population for which the structures are new.

Preferred Types of Early Human Cells

The invention is directed to specific types of early human cells based on the tissue origin of the cells and/or their differentiation status.

The present invention is specifically directed to early human cell populations meaning multipotent cells and cell populations derived thereof based on origins of the cells including the age of donor individual and tissue type from which the cells are derived, including preferred cord blood as well as bone marrow from older individuals or adults.

Preferred differentiation status based classification includes preferably “solid tissue progenitor” cells, more preferably “mesenchymal-stem cells”, or cells differentiating to solid tissues or capable of differentiating to cells of either ectodermal, mesodermal, or endodermal, more preferentially to mesenchymal stem cells.

The invention is further directed to classification of the early human cells based on the status with regard to cell culture and to two major types of cell material. The present invention is preferably directed to two major cell material types of early human cells including fresh, frozen and cultured cells.

Cord Blood Cells, Embryonal-Type Cells and Bone Marrow Cells

The present invention is specifically directed to early human cell populations meaning multipotent cells and cell populations derived thereof based on the origin of the cells including the age of donor individual and tissue type from which the cells are derived.
a) from early age-cells such 1) as neonatal human, directed preferably to cord blood and related material, and 2) embryonal cell-type material
b) from stem and progenitor cells from older individuals (non-neonatal, preferably adult), preferably derived from human “blood related tissues” comprising, preferably bone marrow cells.

Cells Differentiating to Solid Tissues, Preferably to Mesenchymal Stem Cells

The invention is specifically under a preferred embodiment directed to cells, which are capable of differentiating to non-hematopoietic tissues, referred as “solid tissue progenitors”, meaning to cells differentiating to cells other than blood cells. More preferably the cell population produced for differentiation to solid tissue are “mesenchymal-type cells”, which are multipotent cells capable of effectively differentiating to cells of mesodermal origin, more preferably mesenchymal stem cells.

Most of the prior art is directed to hematopoietic cells with characteristics quite different from the mesenchymal-type cells and mesenchymal stem cells according to the invention.

Preferred solid tissue progenitors according to the invention includes selected multipotent cell populations of cord blood, mesenchymal stem cells cultured from cord blood, mesenchymal stem cells cultured/obtained from bone marrow and embryonal-type cells. In a more specific embodiment the preferred solid tissue progenitor cells are mesenchymal stem cells, more preferably “blood related mesenchymal cells”, even more preferably mesenchymal stem cells derived from bone marrow or cord blood.

Under a specific embodiment CD34+ cells as a more hematopoietic stem cell type of cord blood or CD34+ cells in general are excluded from the solid tissue progenitor cells.

Early Blood Cell Populations and Corresponding Mesenchymal Stem Cells

Cord Blood

The early blood cell populations include blood cell materials enriched with multipotent cells. The preferred early blood cell populations include peripheral blood cells enriched with regard to multipotent cells, bone marrow blood cells, and cord blood cells. In a preferred embodiment the present invention is directed to mesenchymal stem cells derived from early blood or early blood derived cell populations, preferably to the analysis of the cell populations.

Bone Marrow

Another separately preferred group of early blood cells is bone marrow blood cells. These cell do also comprise multipotent cells. In a preferred embodiment the present invention is directed to directed to mesenchymal stem cells derived from bone marrow cell populations, preferably to the analysis of the cell populations.

Preferred Subpopulations of Early Human Blood Cells

The present invention is specifically directed to subpopulations of early human cells. In a preferred embodiment the subpopulations are produced by selection by an antibody and in another embodiment by cell culture favouring a specific cell type. In a preferred embodiment the cells are produced by an antibody selection method preferably from early blood cells. Preferably the early human blood cells are cord blood cells.

The CD34 positive cell population is relatively large and heterogenous. It is not optimal for several applications aiming to produce specific cell products. The present invention is preferably directed to specifically selected non-CD34 populations meaning cells not selected for binding to the CD34-marker, called homogenous cell populations. The homogenous cell populations may be of smaller size mononuclear cell populations for example with size corresponding to CD133+ cell populations and being smaller than specifically selected CD34+ cell populations. It is further realized that preferred homogenous subpopulations of early human cells may be larger than CD34+ cell populations.

The homogenous cell population may a subpopulation of CD34+ cell population, in preferred embodiment it is specifically a CD133+ cell population or CD133-type cell population. The “CD133-type cell populations” according to the invention are similar to the CD133+ cell populations, but preferably selected with regard to another marker than CD133. The marker is preferably a CD133-coexpressed marker. In a preferred embodiment the invention is directed to CD133+ cell population or CD133+ subpopulation as CD133-type cell populations. It is realized that the preferred homogeneous cell populations further includes other cell populations than which can be defined as special CD133-type cells.

Preferably the homogenous cell populations are selected by binding a specific binder to a cell surface marker of the cell population. In a preferred embodiment the homogenous cells are selected by a cell surface marker having lower correlation with CD34-marker and higher correlation with CD133 on cell surfaces. Preferred cell surface markers include α3-sialylated structures according to the present invention enriched in CD133-type cells. Pure, preferably complete, CD133+ cell population are preferred for the analysis according to the present invention.

The present invention is directed to essential mRNA-expression markers, which would allow analysis or recognition of the cell populations from pure cord blood derived material. The present invention is specifically directed to markers specifically expressed on early human cord blood cells.

The present invention is in a preferred embodiment directed to native cells, meaning non-genetically modified cells. Genetic modifications are known to alter cells and background from modified cells. The present invention further directed in a preferred embodiment to fresh non-cultivated cells.

The invention is directed to use of the markers for analysis of cells of special differentiation capacity, the cells being preferably human blood cells or more preferably human cord blood cells.

Preferred Purity of Reproducibly Highly Purified Mononuclear Complete Cell Populations from Human Cord Blood

The present invention is specifically directed to production of purified cell populations from human cord blood. As described above, production of highly purified complete cell preparations from human cord blood has been a problem in the field. In the broadest embodiment the invention is directed to biological equivalents of human cord blood according to the invention, when these would comprise similar markers and which would yield similar cell populations when separated similarly as the CD133+ cell population and equivalents according to the invention or when cells equivalent to the cord blood is contained in a sample further comprising other cell types. It is realized that characteristics similar to the cord blood can be at least partially present before the birth of a human. The inventors found out that it is possible to produce highly purified cell populations from early human cells with purity useful for exact analysis of sialylated glycans and related markers.

Preferred Bone Marrow Cells

The present invention is directed to multipotent cell populations or early human blood cells from human bone marrow. Most preferred are bone marrow derived mesenchymal stem cells. In a preferred embodiment the invention is directed to mesenchymal stem cells differentiating to cells of structural support function such as bone and/or cartilage.

A variety of factors previously mentioned influence ability of stem cells to survive, replicate, and differentiate. For example, in terms of nutrients the amino acid taurine under certain conditions preferentially inhibits murine bone marrow cells from forming osteoclasts (Koide, et al., 1999, Arch Oral Biol 44:711-719), the amino acid L-arginine stimulates erythrocyte differentiation and proliferation of erythroid progenitors (Shima, et al., 2006, Blood 107:1352-1356), extracellular ATP acting through P2Y receptors mediates a wide variety of changes to both hematopoietic and non-hematopoietic stem cells (Lee, et al., 2003, Genes Dev 17:1592-1604), arginine-glycine-aspartic acid attached to porous polymer scaffolds increase differentiation and survival of osteoblast progenitors (Hu, et al., 2003, J Biomed Mater Res A 64:583-590), each of which is incorporated by reference herein in its entirety. Accordingly, one skilled in the art would know to use various types of nutrients for inducing differentiation, or maintaining viability, of certain types of stem cells and/or progeny thereof.

Embryonal-Type Cell Populations

The present invention is specifically directed to methods directed to embryonal-type cell populations, preferably when the use does not involve commercial or industrial use of human embryos nor involve destruction of human embryos. The invention is under a specific embodiment directed to use of embryonal cells and embryo derived materials such as embryonal stem cells, whenever or wherever it is legally acceptable. It is realized that the legislation varies between countries and regions.

The present invention is further directed to use of embryonal-related, discarded or spontaneously damaged material, which would not be viable as human embryo and cannot be considered as a human embryo. In yet another embodiment the present invention is directed to use of accidentally damaged embryonal material, which would not be viable as human embryo and cannot be considered as human embryo.

It is further realized that early human blood derived from human cord or placenta after birth and removal of the cord during normal delivery process is ethically uncontroversial discarded material, forming no part of human being.

The invention is further directed to cell materials equivalent to the cell materials according to the invention. It is further realized that functionally and even biologically similar cells may be obtained by artificial methods including cloning technologies.

Mesenchymal Multipotent Cells

The present invention is further directed to mesenchymal stem cells or multipotent cells as preferred cell population according to the invention. The preferred mesencymal stem cells include cells derived from early human cells, preferably human cord blood or from human bone marrow. In a preferred embodiment the invention is directed to mesenchymal stem cells differentiating to cells of structural support function such as bone and/or cartilage, or to cells forming soft tissues such as adipose tissue.

Control of Cell Status and Potential Contaminations by Glycosylation Analysis

Control of Cell Status

Control of Raw Material Cell Population

The present invention is directed to control of glycosylation of cell populations to be used in therapy.

The present invention is specifically directed to control of glycosylation of cell materials, preferably when
1) there is difference between the origin of the cell material and the potential recipient of transplanted material. In a preferred embodiment there are potential inter-individual specific differences between the donor of cell material and the recipient of the cell material. In a preferred embodiment the invention is directed to animal or human, more preferably human specific, individual person specific glycosylation differences. The individual specific differences are preferably present in mononuclear cell populations of early human cells, early human blood cells and embryonal type cells. The invention is preferably not directed to observation of known individual specific differences such as blood group antigens changes on erythrocytes.
2) There is possibility in variation due to disease specific variation in the materials. The present invention is specifically directed to search of glycosylation differences in the early cell populations according to the present invention associated with infectious disease, inflammatory disease, or malignant disease. Part of the inventors have analysed numerous cancers and tumors and observed similar types glycosylations as certain glycosylation types in the early cells.
3) There is for a possibility of specific inter-individual biological differences in the animals, preferably humans, from which the cell are derived for example in relation to species, strain, population, isolated population, or race specific differences in the cell materials.
4) When it has been established that a certain cell population can be used for a cell therapy application, glycan analysis can be used to control that the cell population has the same characteristics as a cell population known to be useful in a clinical setting.

Time Dependent Changes During Cultivation of Cells

Furthermore during long term cultivation of cells spontaneous mutations may be caused in cultivated cell materials. It is noted that mutations in cultivated cell lines often cause harmful defects on glycosylation level.

It is further noticed that cultivation of cells may cause changes in glycosylation. It is realized that minor changes in any parameter of cell cultivation including quality and concentrations of various biological, organic and inorganic molecules, any physical condition such as temperature, cell density, or level of mixing may cause difference in cell materials and glycosylation. The present invention is directed to monitoring glycosylation changes according to the present invention in order to observe change of cell status caused by any cell culture parameter affecting the cells.

The present invention is in a preferred embodiment directed to analysis of glycosylation changes when the density of cells is altered. The inventors noticed that this has a major impact of the glycosylation during cell culture.

It is further realized that if there is limitations in genetic or differentiation stability of cells, these would increase probability for changes in glycan structures. Cell populations in early stage of differentiation have potential to produce different cell populations. The present inventors were able to discover glycosylation changes in early human cell populations.

Differentiation of Cell Lines

The present invention is specifically directed to observe glycosylation changes according to the present invention when differentiation of a cell line is observed. In a preferred embodiment the invention is directed to methods for observation of differentiation from early human cell or another preferred cell type according to the present invention to mesodermal types of stem cell

In case there is heterogeneity in cell material this may cause observable changes or harmful effects in glycosylation.

Furthermore, the changes in carbohydrate structures, even non-harmful or functionally unknown, can be used to obtain information about the exact genetic status of the cells.

The present invention is specifically directed to the analysis of changes of glycosylation, preferably changes in glycan profiles, individual glycan signals, and/or relative abundancies of individual glycans or glycan groups according to the present invention in order to observe changes of cell status during cell cultivation.

Analysis of Supporting/Feeder Cell Lines

The present invention is specifically directed to observe glycosylation differences according to the present invention, on supporting/feeder cells used in cultivation of stem cells and early human cells or other preferred cell type. It is known in the art that some cells have superior activities to act as a support/feeder cells than other cells. In a preferred embodiment the invention is directed to methods for observation of differences on glycosylation on these supporting/feeder cells. This information can be used in design of novel reagents to support the growth of the stem cells and early human cells or other preferred cell type.

Contaminations or Alterations in Cells due to Process Conditions

Conditions and Reagents Inducing Harmful Glycosylation or Harmful Glycosylation Related Effects to Cells During Cell Handling

The inventors further revealed conditions and reagents inducing harmful glycans to be expressed by cells with same associated problems as the contaminating glycans. The inventors found out that several reagents used in a regular cell purification processes caused changes in early human cell materials.

It is realized, that the materials during cell handling may affect the glycosylation of cell materials. This may be based on the adhesion, adsorption, or metabolic accumulation of the structure in cells under processing.

In a preferred embodiment the cell handling reagents are tested with regard to the presence glycan component being antigenic or harmfull structure such as cell surface NeuGc, Neu-O-Ac or mannose structure. The testing is especially preferred for human early cell populations and preferred subpopulations thereof.

The inventors note effects of various effector molecules in cell culture on the glycans expressed by the cells if absortion or metabolic transfer of the carbohydrate structures have not been performed. The effectors typically mediate a signal to cell for example through binding a cell surface receptor.

The effector molecules include various cytokines, growth factors, and their signalling molecules and co-receptors. The effector molecules may be also carbohydrates or carbohydrate binding proteins such as lectins.

Controlled Cell Isolation/Purification and Culture Conditions to Avoid Contaminations with Harmful Glycans or Other Alteration in Glycome Level

Stress Caused by Cell Handling

It is realized that cell handling including isolation/purification, and handling in context of cell storage and cell culture processes are not natural conditions for cells and cause physical and chemical stress for cells. The present invention allows control of potential changes caused by the stress. The control may be combined by regular methods may be combined with regular checking of cell viability or the intactness of cell structures by other means.

Examples of Physical and/or Chemical Stress in Cell Handling Step

Washing and centrifuging cells cause physical stress which may break or harm cell membrane structures. Cell purifications and separations or analysis under non-physiological flow conditions also expose cells to certain non-physiological stress. Cell storage processes and cell preservation and handling at lower temperatures affects the membrane structure. All handling steps involving change of composition of media or other solution, especially washing solutions around the cells affect the cells for example by altered water and salt balance or by altering concentrations of other molecules effecting biochemical and physiological control of cells.

Observation and Control of Glycome Changes by Stress in Cell Handling Processes

The inventors revealed that the method according to the invention is useful for observing changes in cell membranes which usually effectively alter at least part of the glycome observed according to the invention. It is realized that this related to exact organization and intact structures cell membranes and specific glycan structures being part of the organization.

The present invention is specifically directed to observation of total glycome and/or cell surface glycomes, these methods are further aimed for the use in the analysis of intactness of cells especially in context of stressfull condition for the cells, especially when the cells are exposed to physical and/or chemical stress. It is realized that each new cell handling step and/or new condition for a cell handling step is useful to be controlled by the methods according to the invention. It is further realized that the analysis of glycome is useful for search of most effectively altering glycan structures for analysis by other methods such as binding by specific carbohydrate binding agents including especially carbohydrate binding proteins (lectins, antibodies, enzymes and engineered proteins with carbohydrate binding activity).

Controlled Cell Preparation (Isolation or Purification) with Regard to Reagents

The inventors analysed process steps of common cell preparation methods. Multiple sources of potential contamination by animal materials were discovered.

The present invention is specifically directed to carbohydrate analysis methods to control of cell preparation processes. The present invention is specifically directed to the process of controlling the potential contaminations with animal type glycans, preferably N-glycolylneuraminic acid at various steps of the process.

The invention is further directed to specific glycan controlled reagents to be used in cell isolation

The glycan-controlled reagents may be controlled on three levels:
1. Reagents controlled not to contain observable levels of harmful glycan structure, preferably N-glycolylneuraminic acid or structures related to it
2. Reagents controlled not to contain observable levels of glycan structures similar to the ones in the cell preparation
3. Reagent controlled not to contain observable levels of any glycan structures.

The control levels 2 and 3 are useful especially when cell status is controlled by glycan analysis and/or profiling methods. In case reagents in cell preparation would contain the indicated glycan structures this would make the control more difficult or prevent it. It is further noticed that glycan structures may represent biological activity modifying the cell status.

Common Structural Features of all Glycomes and Preferred Common Subfeatures

The present invention reveals useful glycan markers for stem cells and combinations thereof and glycome compositions comprising specific amounts of key glycan structures. The invention is furthermore directed to specific terminal and core structures and to the combinations thereof.

The preferred glycome glycan structure(s) and/or glycomes from cells according to the invention comprise structure(s) according to the formula C0:

R₁Hexβz{R₃}ₙ₁Hex(NAc)ₙ₂XyR₂,

Wherein X is glycosidically linked disaccharide epitope β4(Fucα6)ₙGN, wherein n is 0 or 1, or X is nothing and

Hex is Gal or Man or GlcA,

HexNAc is GlcNAc or GalNAc,

y is anomeric linkage structure α and/or β or linkage from derivatized anomeric carbon,

z is linkage position 3 or 4, with the provision that when z is 4 then HexNAc is GlcNAc and then Hex is Man or Hex is Gal or Hex is GlcA, and

when z is 3 then Hex is GlcA or Gal and HexNAc is GlcNAc or GalNAc;

n1 is 0 or 1 indicating presence or absence of R3;

n2 is 0 or 1, indicating the presence or absence of NAc, with the proviso that n2 can be 0 only when Hexβz is Galβ4, and n2 is preferably 0, n2 structures are preferably derived from glycolipids;

R₁ indicates 1-4, preferably 1-3, natural type carbohydrate substituents linked to the core structures or nothing;

R₂ is reducing end hydroxyl, chemical reducing end derivative or natural asparagine N-glycoside derivative such as asparagine N-glycosides including asparagine N-glycoside aminoacids and/or peptides derived from protein, or natural serine or threonine linked O-glycoside derivative such as serine or threonine linked O-glycosides including asparagine N-glycoside aminoacids and/or peptides derived from protein, or when n2 is 1 R2 is nothing or a ceramide structure or a derivative of a ceramide structure, such as lysolipid and amide derivatives thereof;

R3 is nothing or a branching structure representing a GlcNAcβ6 or an oligosaccharide with GlcNAcβ6 at its reducing end linked to GalNAc (when HexNAc is GalNAc); or when Hex is Gal and HexNAc is GlcNAc, and when z is 3 then R3 is Fucα4 or nothing, and when z is 4 R3 is Fucα3 or nothing.

The preferred disaccharide epitopes in the glycan structures and glycomes according to the invention include structures Galβ4GlcNAc, Manβ4GlcNAc, Glcβ4GlcNAc, Galβ3GlcNAc, Galβ3GalNAc, GlcAβ3GlcNAc, GlcAβ3GalNAc, and Galβ4Glc which may be further derivatized from reducing end carbon atom and non-reducing monosaccharide residues and is in a separate embodiment branched from the reducing end residue. Preferred branched epitopes include Galβ4(Fucα3)GlcNAc, Galβ3(Fucα4)GlcNAc, and Galβ3(GlcNAcβ6)GalNAc, which may be further derivatized from reducing end carbon atom and non-reducing monosaccharide residues.

Preferred Epitopes for Methods According to the Invention

N-Acetyllactosamine Galβ3/4GlcNAc Terminal Epitopes

The two N-acetyllactosamine epitopes Galβ4GlcNAc and/or Galβ3GlcNAc represent preferred terminal epitopes present on stem cells or backbone structures of the preferred terminal epitopes for example further comprising sialic acid or fucose derivatisations according to the invention. In a preferred embodiment the invention is directed to fucosylated and/or non-substituted glycan non-reducing end forms of the terminal epitopes, more preferably to fucosylated and non-substituted forms. The invention is especially directed to non-reducing end terminal (non-substituted) natural Galβ4GlcNAc and/or Galβ3GlcNAc-structures from human stem cell glycomes. The invention is in a specific embodiment directed to non-reducing end terminal fucosylated natural Galβ4GlcNAc and/or Galβ3GlcNAc-structures from human stem cell glycomes.

Preferred Fucosylated N-Acetyllactosamines

The preferred fucosylated epitopes are according to the Formula TF:

(Fucα2)ₙ₁Galβ3/4(Fucα4/3)ₙ₂GlcNAcβ-R

Wherein

n1 is 0 or 1 indicating presence or absence of Fucα2;

n2 is 0 or 1, indicating the presence or absence of Fucα4/3 (branch), and

R is the reducing end core structure of N-glycan, O-glycan and/or glycolipid.

The preferred structures thus include type 1 lactosamines (Galβ3GlcNAc based):

Galβ3(Fucα4)GlcNAc (Lewis a), Fucα2Galβ3GlcNAc H-type 1, structure and, Fucα2Galβ3(Fucα4)GlcNAc (Lewis b) and

type 2 lactosamines (Galβ4GlcNAc based):

Galβ4(Fucα3)GlcNAc (Lewis x), Fucα2Galβ4GlcNAc H-type 2, structure and, Fucα2Galβ4(Fucα3)GlcNAc (Lewis y).

The type 2 lactosamines (fucosylated and/or terminal non-substituted) form an especially preferred group in context of adult stem cells and differentiated cells derived directly from these. Type 1 lactosamines (Galβ3GlcNAc—structures) are especially preferred in context of embryonal-type stem cells.

Lactosamines Galβ3/4GlcNAc and Glycolipid Structures Comprising Lactose Structures (Galβ4Glc)

The lactosamines form a preferred structure group with lactose-based glycolipids. The structures share similar features as products of β3/4Gal-transferases. The β3/4 galactose based structures were observed to produce characteristic features of protein linked and glycolipid glycomes.

The invention revealed that furthermore Galβ3/4GlcNAc-structures are a key feature of differentiation related structures on glycolipids of various stem cell types. Such glycolipids comprise two preferred structural epitopes according to the invention. The most preferred glycolipid types include thus lactosylceramide based glycosphingolipids and especially lacto-(Galβ3GlcNAc), such as lactotetraosylceramide Galβ3GlcNAcβ3Galβ4GlcβCer, preferred structures further including its non-reducing terminal structures selected from the group:

Galβ3(Fucα4)GlcNAc (Lewis a), Fucα2Galβ3GlcNAc (H-type 1), structure and, Fucα2Galβ3(Fucα4)GlcNAc (Lewis b) or sialylated structure SAα3Galβ3GlcNAc or SAα3Galβ3(Fucα4)GlcNAc, wherein SA is a sialic acid, preferably Neu5Ac preferably replacing Galβ3GlcNAc of lactotetraosylceramide and its fucosylated and/or elogated variants such as preferably according to the Formula:

(Sacα3)ₙ₅(Fucα2)ₙ₁Galβ3(Fucα4)ₙ₃GlcNAcβ3[Galβ3/4(Fucα4/3)ₙ₂GlcNAcβ3]ₙ₄Galβ4GlcβCer

wherein

n1 is 0 or 1, indicating presence or absence of Fucα2;

n2 is 0 or 1, indicating the presence or absence of Fucα4/3 (branch),

n3 is 0 or 1, indicating the presence or absence of Fucα4 (branch)

n4 is 0 or 1, indicating the presence or absence of (fucosylated) N-acetyllactosamine elongation;

n5 is 0 or 1, indicating the presence or absence of Sacα3 elongation;

Sac is terminal structure, preferably sialic acid, with α3-linkage, with the proviso that when Sac is present, n5 is 1, then n1 is 0 and

neolacto (Galβ4GlcNAc)-comprising glycolipids such as neolactotetraosylceramide Galβ4GlcNAcβ3Galβ4GlcβCer, preferred structures further including its non-reducing terminal Galβ4(Fucα3)GlcNAc (Lewis x), Fucα2Galβ4GlcNAc H-type 2, structure and, Fucα2Galβ4(Fucα3)GlcNAc (Lewis y) and

its fucosylated and/or elogated variants such as preferably

(Sacα3/6)ₙ₅(Fucα2)ₙ₁Galβ4(Fucα3)ₙ₃GlcNAcβ3[Galβ4(Fucα3)ₙ₂GlcNAcβ3]ₙ₄Galβ4GlcβCer

n1 is 0 or 1 indicating presence or absence of Fucα2;

n2 is 0 or 1, indicating the presence or absence of Fucα3 (branch),

n3 is 0 or 1, indicating the presence or absence of Fucα3 (branch)

n4 is 0 or 1, indicating the presence or absence of (fucosylated) N-acetyllactosamine elongation,

n5 is 0 or 1, indicating the presence or absence of Sacα3/6 elongation;

Sac is terminal structure, preferably sialic acid (SA) with α3-linkage, or sialic acid with α6-linkage, with the proviso that when Sac is present, n5 is 1, then n1 is 0, and when sialic acid is bound by α6-linkage preferably also n3 is 0.

Preferred Stem Cell Glycosphingolipid Glycan Profiles, Compositions, and Marker Structures

The inventors were able to describe stem cell glycolipid glycomes by mass spectrometric profiling of liberated free glycans, revealing about 80 glycan signals from different stem cell types. The proposed monosaccharide compositions of the neutral glycans were composed of 2-7 Hex, 0-5 HexNAc, and 0-4 dHex. The proposed monosaccharide compositions of the acidic glycan signals were composed of 0-2 NeuAc, 2-9 Hex, 0-6 HexNAc, 0-3 dHex, and/or 0-1 sulphate or phosphate esters. The present invention is especially directed to analysis and targeting of such stem cell glycan profiles and/or structures for the uses described in the present invention with respect to stem cells.

The present invention is further specifically directed to glycosphingolipid glycan signals specific to stem cell types as described in the Examples. In a preferred embodiment, glycan signals typical to hESC, preferentially including 876 and 892 are used in their analysis, more preferentially FucHexHexNAcLac, wherein α1,2-Fuc is preferential to α1,3/4-Fuc, and Hex₂HexNAcᵢLac, and more preferentially to Galβ3[Hex₁HexNAc₁]Lac. In another preferred embodiment, glycan signals typical to MSC, especially CB MSC, preferentially including 1460 and 1298, as well as large neutral glycolipids, especially Hex₂₋₃HexNAc₃Lac, more preferentially poly-N-acetyllactosamine chains, even more preferentially β1,6-branched, and preferentially terminated with type II LacNAc epitopes as described above, are used in context of MSC according to the uses described in the present invention.

Terminal glycan epitopes that were demonstrated in the present experiments in stem cell glycosphingolipid glycans are useful in recognizing stem cells or specifically binding to the stem cells via glycans, and other uses according to the present invention, including terminal epitopes: Gal, Galβ4Glc (Lac), Galβ4GlcNAc (LacNAc type 2), Galβ3, Non-reducing terminal HexNAc, Fuc, α1,2-Fuc, α1,3-Fuc, Fucα2Gal, Fucα2Galβ4GlcNAc (H type 2), Fucα2Galβ4Glc (2′-fucosyllactose), Fucα3GlcNAc, Galβ4(Fucα3)GlcNAc (Lex), Fucα3Glc, Galβ4(Fucα3)Glc (3-fucosyllactose), Neu5Ac, Neu5Acα2,3, and Neu5Acα2,6. The present invention is further directed to the total terminal epitope profiles within the total stem cell glycosphingolipid glycomes and/or glycomes.

The inventors were further able to characterize in hESC the corresponding glycan signals to SSEA-3 and SSEA-4 developmental related antigens, as well as their molar proportions within the stem cell glycome. The invention is further directed to quantitative analysis of such stem cell epitopes within the total glycomes or subglycomes, which is useful as a more efficient alternative with respect to antibodies that recognize only surface antigens. In a further embodiment, the present invention is directed to finding and characterizing the expression of cryptic developmental and/or stem cell antigens within the total glycome profiles by studying total glycan profiles, as demonstrated in the Examples for α1,2-fucosylated antigen expression in hESC in contrast to SSEA-1 expression in mouse ES cells.

The present invention revealed characteristic variations (increased or decreased expression in comparison to similar control cell or a contaminating cell or like) of both structure types in various cell materials according to the invention. The structures were revealed with characteristic and varying expression in three different glycome types: N-glycans, O-glycans, and glycolipids. The invention revealed that the glycan structures are a characteristic feature of stem cells and are useful for various analysis methods according to the invention. Amounts of these and relative amounts of the epitopes and/or derivatives varies between cell lines or between cells exposed to different conditions during growing, storage, or induction with effector molecules such as cytokines and/or hormones.

Preferred Epitopes and Antibody Binders Especially for Analysis of Embryonal Stem Cells

The antibody labelling experiment Tables with embryonal stem cells revealed specific of type 1 N-acetyllactosamine antigen recognizing antibodies recognizing non-modified disaccharide Galβ3GlcNAc (Le c, Lewis c), and fucosylated derivatives H type and Lewis b. The antibodies were effective in recognizing hESC cell populations in comparison to mouse feeder cells mEF used for cultivation of the stem cells. Specific different H type 2 recognizing antibodies were revealed to recognize different subpopulations of embryonal stem cells and thus usefulness for defining subpopulations of the cells. The invention further revealed a specific Lewis x and sialyl-Lewis x structures on the embryonal stem cells.

Other preferred binders and/or antibodies comprise of binders which bind to the same epitope than GF 287 (H type 1). In a preferred embodiment, an antibody binds to Fucα2Galβ3GlcNAc epitope. A more preferred antibody comprises of the antibody of clone 17-206 (ab3355) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 279 (Lewis c, Galβ3GlcNAc). In a preferred embodiment, an antibody binds to Galβ3GlcNAc epitope in glycoconjugates, more preferably in glycoproteins and glycolipids such as lactotetraosylceramide. A more preferred antibody comprises of the antibody of clone K21 (ab3352) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 288 (Globo H). In a preferred embodiment, an antibody binds to Fucα2Galβ3GalNAcβ epitope, more preferably Fucα2Galβ3GalNAcβ3GalαLacCer epitope. A more preferred antibody comprises of the antibody of clone A69-A/E8 (MAB-S206) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 284 (H type 2). In a preferred embodiment, an antibody binds to Fucα2Galβ4GlcNAc epitope. A more preferred antibody comprises of the antibody of clone B393 (DM3015) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 283 (Lewis b). In a preferred embodiment, an antibody binds to Fucα2Galβ3(Fucα4)GlcNAc epitope. A more preferred antibody comprises of the antibody of clone 2-25LE (DM3122) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 286 (H type 2). In a preferred embodiment, an antibody binds to Fucα2Galβ4GlcNAc epitope. A more preferred antibody comprises of the antibody of clone B393 (DM258P) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 290 (H type 2). In a preferred embodiment, an antibody binds to Fucα2Galβ4GlcNAc epitope. A more preferred antibody comprises of the antibody of clone A51-B/A6 (MAB-S204) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other binders binding to feeder cells, preferably mouse feeder cells, comprise of binders which bind to the same epitope than GF 285 (H type 2). In a preferred embodiment, an antibody binds to Fucα2Galβ4GlcNAc, Fucα2Galβ3(Fucα4)GlcNAc, Fucα2Galβ4(Fucα3)GlcNAc epitope. A more preferred antibody comprises of the antibody of clone B389 (DM3014) by Acris. This epitope is suitable and can be used to detect, isolate and evaluate of feeder cells, preferably mouse feeder cells in culture with human embryonic stem cells. The detection can be performed in vitro, for FACS purposes and/or for cell lineage specific purposes. This antibody can be used to positively isolate and/or separate and/or enrich feeder cells (negatively select stem cells), preferably mouse embryonic feeder cells from a mixture of cells comprising feeder and stem cells.

Other binders binding to stem cells, preferably human stem cells, comprise of binders which bind to the same epitope than GF 289 (Lewis y). In a preferred embodiment, an antibody binds to Fucα2Galβ4(Fucα3)GlcNAc epitope. A more preferred antibody comprises of the antibody of clone A70-C/C8 (MAB-S201) by Glycotope. This epitope is suitable and can be used to detect, isolate and evaluate of stem cells, preferably human stem cells in culture with feeder cells. The detection can be performed in vitro, for FACS purposes and/or for cell lineage specific purposes. This antibody can be used to positively isolate and/or separate and/or enrich stem cells (negatively select feeder cells), preferably human stem cells from a mixture of cells comprising feeder and stem cells.

Mesenchymal Stem Cells and differentiated Tissue Type Stem Cells Derived Thereof

Antibodies Useful for Evaluation of Differentiation Status of Mesenchymal Stem Cells

Examples and Tables (lower part) shows labelling of mesenchymal stem cells and differentiated mesenchymal stem cells

Invention revealed that structures recognized by antibody GF303, preferably Fucα2Galβ3GlcNAc, and GF276 appear during the differentiation of mesenchymal stem cells to osteogenic stem cells. It was further revealed, that the GalNAcα-group structures GF278, corresponding to Tn-antigen, and GF277, sialyl-Tn increase simultaneously.

The invention is further directed to the preferred uses according to the invention for binders to several target structures, which are characteristic to both mesenchymal stem cells (especially bone marrow derived) and the osteogenically differentiated mesenchymal stem cells. The preferred target structures include one GalNAcα-group structure recognizable by the antibody GF275, the antigen of the antibody is preferably sialylated O-glycan glycopeptide epitope as known for the antibody. The epitopes expressed in both mesenchymal and the osteonically differentiated stem cells further includes two characteristic globo-type antigen structures: the antigen of GF298, which binding correspond to globotriose(Gb3)-type antigens, and the antigen of GF297, which correspond to globotetraose(Gb4) type antigens. The invention has further revealed that terminal type two lactosamine epitopes are especially expressed in both types of mesenchymal stem cells and this was exemplified by staining both cell by antibody recognizing H type II antigen in Examples and Tables.

The invention is further directed to the preferred uses according to the invention for binders to several target structures which are substantially reduced or practically diminished/reduced to non-observable level when mesenchymal stem cells (especially bone marrow derived) differentiates to more differentiated, preferably osteogenic mesenchymal stem cells. These target structures include two globoseries structures, which are preferably Galactosyl-globoside type structure, recognized as antigen SSEA-3, and sialyl-galactosylgloboside type structure, recognized as antigen SSEA-4. The preferred reducing target structures further include two type two N-acetyllactosamine target structures Lewis x and sialyl-Lewis x.

In a preferred embodiment of the present invention, the antibodies or binders which bind to the same epitope than GF275, GF277, GF278, GF297, GF298, GF302, GF305, GF307, GF353, or GF354 are useful to detect/recognize, preferably bone marrow derived, mesenchymal stem cells (corresponding epitopes recognized by the antibodies are listed in Examples). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells.

Other binders binding to stem cells, preferably human stem cells, comprise of binders which bind to the same epitope than GF275 (sialylated carbohydrate epitope of the MUC-1 glycoprotein). A more preferred antibody comprises of the antibody of clone BM3359 by Acris. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow, or differentiated in osteogenic direction from mixture of cells comprising other, bone marrow derived, cells.

Other binders binding to stem cells, preferably human stem cells, comprise of binders which bind to the same epitope than GF305 (Lewis x). A more preferred antibody comprises of the antibody of clone CBL144 by Chemicon. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells.

Other binders binding to stem cells, preferably human stem cells, comprise of binders which bind to the same epitope than GF307 (sialyl lewis x). A more preferred antibody comprises of the antibody of clone MAB2096 by Chemicon. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells.

In a preferred embodiment, the antibodies or binders which bind to the same epitope than GF305, GF307, GF353 or GF354 are useful for positive selection and/or enrichment of mesenchymal stem cells (corresponding epitopes recognized by the antibodies are listed in Examples).

In another preferred embodiment of the present invention, antibodies or binders which bind to the same epitope than GF275, GF276, GF277, GF278, GF297, GF298, GF302, GF303, GF307 or GF353 are useful to detect/recognize differentiated, preferably bone marrow derived, mesenchymal stem cells and/or differentiated in osteogenic direction (corresponding epitopes recognized by the antibodies are listed in Examples). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells.

Other binders binding to stem cells, preferably human stem cells, comprise of binders which bind to the same epitope than GF297 (globoside GL4). A more preferred antibody comprises of the antibody of clone ab23949 by Abcam. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF298 (human CD77; GB3). A more preferred antibody comprises of the antibody of clone SM1160 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF302 (H type 2 blood antigen). In a preferred embodiment, an antibody binds to Fucα2Galβ4GlcNAc epitope. A more preferred antibody comprises of the antibody of clone DM3015 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

In a preferred embodiment of the present invention, antibodies or binders which bind to the same epitope than GF276, GF277, GF278, GF303, GF305, GF307, GF353, or GF354 are useful to detect/recognize, preferably bone marrow derived, mesenchymal stem cells and differentiated in osteogenic direction (corresponding epitopes recognized by the antibodies are listed in Examples). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow, or differentiated in osteogenic direction from mixture of cells comprising other, bone marrow derived, cells.

Further, the binders which bind to the same epitope than GF276 or GF303, or antibodies GF276 and/or GF303 are particularly useful to detect, isolate and evaluate of osteogenically differentiated stem cells, in culture or in vivo (corresponding epitopes recognized by the antibodies are listed in Examples).

Other binders binding to stem cells, preferably human stem cells, comprise of binders which bind to the same epitope than GF276 (oncofetal antigen). A more preferred antibody comprises of the antibody of clone DM288 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF277 (human sialosyl-Tn antigen; STn, sCD 175). A more preferred antibody comprises of the antibody of clone DM3197 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF278 (human sialosyl-Tn antigen; STn, sCD175 B1.1). A more preferred antibody comprises of the antibody of clone DM3218 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF303 (blood group H1 antigen, BG4). In a preferred embodiment, an antibody binds to Fucα2Galβ3GlcNAc epitope. A more preferred antibody comprises of the antibody of clone ab3355 by Abcam. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Further, the antibodies or binders are useful to isolate and enrich stem cells for osteogenic lineage. This can be performed with positive selection, for example, with antibodies GF276, GF277, GF278, and GF303 (corresponding epitopes recognized by the antibodies are listed in Examples). For negative depletion, a preferred epitope is the same as recognized with the antibodies GF305, GF307, GF353, or GF354. For negative depletion, a preferred epitope is the same as recognized with the antibody GF354 (SSEA-4) or GF307 (Sialyl Lewis x).

The staining intensity and cell number of stained stem cells, i.e. glycan structures of the present invention on stem cells indicates suitability and usefulness of the binder for isolation and differentiation marker. For example, low relative number of a glycan structure expressing cells may indicate lineage specificity and usefulness for selection of a subset and when selected/isolated from the colonies and cultured. Low number of expression is less than 5%, less than 10%, less than 15%, less than 20%, less than 30% or less than 40%. Further, low number of expression is contemplated when the expression levels are between 1-10%, 10%-20%, 15-25%, 20-40%, 25-35% or 35-50%. Typically, FACS analysis can be performed to enrich, isolate and/or select subsets of cells expressing a glycan structure(s).

High number of glycan expressing cells may indicate usefulness in pluripotency/multipotency marker and that the binder is useful in identifying, characterizing, selecting or isolating pluripotent or multipotent stem cells in a population of mammalian cells. High number of expression is more than 50%, more preferably more than 60%, even more preferably more than 70%, and most preferably more than 80%, 90 or 95%. Further, high number of expression is contemplated when the expression levels are between 50-60, 55%-65%, 60-70%, 70-80, 80-90%, 90-100 or 95-100%. Typically, FACS analysis can be performed to enrich, isolate and/or select subsets of cells expressing a glycan structure(s).

The epitopes recognized by the binders GF 279, GF 287, and GF 289 and the binders are particularly useful in characterizing pluripotency and multipotency of stem cells in a culture. The epitopes recognized by the binders GF 283, GF 284, GF 286, GF 288, and GF 290 and the binders are particularly useful for selecting or isolating subsets of stem cells. These subset or subpopulations can be further propagated and studied in vitro for their potency to differentiate and for differentiated cells or cell committed to a certain differentiation path.

The percentage as used herein means ratio of how many cells express a glycan structure to all the cells subjected to an analysis or an experiment. For example, 20% stem cells expressing a glycan structure in a stem cell colony means that a binder, eg an antibody staining can be observed in about 20% of cells when assessed visually.

In colonies a glycan structure bearing cells can be distributed in a particular regions or they can be scattered in small patch like colonies. Patch like observed stem cells are useful for cell lineage specific studies, isolation and separation. Patch like characteristics were observed with GF 283, GF 284, GF 286, GF 288, and GF 290.

For positive selection of feeder cells, preferably mouse feeder cells, most preferably embryonic fibroblasts, GF 285 is useful. This antibody has lower specificity and may have binding to e.g. Lewis y, which has been observed also in mEF cells. It stains almost all feeder cells whereas very little if at all staining is found in stem cells. The antibody was however under optimized condition revealed to bind to thin surface of embryonal bodies, this was in complementary to Lewis y antibody to the core of embryoid body. For all percentages of expression, see Tables.

Comparison Between Different Stem Cell Types

The present data revealed that comparison of a group of type 1 and type two N-acetyllactosamines is useful method for characterization stem cells such as mesenchymal stem cells and embryonal stem cells and or separating the cells from contaminating cell populations such as fibroblasts like feeder cells. The non-differentiated mesenchymal cell were devoid of type I N-acetyllactosamine antigens revealed from the hESC cells, while both cell types and and potential contaminating fibroblast have variable labelling with type II N-acetyllactosamine recognizing antibodies.

The term “mainly” indicates preferably at least 60%, more preferably at least 75% and most preferably at least 90%. In the context of stem cells, the term “mainly” indicates preferably at least 60%, more preferably at least 75% and most preferably at least 90% of cells expressing a glycan structure and useful for identifying, characterizing, selecting or isolating pluripotent or multipotent stem cells in a population of mammalian cells.

Revealing Presence Trypsin Sensitive Forms of Glycan Targets

The invention reveals in an example that part of the target structures of present glycan binders, especially monoclonal antibodies are trypsin sensitive. The antigen structures are essentially not observed or these are observed in reduced amount in FACS analysis of cell surface antigens when cells are treated (released from cultivation) by trypsin but observable after Versene treatment (0.02% EDTA in PBS). This was observed for example for labelling of mesenchymal stem cells by the antibody GF354, which has been indicated to bind SSEA-4 antigen. This target antigen structure has been traditionally considered to be sialyl-galactosylgloboside glycolipid, but obviously the antibody recognizes only an epitope at the non-reducing end of glycan sequence. The present invention is now especially directed to methods of isolation and characterization of mesenchymal stem cell glycopeptide bound glycan structure(s), which can be bound and enriched by the SSEA-a antibodies, and to characterization of corresponding glycopeptides and glycoproteins. The invention is further directed to analysis of trypsin insensitive glycan materials from stem cell especially mesenchymal stem cells and embryonal stem cells.

The invention revealed also that major part of the sialyl-mucin type target of ab GF 275 is trypssin sensitive and minor part is not trypsin sensitive. The invention is directed to isolation of both trypsin sensitive and trypsin insensitive glycan fractions, preferably glycoprotein(s) and glycopeptides, by methods according to the invention. The invention is further directed to isolation and characterization of protein degrading enzyme (protease) sensitive likely glycopeptides and glycoproteins bound by antibody GF 302, preferably when the materials are isolated from mesenchymal stem cells.

As used herein, “binder”, “binding agent” and “marker” are used interchangeably.

Antibodies

Information about useful lectin and antibody specificites useful according to the invention and for reducing end elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritsumei.ac.ip/epitope/, which list monoclonal antibody specificties).

HSCs

The methods outlined herein are particularly useful for identifying HSCs or progeny thereof from a population of cells. However, additional markers may be used to further distinguish subpopulations within the general HSC, or stem cell, population.

The various sub-populations may be distinguished by levels of binders to glycan structures of Formula (I) on stem cells. This may manifest on the stem cell surface (or on feeder cell if feeder cell specific binder is used) which may be detected by the methods outlined herein. However, the present invention may be used to distinguish between various phenotypes of the stem cell or HSC population including, but not limited to, the CD34.sup.+, CD38.sup.−, CD90.sup.+(thy1) and Lin. sup.− cells. Preferably the cells identified are selected from the group including, but not limited to, CD34.sup.+, CD38.sup.−, CD90+(thy1), or Lin. sup.−.

The present invention thus encompasses methods of enriching a population for stem and/or HSCs or progeny thereof The methods involve combining a mixture of HSCs or progeny thereof with an antibody or marker or binding protein/agent or binder that recognizes and binds to glycan structure according to Formula (I) on stem cell(s) under conditions which allow the antibody or marker or binder to bind to glycan structure according to Formula (I) on stem cell(s) and separating the cells recognized by the antibody or marker to obtain a population substantially enriched in stem cells or progeny thereof. The methods can be used as a diagnostic assay for the number of HSCs or progeny thereof in a sample. The cells and antibody or marker are combined under conditions sufficient to allow specific binding of the antibody or marker to glycan structure according to Formula (I) on stem cell(s) which are then quantitated. The HSCs or stem cells or progeny thereof can be isolated or further purified.

As discussed above the cell population may be obtained from any source of stem cells or HSCs or progeny thereof including those samples discussed above.

The detection for the presence of glycan structure(s) according to Formula (I) on stem cell(s) may be conducted in any way to identify glycan structure according to Formula (I) on stem cell(s). Preferably the detection is by use of a marker or binding protein for glycan structure according to Formula (I) on stem cell(s). The binder/marker for glycan structure according to Formula (I) on stem cell(s) may be any of the markers discussed above. However, antibodies or binding proteins to glycan structure according to Formula (I) on stem cell(s) are particularly useful as a marker for glycan structure according to Formula (I) on stem cell(s).

Various techniques can be employed to separate or enrich the cells by initially removing cells of dedicated lineage. Monoclonal antibodies, binding proteins and lectins are particularly useful for identifying cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. The particular technique employed will depend upon efficiency of separation, associated cytotoxicity, ease and speed of performance, and necessity for sophisticated equipment and/or technical skill.

Procedures for separation or enrichment can include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation or enrichment techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye, Hoescht 33342).

Techniques providing accurate separation include, but are not limited to, FACS, which can have varying degrees of sophistication, e.g., a plurality of color channels, low angle and obtuse light scattering detecting channels, impedence channels, etc. Any method which can isolate and distinguish these cells according to levels of expression of glycan structure according to Formula (I) on stem cell(s) may be used.

In a first separation, typically starting with about 1.times.10.sup.10, preferably at about 5.times.10.sup.8-9 cells, antibodies or binding proteins or lectins to glycan structure according to Formula (I) on stem cell(s) can be labeled with at least one fluorochrome, while the antibodies or binding proteins for the various dedicated lineages, can be conjugated to at least one different fluorochrome. While each of the lineages can be separated in a separate step, desirably the lineages are separated at the same time as one is positively selecting for glycan structure according to Formula (I) on stem cell markers. The cells can be selected against dead cells, by employing dyes associated with dead cells (including but not limited to, propidium iodide (PI)).

To further enrich for any cell population, specific markers for those cell populations may be used. For instance, specific markers for specific cell lineages such as lymphoid, myeloid or erythroid lineages may be used to enrich for or against these cells. These markers may be used to enrich for HSCs or progeny thereof by removing or selecting out mesenchymal or keratinocyte stem cells.

The methods described above can include further enrichment steps for cells by positive selection for other stem cell specific markers. Suitable positive stem cell markers include, but are not limited to, SSEA-3, SSEA-4, Tra 1-60, CD34.sup.+, Thy-1.sup.+, and c-kit.sup.+. By appropriate selection with particular factors and the development of bioassays which allow for self-regeneration of HSCs or progeny thereof and screening of the HSCs or progeny thereof as to their markers, a composition enriched for viable HSCs or progeny thereof can be produced for a variety of purposes.

Once the stem cells or HSC or progeny thereof population is isolated, further isolation techniques may be employed to isolate sub-populations within the HSCs or progeny thereof. Specific markers including cell selection systems such as FACS for cell lineages may be used to identify and isolate the various cell lineages.

In yet another aspect of the present invention there is provided a method of measuring the content of stem cells or HSC or their progeny said method comprising

obtaining a cell population comprising stem cells or progeny thereof

combining the cell population with a binding protein or binder for glycan structure according to Formula (I) on stem cell(s) thereof

selecting for those cells which are identified by the binding protein for glycan structure according to Formula (I) on stem cell(s) thereof; and

quantifying the amount of selected cells relative to the quantity of cells in the cell population prior to selection with the binding protein.

Manipulation of Cells by Binders

The invention is specifically directed to manipulation of cells by the specific binding proteins. It is realized that the glycans described have important roles in the interactions between cells and thus binders or binding molecules can be used for specific biological manipulation of cells. The manipulation may be performed by free or immobilized binders. In a preferred embodiment cells are used for manipulation of cell under cell culture conditions to affect the growth rate of the cells.

Stem Cell Nomenclature

The present invention is directed to analysis of all stem cell types, preferably human stem cells. A general nomenclature of the stem cells is described in Figs. The alternative nomenclatura of the present invention describe early human cells which are in a preferred embodiment equivalent of adult stem cells (including cord blood type materials) as shown in Figs. Adult stem cells in bone marrow and blood is equivalent for stem cells from “blood related tissues”.

Sorting of Stem Cells by Specific Lectins

The invention revealed use of specific lectin types recognizing cell surface glycan epitopes according to the invention for sorting of stem cells, especially by FACS methods, most preferred cell types to be sorted includes adult stem cells in blood and bone marrow, especially cord blood cells. Preferred lectins for sorting of cord blood cells include GNA, STA, GS-II, PWA, HHA, PSA, RCA, and others as shown in Examples. The relevance of the lectins for isolating specific stem cell populations was demonstrated by double labeling with known stem cells markers, as described in Examples.

Preferred Qualitative and Quantitative Complete N-Glycomes of Stem Cells

Preferred Binders for Stem Cell Sorting and Isolation

As described in the Examples, the inventors found that especially the mannose-specific and especially α1,3-linked mannose-binding lectin GNA was suitable for negative selection enrichment of CD34+ stem cells from CB MNC. In addition, the poly-LacNAc specific lectin STA and the fucose-specific and especially α1,2-linked fucose-specific lectin UEA were suitable for positive selection enrichment of CD34+ stem cells from CB MNC.

The present invention is specifically directed to stem cell binding reagents, preferentially proteins, preferentially mannose-binding or α1,3-linked mannose-binding, poly-LacNAc binding, LacNAc-binding, and/or fucose- or preferentially α1,2-linked fucose-binding; in a preferred embodiment stem cell binding or nonbinding lectins, more preferentially GNA, STA, and/or UEA; and in a further preferred embodiment combinations thereof; to uses described in the present invention taking advantage of glycan-binding reagents that selectively either bind to or do not bind to stem cells.

Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described. In a further preferred embodiment, the present invention is directed to using galectin ligand structures, derivatives thereof, or ligand-mimicking reagents to uses described in the present invention in stem cell type specific fashion.

Analysis and Utilization of poly-N-acetyllactosamine Sequences and Non-Reducing Terminal Epitopes Associated with Different Glycan Types

The present invention is directed to poly-N-acetyllactosamine sequences (poly-LacNAc) associated with cell types accoriding to the present invention. The inventors found that different types of poly-LacNAc are characteristic to different cell types, as described in the Examples of the present invention. In particular, CB MNC are characterized by linear type 2 poly-LacNAc; MSC, especially CB MSC, are characterized by branched type 2 poly-LacNAc; and hESC are characterized by type 1 terminating poly-LacNAc. The present invention is especially directed to the analysis and utilization of these glycan characteristics according to the present invention. The present invention is further directed to the analysis and utilization of the specific cell-type accociated glycan sequences revealed in the present Examples according to the present invention.

The present invention is directed to non-reducing terminal epitopes in different glycan classes including N- and O-glycans, glycosphingolipid glycans, and poly-LacNAc. The inventors found that especially the relative amounts of β1,4-linked Gal, β1,3-linked Gal, α1,2-linked Fuc, α1,3/4-linked Fuc, α-linked sialic acid, and α2,3-linked sialic acid are characteristically different between the studied cell types; and the invention is especially directed to the analysis and utilization of these glycan characteristics according to the present invention.

The present invention is further directed to analyzing fucosylation degree in O-glycans by comparing indicative glycan signals such as neutral O-glycan signals at m/z 771 and 917 as described in the Examples. The inventors found that compared to other cell types analyzed in the present invention, hESC had low relative abundance of neutral O-glycan signal at m/z 917 compared to 771, indicating low fucosylation degree of the 0-glycan sequences corresponding to the signal at m/z 771 and containing terminal β1,4-linked Gal. Another difference was the occurrence of abundant signal at m/z 552 in hESC, corresponding to Hex₁HexNAc₁dHex₁, including α1,2-fucosylated Core 1 O-glycan sequence. In contrast, in CB MNC the glycan signal at m/z 917 is relatively abundant, indicating high fucosylation degree of the O-glycan sequences corresponding to the signal at m/z 771 and containing terminal β1,4-linked Gal. The other cell types analyzed in the present invention also had characteristic fucosylation degree between these two cell types.

Especially, the present invention is directed to analyzing terminal epitopes associated with poly-LacNAc in stem cells, more preferably when these epitopes are presented in the context of a poly-LacNAc chain, most preferably in O-glycans or glycosphingolipids. The present invention is further directed to analyzing such characteristic poly-LacNAc, terminal epitope, and fucosylation profiles according to the methods of the present invention, in glycan structural characterization and specific glycosylation type identification, and other uses of the present invention; especially when this analysis is done based on endo-β-galactosidase digestion, by studying the non-reducing terminal fragments and their profile, and/or by studying the reducing terminal fragments and their profile, as described in the Examples of the present invention. The inventors found that cell-type specific glycosylation features are efficiently reflected in the endo-β-galactosidase reaction products and their profiles. The present invention is further directed to such reaction product profiles and their analysis according to the present invention.

Especially in hESC, the inventors found that characteristic non-reducing poly-LacNAc associated sequences include Fucα2Gal, Galβ3GlcNAc, Fucα2Galβ3GlcNAc, and α3′-sialylated Galβ3GlcNAc. The present invention is especially directed to analysis of such glycan structures according to the present methods, in context of stem cells and differentiation of stem cells, preferably in context of human embryonic stem cells and their differentiation.

The inventors further found that all three most thoroughly analyzed cellular glycan classes, N-glycans, O-glycans, and glycosphingolipid glycans, were differently regulated compared to each other, especially with regard to non-reducing terminal glycan epitopes and poly-LacNAc sequences as described in the Examples and Tables of the present invention. Therefore, combining quantitative glycan profile analysis data from more than one glycan class will yield significantly more information. The present invention is especially directed to combining glycan data obtained by the methods of the present invention, from more than one glycan class selected from the group of N-glycans, O-glycans, and glycosphingolipid glycans; more preferably, all three classes are analyzed; and use of this information according to the present invention. In a preferred embodiment, N-glycan data is combined with O-glycan data; and in a further preferred embodiment, N-glycan data is combined with glycosphingolipid glycan data.

Recognition of Glycans of Stem Cells

General observations. There seems not to be a single specific glycan epitope analyzed absolutely specific only for one total population of MSCs or a cell population differentiated into osteogenic lineage. Instead there seems to be enrichment of certain glycan epitopes in stem cells and in differentiated cells. In some cases the antibodies recognize epitopes, which are highly or several fold enriched in a specific cell type or present above the current FACS detection limit in a part of a cell population but not in the other corresponding cell populations. It is realized that such antibodies are especially useful for specific recognition of the specific cell population. Furthermore, combination of several antibodies recognizing independent populations of specific cell types is useful for recognition of a larger cell population in a positive or negative manner.

The present invention provides reagents common to stem cell populations in general or for specific differentiation stage of stem cells such as stem stem cells, or differentiated stem stem cells in general or specific for the specifically differentiated cell populations such as adipocytes or osteoblasts. Furthermore the invention reveals specific marker structures for stem stem cells derived from specific tissue types such as cord blood or bone marrow.

The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificties in order to find high specificity antibodies.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells. It is realized that tissues comprising stem cells usually contain these in primitive stem cell stage and highly expressed markers according can be optimised or selected for the cell isolation. It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

Combination of several antibodies for specific analysis of a stem cell population would characterize the cell population. In a preferred embodiment at least one “effectively binding antibody”, recognizing major part (over 35%) or most (50%) of the cell population (preferably more than 30%, an in order of increasing preference more than 40%, 50%, 60%, 70%, 80% and most preferably more than 90%) , are selected for the analytic method in combination with at least one “non-binding antibody”, recognizing preferably minor part (preferably from detection limit of the method to low level of recognition, in order of preference less than 10%, 7%, 5%, 2% or 1% of cells, e.g 0.2-10% of cells, more preferably 0.2-5% of the cells, and even more preferably 0.5-2% or most preferably 0.5%-1.0%) or no part of the cell population (under or at the detection limit e.g. in order of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably from 7% to 40% and most preferably from 10 to 35%.

The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificties in order to find high specificity antibodies. Furthermore the invention is directed to the search of the structures from phage display libraries.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells.

It is realized that tissues comprising stem cells usually contain these in primitive stem cell stage and highly expressed markers according can be optimised or selected for the cell isolation. In a preferred embodiment the invention is directed to selection of stem cells by the binders according to the invention such as by or sialyl-Lewis x recognizing proteins including preferably monoclonal antibodies recognizing the glycan epitopes according the invention. In a separate embodiments the invention is directed to the use of selectins or selectin homologous proteins optimized for the reconition.

It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

Combinations

Combination of several antibodies for specific analysis of a hematoppietic or associated population for cell population would characterize the cell population. In a preferred embodiment at least one “effectively binding antibody”, recognizing major part (over 35%) or most (50%) of the cell population (preferably more than 30%, an in order of increasing preference more than 40%, 50%, 60%, 70%, 80% and most preferably more than 90%) , are selected for the analytic method in combination with at least one “non-binding antibody”, recognizing preferably minor part (preferably from detection limit of the method to low level of recognition, in order of preference less than 10%, 7%, 5%, 2% or 1% of cells, e.g 0.2-10% of cells, more preferably 0.2-5% of the cells, and even more preferably 0.5-2% or most preferably 0.5%-1.0%) or no part of the cell population (under or at the detection limit e.g. in order of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably from 7% to 40% and most preferably from 10 to 35%.

The invention is directed to the use of several reagents recognizing terminal epitopes together, preferably at least two reagents, more preferably at least three epitopes, even more preferably at least four, even more preferably at least five, even more preferably at least six, even more preferably at least seven, and most preferably at least 8 to recognize enough positive and negative targets together. It is realized that with high specificity binders selectively and specifically recognizing elongated epitopes, less binders may be needed e.g. these would be preferably used as combinations of at least two reagents, more preferably at least three epitopes, even more preferably at least four, even more preferably at least five, most preferably at least six antibodies. The high specificity binders selectively and specifically recognizing elongated epitopes binds one of the elongated epitopes at least inorder of increasing preference, 5, 10, 20, 50, or 100 fold affinity, methods for measuring the antibody binding affinities are well known in the art. The invention is also directed to the use of lower specificity antibodies capable of effective recognition of one elongated epitope but also at least one, preferably only one additional elongated epitope with same terminal structure

The reagents are preferably used in arrays comprising in order of increasing preference 5, 10, 20, 40 or 70 or all reagents shown in cell labelling experiments.

The invention is further directed to combinations of fucosylated and/or sialylated structures with structures devoid of these modifications. Combinations of type 1 N-acetyllactosamine with type 2 structures with type 1 (Galβ3GlcNAc) structures and/or with mucin type and/or glyccolipids structures. In a preferred combination at least one binding antibody is combined with non-binding antibody recognizing different structure type

The antibodies recognize certain glycan epitopes revealed as target structures according to the invention. It is realized that specificites and affinities of the antibodies vary between the clones. It was realized that certain clones known to recognize certain glycan structure does not necessarily recognize the same cell population.

Release of Binders or Binder Conjugates from the Cells by Carbohydrate Inhibition

The invention is in a preferred embodiment directed to the release of glycans from binders. This is preferred for several methods including:
a) release of cells from soluble binders after enrichement or isolation of cells by a method involving a binder
b) release from solid phase bound binders after enrichment or isolation of cells or during cell cultivation e.g. for passaging of the cells

The inhibitin carbohydrate is selected to correspond to the binding epitope of the lectin or parts) thereof The preferred carbohydrates includes oligosaccharides, monosaccharides and conjugates thereof. The preferred concentrations of carbohydrates includes contrations tolerable by the cells from 1 mM to 500 mM, more preferably 10 mM to 250 mM and even more preferably 10-100 mM, higher concentrations are preferred for monosaccharides and method involving solid phase bound binders. Preferred oligosaccharide sequences including oligosaccharides and reducing end conjugates includes Galβ4Glc, Galβ4GlcNAc, Galβ3GlcNAc, Galβ3GalNAc, and sialylated and fucosylated variants of these as described in TABLEs and formulas according to the invention,

The preferred reducing enstructure in conjugates is AR, wherein A is anomeric structure preferably beta for Galβ4Glc, Galβ4GlcNAc, Galβ3GlcNAc, and alfa for Galβ3GalNAc and R is organic residue linked glycosidically to the saccahride, and preferably alkyl such as method, ethyl or propyl or ring structure such as a cyclohexyl or aromatic ring structure optionally modified with further functional group.

Preferred monosaccharides includes terminal or two or three terminal monosaccharides of the binding epitope such as Fuc, Gal, GalNAc, GlcNAc, Man, preferably as anomeric conjugates: as FucαR, Galβ3R, GalNAcβR, GalNAcαR GlcNAcβR, ManαR. For example PNA lectin is preferably inhibited by Galβ3GalNAc or lactose or Gal, STA is inhibited by Galβ4Glc, Galβ4GlcNAc or oligomers or poly-LacNAc epitopes derived thereof and LTA is inhibited by fucosylalactose Galβ4(Fucα3)Glc, Galβ4(Fucα3)GlcNAc or Fuc or FucαR. Examples of monovalent inhibition condition are shown in Venable A. et al. (2005) BMC Developmental biology, for inhibition when the cells are bound to polyvalently to solid phase larger epitopes and/or concentrations or multi/polyvalent conjugates are preferred.

The invention is further directed to methods of release of binders by protease digestion similarily as known for release of cells from CD34+ magnetic beads.

Immobilized Binders Preferably Binder Proteins Protein

The present invention is directed to the use of the specific binder for or in context of cultivation of the stem cells wherein the binder is immobilized.

The immobilization includes non-covalent immobilization and covalent bond including immobilization method and further site specific immobilization and unspecific immobilization.

A preferred non-covalent immobilization methods includes passive adsorption methods. In a preferred method a surface such as plastic surface of a cell culture dish or well is passively absorbed with the binder. The preferred method includes absorbtion of the binder protein in a solvent or humid condition to the surface, preferably evenly on the surface. The preferred even distribution is produced using slight shaking during the absorption period preferably form 10 min to 3 days, more preferably from 1 hour to 1 day, and most preferably over night for about 8 to 20 hours. The washing steps of the immobilization are preferably performed gently with slow liquid flow to avoid detachment of the lectin.

Specific Immobilization

The specific immobilization aims for immobilization from protein regions which does not disturb the the binding of the binding site of the binder to its ligand glycand such as the specific cell surface glycans of stem cells according to the invention.

Preferred specific immobilization methods includes chemical conjugation from specific aminoacid residues from the surface of the binder protein/peptide. In a preferred method specific amino acid residue such as cysteine is cloned to the site of immobilization and the conjugation is performed from the cystein, in another preferred method N-terminal cytsteine is oxidized by periodic acid and conjugated to aldehyde reactive reagents such as amino-oxy-methyl hydroxylamine or hydrazine structures, further preferred chemistries includes “click” chemistry marketed by Invitrogen and aminoacid specific coupling reagents marketed by Pierce and Molecular probes.

A preferred specific immobilization occurs from protein linked carbohydrate such as O- or N-glycan of the binder, preferably when the glycan is not close to the binding site or longer specar is used.

Glycan Immobilized Binder Protein

Preferred glycan immobilization occurs through a reactive chemoselective ligation group R1 of the glycans, wherein the chemical group can be specifically conjugated to second chemoselective ligation group R2 without major or binding destructutive changes to the protein part of the binder. Chemoselective groups reacting with aldehydes and ketones includes as amino-oxy-methyl hydroxylamine or hydrazine structures. A preferred R1-group is a carbonyl such as an aldehyde or a ketone chemically synthesized on the surface of the protein. Other preferred chemoselective groups includes maleimide and thiol; and “Click”-reagents including azide and reactive group to it.

Preferred synthesis steps includes
a) chemical oxidation by carbohydrate selectively oxidizing chemical, preferably by
periodic acid or
b) enzymatic oxidation by non-reducing end terminal monosaccharide oxidizing enzyme such as galactose oxidase or by transferring a modified monosaccharide residue to the terminal monosaccharide of the glycan.

Use of oxidative enzymes or periodic acid are known in the art has been described in patent application directed conjugating HES-polysaccharide to recombinant protein by Kabi-Frensenius (WO2005EP02637, WO2004EP08821, WO2004EP08820, WO2003EP08829, WO2003EP08858, WO2005092391, WO2005014024 included fully as reference) and a German research institute.

Preferred methods for the transferring the terminal monosaccharide reside includes use of mutant galactosyltransferase as described in patent application by part of the inventors US2005014718 (included fully as reference) or by Qasba and Ramakrishman and colleagues US2007258986 (included fully as reference) or by using method described in glycopegylation patenting of Neose (US2004132640, included fully as reference).

Conjugates Including High Specificity Chemical Tag

In a preferred embodiment the binder is, specifically or non-specifically conjugated to a tag, referred as T, specifically recognizable by a ligand L, examples of tag includes such as biotin biding ligand (strept)avidin or a fluorocarbonyl binding to another fluorocarbonyl or peptide/antigen and specific antibody for the peptide/antigen

Preferred Conjugate Structures

The preferred conjugate structures are according to the Formula CONJ

B-(G-)ₘR1-R2-(S1-)ₙT-,

wherein B is the binder, G is glycan (when the binder is glycan conjugated),

R1 and R2 are chemoselective ligation groups, T is tag, preferably biotin, L is specifically binding ligand for the tag; S1 is an optional spacer group, preferably C₁-C₁₀ alkyls, m and n are integers being either 0 or 1, independently.

Complex of Binder

The invention id further directed to complexes in of the binders involving conjugation to surface including solid phase or a matrix including polymers and like. It is realized that it is especially useful to conjugate the binder from the glycan because preventing cross binding of of binders or effects of the binders to cells.

Stem Cell Glycan Binder Target Table for Selecting Effective Positive and/or Negative Binders and Combinations Thereof

Table 27 describes combined results of the inventors' structural assignments of stem cell and differentiated cell specific glycosylation (Examples of the present invention describing mass spectrometric profiling, NMR, glycosidase, and glycan fragmentation experiments, as well as structure-revealing comparison of N-glycan profiles including Tables, Figures, and Examples of the present invention), biosynthetic information including knowledge of biosynthetic pathways and glycosylation gene expression, as well as binder specificities as described in the present invention (Examples of the present invention describing lectin, antibody, and other binder molecule binding to specific cell types and molecule classes).

Table 27 describes suitable binder targets in specific cell types by q, +/−, +, and ++ codes, especially preferably by + and ++ codes; as well as useful absence or low expression by −, q, and +/− codes, especially preferably by − and +/− codes. The inventors realized that such data can be used to recognize specifically selected cell types. The invention is directed to such use with various different principles as specific embodiments of the present invention: positive selection using binders recognizing specific cell type associated targets, negative selection by utilizing targets with low abundance on specific cells, as well as combined positive and negative selection, or further combined use of more than one positive and/or negative targets to increase specificity and/or efficiency according to the present invention.

### Example 1

### MALDI-TOF Mass Spectrometric N-Glycan Profiling, Glycosidase and Lectin Profiling of Cord Blood Derived and Bone Marrow Derived Mesenchymal Stem Cell Lines

Examples of Cell Sample Production

Cord Blood Derived Mesenchymal Stem Cell Lines

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min) The mononuclear cell fragment was collected from the gradient and washed twice with PBS.

Umbilical cord blood cell isolation and culture. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http://www.invitrogen.com) 40% MCDB-201 (Sigma-Aldrich) 2% fetal calf serum (FCS), 1× penicillin-streptomycin (both form Gibco), 1× ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/ cm². In later passages the cells were detached with trypsin/EDTA from defined area at defined time points, counted with hematocytometer and replated at density of 2000-3000 cells/cm².

Bone Marrow Derived Mesenchymal Stem Cell Lines

Isolation and culture of bone marrow derived stem cells. Bone marrow (BM)-derived MSCs were obtained as described by Leskela et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

Experimental Procedures

Flow cytometric analysis of mesenchymal stem cell phenotype. Both UBC and BM derived mesenchymal stem cells were phenotyped by flow cytometry (FACSCalibur, Becton Dickinson). Fluorescein isothicyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abeam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD14, CD34, CD45 and HLA-DR and positive for CD13, CD29, CD44, CD90, CD105 and HLA-ABC.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Osteogenic differentiation. To induce the osteogenic differentiation of the BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

Cell harvesting for glycome analysis. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times.

The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

Lectin stainings. FITC-labeled Maackia amurensis agglutinin (MAA) was purchased from EY Laboratories (USA) and FITC-labeled Sambucus nigra agglutinin (SNA) was purchased from Vector Laboratories (UK). Bone marrow derived mesenchymal stem cell lines were cultured as described above. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10 minutes. After fixation, cells were washed 3 times with PBS and non-specific binding sites were blocked with 3% HSA-PBS (FRC Blood Service, Finland) or 3% BSA-PBS (>99% pure BSA, Sigma) for 30 minutes at RT. According to manufacturers' instructions cells were washed twice with PBS, TBS (20 mM Tris-HCl, pH 7.5, 150 mM NaCl, 10 mM CaCl₂) or HEPES-buffer (10 mM HEPES, pH 7.5, 150 mM NaCl) before lectin incubation. FITC-labeled lectins were diluted in 1% HSA or 1% BSA in buffer and incubated with the cells for 60 minutes at RT in the dark. Furthermore, cells were washed 3 times 10 minutes with PBS/TBS/HEPES and mounted in Vectashield mounting medium containing DAPI-stain (Vector Laboratories, UK). Lectin stainings were observed with Zeiss Axioskop 2 plus—fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Results

Glycan isolation from mesenchymal stem cell populations. The present results are produced from two cord blood derived mesenchymal stem cell lines and cells induced to differentiate into adipogenic direction, and two marrow derived mesenchymal stem cell lines and cells induced to differentiate into osteogenic direction. The characterization of the cell lines and differentiated cells derived from them are described above. N-glycans were isolated from the samples, and glycan profiles were generated from MALDI-TOF mass spectrometry data of isolated neutral and sialylated N-glycan fractions as described in the preceding examples.

Cord Blood Derived Mesenchymal Stem Cell (CB MSC) Lines

Neutral N-glycan structural features. Neutral N-glycan groupings proposed for the two CB MSC lines resemble each other closely, indicating that there are no major differences in their neutral N-glycan structural features. However, CB MSCs differ from the CB mononuclear cell populations, and they have for example relatively high amounts of neutral complex-type N-glycans, as well as hybrid-type or monoantennary neutral N-glycans, compared to other structural groups in the profiles.

Identification of soluble glycan components. Similarly to CB mononuclear cell populations, in the present analysis neutral glycan components were identified in all the cell types that were assigned as soluble glycans based on their proposed monosaccharide compositions including components from the glycan group Hex₂₋₁₂HexNAc₁ (see Figures). The abundancies of these glycan components in relation to each other and in relation to the other glycan signals vary between individual samples and cell types.

Sialylated N-glycan profiles. Sialylated N-glycan profiles obtained from two CB MSC lines resemble closely each other with respect to their overall sialylated N-glycan profiles. However, minor differences between the profiles are observed, and some glycan signals can only be observed in one cell line, indicating that the two cell lines have glycan structures that differ them from each other. The analysis revealed in each cell type the relative proportions of about 50-70 glycan signals that were assigned as acidic N-glycan components. Typically, significant differences in the glycan profiles between cell populations are consistent throughout multiple experiments.

Differentiation-associated changes in glycan profiles. Neutral N-glycan profiles of CB MSCs change upon differentation in adipogenic cell culture medium. The present results indicate that relative abundancies of several individual glycan signals as well as glycan signal groups change due to cell culture in differentiation medium. The major change in glycan structural groups associated with differentation is increase in amounts of neutral complex-type N-glycans, such as signals at m/z 1663 and m/z 1809, corresponding to the Hex₅HexNAc₄ and Hex₅HexNAc₄dHex₁ monosaccharide compositions, respectively. Changes were also observed in sialylated glycan profiles.

Glycosidase analyses of neutral N-glycans. Specific exoglycosidase digestions were performed on isolated neutral N-glycan fractions from CB MSC lines as described in Examples. The results of α-mannosidase analysis show in detail which of the neutral N-glycan signals in the neutral N-glycan profiles of CB MSC lines are susceptible to α-mannosidase digestion, indicating for the presence of non-reducing terminal α-mannose residues in the corresponding glycan structures. As an example, the major neutral N-glycan signals at m/z 1257, 1419, 1581, 1743, and 1905, which were preliminarily assigned as high-mannose type N-glycans according to their proposed monosaccharide compositions Hex₅₋₉HexNAc₂, were shown to contain terminal α-mannose residues thus confirming the preliminary assignment. The results indicate for the presence of non-reducing terminal β1,4-galactose residues in the corresponding glycan structures. As an example, the major neutral complex-type N-glycan signals at m/z 1663 and m/z 1809 were shown to contain terminal β1,4-linked galactose residues.

Bone Marrow Derived Mesenchymal Stem Cell (BM MSC) Lines

Neutral N-glycan profiles and differentiation-associated changes in glycan profiles. Neutral N-glycan profiles obtained from a BM MSC line, grown in proliferation medium and in osteogenic medium resemble CB MSC lines with respect to their overall neutral N-glycan profiles. However, differences between cell lines derived from the two sources are observed, and some glycan signals can only be observed in one cell line, indicating that the cell lines have glycan structures that differ them from each other. The major characteristic structural feature of BM MSCs is even more abundant neutral complex-type N-glycans compared to CB MSC lines. Similarly to CB MSCs, these glycans were also the major increased glycan signal group upon differentiation of BM MSCs. The analysis revealed in each cell type the relative proportions of about 50-70 glycan signals that were assigned as non-sialylated N-glycan components. Typically, significant differences in the glycan profiles between cell populations are consistent throughout multiple experiments.

Sialylated N-glycan profiles. Sialylated N-glycan profiles obtained from a BM MSC line, grown in proliferation medium and in osteogenic medium. The undifferentiated and differentiated cells resemble closely each other with respect to their overall sialylated N-glycan profiles. However, minor differences between the profiles are observed, and some glycan signals can only be observed in one cell line, indicating that the two cell types have glycan structures that differ them from each other. The analysis revealed in each cell type the relative proportions of about 50 glycan signals that were assigned as acidic N-glycan components. Typically, significant differences in the glycan profiles between cell populations are consistent throughout multiple experiments.

Sialidase analysis. The sialylated N-glycan fraction isolated from BM MSCs was digested with broad-range sialidase as described in the preceding Examples. After the reaction, it was observed by MALDI-TOF mass spectrometry that the vast majority of the sialylated N-glycans were desialylated and transformed into corresponding neutral N-glycans, indicating that they had contained sialic acid residues (NeuAc and/or NeuGc) as suggested by the proposed monosaccharide compositions. Glycan profiles of combined neutral and desialylated (originally sialylated) N-glycan fractions of BM MSCs grown in proliferation medium and in osteogenic medium correspond to total N-glycan profiles isolated from the cell samples (in desialylated form). It is calculated that in undifferentiated BM MSCs (grown in osteogenic medium), approximately 53% of the N-glycan signals correspond to high-mannose type N-glycan monosaccharide compositions, 8% to low-mannose type N-glycans, 31% to complex-type N-glycans, and 7% to hybrid-type or monoantennary N-glycan monosaccharide compositions. In differentiated BM MSCs (grown in osteogenic medium), approximately 28% of the N-glycan signals correspond to high-mannose type N-glycan monosaccharide compositions, 9% to low-mannose type N-glycans, 50% to complex-type N-glycans, and 11% to hybrid-type or monoantennary N-glycan monosaccharide compositions.

Lectin binding analysis of mesenchymal stem cells. As described under Experimental procedures, bone marrow derived mesenchymal stem cells were analyzed for the presence of ligands of α2,3-linked sialic acid specific (MAA) and α2,6-linked sialic acid specific (SNA) lectins on their surface. It was revealed that MAA bound strongly to the cells whereas SNA bound weakly, indicating that in the cell culture conditions, the cells had significantly more α2,3-linked than α2,6-linked sialic acids on their surface glycoconjugates. The present results suggest that lectin staining can be used as a further means to distinguish different cell types and complements mass spectrometric profiling results.

Detection of Potential Glycan Contaminations from Cell Culture Reagents

In the sialylated N-glycan profiles of MSC lines, specific N-glycan signals were observed that indicated contamination of mesenchymal stem cell glycoconjugates by abnormal sialic acid residues. First, when the cells were cultured in cell culture media with added animal sera, such as bovine of equine sera, potential contamination by N-glycolylneuraminic acid (Neu5Gc) was detected. The glycan signals at m/z 1946, corresponding to the [M−H]⁻ ion of NeuGc₁Hex₅HexNAc₄, as well as m/z 2237 and m/z 2253, corresponding to the [M−H]⁻ ions of NeuGc₁NeuAc₁Hex₅HexNAc₄ and NeuGc₂Hex₅HexNAc₄, respectively, were indicative of the presence of Neu5Gc, i.e. a sialic acid residue with 16 Da larger mass than N-acetylneuraminic acid (Neu5Ac). Moreover, when the cells were cultured in cell culture media with added horse serum, potential contamination by O-acetylated sialic acids was detected. Diagnostic signals used for detection of O-acetylated sialic acid containing sialylated N-glycans included [M−H]⁻ ions of Ac₁NeuAc₁Hex₅HexNAc₄, Ac₁NeuAc₂Hex₅HexNAc₄, and Ac₂NeuAc₂Hex₅HexNAc₄, at calculated m/z 1972.7, 2263.8, and 2305.8, respectively.

Conclusions

Uses of the glycan profiling method. The results indicate that the present glycan profiling method can be used to differentiate CB MSC lines and BM MSC lines from each other, as well as from other cell types such as cord blood mononuclear cell populations. Differentation-induced changes as well as potential glycan contaminations from e.g. cell culture media can also be detected in the glycan profiles, indicating that changes in cell status can be detected by the present method. The method can also be used to detect MSC-specific glycosylation features including those discussed below.

Differences in glycosylation between cultured cells and native human cells. The present results indicate that BM MSC lines have more high-mannose type N-glycans and less low-mannose type N-glycans compared to the other N-glycan structural groups than mononuclear cells isolated from cord blood. Taken together with the results obtained from cultured human embryonal stem cells in the following Examples, it is indicated that this is a general tendency of cultured stem cells compared to native isolated stem cells. However, differentiation of BM MSCs in osteogenic medium results in significantly increased amounts of complex-type N-glycans and reduction in the amounts of high-mannose type N-glycans.

Mesenchymal stem cell line specific glycosylation features. The present results indicate that mesenchymal stem cell lines differ from the other cell types studied in the present study with regard to specific features of their glycosylation, such as:
1) Both CB MSC lines and BM MSC lines have unique neutral and sialylated N-glycan profiles;
2) The major characteristic structural feature of both CB and BM MSC lines is abundant neutral complex-type N-glycans;
3) An additional characteristic feature is low sialylation level of complex-type N-glycans.

### Example 2

### Lectin and Antibody Profiling of Human Embryonic Stem Cells

Experimental Procedures

Cell samples. Human embryonic stem cell (hESC) lines FES 22 and FES 30 (Family Federation of Finland) were propagated on mouse feeder cell (mEF) layers as described above.

FITC-labeled lectins. Fluorescein isotiocyanate (FITC) labeled lectins were purchased from several manufacturers: FITC-GNA, -HHA, -MAA, -PWA, -STA and -LTA were from EY Laboratories (USA); FITC-PSA and -UEA and biotin-labelled WFA were from Sigma (USA); and FITC-RCA, -PNA and -SNA were from Vector Laboratories (UK).

Fluorescence microscopy labeling experiments were conducted essentially as described in the preceding Examples. Biotin label was visualized by fluorescein-conjugated streptavidin.

Results

Table 1 shows the tested FITC-labelled lectins and antibodies, examples of their target saccharide sequences, and the graded lectin binding intensities as described in the Table legend, in fluorescence microscopy of fixed cells grown on microscopy slides. Multiple binding specificities for the used lectins are described in the art and in general the binding of a lectin in the present experiments means that the cells express specific ligands for the lectin on their surface, but does not exclude the presence of also other ligands that are recognized by the lectin. See Example 14 for specificities for GF antibodies.

α-linked mannose and core Fuca6-eptopes. Abundant labelling of mEF by Pisum sativum (PSA) lectins suggests that they express mannose, more specifically α-linked mannose residues and core Fucα6-eptopes on their surface (or intracellular) glycoconjugates such as N-glycans. The results further suggest that the both hESC lines do not express these ligands at as high concentrations as mEF on their surface.

β-linked galactose. Abundant labelling of hESC by peanut lectin (PNA) and less intense labelling by Ricinus communis lectin I (RCA-I) suggests that hESC express β-linked non-reducing terminal galactose residues on their surface glycoconjugates such as N- and/or O-glycans. More specifically, RCA-I binding suggests that the cells contain high amounts of unsubstituted Galβ epitopes on their surface. PNA binding suggests for the presence of unsubstituted Galβ, and the absence of specific binding of PNA to mEF suggests that the binding epitopes for this lectin are less abundant in mEF.

Sialic acids. Specific labelling of hESC by both Maackia amurensis (MAA) and Sambucus nigra (SNA) lectins suggests that the cells express sialic acid residues on their surface glycoconjugates such as N- and/or O-glycans and/or glycolipids. More specifically, the specific MAA binding of hESC suggests that the cells contain high amounts of α2,3-linked sialic acid residues. In contrast, the results suggest that these epitopes are less abundant in mEF. SNA binding in both cell types suggests for the presence of also α2,6-linkages in the sialic acid residues on the cell surface.

Poly-N-acetyllactosamine sequences. Labelling of the cells by pokeweed (PWA) and less intense labelling by Solanum tuberosum (STA) lectins suggests that the cells express poly-N-acetyllactosamine sequences on their surface glycoconjugates such as N- and/or O-glycans and/or glycolipids. The results further suggest that cell surface poly-N-acetyllactosamine chains contain both linear and branched sequences.

β-linked N-acetylgalactosamine. Abundant labelling of hESC by Wisteria floribunda lectin (WFA) suggests that hESC express β-linked non-reducing terminal N-acetylgalactosamine residues on their surface glycoconjugates such as N- and/or O-glycans. The absence of specific binding of WFA to mEF suggests that the lectin ligand epitopes are less abundant in mEF.

Fucosylation. Labelling of the cells by Ulex europaeus (UEA) and less intense labelling by Lotus tetragonolobus (LTA) lectins suggests that the cells express fucose residues on their surface glycoconjugates such as N- and/or O-glycans and/or glycolipids. More specifically, the UEA binding suggests that the cells contain α-linked fucose residues including α1,2-linked fucose residues. LTA binding suggests for the presence of α-linked fucose residues including α1,3- or α1,4-linked fucose residues on the cell surface.

The specific antibody anti-Lex and anti-sLex antibody binding results indicate that the hESC samples contain Galβ4(Fucα3)GlcNAcβ and SAα3Galβ4(Fucα3)GlcNAcβ carbohydrate epitopes on their surface, respectively.

Taken together, in the present experiments the lectins PNA, MAA, and WFA as well as the antibodies anti-Lex and anti-sLex bound specifically to hESC but not to mEF. In contrast, the lectin PSA bound specifically to mEF but not to hESC. This suggests that the glycan epitopes that these reagents recognize have hESC or mEF specific expression patterns. On the other hand, other reagents in the tested reagent panel bound differentially to the two hESC lines FES 22 and FES 30, indicating cell line specific glycosylation of the hESC cell surfaces (Table 1).

Discussion

Venable, A., et al. (2005 BMC Dev. Biol.) have previously described lectin binding profiles of SSEA-4 enriched human embryonic stem cells (hESC) grown on mouse feeder cells. The lectins used were Lycopersicon esculenturn (LEA, TL), RCA, Concanavalin A (ConA), WFA, PNA, SNA, Hippeastrum hybrid (HHA, HHL), Vicia villosa (VVA), UEA, Phaseolus vulgaris (PHA-L and PHA-E), MAA, LTA (LTL), and Dolichos biflorus (DBA) lectins. In FACS and cytochemistry analysis, four lectins were found to have similar binding percentage as SSEA-4 (LEA, RCA, ConA, and WFA) and in addition two lectins also had high binding percentage (PNA and SNA). Two lectins did not bind to hESCs (DBA and LTA). Six lectins were found to partially bind to hESC (PHA-E, VVA, UEA, PHA-L, MAA, and HHA). The authors suggested that the differential lectin binding specificities can be used to distinguish hESC and differentiated hESC types based on carbohydrate presentation.

Venable et al. (2005) discuss some carbohydrate structures that they claim to have high expression on the surface of pluripotent SSEA-4 hESC (corresponding lectins according to Venable et al. in parenthesis): α-Man (ConA, HHA), Glc (ConA), Galβ3GalNAcβ (PNA), non-reducing terminal Gal (RCA), non-reducing terminal β-GalNAc (RCA), GalNAcβ4Gal (WFA), GlcNAc (LEA), and SAα6GalNAc (SNA). In addition, Venable et al. discuss some carbohydrate structures that they claim to have expression on surface of a proportion of pluripotent SSEA-4 hESC (corresponding lectins according to Venable et al. in parenthesis): Gal (PHA-L, PHA-E, MAA), GalNAc (VVA) and Fuc (UEA). However, based on the monosaccharide specificities oligosaccharide specificities on the target cannot be known e.g. ConA is not easily assigned to any specific to Glc or Man-structure and our MAA has no specificity to Gal residues, but SAα3-structures; it is realized that large differences exist between often numerous isolectins of a plant species and Venable did not disclose the exact lectins used. Technical problems avoiding exact interpretation is Background section.

In the present experiments, RCA binding was observed on both hESC line FES 22 and mEF, but not on FES 30. This suggests that RCA binding specificity in hESC varies from cell line to another. The present experiments also show other lectins to be expressed on only one out of the two hESC lines (Table 1), suggesting that there is individual variation in binding of some lectins.

Based on LTA not binding to hESC in their experiments, Venable et al. (2005) suggest that on hESC surface there are no non-modified fucose residues that are α-linked to GlcNAc. However, in the present experiments LTA as well as anti-Lex and anti-sLex monoclonal antibodies were found to bind to the hESC line FES 22. The present antibody binding results indicate that FucαGlcNAc epitopes, specifically Galβ4(Fucα3)GlcNAc sequences, are present on hESC surface.

Venable et al. (2005) describe that PNA recognizes in their hESC samples specifically Galβ3GalNAc structures, wherein the GalNAcresidue is β-linked. In the present experiments, PNA was used to recognize carbohydrate structures generally including β-linked galactose residues and without β-linkage requirement for the GalNAc residue.

Venable et al. (2005) describe that SNA recognizes in their hESC samples specifically SAα6GalNAc structures. In the present experiments, SNA was used to recognize α2,6-linked sialic acids in general and its ligands were also found on mEF.

Inhibition of MAA binding by 200 mM lactose in the experiments described by Venable et al. (2005) suggests non-specific binding of their MAA with respect to sialic acids. According to the present experiments, our MAA can recognize α2,3-linked sialic acid residues on hESC surface and differentiate between hESC and mEF.

### Example 3

### Lectin and Antibody Profiling of Human Mesenchymal Stem Cells

Experimental Procedures

Cell samples. Bone marrow derived human mesenchymal stem cell lines (MSC) were generated and cultured in proliferation medium as described above.

FITC-labeled lectins. Fluorescein isotiocyanate (FITC) labelled lectins were purchased from several manufacturers: FITC-GNA, -HHA, -MAA, -PWA, -STA and -LTA were from EY Laboratories (USA); FITC-PSA and -UEA were from Sigma (USA); and FITC-RCA, -PNA and -SNA were from Vector Laboratories (UK). Lectins were used in dilution of 5 μg/10⁵ cells in 1% human serum albumin (HSA; FRC Blood Service, Finland) in phosphate buffered saline (PBS).

Flow cytometry. Flow cytometric analysis of lectin binding was used to study the cell surface carbohydrate expression of MSC. 90% confluent MSC layers on passages 9-11 were washed with PBS and harvested into single cell suspensions by 0.25% trypsin-1 mM EDTA solution (Gibco). The trypsin treatment was aimed to gentle, but it is realized that part of the structures recognized when compared to experiments by antibodies may be partially lost or reduced. Detached cells were centrifuged at 600g for five minutes at room temperature. Cell pellet was washed twice with 1% HSA-PBS, centrifuged at 600 g and resuspended in 1% HSA-PBS. Cells were placed in conical tubes in aliquots of 70000-83000 cells each. Cell aliquots were incubated with one of the FITC labelled lectin for 20 minutes at room temperature. After incubation cells were washed with 1% HSA-PBS, centrifuged and resuspended in 1% HSA-PBS. Untreated cells were used as controls. Lectin binding was detected by flow cytometry (FACSCalibur, Becton Dickinson). Data analysis was made with Windows Multi Document Interface for Flow Cytometry (WinMDI 2.8). Two independent experiments were carried out.

Fluorescence microscopy labeling experiments were conducted as described in the preceding Examples.

Results and Discussion

Table 2 shows the tested FITC-labelled lectins, examples of their target saccharide sequences, and the amount of cells showing positive lectin binding (%) in FACS analysis after mild trypsin treatment. Table 3 shows the tested FITC-labelled lectins, examples of their target saccharide sequences, and the graded lectin binding intensities as described in the Table legend, in fluorescence microscopy of fixed cells grown on microscopy slides. Binding specificities of the used lectins are described in the art and in general the binding of a lectin in the present experiments means that the cells express specific ligands for the lectin on their surface. The examples of some of the specificities discussed below and those marked in the Tables are therefore non-exclusive in nature.

α-linked mannose. Abundant labelling of the cells by both Hippeastrum hybrid (HHA) and Pisum sativum (PSA) lectins suggests that they express mannose, more specifically α-linked mannose residues on their surface glycoconjugates such as N-glycans. Possible α-mannose linkages include al α1→2, α1→3, and α1→6. The lower binding of Galanthus nivalis (GNA) lectin suggests that some α-mannose linkages on the cell surface are more prevalent than others.

β-linked galactose. Abundant labelling of the cells by Ricinus communis lectin I (RCA-I) and less intense labelling by peanut lectin (PNA) suggests that the cells express β-linked non-reducing terminal galactose residues on their surface glycoconjugates such as N- and/or O-glycans. More specifically, the intense RCA-I binding suggests that the cells contain high amounts of unsubstituted Galβ epitopes on their surface. The binding of RCA-I was increased by sialidase treatment of the cells before lectin binding, indicating that the ligands of RCA-I on MSC were originally partly covered by sialic acid residues. PNA binding suggests for the presence of another type of unsubstituted Galβ epitopes such as Core 1 O-glycan epitopes on the cell surface. The binding of PNA was also increased by sialidase treatment of the cells before lectin binding, indicating that the ligands of PNA on MSC were originally mostly covered by sialic acid residues. These results suggest that both RCA-I and PNA can be used to assess the amount of their specific ligands on the cell surface of BM MSC, and with or without conjunction with sialidase treatment to assess the sialylation level of their specific epitopes.

Sialic acids. Abundant labelling of the cells by Maackia amurensis (MAA) and less intense labelling by Sambucus nigra (SNA) lectins suggests that the cells express sialic acid residues on their surface glycoconjugates such as N- and/or O-glycans and/or glycolipids. More specifically, the intense MAA binding suggests that the cells contain high amounts of α2,3-linked sialic acid residues on their surface. SNA binding suggests for the presence of also α2,6-linked sialic acid residues on the cell surface, however in lower amounts than α2,3-linked sialic acids. Both of these lectin binding activities could be reduced by sialidase treatment, indicating that the specificities of the lectins in BM MSC are mostly targeted to sialic acids.

Poly-N-acetyllactosamine sequences. Labelling of the cells by Solanum tuberosum (STA) and less intense labelling by pokeweed (PWA) lectins suggests that the cells express poly-N-acetyllactosamine sequences on their surface glycoconjugates such as N- and/or O-glycans and/or glycolipids. Higher intensity labelling with STA than with PWA suggests that most of the cell surface poly-N-acetyllactosamine sequences are linear and not branched or substituted chains.

Fucosylation. Labelling of the cells by Ulex europaeus (UEA) and less intense labelling by Lotus tetragonolobus (LTA) lectins suggests that the cells express fucose residues on their surface glycoconjugates such as N- and/or O-glycans and/or glycolipids. More specifically, the UEA binding suggests that the cells contain α-linked fucose residues, including α1,2-linked fucose residues, on their surface. LTA binding suggests for the presence of also α-linked fucose residues, including α1,3-linked fucose residues on the cell surface, however in lower amounts than UEA ligand fucose residues.

Mannose-binding lectin labelling. Low labelling intensity was also detected with human serum mannose-binding lectin (MBL) coupled to fluorescein label, suggesting that ligands for this innate immunity system component may be expressed on in vitro cultured BM MSC cell surface.

Binding of a NeuGc polymeric probe (Lectinity Ltd., Russia) to non-fixed hESC indicates the presence of NeuGc-specific lectin on the cell surfaces. In contrast, polymeric NeuAc probe did not bind to the cells with same intensity in the present experiments.

The binding of the specific antibodies to hESC indicates the presence of Lex and sialyl-Lewis x epitopes on their surfaces, and binding of NeuGc-specific antibody to hESC indicates the presence of NeuGc epitopes on their surfaces.

### Example 4

### Lectin and Antibody Profiling of Human Cord Blood Cell Populations

Results and Discussion

FIG. 1 shows the results of FACS analysis of FITC-labelled lectin binding to seven individual cord blood mononuclear cell (CB MNC) preparations (experiments performed as described above). Strong binding was observed in all samples by GNA, HHA, PSA, MAA, STA, and UEA FITC-labelled lectins, indicating the presence of their specific ligand structures on the CB MNC cell surfaces. Also mediocre binding (PWA), variable binding between CB samples (PNA), and low binding (LTA) was observed, indicating that the ligands for these lectins are either variable or more rare on the CB MNC cell surfaces as the lectins above.

### Example 5

### Analysis of the Human Embryonic Stem Cell N-Glycome

Structural proposals for N-glycan signals characterized by m/z values as the other Tables of the present invention, is presented in Tables 9 and 10a. The N-glycan schematic structures are according to the recommendations of the Consortium for Functional Glycomics (www.functionalglycomics.org) and as described e.g. in Goldberg et al. (2005) Proteomics 5, 865-875.

Materials and Methods

Human embryonic stem cell lines (hESC)—Generation of the Finnish hESC lines FES 21, FES 22, FES 29, and FES 30 has been described (17) and they were cultured according to the previous report. Briefly, two of the analysed cell lines were initially derived and cultured on mouse embryonic fibroblast (MEF) feeders, and two on human foreskin fibroblast (HFF) feeder cells. For the present studies all of the lines were transferred on HFF feeder cells and cultured in serum-free medium supplemented with Knockout serum replacement (Gibco). To induce the formation of embryoid bodies (EB) the hESC colonies were first allowed to grow for 10-14 days whereafter the colonies were cut in small pieces and transferred on non-adherent Petri dishes to form suspension cultures. The formed EBs were cultured in suspension for the next 10 days in standard culture medium without bFGF. For further differentiation (into stage 3 differentiated cells) EB were transferred onto gelatin-coated culture dishes in media supplemented with insulin-transferrin-selenium and cultured for 10 days.

For glycan analysis, the cells were collected mechanically, washed, and stored frozen until the analysis. In fluorescence-assisted cell sorting (FACS) analyses 70-90% of cells from mechanically isolated hESC colonies were typically Tra 1-60 and Tra 1-81 positive (not shown). The differentiation protocol favors the development of neuroepithelial cells while not directing the differentiation into distinct terminally differentiated cell types (18). Stage 3 cultures consisted of a heterogenous population of cells dominated by fibroblastoid and neuronal morphologies.

Glycan isolation—Asparagine-linked glycans were detached from cellular glycoproteins by F. meningosepticum N-glycosidase F digestion (Calbiochem, USA) essentially as described (19). Cellular contaminations were removed by precipitating the glycans with 80-90% (v/v) aqueous acetone at −20° C. and extracting them with 60% (v/v) ice-cold methanol (20). The glycans were then passed in water through C₁₈ silica resin (BondElut, Varian, USA) and adsorbed to porous graphitized carbon (Carbograph, Alltech, USA) (21). The carbon column was washed with water, then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C₁₈ silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used.

Mass spectrometry and data analysis—MALDI-TOF mass spectrometry was performed with a Bruker Ultraflex TOF/TOF instrument (Bruker, Germany) essentially as described (22). Relative molar abundancies of neutral and sialylated glycan components can be accurately assigned based on their relative signal intensities in the mass spectra when analyzed separately as the neutral and sialylated N-glycan fractions (22-25). Each step of the mass spectrometric analysis methods was controlled for reproducibility by mixtures of synthetic glycans or glycan mixtures extracted from human cells.

The mass spectrometric raw data was transformed into the present glycan profiles by carefully removing the effect of isotopic pattern overlapping, multiple alkali metal adduct signals, products of elimination of water from the reducing oligosaccharides, and other interfering mass spectrometric signals not arising from the original glycans in the sample. The resulting glycan signals in the presented glycan profiles were normalized to 100% to allow comparison between samples.

Quantitative difference between two glycan profiles (%) was calculated according to Equation 1:

$$difference = \frac{1}{2} {\sum }_{i = 1}^{n} \vert {p}_{i , a} - {p}_{i , b} \vert , \qquad ( 1 )$$

wherein p is the relative abundance (%) of glycan signal i in profile a or b, and n is the total number of glycan signals.

Relative difference between a glycan feature in two profiles was calculated according to Equation 2:

$$relative difference = {x ( \frac{{P}_{a}}{{P}_{b}} )}^{x} , \qquad ( 2 )$$

wherein P is the sum the relative abundancies of the glycan signals with the glycan feature in profile a or b, x is 1 when a≧b, and x is −1 when a<b.

The glycan analysis method was validated by subjecting human cell samples to blinded analysis by five different persons. The results were highly comparable (data not shown), especially by the terms of detection of individual glycan signals and their relative signal intensities, showing that the present method reliably produced glycan profiles suitable for comparison of analysis results from different cell types.

Glycosidase analysis—The neutral N-glycan fraction was subjected to digestion with Jack bean α-mannosidase (Canavalia ensiformis; Sigma, USA) essentially as described (22).

NMR methods—For NMR spectroscopic analyses, larger amounts of hESC were grown on mouse feeder cell (MEF) layers. The isolated glycans were purified for the analysis by gel filtration high-pressure liquid chromatography in a column of Superdex peptide HR 10/30 (Amersham), with water (neutral glycans) or 50 mM NH₄HCO₃ (sialylated glycans) as the eluant at a flow rate of 1 ml/min. The eluant was monitored at 214 nm, and oligosaccharides were quantified against external standards. The amount of N-glycans in NMR analysis was below five nanomoles. Prior to NMR analysis the purified glycome fractions were repeatedly dissolved in 99.996% deuterium oxide and dried to omit H₂O and to exchange sample protons. The proton NMR spectra at 800 MHz were recorded using a cryo-probe for enhanced sensitivity.

Statistical procedures—Glycan score distributions of all three differentiation stages (hESC, EB, and stage 3 differentiated cells) were analyzed by the Kruskal-Wallis test. Pairwise comparisons were performed by the 2-tailed Student's t-test with Welch's approximation and 2-tailed Mann-Whitney U test. A p value less than 0.05 was considered significant. The statistical analyses are described in more detail in Supplementary data.

Lectin staining—Fluorescein-labelled lectins used in lectin histochemistry were from EY Laboratories (USA). Specificity of binding was controlled by inhibition experiments with α3′-sialyllactose and D-mannose for Maackia amurensis agglutinin (MAA) and Pisum sativum agglutinin (PSA), respectively.

Results

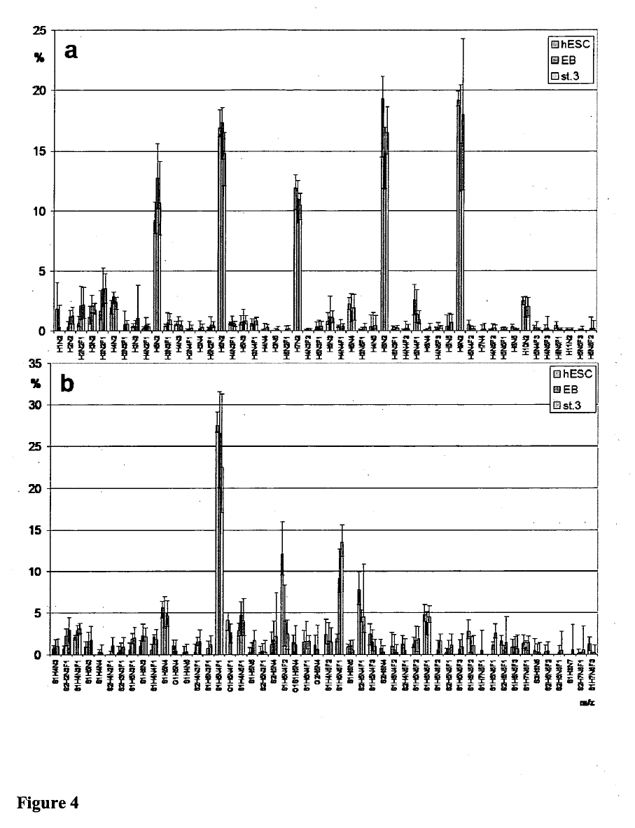

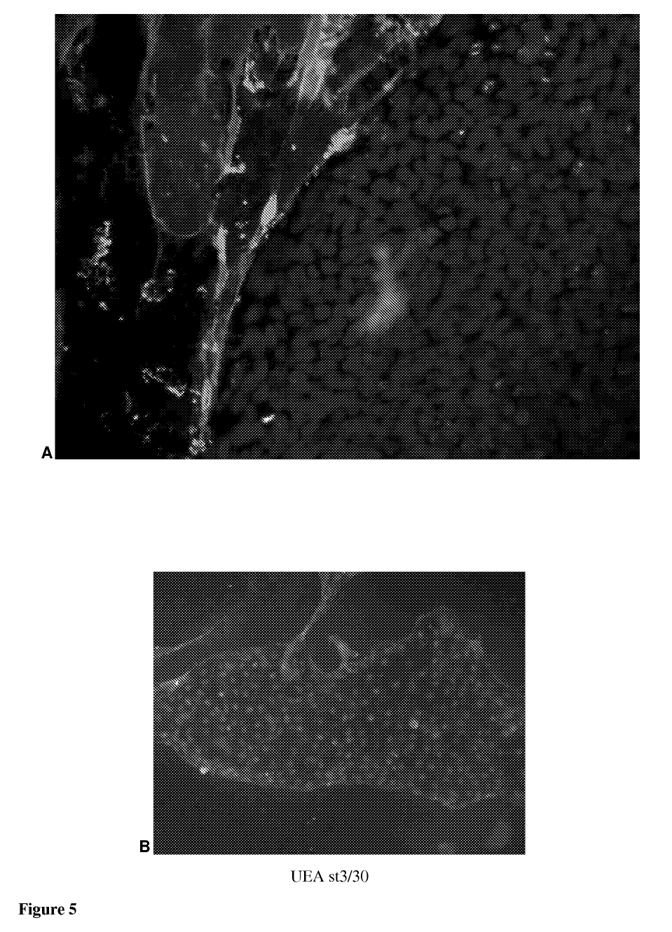

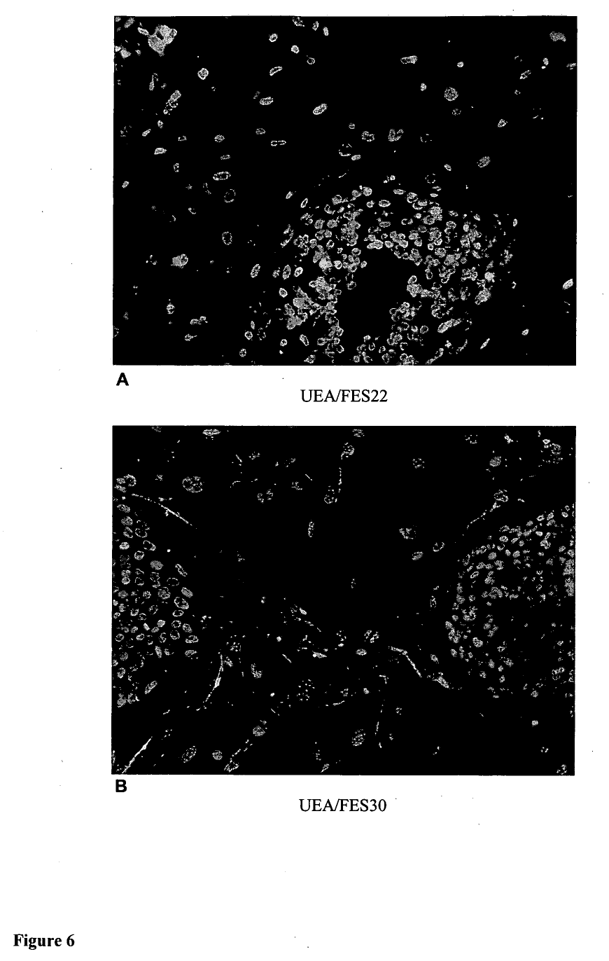

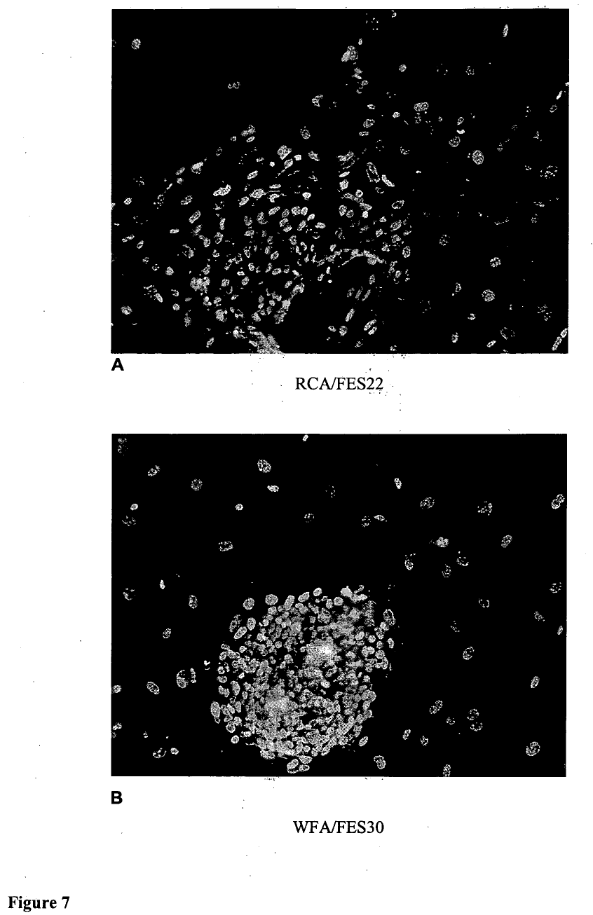

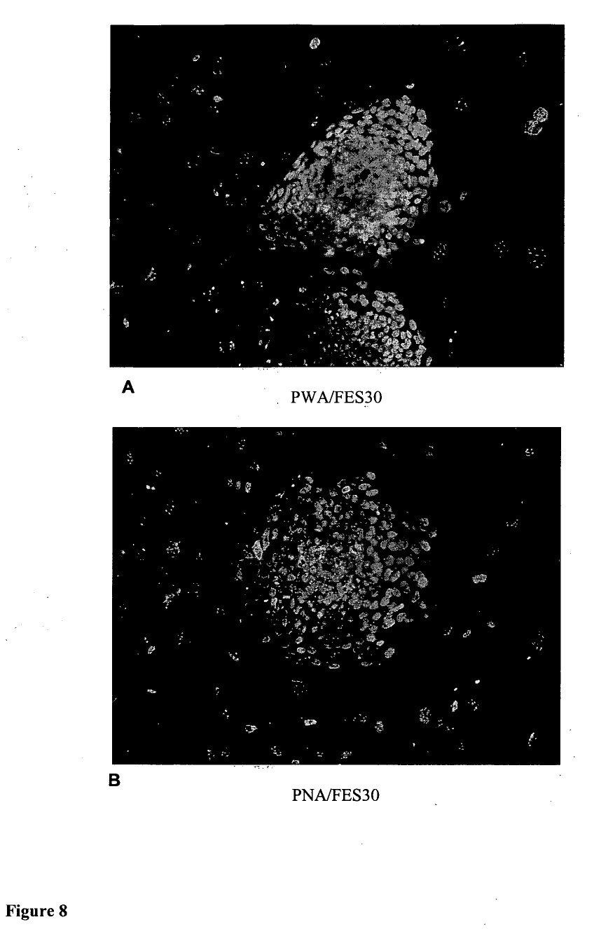

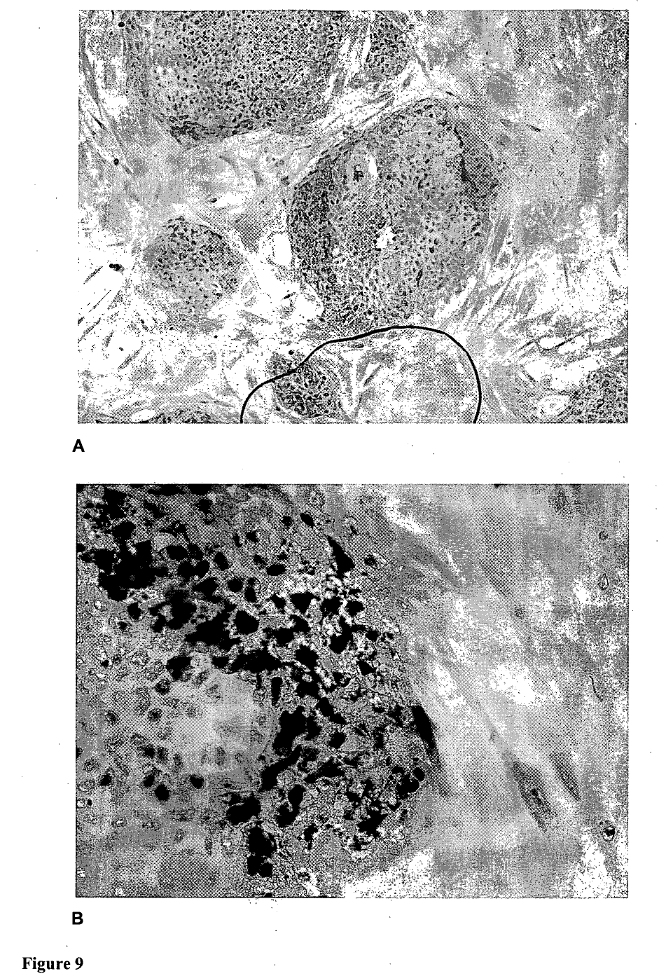

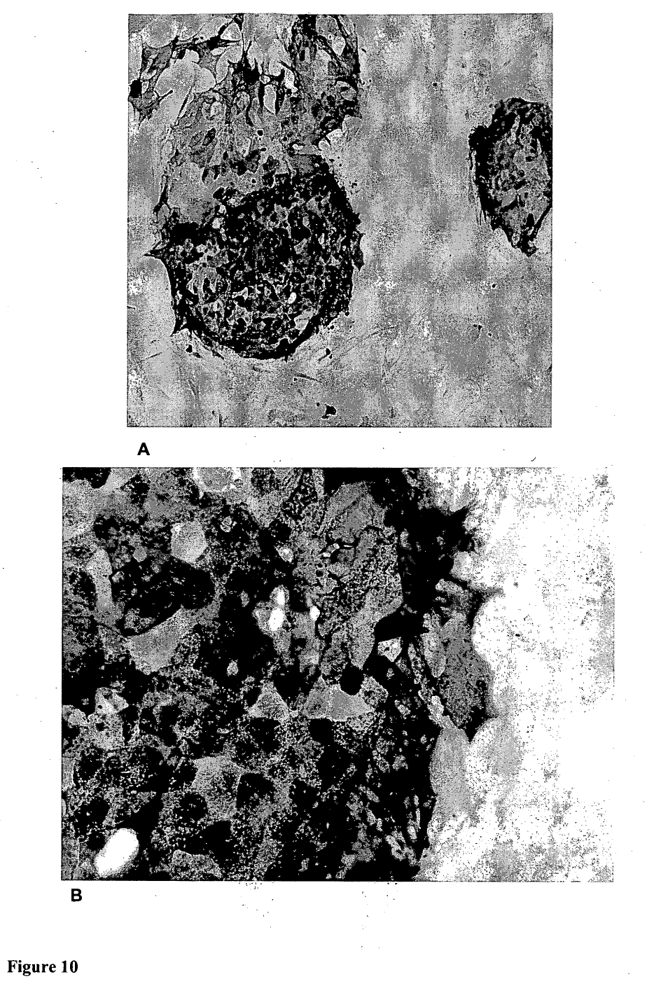

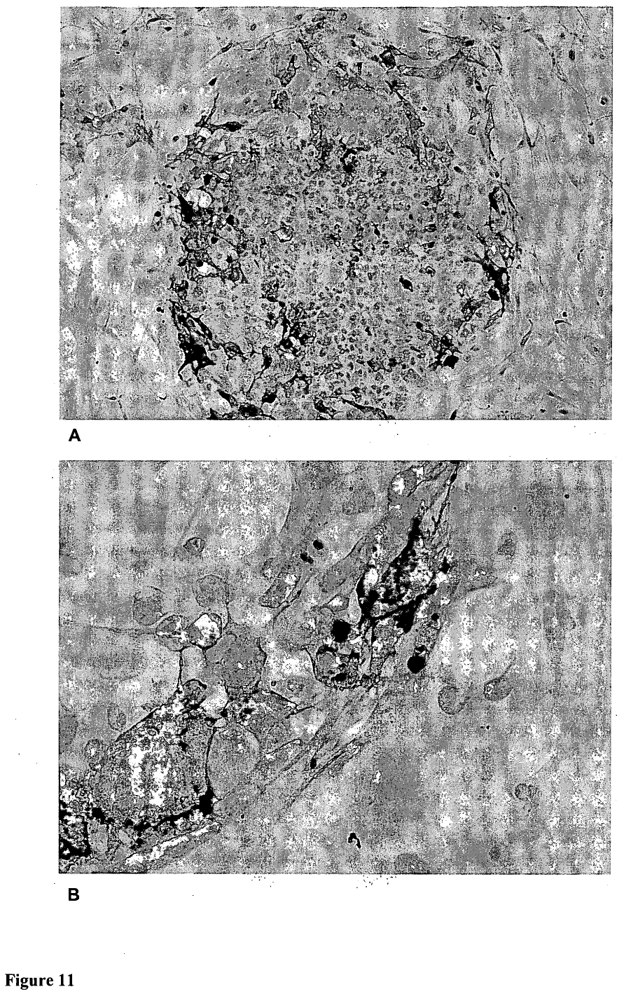

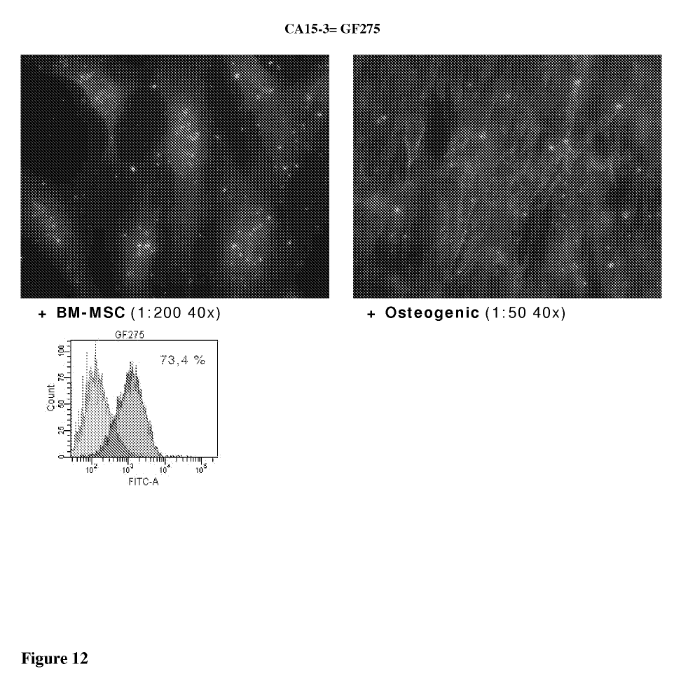

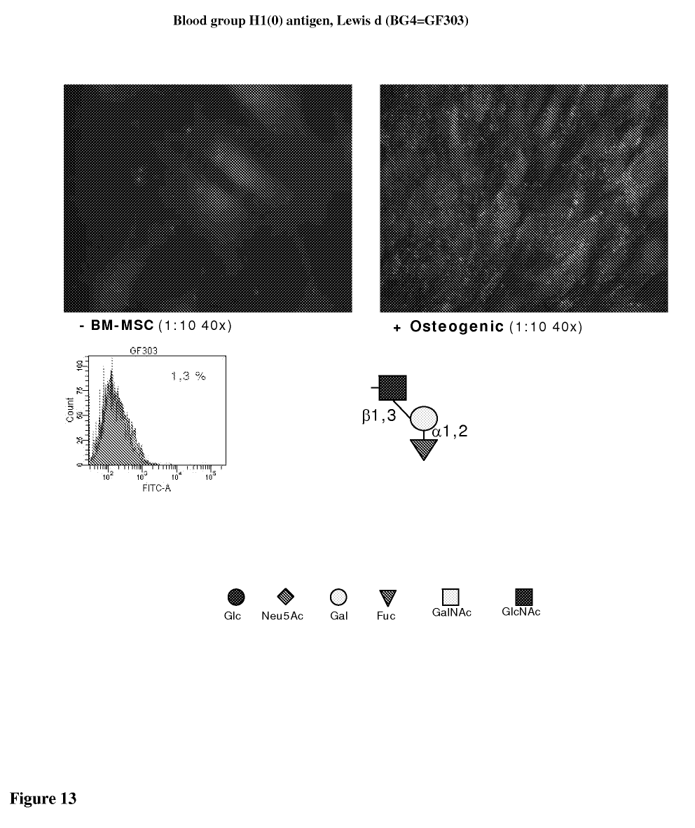

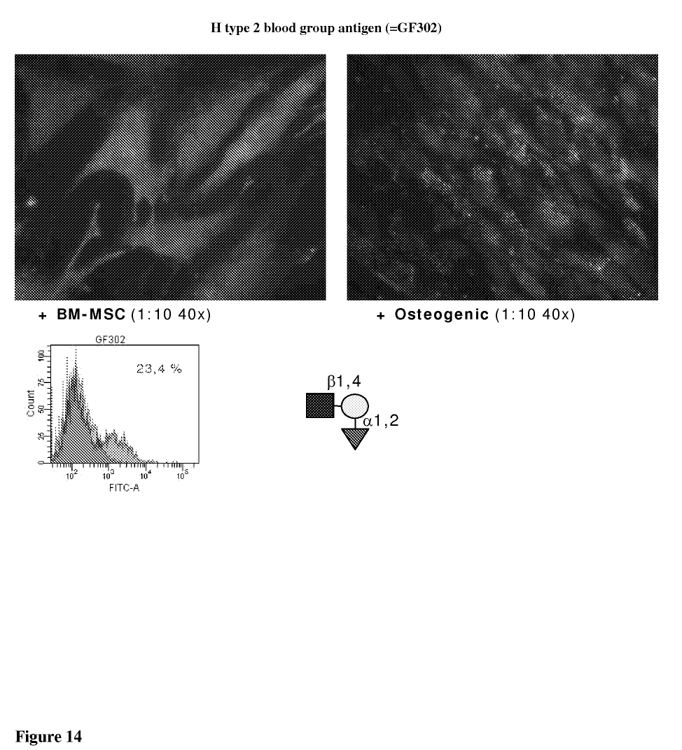

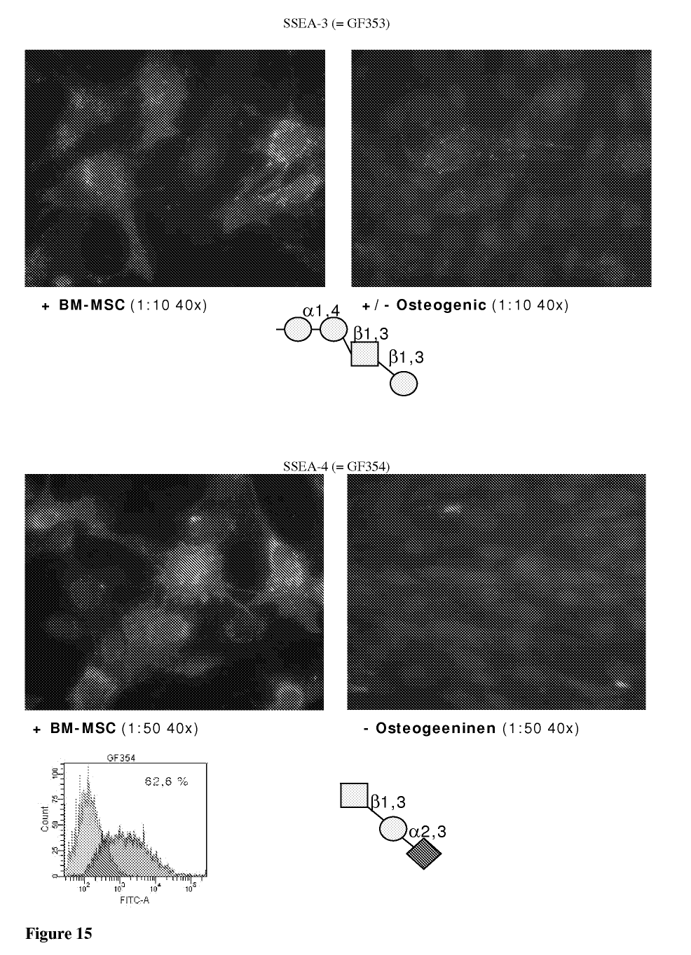

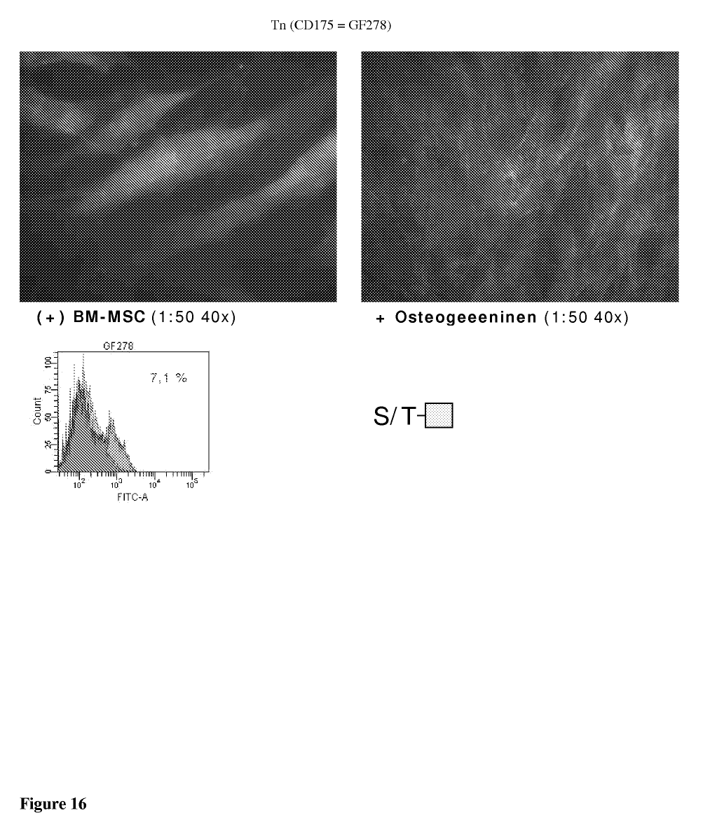

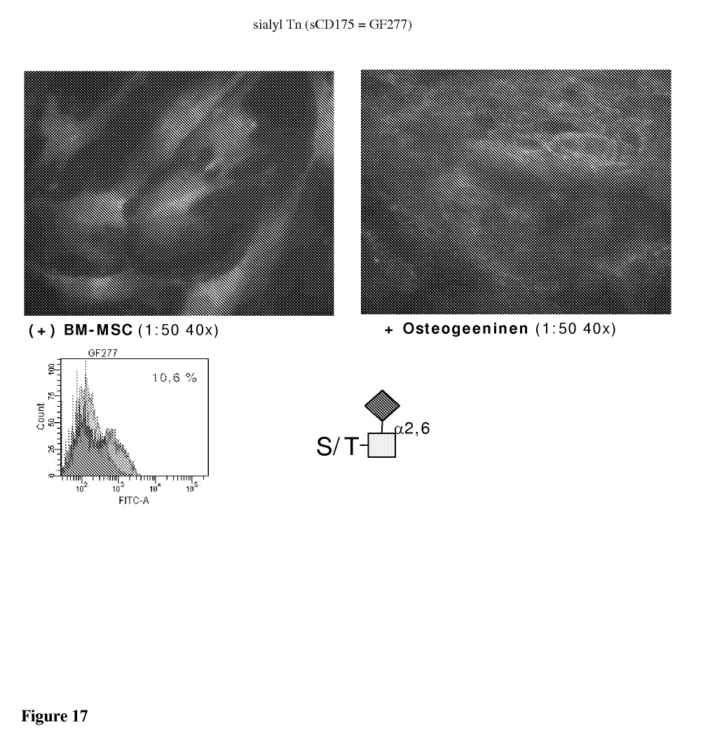

In order to generate mass spectrometric glycan profiles of hESC, embryoid bodies (EB), and further differentiated cells, a matrix-assisted laser desorption-ionization (MALDI-TOF) mass spectrometry based analysis was performed. We focused on the most common type of protein post-translational modifications, N-glycans, which were enzymatically released from cellular glycoproteins. During glycan isolation and purification, the total N-glycan pool was separated by an ion-exchange step into neutral N-glycans and sialylated N-glycans. These two glycan fractions were then analyzed separately by mass spectrometric profiling (FIG. 4), which yielded a global view of the N-glycan repertoire. Over one hundred N-glycan signals were detected from each cell type demonstrating that N-glycosylation is equally sophisticated in stem cells and cells differentiated from them. The proposed monosaccharide compositions corresponding to the detected masses of each individual signal in FIG. 4 are indicated by letter code. However, it is important to realize that many of the mass spectrometric signals in the present analyses include multiple isomeric structures and the one hundred most abundant signals very likely represent hundreds of different molecules.

The relative abundances of the observed glycan signals were determined based on their relative signal intensities (22,24-25), which allowed analysis of N-glycan profile differences between samples. The present data demonstrate that mass spectrometric profiling can be used in effective quantitative comparison of total glycan profiles, especially to pin-point the major glycosylation differences between related samples. In the following, we have expressed relative abundancies of glycan signals as molar proportions of the total detected N-glycans. However, these figures should be recognized as practical approximations based on the present data instead of absolutely quantitative percentages of the N-glycome.

In most of the previous glycomic studies of mammalian cells and tissues the isolated glycans have been derivatized (permethylated) prior to mass spectrometric profiling (26-29) or chromatographic analysis (30). However, we chose to directly analyze the picomolar quantities of unmodified glycans and increased sensitivity was achieved by omitting the derivatization and the subsequent additional purification steps. Our glycan purification scheme enabled N-glycan profiling analysis from samples as small as 100 000 cells showing that sensitivity of the analysis step is not a limiting factor in glycomic studies with scarce biological samples.

Overview of the hESC N-glycome: Neutral N-glycans Neutral N-glycans comprised approximately two thirds of the combined neutral and sialylated N-glycan pools of hESC. The 50 most abundant neutral N-glycan signals detected in the four hESC lines are presented in FIG. 4A (blue columns). The similarity of the profiles, which is indicated by the minor variation in the glycan signals, suggests that the four cell lines closely resemble each other. For example, 15 of the 20 most abundant glycan signals were the same in every hESC line. These 15 neutral N-glycan signals characteristic of the hESC N-glycome are listed in Table 6. The five most abundant signals (H₅N₂, H₆N₂, H₇N₂, H₈N₂, and H₉N₂; for abbreviations see FIG. 4) comprised 76% of the neutral N-glycans of hESC and dominated the profile.

Sialylated N-glycans—All N-glycan signals in the sialylated N-glycan fraction (FIG. 4B, blue columns) contained sialic acid residues (S: N-acetylneuraminic acid, or G: N-glycolylneuraminic acid). There was more variation between individual cell lines in the 50 most abundant sialylated N-glycans than in the neutral N-glycans. However, the four cell lines again resembled each other. The five most abundant sialylated N-glycan signals were the same in every cell line: S₁H₅N₄F₁, S₁H₅N₄F₂, S₂H₅N₄F₁, S₁H₅N₄, and S₁H₆N₅F₁. The 15 sialylated N-glycan signals common to all the hESC lines are listed in Table 6.

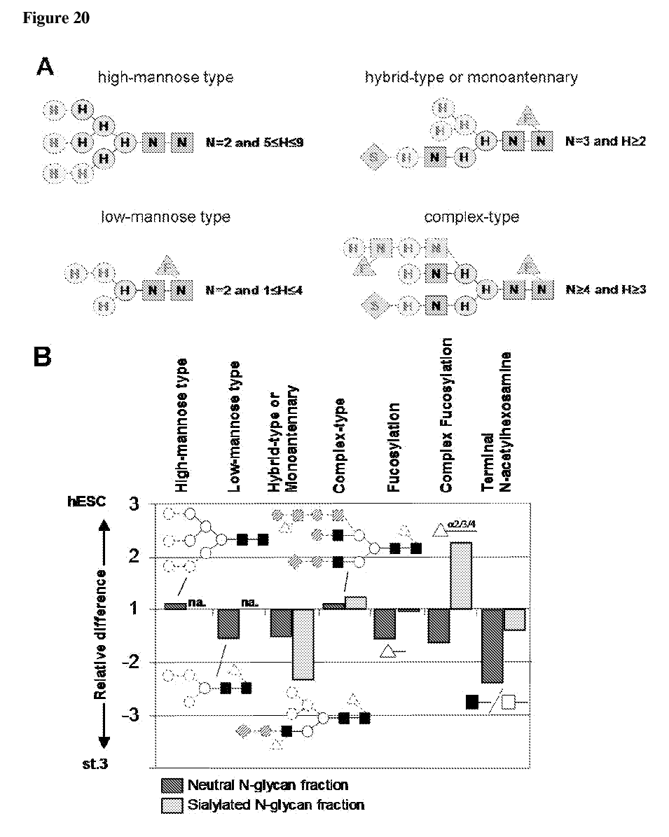

The most abundant sialylated glycan signals contained the H₅N₄ core composition and differed only by variable number of sialic acid (S or G) and deoxyhexose (F) residues. These comprised 61% of the total glycan signal intensity in FIG. 4B. Similarly, another common core structure was H₆N₅ that was present in seven signals comprising 12% of the total glycan signal intensity. These examples highlight the biosynthetic mechanism that leads to the complex spectra of N-glycan structures in cells: N-glycans typically consist of common core structures that are modified by the addition of variable epitopes (FIG. 20A).

Importantly, we detected N-glycans containing N-glycolylneuraminic acid (G) in the hESC samples, for example glycans G₁H₅N₄, G₁S₁H₅N₄, and G₂H₅N₄. N-glycolylneuraminic acid has previously been reported in hESC as an antigen transferred from culture media containing animal-derived materials (31). Accordingly, the serum replacement medium used in the present experiments contained bovine serum proteins. We have recently detected Neu5Gc in N-glycans of hESC and in vitro cultured human mesenchymal stem cells by mass spectrometric N-glycan analysis (32).

Variation between individual cell lines—Although the four hESC lines shared the same overall N-glycan profile, there was cell line specific variation within the profiles. Individual glycan signals unique to each cell line were detected, indicating that every cell line was slightly different from each other with respect to the approximately one hundred most abundant N-glycan structures. Importantly, the 30 most common N-glycan signals in all the hESC lines accounted for circa 85% of the total detected N-glycans, and they represent a useful approximation of the hESC N-glycome (Table 6).

Transformation of the N-glycome during hESC differentiation—A major goal of the present study was to identify glycan structures that would be specific to either stem cells or differentiated cells, and could therefore serve as differentiation stage markers. In order to determine whether the hESC N-glycome undergoes changes during differentiation, the N-glycan profiles obtained from hESC, EB, and stage 3 differentiated cells were compared (FIG. 4). The profiles of the differentiated cell types (EB and stage 3 differentiated cells) were clearly different compared to the profiles of undifferentiated hESC, as indicated by non-overlapping distribution bars in many glycan signals. Further, there were many signals present in both hESC and EB that were not detected in stage 3 differentiated cells. Overall, 10% of the glycan signals present in hESC had disappeared in stage 3 differentiated cells. Simultaneously numerous new signals appeared in EB and stage 3 differentiated cells. The proportion of these differentiation-associated N-glycan signals in EB and stage 3 differentiated cells was 14% and 16%, respectively.

Taken together, differentiation induced the appearance of new N-glycan types while earlier glycan types disappeared. Further, we found that the major hESC-specific N-glycosylation features were not expressed as discrete glycan signals, but instead as glycan signal groups that were characterized by specific monosaccharide composition features. In other words, differentiation of hESC into EB induced the disappearance of not only one but multiple glycan signals with hESC-associated features, and simultaneously also the appearance of glycan signal groups with other, differentiation-associated features.

The N-glycan profiles of the differentiated cells were also quantitatively different from the undifferentiated hESC profiles. A practical way of quantifying the differences between glycan profiles is to calculate the sum of the signal intensity differences between two samples (see Experimental procedures, Equation 1). According to this method, the EB neutral and sialylated N-glycan profiles had undergone a quantitative change of 14% and 29% from the hESC profiles, respectively. Similarly, the stage 3 differentiated cell neutral and sialylated N-glycan profiles had changed by 15% and 43%, respectively. Taking into account that the proportion of sialylated to neutral N-glycans in hESC was approximately 1:2, the total N-glycan profile change was approximately 25% during the transition from hESC to stage 3 differentiated cells.

The present data indicated that the mass spectrometric profile of the hESC N-glycome consisted of two discrete parts regarding propensity to change during hESC differentiation—a constant part of circa 75% and a changing part of circa 25%. In order to characterize the associated N-glycan structures, and to identify the potential biological roles of the constant and changing parts of the N-glycome, we performed structural analyses of the isolated hESC N-glycan samples.

Structural analyses of the major hESC N-glycans: Preliminary structure assignment based on monosaccharide compositions—Human N-glycans can be divided into biosynthetic groups of high-mannose type, hybrid-type, and complex-type N-glycans (33-34). Due to abundant expression of mannosylated N-glycans smaller than the classical high-mannose type structures in hESC, we added a new group called low-mannose N-glycans into this classification. To determine the presence of these N-glycan groups in the cells, assignment of probable structures matching the monosaccharide compositions of each individual signal was performed utilizing the established pathways of human N-glycan biosynthesis. Here, the detected N-glycan signals were classified into four N-glycan groups according to the number of N and H residues in the proposed compositions as shown in FIG. 20A: 1) high-mannose type and 2) low-mannose type N-glycans, which are both characterized by two N residues (N=2), 3) hybrid-type or monoantennary N-glycans, which are classified by three N residues (N=3), and 4) complex-type N-glycans, which are characterized by four or more N residues (N≧4) in their proposed monosaccharide compositions. However, this is an approximation and in addition to complex-type N-glycans also hybrid-type or monoantennary N-glycans may contain more than three N residues.

The data was analyzed quantitatively by calculating the percentage of glycan signals in the total N-glycome belonging to each structure group and comparing the hESC and differentiated cell glycan classification data (FIG. 20B). The relative differences in the structural groups reflect the activities of different biosynthetic pathways in each cell type. For example, the proportion of hybrid-type or monoantennary N-glycans was increased when hESC differentiated into EB, indicating that different glycan biosynthesis routes were favored in EB than in hESC. However, no glycan structure classes disappeared or appeared in the hESC differentiation process, which indicated that the fundamental N-glycan biosynthesis routes were not changed during differentiation. The proportion of low-mannose type N-glycans was surprisingly high in the light of earlier published studies of human N-glycosylation. However, according to our studies this is not specific to hESC (T. Satomaa, A. Heiskanen, J. Natunen, J. Saarinen, N. Salovuori, A. Olonen, J. Helin, M. Blomqvist, O. Carpén, unpublished results).

Verification of structure assignments by enzymatic glycan degradation and nuclear magnetic resonance spectroscopy—In order to validate the glycan structure assignments made based on the mass spectrometric analysis and the proposed monosaccharide compositions, we performed enzymatic degradation and proton NMR spectroscopy analyses of selected neutral and sialylated N-glycans.

For the validation of neutral N-glycans we chose the glycans H₅N₂, H₆N₂, H₇N₂, H₈N₂, and H₉N₂, which were the most abundant N-glycans in all studied cell types (FIG. 4A). The monosaccharide compositions of these glycans had already suggested (FIG. 20A) that they were high-mannose type N-glycans (33). To test this hypothesis, neutral N-glycans from hESC and the differentiated cell samples were treated with α-mannosidase, and analyzed both before and after the enzymatic treatment by MALDI-TOF mass spectrometry (data not shown). The glycans in question were degraded and the corresponding signals disappeared from the mass spectra, indicating that they had contained α-linked mannose residues.

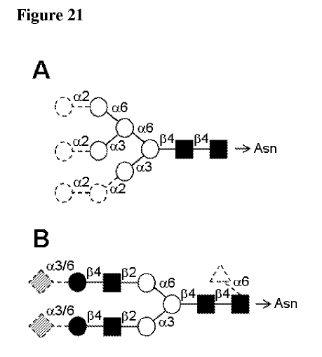

The neutral N-glycan fraction was further analyzed by nanoscale proton NMR spectroscopy. In the obtained NMR spectrum of the hESC neutral N-glycans signals consistent with high-mannose type N-glycans were abundant (FIG. 21A and Table 7), supporting the conclusion that they were the major glycan components in the sample. In proton NMR spectroscopic analysis of the sialylated N-glycan fraction, N-glycan backbone signals consistent with biantennary complex-type N-glycans were the major detected signals (FIG. 21B and Table 8), in line with the preliminary assignment made based on the proposed monosaccharide compositions. The present results indicated that the classification of the glycan signals within the total N-glycome data could be used to construct an approximation of the whole N-glycome.

Complex fucosylation of N-glycans is characteristic of hESC—Differentiation stage associated changes in the sialylated N-glycan profile of hESC were more drastic than in the neutral N-glycan fraction and the group of five most abundant sialylated N-glycan signals was different at every differentiation stage (FIG. 4B). In particular, there was a significant differentiation-associated decrease in the relative amounts of glycans S₁H₅N₄F₂ and S₁H₅N₄F₃ as well as other glycan signals that contained at least two deoxyhexose residues (F≧2). In contrast, glycan signals such as S₂H₅N₄ that contained no F were increased in the differentiated cell types. The results suggested that sialylated N-glycans in undifferentiated hESC were subject to more complex fucosylation than in the differentiated cell types (FIG. 20B). The most common fucosylation type in human N-glycans is α1,6-fucosylation of the N-glycan core structure (35). The NMR analysis of the sialylated N-glycan fraction of hESC also revealed α1,6-fucosylation of the N-glycan core as the most abundant type of fucosylation (Table 8). In N-glycans containing more than one fucose residue there has to be other fucose linkages in addition to the α1,6-linkage (35). The F≧2 structural feature decreased as the cells differentiated, indicating that complex fucosylation was characteristic of undifferentiated hESC.

N-glycans with terminal N-acetylhexosamine residues become more common with differentiation—A major group of N-glycan signals which increased during differentiation contained equal amounts of N-acetylhexosamine and hexose residues (N=H) in their monosaccharide composition (e.g. S₁H₅N₅F₁). This was consistent with N-glycan structures containing non-reducing terminal N-acetylhexosamine residues since such complex-type N-glycans generally have monosaccharide compositions of either N=H or N>H (FIG. 20A). EB and stage 3 differentiated cells showed increased amounts of potential terminal N-acetylhexosamine structures (FIG. 20B).

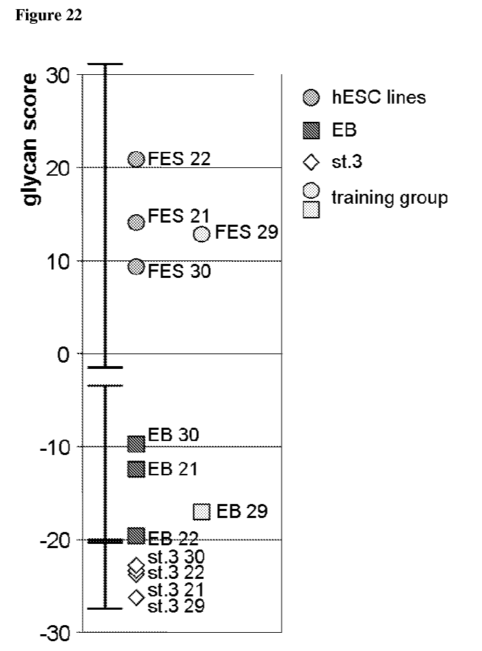

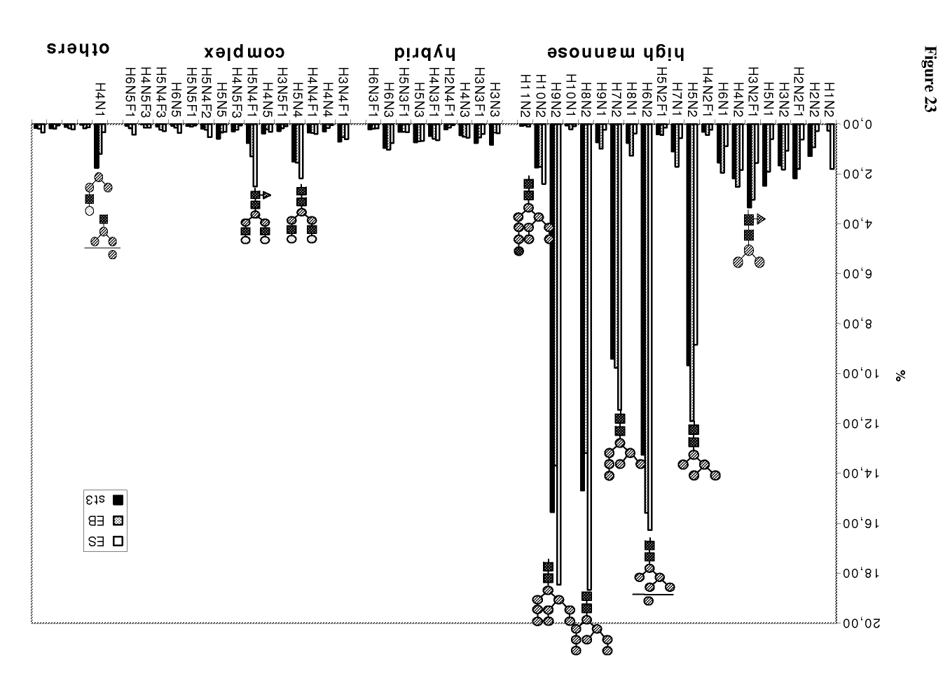

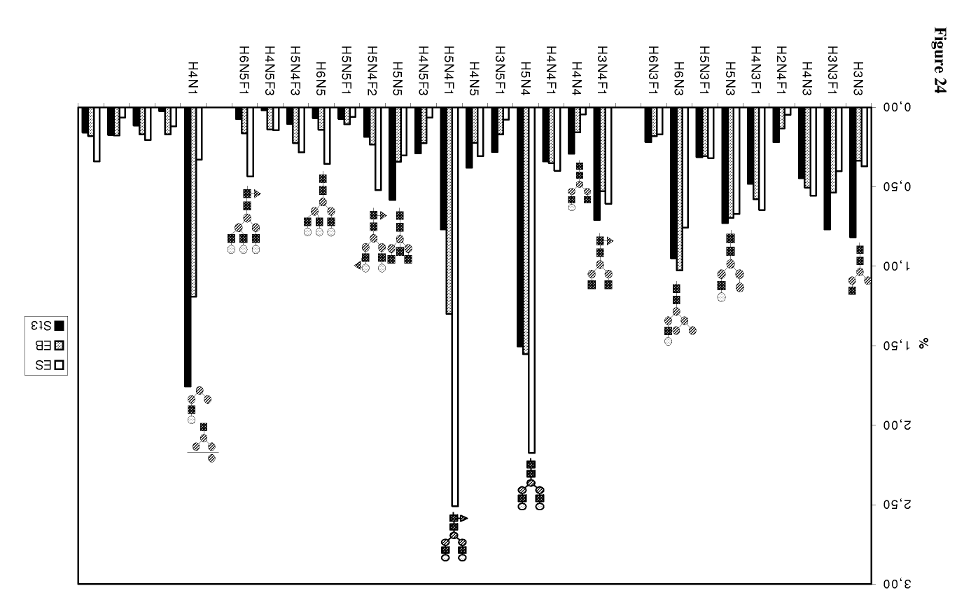

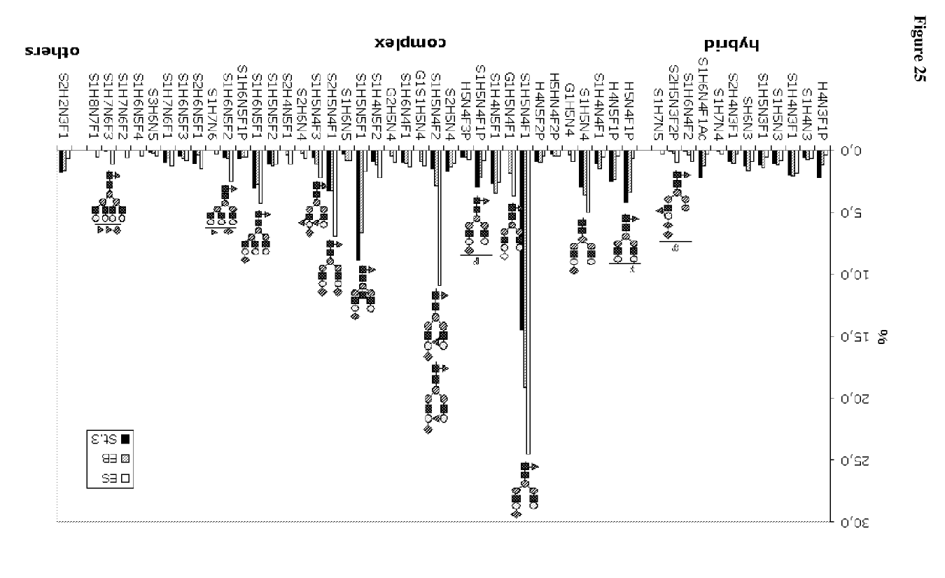

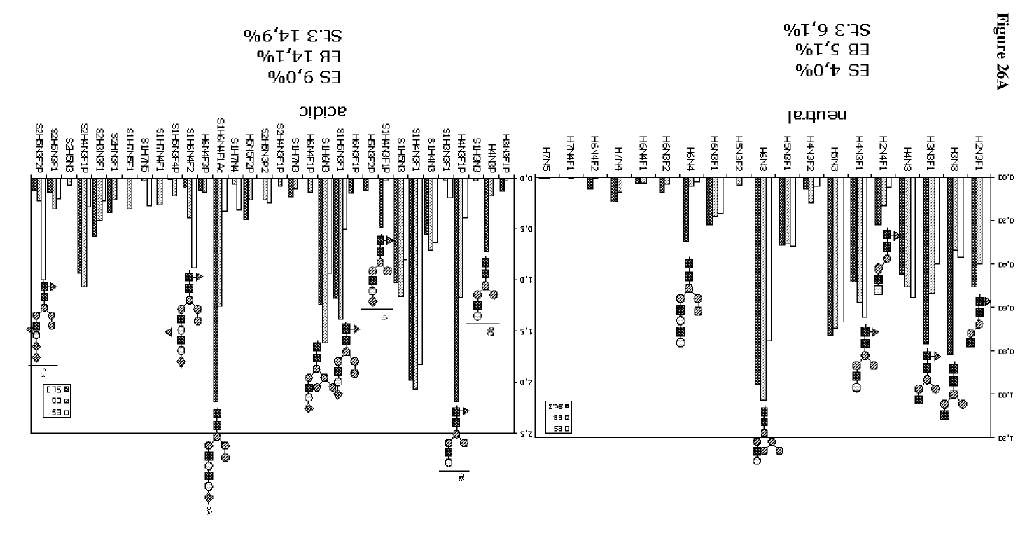

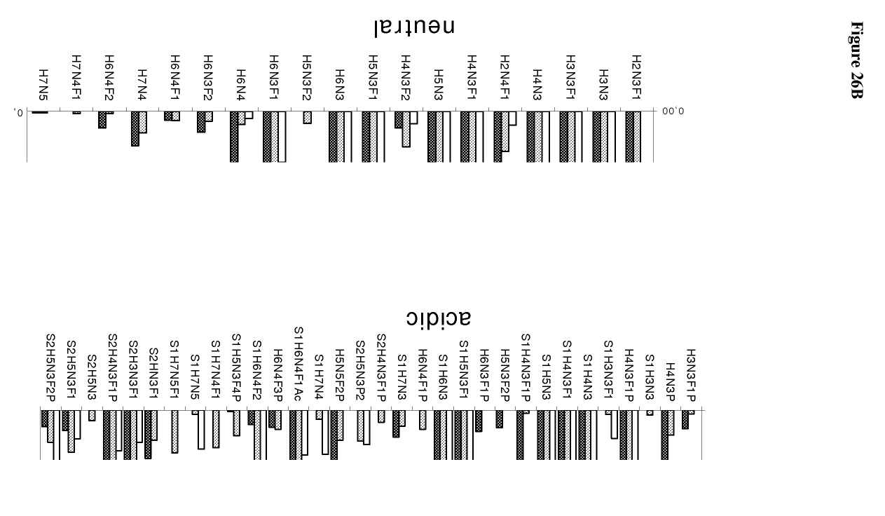

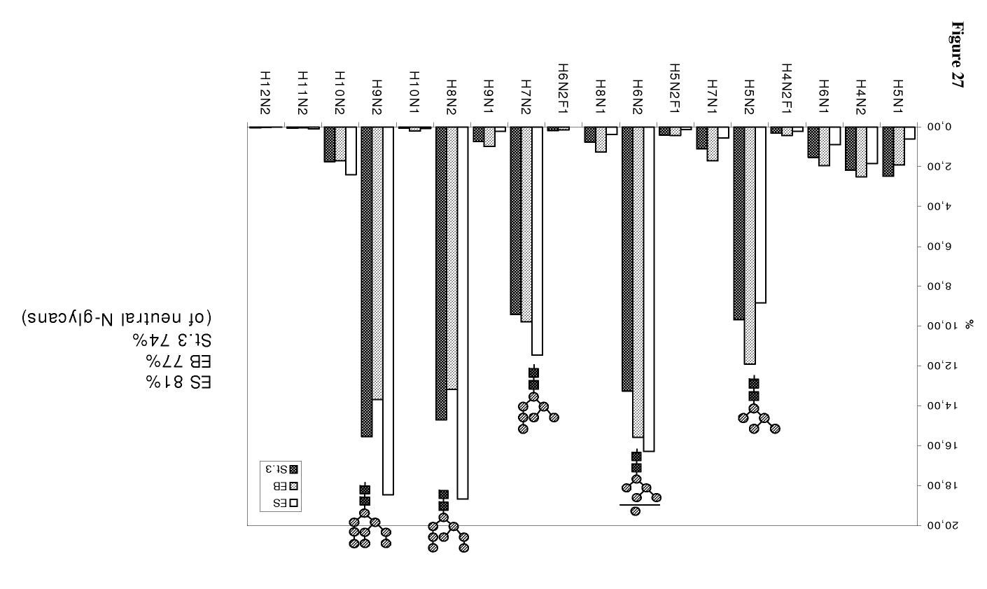

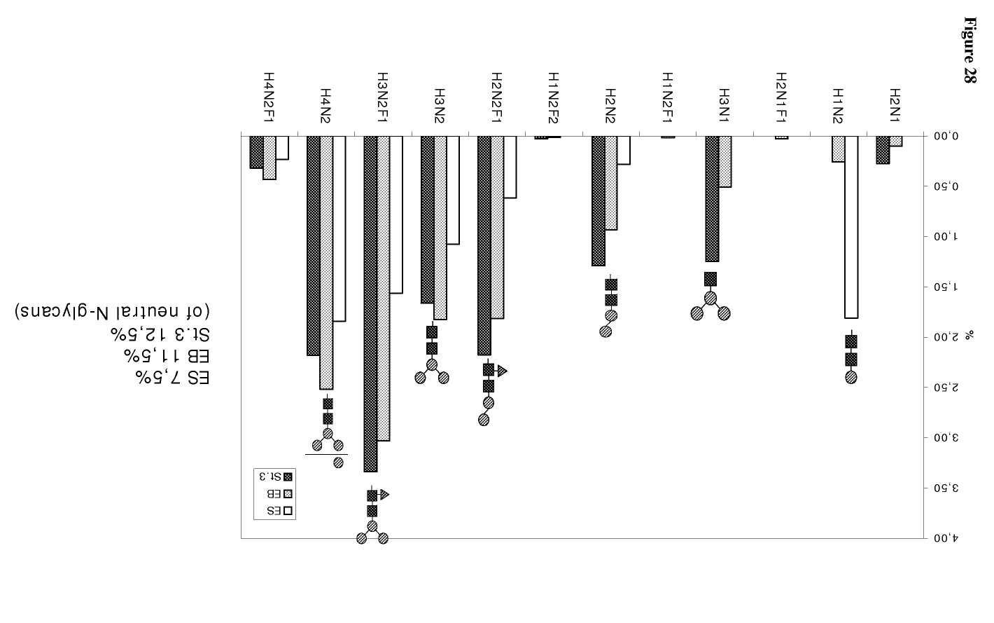

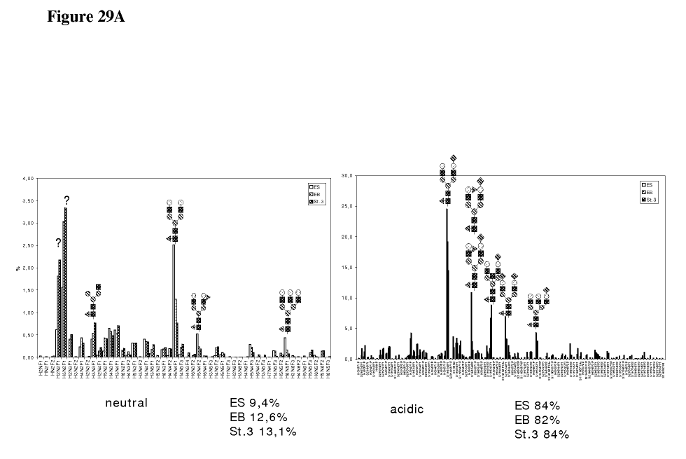

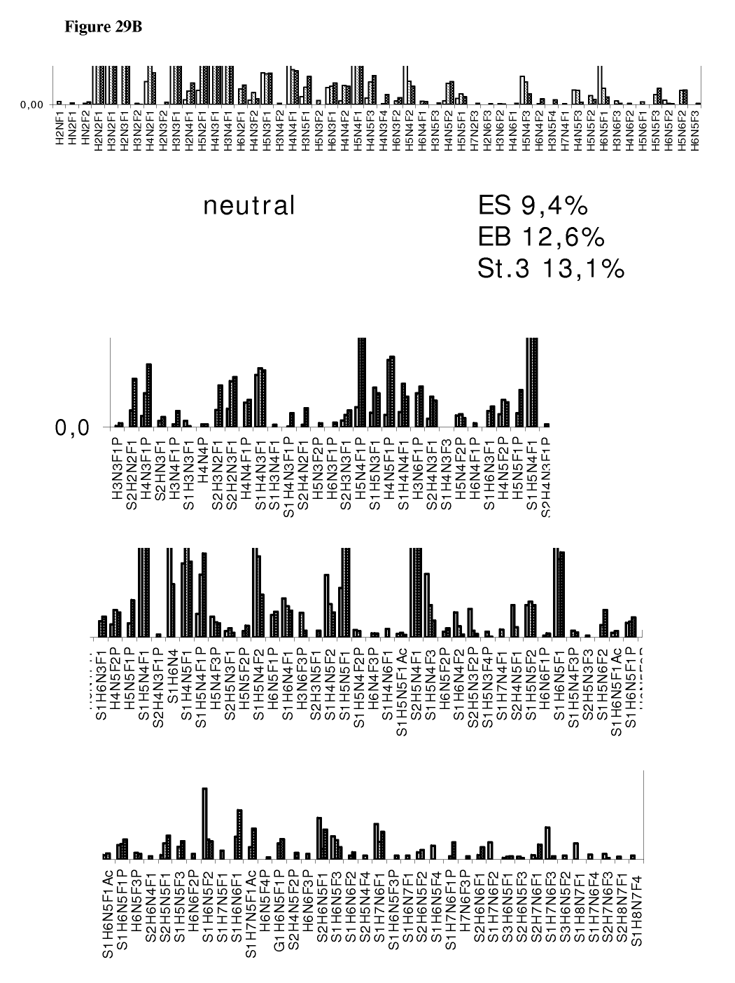

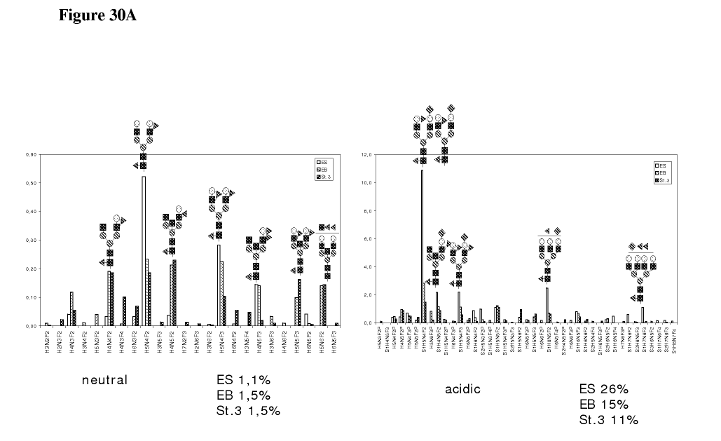

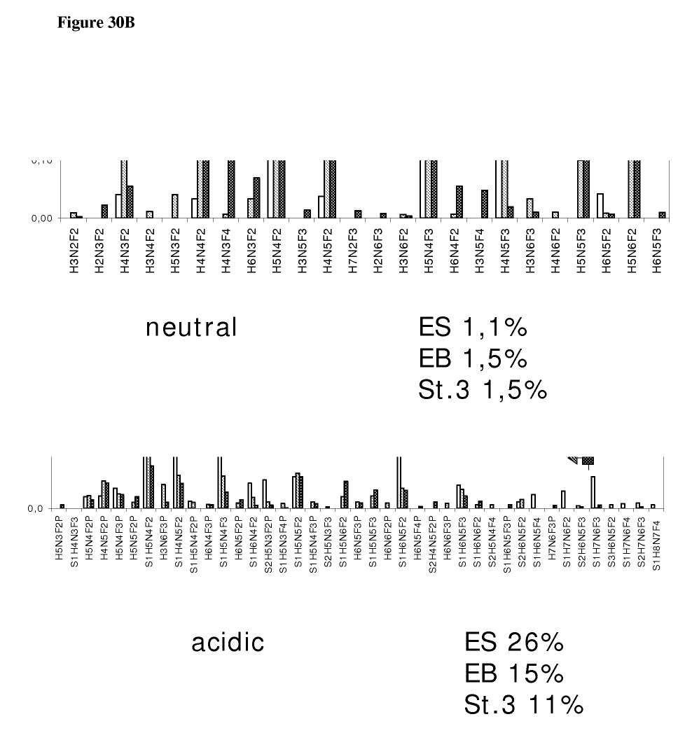

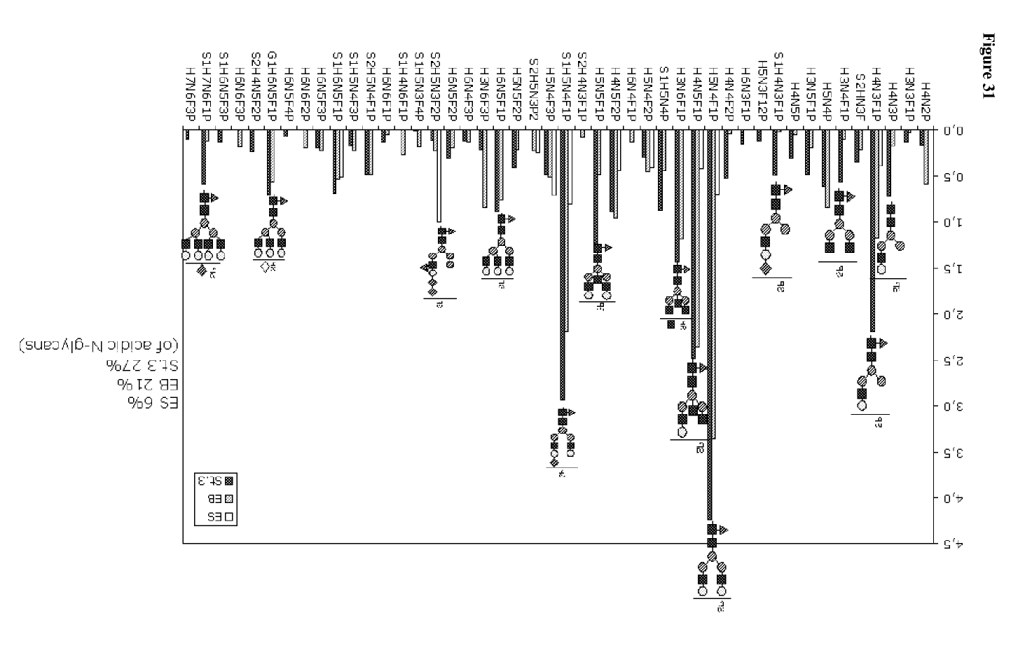

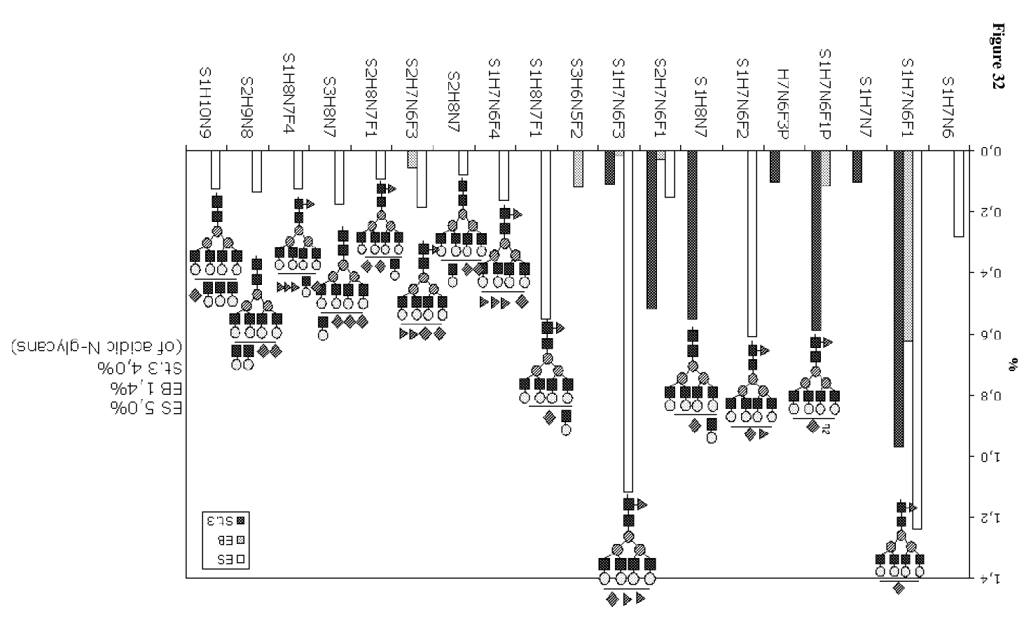

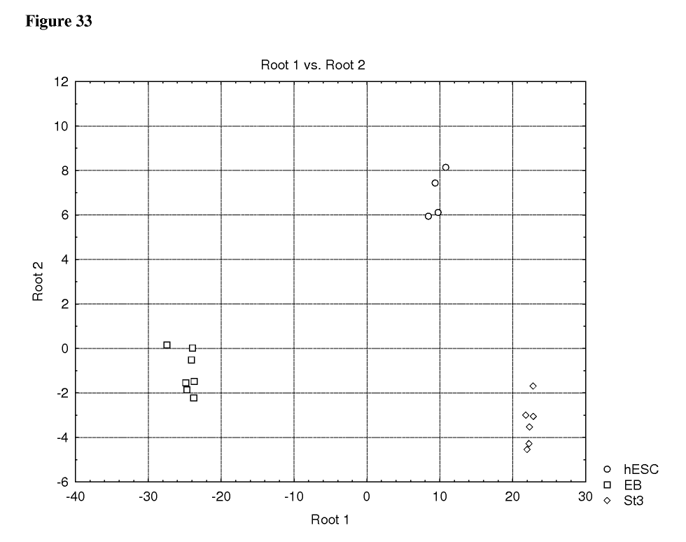

Glycome profiling can identify the differentiation stage of hESC—The glycome profile analyses indicated that the studied hESC lines and differentiated cells had differentiation stage specific N-glycosylation features. However, the data also demonstrated variation between individual cell lines. To test whether the obtained N-glycan profiles could be used to generate an efficient discrimination algorithm that would discriminate between hESC and differentiated cells, we performed a statistical evaluation of the mass spectrometric data (see Supplementary data for details). The results are described graphically in FIG. 22. The differentiated cell samples (EB and stage 3 differentiated cells) were significantly discriminated from hESC with p<0.01. The stage 3 differentiated cell samples were also significantly separated from the EB samples with p<0.01. This suggested that the hESC N-glycan profiles were similar at the glycome level despite of individual differences at the level of individual glycan signals. The result also suggested that glycome profiling is a potential tool for monitoring the differentiation status of stem cells.

The identified hESC glycans can be targeted at the cell surface—From a practical perspective stem cell research would be best served by reagents that recognize cell-type specific target structures on cell surface. To investigate whether individual glycan structures we had identified would be accessible to reagents targeting them at the cell surface we performed lectin labelling of two candidate structure types. Lectins are proteins that recognize glycans with specificity to certain glycan structures also in hESC (36-37). hESC colonies grown on mouse feeder cell layers were labeled in vitro by fluorescein-labelled lectins (FIG. 1). The hESC cell surfaces were clearly labeled by Maackia amurensis agglutinin (MAA) that recognizes structures containing α2,3-linked sialic acids, indicating that sialylated glycans were abundant on the hESC cell surface (FIG. 1A). Such glycans would thus be available for recognition by more specific glycan-recognizing reagents such as antibodies. In contrast, the cell surfaces were not labelled by Pisum sativum agglutinin (PSA) that recognizes α-mannosylated glycans (FIG. 1B). However, PSA labelled the cells after permeabilization (data not shown), suggesting that the majority of the mannosylated N-glycans in hESC were localized in intracellular cell compartments such as ER or Golgi (FIG. 1C). Interestingly, the mouse fibroblast cells showed complementary staining patterns compared to hESC, suggesting that these lectin reagents efficiently discriminated between hESC and feeder cells. Together the results suggested that the glycan structures we identified could be utilized to design reagents specifically targeting undifferentiated hESC.

Discussion

In the present study, novel glycan analysis methods were applied in the first structural analysis of hESC N-glycan profiles. By employing efficient purification of non-derivatized glycans we demonstrated mass spectrometric N-glycan profiles of the scarce hESC and differentiated cell samples from approximately 100 000 cells. As a result, dramatic glycan profile differences were discovered between the analyzed cell types. The objective in the present study was to provide a global view on the N-glycome profile, or a “fingerprint” of hESC N-glycosylation, rather than to present the stem cell glycome in terms of the molecular structures of each glycan component. The structural information already allowed us to determine the most abundant N-glycan structures of hESC. Furthermore, changes observed in the N-glycan profiles provided vast amount of information regarding hESC N-glycosylation and its changes during differentiation, allowing rational design of detailed structural studies of selected glycan components. It will be of great interest to apply these glycan analysis methods to other stem cell and differentiated cell types.

The results indicated that a defined group of N-glycan signals dominates the hESC N-glycome forming a unique stem cell glycan profile. For example, the fifteen most abundant neutral N-glycan signals and fifteen most abundant sialylated N-glycan signals in hESC together comprised over 85% of the N-glycome. On the other hand, structurally different glycan structures were favored during hESC differentiation. This suggests that N-glycan biosynthesis in hESC is a controlled and predetermined process.

Based on our results the hESC N-glycome seems to contain both a constant part consisting of “housekeeping glycans”, and a changeable part that is altered when the hESC differentiate (FIG. 4). The constant part seems to contain mostly high-mannose type and biantennary complex-type N-glycans, which may need to be present at all times for the maintenance of fundamental cellular processes. Significantly, 25% of the total N-glycan profile of hESC changed during their differentiation. This indicates that during differentiation hESC dramatically change both their appearance towards their environment and possibly also their own capability to sense and respond to exogenous signals.

Our data show that the differentiation-associated change in the N-glycome was mostly generated by the addition or removal of variable epitopes on similar N-glycan core compositions. The present lectin staining experiments demonstrated that sialylated glycans were abundant on the cell surface of hESC, indicating that cell type specific N-glycan structures are potential targets for development of more specific recognition reagents. It seems plausible that knowledge of the changing surface glycan epitopes could be utilized as a basis in developing reagents and culture systems that would allow improved identification, selection, manipulation, and culture of hESC and their progeny.

Protein-linked glycans perform their functions in cells by acting as ligands for specific glycan receptors (38-39), functioning as structural elements of the cell (40), and modulating the activity of their carrier proteins and lipids (2). More than half of all proteins in a human cell are glycosylated. Consequently, a global change in protein-linked glycan biosynthesis can simultaneously modulate the properties of multiple proteins. It is likely that the large changes in N-glycans during hESC differentiation have major influences on a number of cellular signaling cascades and affect in profound fashion biological processes within the cells.

The major hESC specific glycosylation feature we identified was the presence of more than one deoxyhexose residue in N-glycans, indicating complex fucosylation. Fucosylation is known to be important in cell adhesion and signalling events as well as being essential for embryonic development (41). Knock-out of the N-glycan core α1,6-fucosyltransferase gene FUT8 leads to postnatal lethality in mice (42), and mice completely deficient in fucosylated glycan biosynthesis do not survive past early embryonic development (43).

Fucosylated glycans such as the SSEA-1 antigen (7, 44-45) have previously been associated with both mouse embryonic stem cells (mESC) and human embryonic carcinoma cells (EC; 16), but not with hESC. The published gene expression profiles for the same hESC lines as studied here (46) have demonstrated that three human fucosyltransferase genes, FUT1, FUT4, and FUT8 are expressed in hESC, and that FUT1 and FUT4 are overexpressed in hESC when compared to EB. FUT8 encodes the N-glycan core α1,6-fucosyltransferase whose product was identified as the major fucosylated epitope in hESC N-glycans (FIG. 21B). The hESC-specific expression of FUT1 and FUT4, encoding for α1,2-fucosyltransferase and α1,3-fucosyltransferase enzymes (47), respectively, correlate with our findings of simple fucosylation in EB and complex fucosylation in hESC. Interestingly, the FUT4-encoded enzyme is capable of synthesizing the SSEA-1 antigen (48-49). Although hESC do not express the specific glycolipid antigen recognized by the SSEA-1 antibody, they share with mESC the characteristic feature of complex fucosylation and may also share the conserved essential biological functions of fucosylated glycan epitopes.

New N-glycan forms also emerged in EB and stage 3 differentiated cells. These structural features included additional N-acetylhexosamine residues, potentially leading to new N-glycan terminal epitopes. Another differentiation-associated feature was increase in the molar proportions of hybrid-type or monoantennary N-glycans. Biosynthesis of hybrid-type and complex-type N-glycans has been demonstrated to be biologically significant for embryonic and postnatal development in the mouse (50-51). The preferential expression of complex-type N-glycans in hESC and then the change in the differentiating EB to express more hybrid-type or monoantennary N-glycans may be significant for the process of stem cell differentiation.

Human embryonic stem cell lines have previously been demonstrated to have a common genetic stem cell signature that can be identified using gene expression profiling techniques (17,52-54). Such signatures have been proposed to be useful in hESC characterization. In the present report we provide the first glycomic signatures for hESC. The profile of the expressed N-glycans might be a useful tool for analyzing and classifying the differentiation stage in association with gene and protein expression analyses. Here we demonstrated that a glycan score algorithm was able to reliably differentiate the cell samples in separate differentiation stages (FIG. 22). Glycome profiling might be more sensitive than the use of any single cell surface marker and especially useful for the quality control of hESC-based cell products. However, further analysis of the hESC glycome may also lead to discovery of novel glycan antigens that could be used as stem cell markers in addition to the commonly used SSEA and Tra glycan antigens.

In conclusion, hESC have a unique N-glycome which undergoes major changes when the cells differentiate. Information regarding the specific glycan structures may be utilized in developing reagents for targeting these cells and their progeny. Future studies investigating the developmental and molecular regulatory processes resulting in the observed N-glycan profiles may provide significant insight into mechanisms of human development and regulation of glycosylation.

### REFERENCES FOR THE EXAMPLE

1. Shriver, Z., Raguram, S., and Sasisekharan, R. (2004) Nat. Rev. Drug Disc. 3, 863-873

2. Varki, A. (1993) Glycobiology 3, 97-130

3. Apweiler, R., Hermjakob, H., and Sharon, N. (1999) Biochim. Biophys. Acta 1473, 4-8

4. Lowe, J. B. (2002) Immunol. Rev. 186, 19-36

5. Fukuda, M. (2002) Biochim. Biophys. Acta 1573, 394-405

6. Dell, A., Morris, H. R., Easton, R. L., Patankar, M., and Clark, G. F. (1999) Biochim. Biophys. Acta 1473, 196-205

7. Fenderson, B. A., Zehavi, U., and Hakomori, S. (1984) J. Exp. Med. 160, 1591-1596

8. Handel, T. M., Johnson, Z., Crown, S. E., Lau, E. K., and Proudfoot, A. E. (2005) Annu. Rev. Biochem. 74, 385-410

9. Helenius, A., and Aebi, M. (2001) Science 291, 2364-2369

10. Helenius, A., and Aebi, M. (2004) Annu. Rev. Biochem. 73, 1019-1049

11. Kornfeld, S. (1986) J. Clin. Invest. 77, 1-6

12. Thomson, J. A., Itskovitz-Eldor, J., Shapiro, S. S., Waknitz, M. A., Swiergiel, J. J., Marshall, V. S., and Jones, J. M. (1998) Science 282, 1145-1147

13. Wobus, A. M., and Boheler, K. R. (2005) Physiol. Rev. 85, 635-678

14. Kannagi, R., Cochran, N. A., Ishigami, F., Hakomori, S., Andrews, P. W., Knowles, B. B., and Solter, D. (1983) EMBO J 2, 2355-2361

15. Badcock, G., Pigott, C., Goepel, J., and Andrews, P. W. (1999) Cancer Res. 59, 4715-4719

16. Muramatsu, T., and Muramatsu, H. (2004) Glycoconj. J. 21, 41-45

17. Mikkola, M., Olsson, C., Palgi, J., Ustinov, J., Palomäki, T., Horelli-Kuitunen, N., Knuutila, S., Lundin, K., Otonkoski, T., Tuuri, T. (2006) BMC Dev. Biol. 6, 40

18. Okabe, S., Forsberg-Nilsson, K., Spiro, A. C., Segal, M., and McKay, R. D. (1996) Mech. Dev. 59, 89-102

19. Nyman, T. A., Kalkkinen, N., Tölö, H., and Helin, J. (1998) Eur. J. Biochem. 253, 485-493

20. Verostek, M. F., Lubowski, C., and Trimble, R. B. (2000) Anal. Biochem. 278, 111-122

21. Davies, M. J., Smith, K. D., Carruthers, R. A., Chai, W., Lawson, A. M., and Hounsell, E. F. (1993) J. Chromatogr. 646, 317-326

22. Saarinen, J., Welgus, H. G., Flizar, C. A., Kalkkinen, N., and Helin, J. (1999) Eur. J. Biochem. 259, 829-840

23. Harvey, D. J. (1993) Rapid Commun. Mass Spectrom. 7, 614-619

24. Naven, T. J., and Harvey, D. J. (1996) Rapid Commun. Mass Spectrom. 10, 1361-1366

25. Papac, D. I., Wong, A., and Jones, A. J. (1996) Anal. Chem. 68, 3215-3223

26. Dell, A., and Morris, H. R. (2001) Science 291, 2351-2356

27. Sutton-Smith, M., Morris, H. R., Grewal, P. K., Hewitt, J. E., Bittner, R. E., Goldin, E., Schiffmann, R., and Dell, A. (2002) Biochem. Soc. Symp. 69, 105-115

28. Novotny, M. V., and Mechref, Y. J. (2005) Sep. Sci. 28, 1956-1968

29. Uematsu, R., Furukawa, J., Nakagawa, H., Shinohara, Y., Deguchi, K., Monde, K., and Nishimura, S. (2005) Mol. Cell. Proteomics 4, 1977-1989

30. Callewaert, N., Van Vlierberghe, H., Van Hecke, A., Laroy, W., Delanghe, J., and Contreras, R. (2004) Nat. Med. 10, 429-434

31. Martin, M. J., Muotri, A., Gage, F., and Varki, A. (2005) Nat. Med. 11, 228-232

32. Heiskanen, A., Satomaa, T., Tiitinen, S., Laitinen, A., Mannelin, S., Impola, U., Mikkola, M., Olsson, C., Miller-Podraza, H., Blomqvist, M., Olonen, A., Salo, H., Lehenkari, P., Tuuri, T., Otonkoski, T., Natunen, J., Saarinen, J., Laine, J. Stem Cells, in press.

33. Kornfeld, R., and Kornfeld, S. (1985) Annu. Rev. Biochem. 54, 631-664

34. Schachter, H. (1991) Glycobiology 1, 453-461

35. Staudacher, E., Altmann, F., Wilson, I. B. H., and März, L. (1999) Biochim. Biophys. Acta 1473, 216-346

36. Venable, A., Mitalipova, M., Lyons, I., Jones, K., Shin, S., Pierce, M., and Stice, S. (2005) BMC Dev. Biol. 5, 15.

37. Wearne, K. A., Winter, H. C., O'Shea, K., and Goldstein, I. J. (2006) Glycobiology, in press

38. Kilpatrick, D. C. (2002) Biochim. Biophys. Acta 1572, 187-197

39. Zanetta, J. P., and Vergoten, G. (2003) Adv. Exp. Med. Biol. 535, 107-124

40. Imperiali, B., and O'Connor, S. E. (1999) Curr. Opin. Chem. Biol. 3, 643-649

41. Becker, D. J., and Lowe, J. B. (2003) Glycobiology 13:41R-53R

42. Wang, X., Inoue, S., Gu, J., Miyoshi, E., Noda, K., Li, W., Mizuno-Horikawa, Y., Nakano, M., Asahi, M., Takahashi, M., Uozumi, N., Ihara, S., Lee, S. H., Ikeda, Y., Yamaguchi, Y., Aze, Y., Tomiyama, Y., Fujii, J., Suzuki, K., Kondo, A., Shapiro, S. D., Lopez-Otin, C., Kuwaki, T., Okabe, M., Honke, K., and Taniguchi, N. (2005) Proc. Natl. Acad. Sci. U.S.A. 102:15791-15796

43. Smith, P. L., Myers, J. T., Rogers, C. E., Zhou, L., Petryniak, B., Becker, D. J., Homeister, J. W., and Lowe, J. B. (2002) J. Cell Biol. 158, 801-815

44. Solter, D., and Knowles, B. B. (1978) Proc. Natl. Acad. Sci. U.S.A. 75, 5565-5569

45. Gooi, H. C., Feizi, T., Kapadia, A., Knowles, B. B., Solter, D., and Evans, M. J. (1981) Nature 292, 156-158

46. Skottman, H., Mikkola, M., Lundin, K., Olsson, C., Strömberg, A. M., Tuuri, T., Otonkoski, T., Hovatta, O., and Lahesmaa, R. (2005) Stem cells 23, 1343-1356

47. Mollicone, R., Cailleau, A., and Oriol, R. (1995) Transfusion Clin. Biol. 4:235-242

48. Nakayama, F., Nishihara, S., Iwasaki, H., Kudo, T., Okubo, R., Kaneko, M., Nakamura, M., Karube, M., Sasaki, K., and Narimatsu, H. (2001) J. Biol. Chem. 276, 16100-16106

49. Kudo, T., Kaneko, M., Iwasaki, H., Togayachi, A., Nishihara, S., Abe, K., and Narimatsu, H. (2004) Mol. Cell. Biol. 24, 4221-4228

50. Ioffe, E., and Stanley, P. (1994) Proc. Natl. Acad. Sci. U.S.A. 91, 728-732

51. Metzler, M., Gertz, A., Sarkar, M., Schachter, H., Schrader, J. W., and Marth, J. D. (1994) EMBO J. 13, 2056-2065

52. Wang, Y., Tan, J., Sutton-Smith, M., Ditto, D., Panico, M., Campbell, R. M., Varki, N. M., Long, J. M., Jaeken, J., Levinson, S. R., Wynshaw-Boris, A., Morris, H. R., Le, D., Dell, A., Schachter, H., and Marth, J. D. (2001) Glycobiology 11, 1051-1070

53. Akama, T. O., Nakagawa, H., Wong, N. K., Sutton-Smith, M., Dell, A., Morris, H. R., Nakayama, J., Nishimura, S., Pai, A., Moremen, K. W., Marth, J. D., and Fukuda, M. N. (2006) Proc. Natl. Acad. Sci. U.S.A. 103, 8983-8988

54. Sato, N., Sanjuan, I. M., Heke, M., Uchida, M., Naef, F., and Brivanlou, A. H. (2003) Dev. Biol. 260, 404-413

55. Abeyta, M. J., Clark, A. T., Rodriguez, R. T., Bodnar, M. S., Pera, R. A., and Firpo, M. T. (2004) Hum. Mol. Genet. 13, 601-608

56. Bhattacharya, B., Miura, T., Brandenberger, R., Mejido, J., Luo, Y., Yang, A. X., Joshi, B. H., Ginis, I., Thies, R. S., Amit, M., Lyons, I., Condie, B. G., Itskovitz-Eldor, J., Rao, M. S., and Puri, R. K. (2004) Blood 103, 2956-2964

### Example 6

### The Glycome of Human Embryonic Stem Cells Reflects their Differentiation Stage

In the present study, we analyzed the N-glycome profiles of hESC, EB, and st.3 differentiated cells (FIG. 2).

The similarity of the N-glycan profiles within the group of four hESC lines suggested that the obtained N-glycan profiles are a description of the characteristic N-glycome of hESC. Overall, 10% of the 100 most abundant N-glycan signals present in hESC disappeared in st.3 differentiated cells, and 16% of the most abundant signals in st.3 differentiated cells were not present in hESC. This indicates that differentiation induced the appearance of new N-glycan types while earlier glycan types disappeared. In quantitative terms, the differences between the glycan profiles of hESC, EB, and st.3 differentiated cells were: hESC vs. EB 19%, hESC vs. st.3 24%, and EB vs. st.3 12%.

The glycome profile data was used to design glycan-specific labeling reagents for hESC. The most interesting glycan types were chosen to study their expression profiles by lectin histochemistry as exemplified in FIG. 3 for the lectins that recognize either α2,3-sialylated (MAA-lectin, FIG. 3A.) binding to the hESC cells or α-mannosylated glycans (PSA-lectin, FIG. 3B.) binding to the surfaces of feeder cells (MEF). The binding of the lectin reagents was inhibited by specific carbohydrate inhibitors, sialylα2-lactose and mannose, respectively (FIGS. 3C. and 3D.). The results are summarized in Table 4.

Table 4 further represent differential recognition feeder and stem cells by two other lectins, Ricinus communis agglutinin (RCA, ricin lectin), known to recognize especially terminal Galβ-structures, especially Galβ4Glc(NAc)-type structures and peanut agglutinin (PNA) recognizing Gal/GalNAc structures. The cell surface expression of ligand for two other lectin RCA and PNA on hESC cells, but only RCA ligands of feeder cells.

The present results indicate and the invention is directed to the hESC glycans are potential targets for recognition by stem cell specific reagents. The invention is further directed to methods of specific recognition and/or separation of hESC and differentiated cells such as feeder cells by glycan structure specific reagents such as lectins. Human embryonic stem cells have a unique glycome that reflects their differentiation stage. The invention is specifically directed to analysis of cells according to the invention with regard to differentiation stage.

Conclusions

The present data represent the glycome profiling of hESC:
hESC have a unique N-glycome comprising of over 100 glycan components
Differentiation induces a major change in the N-glycome and the cell surface molecular landscape of hESC

Utility of hESC glycome data:
Identification of new stem cell markers for e.g. antibody development
Quality control of stem cell products
Identification of hESC differentiation stage
Control of variation between hESC lines
Effect of external factors and culture conditions on hESC status

Use of the hESC glycome for identification of specific cell surface markers characteristic for the pluripotent hESCs. The invention is directed to further analysis and production of present and analogous glycome data and use of the methods for further identification of novel stem cell specific glycosylation features and form the basis for studies of hESC glycobiology and its eventual applications according to the invention

### Example 7

### Lectin Based Selection of CB MNC Cell Populations

The FACS experiments with fluorescein-labeled lectins and CB MNC were performed essentially similarly to Example 4. Double stainings were performed with CD34 specific monoclonal antibody (Jaatinen et al., 2006) with complementary fluorescent dye. Erythroblast depletion from CD MNC fraction was performed by anti-glycophorin A (GlyA) monoclonal antibody negative selection.

Results and Discussion

Compared to the CB MNC fraction, GlyA depleted CB MNC showed decreased staining in FACS with the following lectins (the decrease in % in parenthesis): PWA (48%), LTA (59%), UEA (34%), STA, MAA, and PNA (all latter three less than 23%); indicating that GlyA depletion increased the resolving power of the lectins in cell sorting.

In FACS double staining with both fluorescein-labeled lectins and anti-CD34 antibody, the following lectins colocalized with CD34+ cells: STA (3/3 samples), HHA (3/3 samples), PSA (3/3 samples), RCA (3/3 samples), and partly also NPA (2/3 samples). In contrast, the following lectins did not colocalize with CD34+ cells: GNA (3/3 samples) and PWA (3/3 samples), and partly also LTA (2/3 samples), WFA (2/3 samples), and GS-II (2/3 samples).

Taken together with the results of Example 5, the present results indicate that lectins can enrich CD34+ cells from CB MNC by both negative and positive selection, for example:
1) GNA binds to about 70% of CB MNC but not to CD34+ cells, leading to about 3× enrichment in negative selection of CB MNC in CD34+ cell isolation.
2) STA binds to about 50% of CB MNC and also to CD34+ cells, leading to about 2× enrichment in positive selection of CB MNC in CD34+ cell isolation.
3) UEA binds to about 50% of CB MNC and also to CD34+ cells, leading to about 2× enrichment in positive selection of CB MNC in CD34+ cell isolation.

### Example 8

### Immunohistochemical Staining of Stem Cells

Immunohistochemical studies of embryonic stem cells (in culture) (GF series of stainings)

hESC were cultured as described in the Examples, fixed and after rinsing with PBS the stem cell cultures/sections were incubated in 3% highly purified BSA in PBS for 30 minutes at RT to block nonspecific binding sites. Primary antibodies (GF279, 288, 287, 284, 285, 283,286,290 and 289) were diluted (1:10) in PBS containing 1% BSA-PBS and incubated 1 hour at RT. After rinsing three times with PBS, the sections were incubated with biotinylated rabbit anti-mouse, secondary antibody (Zymed Laboratories, San Francisco, Calif., USA) in PBS for 30 minutes at RT, rinsed in PBS and incubated with peroxidase conjugated streptavidin (Zymed Laboratories) diluted in PBS. The sections were finally developed with AEC substrate (3-amino-9-ethyl carbazole; Lab Vision Corporation, Fremont, Calif., USA). After rinsing with water counterstaining was performed with Mayer's hemalum solution.

Antibodies, their antigens/epitopes and codes used in the immunostainings. See also Table 1 for results.

| Producer code | Manufact | Clone | Specificity | Code | Target stucture(s) | Host/isotype |
|---|---|---|---|---|---|---|
| MAB-S206 (Globo-H) | Glycotope | A69-A/E8 | Globo-H | GF288 | Fucα2Galβ3GalNAcβ3GalαLacCer | mouse/IgM |
| MAB-S201 CD174 | Glycotope | A70-C/C8 | CD174 | GF289 | Fucα2Galβ4(Fucα3)GlcNAc | mouse/IgM |
| (Lewis y) | | | (Lewis y) | | | |
| MAB-S204 H type 2 | Glycotope | A51-B/A6 | H type 2 | GF290 | Fucα2Galβ4GlcNAc | mouse/IgA |
| DM3122: 0.1 mg | Acris | 2-25LE | Lewis b | GF283 | Fucα2Galβ3(Fucα4)GlcNAc | mouse/IgG |
| (Lewis b) | | | | | | |
| DM3015: 0.15 mg | Acris | B393 | H Type 2 | GF284 | Fucα2Galβ4GlcNAc | mouse/IgM |
| DM3014: 0.15 mg | Acris | B389 | H Type 2, | GF285 | Fucα2Galβ4GlcNAc, | mouse/IgG1 |
| | | | Le b, Ley | | Fucα2Galβ3(Fucα4)GlcNAc, | |
| | | | | | Fucα2Galβ4(Fucα3)GlcNAc | |
| BM258P: 0.2 mg | Acris | BRIC 231 | H Type 2 | GF286 | Fucα2Galβ4GlcNAc | mouse/IgG1 |
| ab3355 (blood group | Abcam | 17-206 | H type 1 | GF287 | Fucα2Galβ3GlcNAc | mouse/IgG3 |
| antigen H1) | | | | | | |
| ab3352 (pLN) | Abcam | K21 | Lewis c | GF279 | Galβ3GlcNAcβ(3Lac) | mouse/IgM |
| | | | Gb3GN | | | |

Detection of Carbohydrate Structures on Cell Surface in Stem Cell Samples by Specific Antibodies

Materials and Methods

Cell samples. Mesenchymal stem cells (MSCs) from bone marrow were generated and cultured in proliferation medium as described above. MSCs were cultured in differentiation medium (proliferation medium including 4 ng/ml dexamethasone, 10 mmol/L β-glycerophosphate, and 50 μmol/L ascorbic acid) for 6 weeks to induce osteogenic differentiation. Differentiation medium was refreshed twice a week throughout the differentiation period.

Antibodies.

Immunostainings. Bone-marrow derived mesenchymal stem cells on passages 9-12 were grown on 0.01% poly-L-lysine (Sigma, USA) coated glass 8-chamber slides (Lab-TekII, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK) Immunostainings were observed with Zeiss Axioskop 2 plus—fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Fluorescence activated cell sorting (FACS) analysis. Proliferating MSCs on passage 12 were detached from culture plates by 0.02% Versene solution (pH 7.4) for 45 minutes at 37° C. Cells were washed twice with 0.3% HSA-PBS solution before antibody labelling. Primary antibodies were incubated (4 μl/100 μl cell suspension/50 000 cells) for 30 minutes at RT and washed once with 0.3% HSA-PBS before secondary antibody detection with Alexa Fluor 488 goat anti-mouse (1:500) for 30 minutes at RT in the dark. As a negative control cells were incubated without primary antibody and otherwise treated similar to labelled cells. Cells were analysed with BD FACSAria (Becton Dickinson) using FITC detector at wavelength 488. Results were analysed with BD FACSDiva software version 5.0.1 (Becton Dickinson).

Antibodies, their antigens/epitopes and codes used in the immunostainings. See also Table 1 for results.

| | | | Dilution | | | |
|---|---|---|---|---|---|---|
| Code | Antigen | Host | I HC | Class | Manufact | Cat No |
| GF274 | PNAd (peripheral lymph node addressin; | Rat anti- | 5-20 μg/ml | IgM, κ | BD | 553863 |
| | CD62L ligand) closely associated with L- | mouse | | | Pharmingen | |
| | selectin (CD34, GlyCAM-1, MAdCAM-1), | | | | | |
| | sulfo-mucin | | | | | |
| GF275 | CA15-3 (Cancer antigen 15-3; sialylated | Mouse anti- | | IgG1 | Acris | BM3359 |
| | carbohydrate epitope of the MUC-1 | human | | | Antibodies | |
| | glycoprotein) | | | | | |
| GF276 | oncofetal antigen, tumor associated | Mouse anti- | 1:20-1:50 | IgG1 | Acris | DM288 |
| | glycoprotein (TAG-72) or CA 72-4 | human | | | Antibodies | |
| GF277 | human sialosyl-Tn antigen (STn, | Mouse anti- | 1:50-1:100 | IgG1 | Acris | DM3197 |
| | sCD175) | human | (4-8 μg/ml) | | Antibodies | |
| GF278 | human Tn antigen (Tn, CD175 B1.1) | Mouse anti- | 1:50 (4 μg/ml) | IgM | Acris | DM3218 |
| | | human | | | Antibodies | |

| | | | Dilution | | | |
|---|---|---|---|---|---|---|
| Koodi | Antigen | Host | I HC | Class | Manufact | Cat No |
| GF295 | Blood group antigen precursor (BG1), | Mouse anti- | 01:40 | IgM | Abcam | ab3352 |
| | Lewis c Gb3GN (pLN) | human | | | | |
| GF280 | TF-antigen isoform (Nemod TF2) | Mouse anti-? | | IgM | | MAB- |
| | | | | | | S301 |
| GF281 | TF-antigen isoform (A68-E/E3) | Mouse anti-? | | IgG1 | | MAB- |
| | | | | | | S305 |
| GF296 | asialoganglioside GM1 | Rabbit anti- | 1:100-1:400 | polycl. | Acris | BP282 |
| | | bovine | ELISA | | Antibodies | |
| GF297 | Globoside GL4 | Rabbit anti- | 1:50-1:100 | polycl. | Abcam | ab23949 |
| | | several | ELISA | IgG | | |
| | | species | | | | |
| GF298 | Human CD77 (=blood group substance | Rat anti- | | IgM | Acris | SM1160P |
| | pk), GB3 | human | | | Antibodies | |
| GF299 | Forssman antigen, glycosphingolipid (FOGSL) | Rat anti- | 1:100-1:1000 | IgG | Acris | BM4091 |
| | differentiation ag | mouse | | | Antibodies | |
| | | (human ??) | | | | |
| GF300 | Asialo GM2 | Rabbit anti- | 1:100-1:400 | polycl. | Acris | BP283 |
| | | bovine | ELISA | | Antibodies | |

| | | | Dilution | | | |
|---|---|---|---|---|---|---|
| Code | Antigen | Host | I HC | Class | Producer | Cat no |
| GF301 | Lewis b blood group antigen | Mouse anti- | | IgG1 | Acris | SM3092P |
| | | human | | | Antibodies | |
| GF302 | H type 2 blood group antigen | Mouse anti- | | IgM | Acris | DM3015 |
| | | human | | | Antibodies | |
| GF303 | Blood group H1(O) antigen (BG4) | Mouse anti- | | IgG3 | Abcam | ab3355 |
| | | human | | | | |
| GF288 | Globo-H | Mouse anti-? | | IgM | | MAB- |
| | | | | | | S206 |
| GF304 | Lewis a | Mouse anti- | | IgG1 | Chemicon int. | CBL205 |
| | | human | | | | |
| GF305 | Lewis x, CD15, 3-FAL, SSEA-1,3- | Mouse anti- | | IgM | Chemicon int. | CBL144 |
| | fucosyl-N-acetyllactosamine | human | | | | |
| GF306 | Sialyl Lewis a | Mouse anti- | 01:40 | IgG1 | Chemicon int. | MAB2095 |
| | | human | | | | |
| GF307 | Sialyl Lewis x | Mouse anti- | 01:40 | IgM | Chemicon int. | MAB2096 |
| | | human | | | | |
| GF353 | SSEA-3 (stage-specific embryonic | Rat anti- | 10-20 μg/ml | IgM | Chemicon int. | MAB4303 |
| | antigen-3) | mouse/human | | | | |
| GF354 | SSEA-4 (stage-specific embryonic | Mouse anti- | 10-20 μg/ml | IgG3 | Chemicon int. | MAB4304 |
| | antigen-4) | human | | | | |
| GF355 | Galactose-a(1,3)galactose | Baboon | 1:500 | serum | Chemicon int. | AB2052 |
| | | anti- | | | | |
| | | porcine/rat | | | | |
| GF365 | Nemod TF1, DC176, GalB1-3GalNAc | Mouse anti- | | IgM, k | Glycotope | Lot 31-2006 |
| | | human | | | | |

### Example 9

Isolation of Subset Expressing Glycan Structures of Formula (I) on Human Embryonic Stem Cells

Cell Culture and Passaging

FES hESC lines with normal karyotypes are obtained and grown as described in Mikkola et al. (2006; Distinct differentiation characteristics of individual human embryonic stem cell lines, BMC Dev Biol. 2006; 6: 40).

Human ESCs are maintained on mitotically inactivated primary mouse embryonic fibroblasts (MEF) feeder layers for routine maintenance. Cells are grown in tissue culture treated dishes (Corning Incorporated). Cells are passaged every 6 days using either a pretreatment with 10 mg/ml collagenase 5 minutes or manual dissection with a fire pulled Pasteur pipette.

Immunocytochemistry is performed on routinely maintained adherent hESC colonies, and flow cytometry is performed using routinely maintained hESC colonies that are stained for antibodies, lectins or glycosidases of the present invention.

Enrichment of Glycan Structure of Formula (I) Expressing Stem Cells

The FACS analysis is performed essentially as described in Venable et al. (2005) but living cells are used instead and FACSAria™ cell sorter (BD).

Human ESCs are harvested into single cell suspensions using collagenase and cell dissociation solution (Sigma). Then, cells are placed in sterile tube in aliquots 10⁶ cells each and stained with one of the GF antibody in 1:100 solution. Cells are washed 3 times with PBS and then stained with secondary antibodies (antigoat mouse IgG or IgM FITC conjugated). Unstained FES used as control. The FITC positive cells are collected into cell culture media (in +4° C.) (according to BD instructions).

Then, cells are placed on MEF or HHF feeder layers and monitored for clonal or cell lineage. To check the undifferentiation stage, the gene expression of sorted cells are analyzed with real-time PCR.

Alternatively, FACS enriched cells are let to spontaneously differentiate on gelatin. Immunohistochemistry is performed with various tissue specific antibodies as described in Mikkola et al. (2006) or analysed with PCR.

### Example 10

Revealing Protease Sensitive and Insensitive Antibody Target Structures

Bone marrow mesenchymal stem cells as described in examples above were analyzed by FACS analysis. Several antigen structures are essentially not observed or these are observed in reduced amount in FACS analysis of cell surface antigens when cells are treated (released from cultivation) by trypsin but observable after Versene treatment (0.02% EDTA in PBS). This was observed for example by labelling of the mesenchymal stem cells by the antibody GF354, and GF275, with major part trypsin sensitive target structures and by the antibody GF302, which target structure is practically totally trypsin sensitive.

### Example 11

Isolation and characterization of protease released glycopeptides comprising specific binder target structures.

Glycopeptides are released by treatment of stem cells by protease such as trypsin. The glycopeptides are isolated chromatographically, a preferred method uses gel filtration chromatography in Superdex (Amersham Pharmacia(GE)) column (Superdex peptide or superdex 75), the peptides can be observed in chromatogram by tagging the peptides with specific labels or by UV absorbance of the peptide (or glycans). Preferred samples for the method includes mesenchymal stem cells in relatively large amounts (millions of cells) and preferred antibodies, which are used in this example includes antibodies GF354, GF275 or GF 302 or antibodies or other binders such as lectins with similar specificty.

The isolated glycopeptides are then run through a column of immobilized antibody (e.g. antibody immobilized to cyanogens promide activated column of Amersham Pharmacia(GE healthcare division or antibody immobilized as described by Pierce catalog)). The bound and/or weakly bound and chromatographically retarded fraction(s) is(are) collected as target peptide fraction. In case of high affinity binding the glycan is eluted with 100-1000 mM monosaccharide or monosaccharides corresponding to the target epitope of the antibody or by mixture of monosaccharides or oligosaccharides and/or with high salt concentration such as 500-1000 mM NaCl. The glycopeptides are analysed by glycoproteomic methods using mass spectrometry to obtain molecular mass and preferably also fragmentation mass spectrometry in order to sequence the peptide and/or the glycan of the glycopeptide.

In alternative method the glycopeptides are isolated by single affinity chromatography step by the binder affinity chromatography and analysed by mass spectrometry essentially similarily as described e.g. in Wang Y et al (2006) Glycobiology 16 (6) 514-23, but lectin affinity chromatography is replaced by affinity chromatography by immobilized antibodies, such as preferred antibodies or binder described above in this example.

### Example 12

### The Glycome of Human Embryonic Stem Cells Reflects their Differentiation Stage

### SUMMARY

Complex carbohydrate structures, glycans, are elementary components of glycoproteins, glycolipids, and proteoglycans. These glycoconjugates form a layer of glycans that covers all human cell surfaces and forms the first line of contact towards the cell's environment. Glycan structures called stage specific embryonic antigens (SSEA) are used to assess the undifferentiated stage of embryonic stem cells. However, the whole spectrum of stem cell glycan structures has remained unknown, largely due to lack of suitable analysis technology. We describe the first global study of glycoprotein glycans of human embryonic stem cells, embryoid bodies, and further differentiated cells by MALDI-TOF mass spectrometric profiling. The analysis reveals how certain asparagine-linked glycan structures characteristic to stem cells are lost during differentiation while new structures emerge in the differentiated cells. The results indicate that human embryonic stem cells have a unique glycome and that their differentiation stage can be identified by glycome analysis. We suggest that knowledge about stem cell specific glycan structures can be used for e.g. purification, manipulation, and quality control of stem cells.

Materials & Methods

Human embryonic stem cell lines. Five Finnish hESC lines, FES 21, FES 22, FES 29, FES 30 (Skottman et al., 2005. Stem cells 23:1343-56) and FES 61 were used in the present study. These lines are included in the International Stem Cell Initiative (Andrews et al., 2005. Nat. Biotechnol. 23:795-7). The cells were propagated on human foreskin fibroblast (hFF) feeder cells in serum-free medium (Knockout™, Gibco/Invitrogen). In FACS analyses 70-90% of cells from mechanically isolated colonies were typically Tra 1-60 and Tra 1-81 positive (not shown). Cells differentiated into embryoid bodies (EB, stage 2 differentiated) and further differentiated cells grown out of the EB as monolayers (stage 3 differentiated) were used for comparison against hESC. The differentiation protocol favors the development of neuroepithelial cells while not directing the differentiation into distinct terminally differentiated cell types (Okabe et al., 1996. Mech. Dev. 59:89-102). EB derived from FES 30 had less differentiated cell types than the other three EB. Stage 3 cultures consisted of a heterogenous population of cells dominated by fibroblastoid and neuronal morphologies. For the glycome studies the cells were collected mechanically, washed, and stored frozen until analysis.

In a preferred embodiment the invention is directed to the use of data obtained embryoid bodies or ESC-cell line cultivated under conditions favouring neuroepithelial cells for search of specific structures indicating neuroepithelial development, preferably by comparing the material with cell materials comprising neuronal and/or epithelial type cells.

Asparagine-linked glycome profiling. Total asparagine-linked glycan (N-glycan) pool was enzymatically isolated from about 100 000 cells. The total N-glycan pool (picomole quantities) was purified with microscale solid-phase extraction and divided into neutral and sialylated N-glycan fractions. The N-glycan fractions were analyzed by MALDI-TOF mass spectrometry either in positive ion mode for neutral N-glycans or in negative ion mode for sialylated glycans (Saarinen et al., 1999, Eur. J. Biochem. 259, 829-840). Over one hundred N-glycan signals were detected from each cell type revealing the surprising complexity of hESC glycosylation. The relative abundances of the observed glycan signals were determined based on relative signal intensities (Harvey, 1993. Rapid Commun. Mass Spectrom. 7:614-9; Papac et al., 1996. Anal. Chem. 68:3215-23).

Results

In the present study, we analyzed the N-glycome profiles of hESC, EB, and st.3 differentiated cells (FIG. 2).

The similarity of the N-glycan profiles within the group of four hESC lines suggested that the obtained N-glycan profiles are a description of the characteristic N-glycome of hESC. Overall, 10% of the 100 most abundant N-glycan signals present in hESC disappeared in st.3 differentiated cells, and 16% of the most abundant signals in st.3 differentiated cells were not present in hESC. This indicates that differentiation induced the appearance of new N-glycan types while earlier glycan types disappeared. In quantitative terms, the differences between the glycan profiles of hESC, EB, and st.3 differentiated cells were: hESC vs. EB 19%, hESC vs. st.3 24%, and EB vs. st.3 12%.

The glycome profile data was used to design glycan-specific labeling reagents for hESC. The most interesting glycan types were chosen to study their expression profiles by lectin histochemistry as exemplified in FIG. 3 for the lectins that recognize either α2,3-sialylated (MAA-lectin, FIG. 3A.) binding to the hESC cells or α-mannosylated glycans (PSA-lectin, FIG. 3B.) binding to the surfaces of feeder cells (MEF). The binding of the lectin reagents was inhibited by specific carbohydrate inhibitors, sialylα2-lactose and mannose, respectively (FIGS. 3C. and 3D.). The results are summarized in Table 5.

Table 5 further represent differential recognition feeder and stem cells by two other lectins, Ricinus communis agglutinin (RCA, ricin lectin), known to recognize especially terminal Galβ-structures, especially Galβ4Glc(NAc)-type structures and peanut agglutinin (PNA) recognizing Gal/GalNAc structures. The cell surface expression of ligand for two other lectin RCA and PNA on hESC cells, but only RCA ligands of feeder cells.

The present results indicate and the invention is directed to the hESC glycans are potential targets for recognition by stem cell specific reagents. The invention is further directed to methods of specific recognition and/or separation of hESC and differentiated cells such as feeder cells by glycan structure specific reagents such as lectins. Human embryonic stem cells have a unique glycome that reflects their differentiation stage. The invention is specifically directed to analysis of cells according to the invention with regard to differentiation stage.

The results were also used to generate an algorithm for identification of hESC differentiation stage (FIG. 22). To test whether the obtained N-glycan profiles could be used for reliable identification of hESC and differentiated cells even with the presence of sample-to-sample variation, a discrimination analysis was performed on the data. The hESC line FES 29 and embryoid bodies derived from it (EB 29) were selected as the training group for the calculation that effectively discriminated the two samples (FIG. 22):

glycan score=a—b—c,

wherein a is the sum of the relative abundances (%) of all signals with proposed compositions with two or more dHex (F≧2) in the sialylated N-glycan fraction, b is the sum of the relative abundances (%) of all signals with hybrid-type structures (ST=H), and c is the sum of the relative abundances (%) of all signals with proposed compositions with five or more HexNAc and equal amounts of Hex and HexNAc (H=N≧5); see Table 13 for structure codes and FIG. 2 for the dataset.

The resulting equation was applied to the other samples that served as the test group in the analysis and the results are described graphically in FIG. 22. hESC and the differentiated cell samples were clearly discriminated from each other (p<0.01, Student's t test). Furthermore, the st.3 differentiated cell samples were separated from the EB samples (p<0.05, Mann-Whitney test). The predicted 95% confidence intervals (assuming normal distribution of glycan scores within each cell type) are shown for the three cell types, indicating that a calculated glycan score has potential to discriminate all three cell types. At 96% confidence interval, hESC and the differentiated cell types (EB and st.3) were still discriminated from each other (not shown in the figure). The results indicate that glycome profiling is a tool for monitoring the differentiation status of stem cells.

### CONCLUSIONS

The present data represent the glycome profiling of hESC:
hESC have a unique N-glycome comprising of over 100 glycan components
Differentiation induces a major change in the N-glycome and the cell surface molecular landscape of hESC

Utility of hESC glycome data:
Identification of new stem cell markers for e.g. antibody development
Quality control of stem cell products
Identification of hESC differentiation stage
Control of variation between hESC lines
Effect of external factors and culture conditions on hESC status

Especially preferred uses of the data are Use of the hESC glycome for identification of specific cell surface markers characteristic for the pluripotent hESCs.

The invention is directed to further analysis and production of present and analogous glycome data and use of the methods for further indentification of novel stem cell specific glycosylation features and form the basis for studies of hESC glycobiology and its eventual applications according to the invention

### Example 13

FACS and immunohistochemical analysis of embryonic stem cells

Immunohistochemical staining of stem cells. Immunohistochemical studies of embryonic stem cells (in culture)(GF series of stainings). hESC were cultured as described in the Examples, fixed and after rinsing with PBS the stem cell cultures/sections were incubated in 3% highly purified BSA in PBS for 30 minutes at RT to block nonspecific binding sites. Primary antibodies (GF279, 288, 287, 284, 285, 283,286,290 and 289) were diluted (1:10) in PBS containing 1% BSA-PBS and incubated 1 hour at RT. Other antibodies indicated in the Tables were used similarily. After rinsing three times with PBS, the sections were incubated with biotinylated rabbit anti-mouse, secondary antibody (Zymed Laboratories, San Francisco, Calif., USA) in PBS for 30 minutes at RT, rinsed in PBS and incubated with peroxidase conjugated streptavidin (Zymed Laboratories) diluted in PBS. The sections were finally developed with AEC substrate (3-amino-9-ethyl carbazole; Lab Vision Corporation, Fremont, Calif., USA). After rinsing with water counterstaining was performed with Mayer's hemalum solution.

Antibodies, their antigens/epitopes and codes used in the immunostainings. Table 11 shows antibody binding to purified glycosphingolipid fractions from small amounts of cells (corresponding to hundreds of thousands of cells). The binding was analysed by TLC overlay assay using radiolabelled antibodies. The positive signals indicate presence of substantial amounts of the glycolipids and minus no signal due to too low amount for analysis.

Flow cytometry. Flow cytometric analysis of lectin binding was used to study the cell surface carbohydrate expression of hESC. The cells were washed with PBS. The cells were harvested into single cell suspensions by 0.02% Versene solution (pH 7.4). Detached cells were centrifuged at 1100 g for five minutes at room temperature. Cell pellet was washed twice with 1% HSA-PBS, centrifuged at 1100 g and resuspended in 1% HSA-PBS. Cells were placed in conical tubes in aliquots of approximately 100000 cells each. Cell aliquots were incubated with one of the FITC labelled lectin for 30 minutes +4 C. After incubation cells were washed with 1% HSA-PBS, centrifuged and resuspended in 1% HSA-PBS. Untreated cells were used as controls. Lectin binding was detected by flow cytometry (FACSCalibur, Becton Dickinson).

In antibody analysis primary antibodies were incubated with suitable dilution based on recommendation of the producer for 30 minutes at +4 C and washed once with 0.3% HSA-PBS before secondary antibody detection with FITC secondary antibody for 30 minutes at +4 C in the dark. As a negative control cells were incubated without primary antibody and otherwise treated similar to labelled cells. Cells were analysed with BD FACS Calibur (Becton Dickinson). Results were analysed with Cell Quest Pro software (Becton Dickinson).

Fluorecently labeled lectins were from EY Laboratories (USA) or Vector Laboratories (UK). Antibody origin and codes are indicated in Table 12.

Results from FACS Analysis

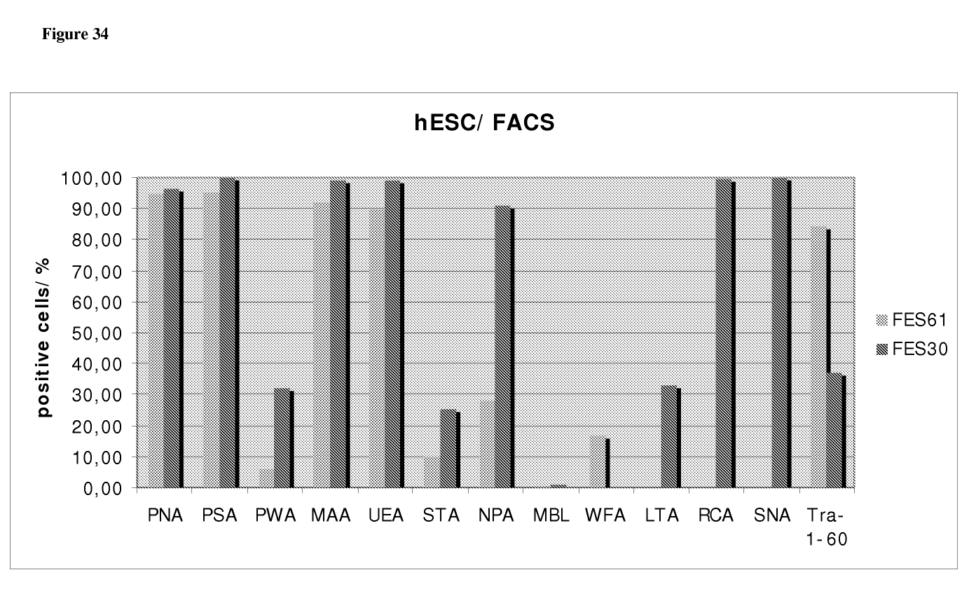

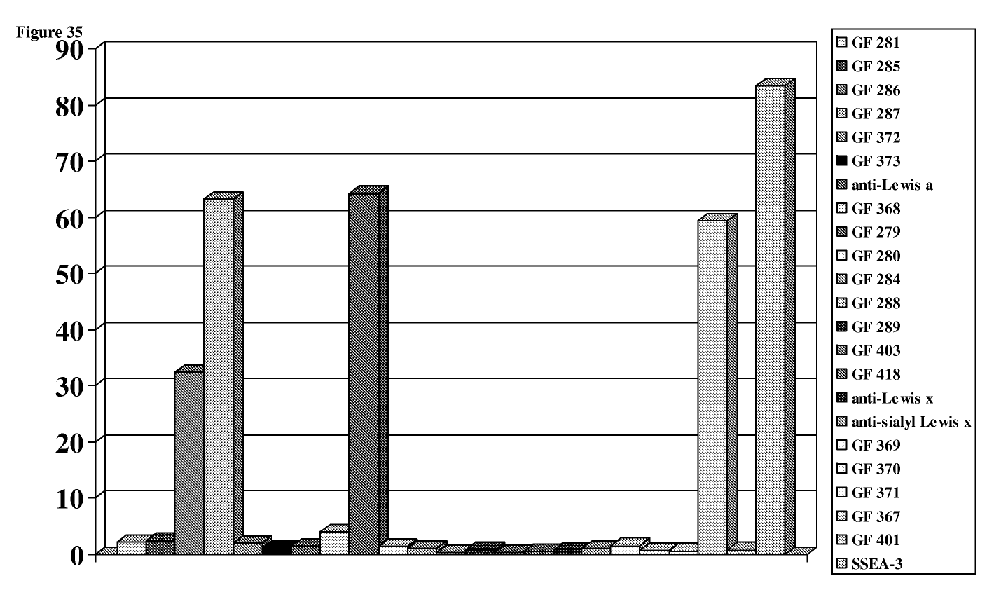

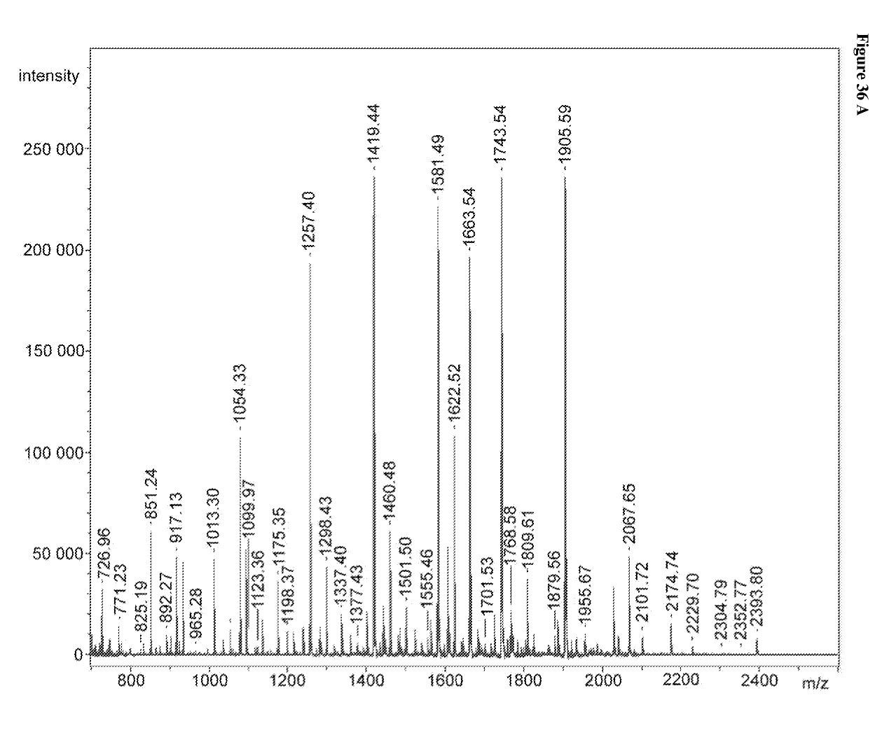

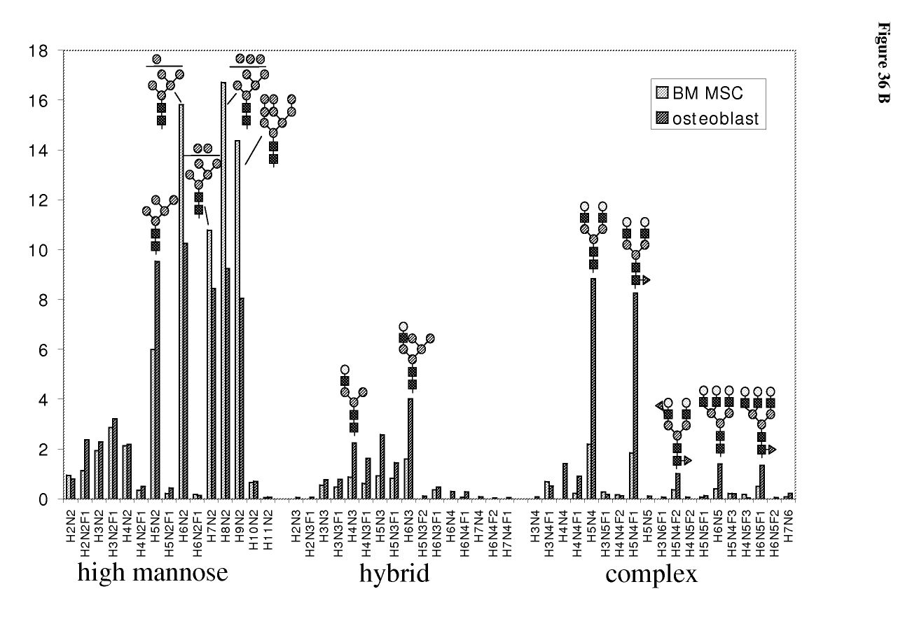

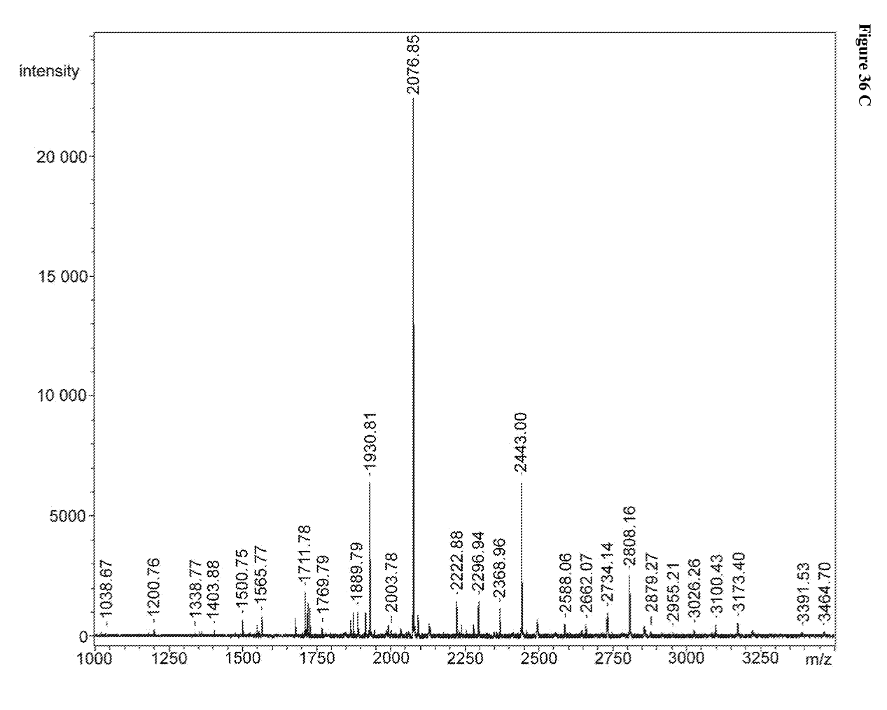

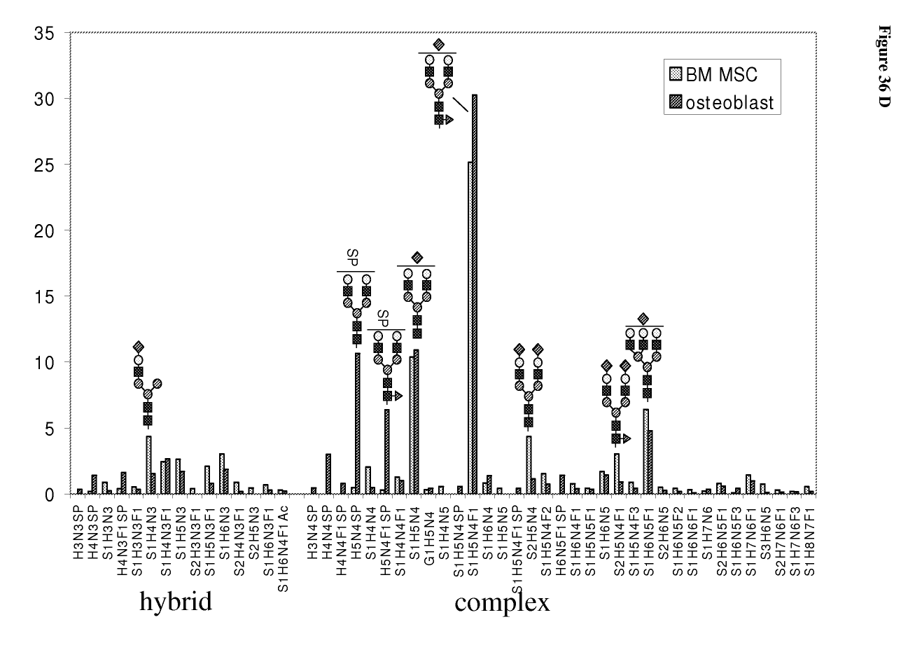

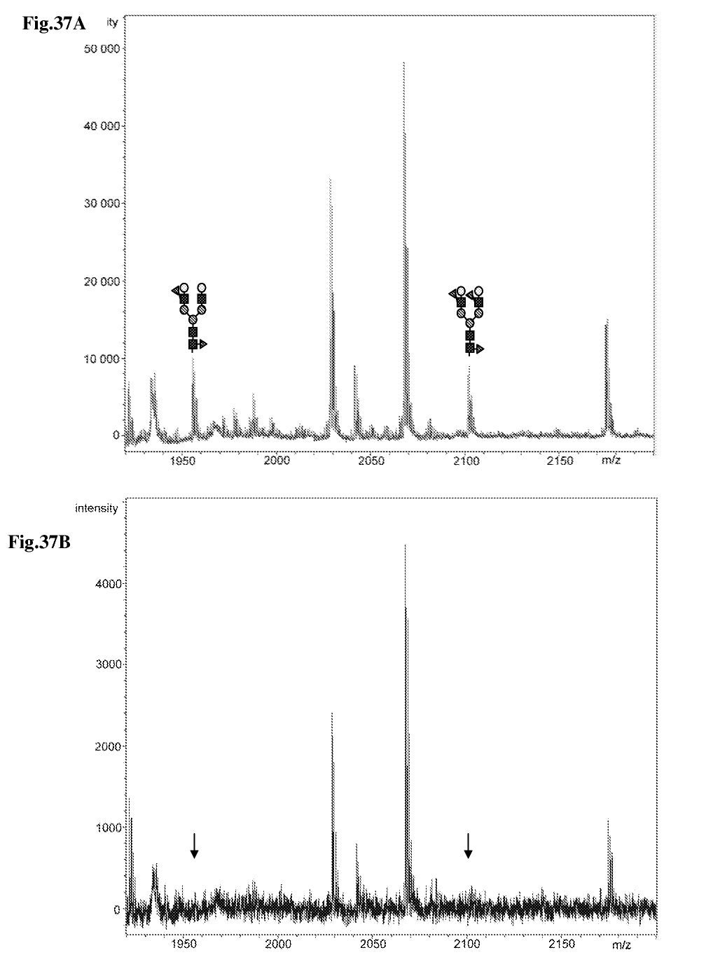

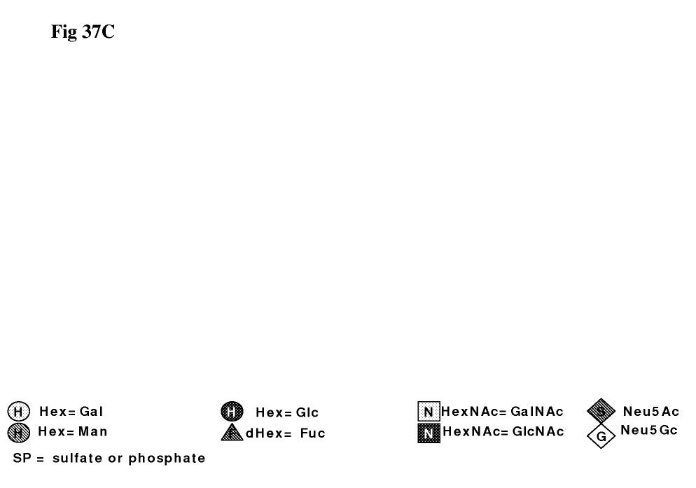

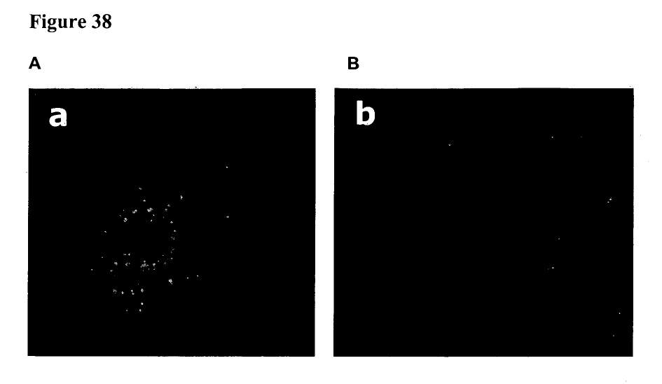

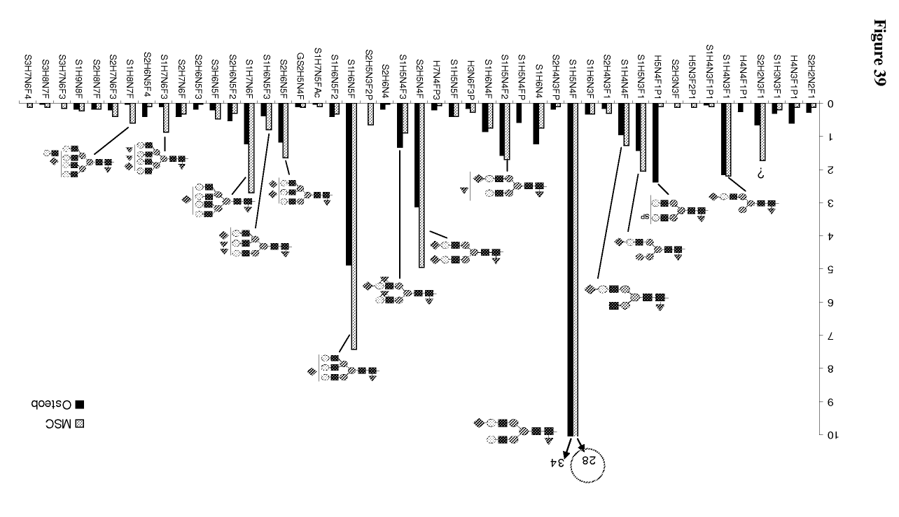

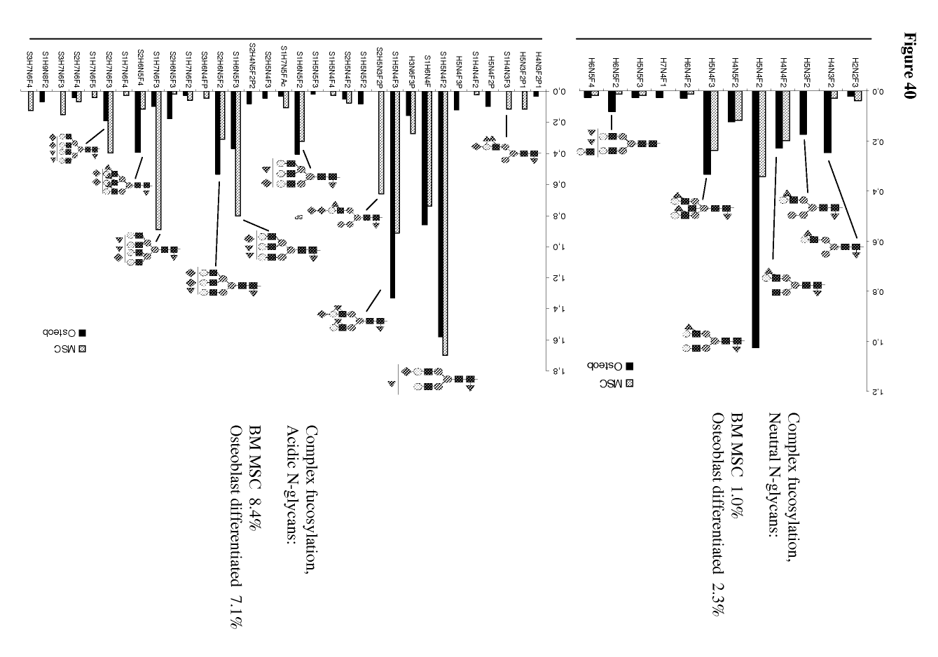

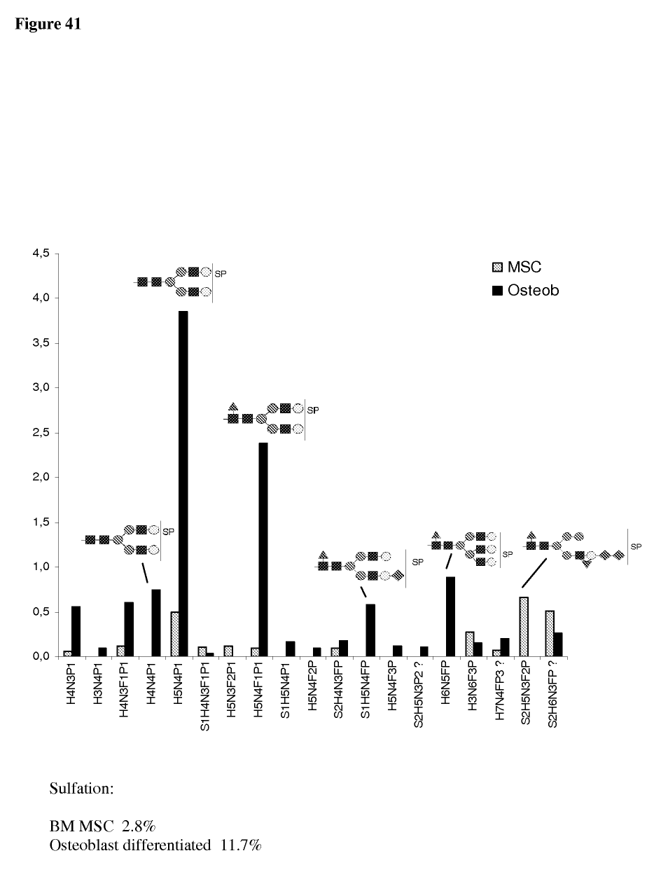

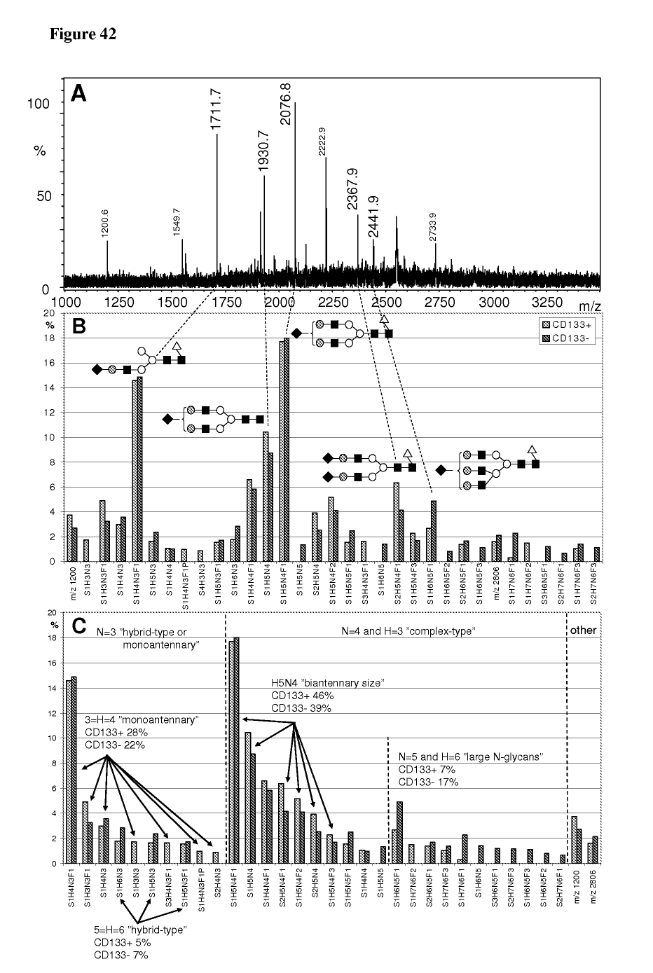

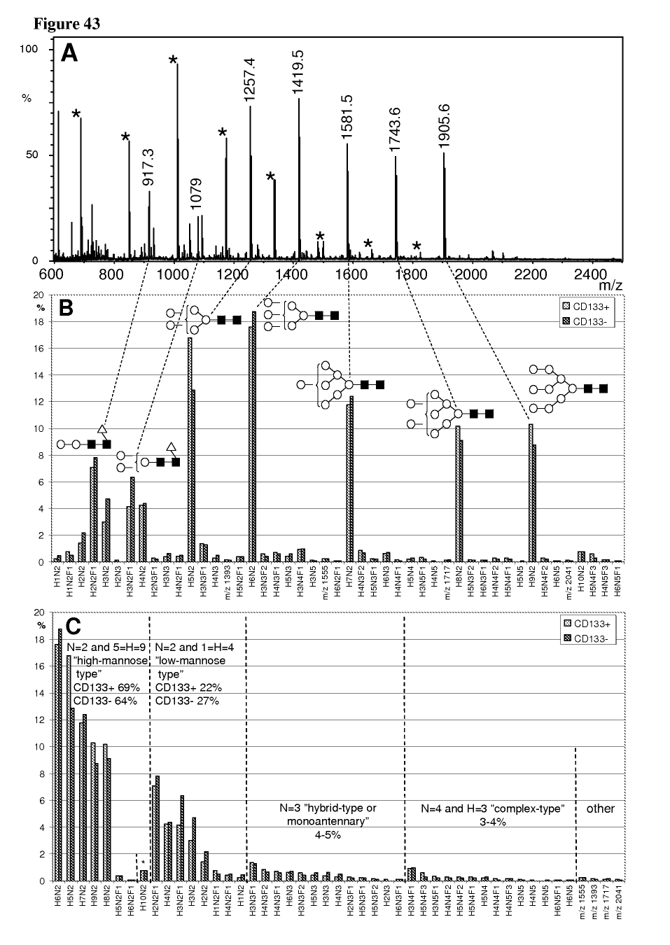

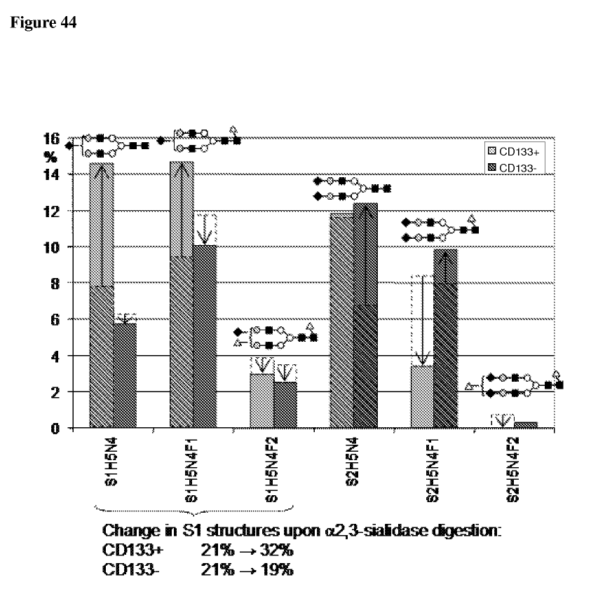

The lectin labelling results are present in Table 14 and FIGS. 34 and 3 from separate experiment for comparision. The symbol + indicates labelling majority of cell, +/− indicates labelling of substantial subpopulation and (+/−) indicates weak labelling or labelling of minor cell population/few individual cells.

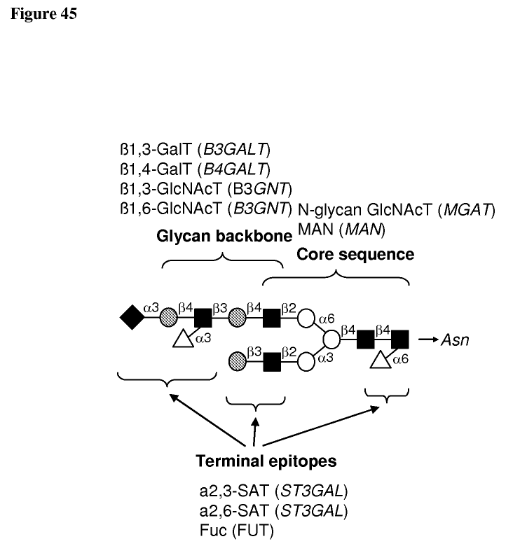

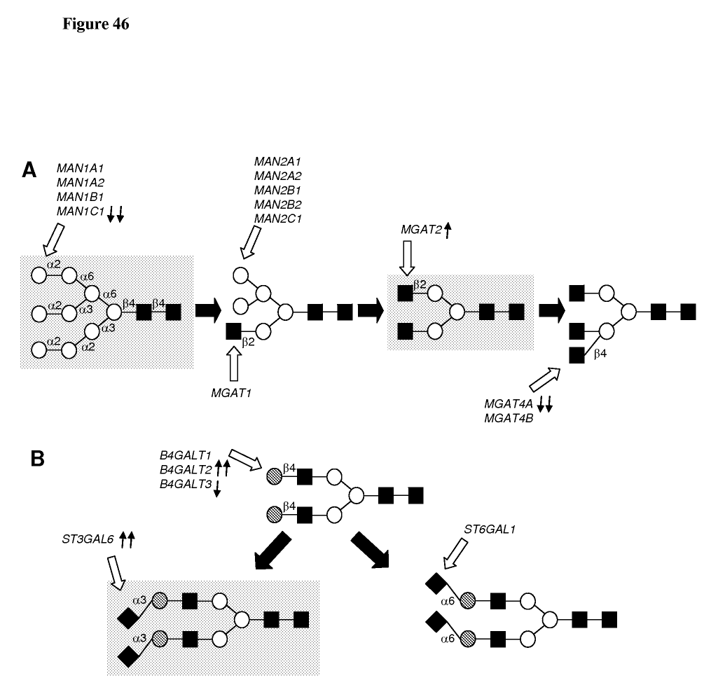

The antibody labelling results are present in Tables 15-17 and FIG. 35 with comparison to immunohistochemistry (immuno) results. The negativity − indicates negative or low labelling of less than 10% of cells when labelling with the specific antibody clone (defined in Table 12). The four most effective binders (for antigens H type II, H type I, type I LacNAc (Lewis c) and globotriose) were indicated with + in FACS Tables 15-17. These antibodies are especially preferred for recognition of the glycans under FACS conditions.

It is further realized that part of the structures indicated to be present can be recognized with other antibodies specific for the correct elongated glycan epitopes (e.g. Lewis x structures). The binding of LTA lectin verified the structural analysis of Lewis x on the specific N-glycan structures and the invention is specifically directed to known regents for the recognition of the N-glycan linked Lex according of the invention. The schistosoma directed LacdiNAc specific antibodies form Leiden university appear not to be very effective in the recognition of the preferred N-glycan linked LacdiNAcs.

The comparision of the immunohistochemistry and FACS results indicates that the due to technical reasons FACS may be as effective for recognition of glycans observable by immunohistochemistry. The immunohistochemistry further reveals structures present in a few cells observable as very weak signals in FACS.

### Example 14

### Immunohistochemical Stainings of Mesenchymal Stem Cells and Osteogenic Cells Differentiated from them

Experimental Procedures

Cell samples. Mesenchymal stem cells (MSCs) from bone marrow were generated and cultured in proliferation medium as described above. MSCs were cultured in differentiation medium (proliferation medium including 0.1 μmol/L dexamethasone, 10 mmol/L β-glycerophosphate, and 50 μmol/L ascorbic acid) for 6 weeks to induce osteogenic differentiation. Differentiation medium was refreshed twice a week throughout the differentiation period.

Antibodies. Antibodies, their antigens/epitopes and codes used in the immunostainings.

| Code | Antigen | Host/Isotype | Manufacturer | Cat no |
|---|---|---|---|---|
| GF274 | PNAd (peripheral lymph node addressin; CD62L ligand) | Rat/IgM, κ | BD | 553863 |
| | closely associated with L-selectin (CD34, GlyCAM-1, | | Pharmingen | |
| | MAdCAM-1), sulfo-mucin | | | |
| GF275 | CA15-3 (Cancer antigen 15-3; sialylated carbohydrate epitope | Mouse/IgG1 | Acris | BM3359 |
| | of the MUC-1 glycoprotein) | | Antibodies | |
| GF276 | oncofetal antigen, tumor associated glycoprotein (TAG-72) or | Mouse/IgG1 | Acris | DM288 |
| | CA 72-4 | | Antibodies | |
| GF277 | human sialosyl-Tn antigen (STn, sCD175) | Mouse/IgG1 | Acris | DM3197 |
| | | | Antibodies | |
| GF278 | human Tn antigen (Tn, CD175 B1.1) | Mouse/IgM | Acris | DM3218 |
| | | | Antibodies | |
| GF295 = | Blood group antigen precursor (BG1), Lewis c Gb3GN (pLN) | Mouse/IgM | Abcam | ab3352 |
| GF279 | | | | |
| GF280 | TF-antigen isoform (Nemod TF2) | Mouse/IgM | | MAB-S301 |
| GF281 | TF-antigen isoform (A68-E/E3) | Mouse/IgG1 | | MAB-S305 |
| GF296 | asialoganglioside GM1 | Rabbit/polycl. | Acris | BP282 |
| | | | Antibodies | |
| GF297 | Globoside GL4 | Rabbit/polycl. | Abcam | ab23949 |
| | | IgG | | |
| GF298 | Human CD77 (= blood group substance pk), GB3 | Rat/IgM | Acris | SM1160P |
| | | | Antibodies | |
| GF299 | Forssman antigen, glycosphingolipid (FO GSL) differentiation | Rat/IgG | Acris | BM4091 |
| | ag | | Antibodies | |
| GF300 | Asialo GM2 | Rabbit/polycl. | Acris | BP283 |
| | | | Antibodies | |
| GF301 | Lewis b blood group antigen | Mouse/IgG1 | Acris | SM3092P |
| | | | Antibodies | |
| GF302 = | H type 2 blood group antigen | Mouse/IgM | Acris | DM3015 |
| GF284 | | | Antibodies | |
| GF303 | Blood group H1(O) antigen (BG4) | Mouse/IgG3 | Abcam | ab3355 |
| GF287 | | | | |
| GF288 | Globo-H | Mouse/IgM | | MAB-S206 |
| GF304 | Lewis a | Mouse/IgG1 | Chemicon int. | CBL205 |
| GF305 | Lewis x, CD15, 3-FAL, SSEA-1,3-fucosyl-N- | Mouse/IgM | Chemicon int. | CBL144 |
| | acetyllactosamine | | | |
| GF306 | Sialyl Lewis a | Mouse/IgG1 | Chemicon int. | MAB2095 |
| GF307 | Sialyl Lewis x | Mouse/IgM | Chemicon int. | MAB2096 |
| GF353 | SSEA-3 (stage-specific embryonic antigen-3) | Rat/IgM | Chemicon int. | MAB4303 |
| GF354 | SSEA-4 (stage-specific embryonic antigen-4) | Mouse/IgG3 | Chemicon int. | MAB4304 |
| GF365 | Nemod TF1, DC176, GalB1-3GalNAc | Mouse/IgM, k | Glycotope | Lot 31-2006 |
| GF374 | Glycodelin A, GdA, PP14 (A87-D/F4) | Mouse/IgG1, k | Glycotope | Lot 2P-2006 |
| GF375 | Glycodelin A, GdA, PP14 (A87-D/C5) | Mouse/IgG1, | Glycotope | Lot 22-2006 |
| | | IgG2b, IgM, k | | |
| GF376 | Glycodelin A, GdA, PP14 (A87-B/D2) | Mouse/IgG1, k | Glycotope | Lot 25A- |
| | | | | 2006 |
| GF393 | Lewis y | Mouse/IgM | Glycotope | MAB-S201 |
| GF394 | H disaccharide | Mouse/IgA | Glycotope | MAB-S204 |

Immunohistochemistry (IHC). Bone-marrow derived mesenchymal stem cells on passages 9-12 were grown on CC2 treated glass 8-chamber slides (Lab-TekII, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) were diluted in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK) Immunostainings were observed with Zeiss Axioskop2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Fluorescence activated cell sorting (FACS) analysis. Proliferating MSCs on passage 12 were detached from culture plates by 0.02% Versene solution (pH 7.4) for 45 minutes at 37° C. Cells were washed twice with 0.3% HSA-PBS solution before antibody labelling. Primary antibodies were incubated (4 μl /100 μl al cell suspension/50 000 cells) for 30 minutes at RT and washed once with 0.3% HSA-PBS before secondary antibody detection with Alexa Fluor 488 goat anti-mouse (1:500) for 30 minutes at RT in the dark. As a negative control cells were incubated without primary antibody and otherwise treated similar to labelled cells. Cells were analysed with BD FACSAria (Becton Dickinson) using FITC detector at wavelength 488. Results were analysed with BD FACSDiva software version 5.0.1 (Becton Dickinson).

Results and Discussion

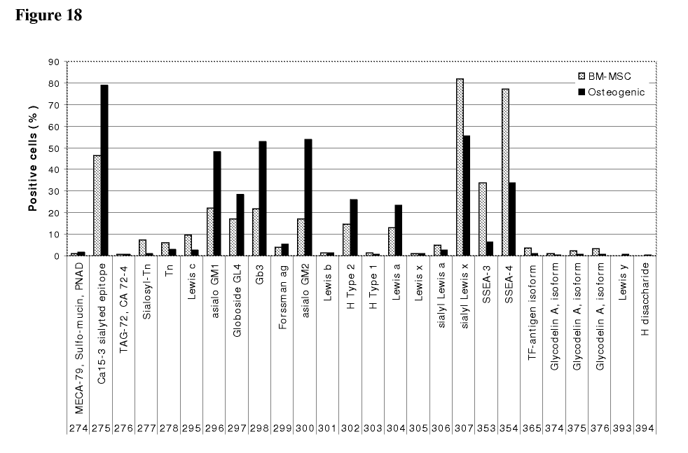

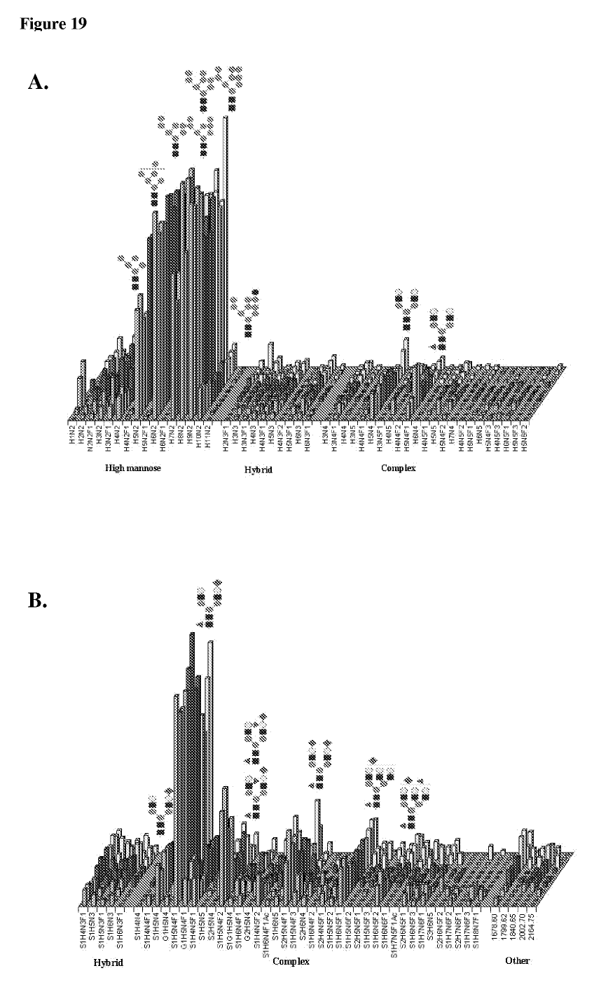

Based on both FACS and IHC results, antibodies GF307 (sLex), GF353 (SSEA-3) and GF354 (SSEA-4) are markers for mesenchymal stem cells, since their expression on the cell surface clearly decreases during osteogenic differentiation (Table 18, FIG. 18). Additionally, in FACS analysis antibodies GF277 (sTn), GF278 (Tn), GF295 (pLN) and GF306 (sLea) show more reactivity with MSCs than with osteogenic cells, indicating that these markers would also be associated with mesenchymal stem cells.

When BM-MSCs were differentiated for osteogenic direction for 6 weeks, their cell surface expressed more of the following glycans: GF275 (CA15-3), GF296 (asialo GM1), GF297 (GL4), GF298 (Gb3), GF300 (asialo GM2), GF302 (H type 2), and GF304 (Lea) based on FACS analysis (Table 18, FIG. 18). On the other hand, IHC results showed that staining of GF276 (oncofetal antigen), GF277 (sTn), GF278 (Tn), and GF303 (H Type 1) clearly increased during osteogenic differentiation (Table 18). Interestingly, antibodies GF276 (oncofetal antigen) and GF303 (H Type 1) showed no reactivity when used in FACS, but instead showed clear staining in IHC only in osteogenic cells, being therefore markers for osteogenic differentiation. Additionally, antibodies GF296 (asialo GM1), GF300 (asialo GM2) and GF304 (Lea) were totally negative in IHC, but showed reactivity in FACS analysis, being markers for osteogenic lineage.

The discrepancy between FACS and IHC with some antibodies may result from several reasons. First, cells undergo different treatments before incubation with antibodies, e.g. cells are fixed for IHC, but not for FACS, and cells are adherent in IHC and in suspension for FACS analysis. Furthermore, glycan epitopes that are usually attached to lipids, e.g. GF296 (asialo GM1) and GF300 (asialo GM2), may behave differently in IHC and FACS due to the biochemical differences in experimental procedures. Additionally, the affinity and avidity of the antibodies may be different affecting to the results in stable IHC compared to fluidic system in FACS analysis. However, both methods are widely used in biological studies and the results should be considered valid with both methodologies.

### Example 15

Revealing Protease Sensitive and Insensitive Antibody Target Structures

Bone marrow mesenchymal stem cells and osteogenic cells derived thereof as described in examples above were analyzed by FACS analysis. Several antigen structures are essentially not observed or these are observed in reduced amount in FACS analysis of cell surface antigens when cells are treated (released from cultivation) by trypsin (0.25%), but observable after Versene treatment (0.02% EDTA in PBS). Several glycan epitopes, e.g. GF277 (sTn), GF278 (Tn), GF295 (pLN), GF296 (asialo GM1), GF299 (Forssman antigen), GF300 (asialo GM2), GF302 (H Type 2), GF304 (Lea), and GF306 (sLea), were practically totally destroyed by trypsin treatment in both BM-MSCs and osteogenic cells derived thereof (Table 19). Some glycan epitopes, such as GF275 (CA15-3), GF307 (sLex), and GF354 (SSEA-4) were partially sensitive for trypsin treatment.

### Example 16

### Comparision of Differentiated and Non-Differentiated MSCs and Identification a Fucosyl Glycan Marker

Mesenchymal Stem Cells

Mesenchymal stem cells (MSC:s) are fibroblast-like adult multipotent progenitor cells that can be isolated from various sources such as bone marrow or cord blood. MSC:s are capable of differentiating into mesenchymal cell types like osteoblasts, chondroblasts and adipocytes.

Objectives

This study was carried out to characterize the N-glycome of human mesenchymal stem cells. Stem cells hold an enormous therapeutic potential in regenerative medicine. However, before stem cells can be used in the clinical practice, there is a need for methods to thoroughly characterize them, to distinguish them from other cells, and to control variation within and between different cell lines. A glycomic approach to study stem cells provides an ideal platform to solve these issues. Modern mass spectrometric methods provide the means to characterize the glycome even when the amount of sample available is very limited.

Materials and Methods

Human mesenchymal stem cells were isolated from bone marrow and cultured. Osteogenic differentiation was induced by placing the cells in osteogenic induction medium. The N-linked glycans were enzymatically released with protein N-glycosidase F from about 100 000-1 000 000 cells. The total glycan pools (picomole quantities) were purified with microscale solid-phase extraction and divided into neutral and sialylated glycan fractions. The glycan fractions were analyzed by MALDI-TOF mass spectrometry either in positive ion mode for neutral N-glycans or in negative ion mode for sialylated glycans (Saarinen et al., 1999, Eur. J. Biochem. 259, 829-840). Over one hundred N-glycan signals were detected from each cell type revealing the surprising complexity of hESC glycosylation. The relative abundances of the observed glycan signals were determined based on relative signal intensities (Harvey, 1993. Rapid Commun. Mass Spectrom. 7:614-9; Papac et al., 1996. Anal. Chem. 68:3215-23)with a Bruker Ultraflex TOF/TOF instrument. Exoglycosidase digestions were carried out to further characterize terminal epitopes. In addition, carbohydrate epitopes were studied by immunofluorescent staining to support the mass spectrometric data.

Results and Conclusions

More than one hundred glycan signals were detected for both cell types. Of these some signals were characteristic of stem cells and decreased upon differentiation, whereas other signals became more prominent upon differentiation. Specific structural features associated with either stem cells or differentiated cells could be seen by exoglycosidase digestions and immunofluorescent stainings. In conclusion, mesenchymal stem cells have a characteristic N-glycan profile that changes upon differentiation. The information on the stem cell glycome can be used to evaluate the differentiation stage of stem cells and to develop new stem cell markers (e.g. for antibody development) as well as to study the interactions of stem cells with their niches and thus develop improved in vitro culture systems.

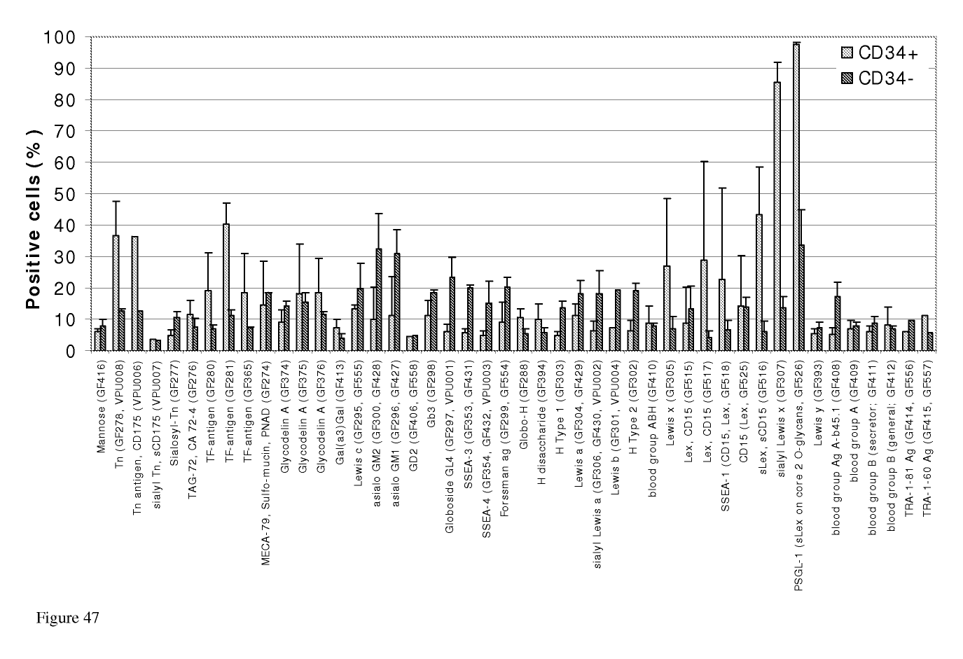

The FIG. 36 shows difference in N-glycan profiles of MSC cells and their differentiated variant. The differences of signals in FIG. 36b for neutral glycans and FIG. 36d for acidic glycans were used to identify key structures altering during differentiation. FIG. 37 shows cleavage of fucosylresidue by specific fucosidase from di- and trifucosylated biantennary neutral N-glycans. Combination of the result with cleavage by β-galactosidase indicates presence of Lewis x structure on N-glycans. FIG. 38 shows staining by an anti-sialyl-Lewis x antibody binding to the sialylated terminal epitope analogous to Lewis x, see Example 44 for details.

### Example 17

### Immunostaining

Immunohistochemistry (IHC). Bone-marrow derived mesenchymal stem cells on passages 9-12 were grown on CC2 treated glass 8-chamber slides (Lab-TekII, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) were diluted in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK) Immunostainings were observed with Zeiss Axioskop2 plus—fluorescence microscope (Carl Zeiss Vision GmbH, Germany) with FITC and DAPI filters. Images were taken with Zeiss AxioCam MRc -camera and with AxioVision Software 3.1/4.0 (Carl Zeiss) with the 400× magnification.

The results with staining mesenchymal cells by specific clone of antibody to sialyl Lewis x (GF307) are shown in FIG. 38. The specific antibody type show specificity for non-differentiated hMSCs. The specification of antibody is:

| | | | | |
|---|---|---|---|---|
| GF307 | Sialyl Lewis x | Mouse/IgM | Chemicon int. | MAB2096 |

### Example 18

### N-Glycosylation of Human Cord Blood-Derived Stem Cells

Abstract

Cell surface glycans contribute to the adhesion capacity of cells and are essential in cellular signal transduction. Yet, the glycosylation of hematopoietic stem cells, such as CD133+ cells, is poorly explored. In this study, we analyzed N-glycan structures of CD133+ and CD133− cells with mass spectrometric profiling and exoglycosidase digestion; cell surface glycan epitopes with lectin binding assay; and expression of N-glycan biosynthesis-related genes with microarray. Over 10% difference was demonstrated in the N-glycan profiles of CD133+ and CD133− cells. Biantennary complex-type N-glycans were enriched in CD133+ cells. Of the genes regulating the synthesis of these structures, CD133+ cells overexpressed MGAT2 and underexpressed MGAT4. Moreover, the amount of high-mannose type N-glycans and terminal α2,3-sialylation was increased in CD133+ cells. Elevated α2,3-sialylation was supported by the overexpression of ST3GAL6. The new knowledge of hematopoietic stem cell-specific N-glycosylation advances their identification and provides tools promote stem cell homing and mobilization or targeting to specific tissues.

Introduction

More than half of human proteins are estimated to be glycosylated. In other words, glycosylation is more common post-translational modification than phosphorylation (1). Glycans cover the entire cell surface as the glycocalyx and they function as structural components and signal transducers. Glycans are essential for many biological processes including cellular response to oxidative stress, resistance to innate immunity and cell-cell or cell-matrix communication (2,3). In hematopoietic stem cells, such as CD133+ cells, cell type-specific glycosylation may contribute to maintenance, differentiation, homing and mobilization.

Cord blood is a convenient source of stem cells; they are easy to obtain and they have better tolerance for histocompatibility mismatches than stem cell grafts from other sources. Cord blood transplantations are often used when perfect HLA-matched donor is not available. The number of cells available in one cord blood unit is often considered adequate only for pediatric patients and numerous methods have been attempted to expand stem cells in vitro. The hematopoietic stem cells essential for therapy are often characterized based on the expression of cell surface glycoproteins CD34 and CD133. Nearly all (99,8%) of CD133+ cells are also CD34 positive (4). During differentiation, the CD133 molecule is lost from the cell surface earlier than CD34.

Understanding hematopoietic stem cell glycobiology offers new techniques for better stem cell engraftment, ex vivo or in vivo expansion and targeting to specific tissue (5-7). Characterization of CD133+ cell N-glycome would also better the identification of hematopoietic stem cells. However, N-glycosylation is a complex event, and so far the analysis of human stem cell glycome has been lacking suitable technology to analyze samples with limited cell number. N-glycan biosynthesis is controlled by expression of glycosyltransferase and glycosidase enzymes and isozymes which compete for the same glycan substrates. In addition, formation of glycan molecules, their precursor biosynthesis, transport, and localization mechanisms, are entwined with other biosynthetic pathways (8,9). A change in the activity of one single glycan biosynthetic enzyme can have a drastic effect on the appearance and the function of the cell. However, the identification of specific genes involved in the certain glycosylation process requires that the expression level of glycosylation-related genes are compared to glycan structures. Recently, dramatic N-glycome changes with differential expression of only few genes have been described in activated murine T cells (10-12). Differential expression of genes encoding sialyltransferases have been shown to differentially contribute to the B lymphocyte response to immune signaling (13).

In the present study, we characterized N-glycosylation events typical for CD133+ cells by combining data from N-glycan structure analysis and expression profiling of genes encoding glycosyltransferases and glycosidases. The results of CD133+ cells were compared to mature leucocytes (CD133−) to identify N-glycosylation specific for CD133+ cells. Our work presents new information on the characters of stem cells. The results may help to develop their use in therapeutic applications. Engineering cell glycosylation could be used to enhance stem cell homing and mobilization or to design cell products targeted to specific tissues.

Materials and Methods

Cells

Cord blood was obtained from the Helsinki University Central Hospital, Department of Obstetrics and Gynaecology, and Helsinki Maternity Hospital. All donors gave informed consent and the study was approved by ethical review board of the Helsinki University Central Hospital and the Finnish Red Cross Blood Service. Collection and processing of the fresh cord blood was performed as described earlier (14). Ficoll-Hypaque density gradient (Amersham Biosciences, New Jersey, USA, www1.amerschambiosciences.com) was used to isolate leucocytes that are mononuclear cells. Leucocytes can be obtained in quantities adequate for NMR analysis. In addition, leucocytes were used in lectin labeling assay. Stem cell fraction was sorted from the leucocyte fraction with anti-CD133 microbeads in magnetic affinity cell sorting (Miltenyi Biotec, Bergisch Gladbach, Germany, www.miltenyibiotec.com) (15). Mature leucocytes (CD133− cells) were collected for control purposes. Altogether 11 cord blood units were used. In the preparation of samples to mass spectrometric analysis, to avoid olicosaccharide contamination, ultra pure bovine serum albumin (at least 99% pure, Sigma-Aldrich Chemie GmbH, Steinheim, Germany, www.sigmaaldrich.com) was used.

N-Glycan Isolation

N-glycans were detached from cellular glycoproteins by F. meningosepticum N-glycosidase F digestion (Calbiochem, USA) essentially as described (Nyman et al., 1998). Cellular contaminations were removed by precipitating the glycans with 80-90% (v/v) aqueous acetone at −20° C. and extracting them with 60% (v/v) ice-cold methanol (Verostek et al., 2000). The glycans were then passed in water through C18 silica resin (BondElut, Varian, USA) and adsorbed to porous graphitized carbon (Carbograph, Alltech, USA). The carbon column was washed with water, and then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C18 silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used.

Mass Spectrometry

MALDI-TOF mass spectrometry was performed with a Bruker Ultraflex TOF/TOF instrument (Bruker, Germany) and the samples were prepared for the analysis essentially as described (22). Neutral N-glycans were detected in positive ion reflector mode as [M+Na]+ ions and sialylated N-glycans were detected in positive ion reflector or linear mode as [M-H]− ions. Relative molar abundances of neutral and sialylated glycan components were assigned based on their relative signal intensities in the mass spectra when analyzed separately as the neutral and sialylated N-glycan fractions (Saarinen, 1999. Harvey, 1993, Naven, 1996, Papac, 1996). The mass spectrometric raw data was transformed into the present glycan profiles by removing the effect of isotopic pattern overlapping, multiple alkali metal adduct signals, products of elimination of water from the reducing oligosaccharides, and other interfering mass spectrometric signals not arising from the glycan components in the sample. The resulting glycan signals in the presented glycan profiles were normalized to 100% to allow comparison between samples.

Quantitative difference between two glycan profiles (%) was calculated according to Equation 1:

$$difference = \frac{1}{2} {\sum }_{i = 1}^{n} \vert {p}_{i , a} - {p}_{i , b} \vert , \qquad ( 1 )$$

wherein p is the abundance (%) of glycan signal i in profile a or b, and n is the total number of glycan signals.

Relative difference in a glycan feature between two glycan profiles was calculated according to Equation 2:

$$relative difference = {x ( \frac{{P}_{a}}{{P}_{b}} )}^{x} , \qquad ( 2 )$$

wherein P is the sum of the abundance (%) of the glycan signals with the glycan feature in profile a or b, x is 1 when a≧b, and x is −1 when a<b.

NMR Spectroscopy

The isolated glycans were further purified for NMR spectroscopy by gel filtration high-pressure liquid chromatography in water or 50 mM ammonium bicarbonate to separate neutral and sialylated glycan fractions, respectively. The NMR analysis was performed as previously descripted (Weikkolainen et al. Glycoconj. J. 2007 in press) with Variant Unity NMR spectrometer at 800 MHz using a cryo-probe for enhanced sensitivity. Prior to proton NMR analysis, the purified glycans were dissolved in 99.996% deuterium oxide and dried to omit water and to exchange sample protons.

Exoglycosidase Analysis

Analysis of non-reducing glycan epitopes present in N-glycan fractions was performed by digestion with specific exoglycosidase enzymes. Enzyme specificity towards isomeric structures was controlled in parallel reactions with defined oligosaccharides as detailed below. The employed exoglycosidase enzymes were: β1,4-galactosidase from S. pneumoniae (recombinant in E. coli, Calbiochem) digested the β1,4-linked galactose of lacto-N-hexaose, β1,3-galactosidase from X. manihotis (recombinant in E. coli, Calbiochem) digested the β1,3-linked galactose of lacto-N-hexaose, α2,3-sialidase from S. pneumoniae (recombinant in E. coli, Calbiochem) digested α2,3-but not α2,6-sialyl N-acetyllactosamine, broad-range sialidase from A. ureafaciens (recombinant in E. coli, Calbiochem) digested both α2,3- and α2,6-sialyl N-acetyllactosamine, and α-mannosidase from Jack beans (C. ensiformis; Sigma-Aldrich) digested the Man5-Mang high-mannose type N-glycans present in oligosaccharide mixture isolated from human cells. The reactions were carried out by overnight digestion at +37° C. in 50mM sodium acetate buffer, pH 5.5. The digested glycan fractions were purified for analysis by solid-phase extraction with graphitized carbon and analyzed by MALDI-TOF mass spectrometry as described above.

Microarray

RNA purified from CD133+ and CD133− cells was hybridized on Affymetrix Human Genome U133 Plus 2.0 arrays, and the data was analyzed using Affymetrix GeneChip Operating Software as previously described (14). When applicable, the same probes were selected for analysis that are represented on the Affymetrix glycogene chip provided by the Gene Microarray Core of Consortium for Functional Glycomics. A transcript was considered differentially expressed when at least 1.5-fold increase or decrease in the expression was demonstrated.

Lectin Binding Analysis by Flow Cytometry

To prevent hemolysis or hemagglutination of erythrocyte precursors by lectins which would disturb the flow cytometric analysis, MNCs were GlyA depleted using Glycophorin A MicroBeads (Miltenyi Biotec). The cells were labeled with phycoerythrin (PE)-conjugated CD34 monoclonal antibody (Miltenyi Biotec) to show the stem cell population and with one of the fluorescein isothiocyanate (FITC)-conjugated lectins PSA from Pisum sativum for α-mannose and glucose; HHA from Hippeastrum hybrid for internal and terminal α1,3- or α1,6-linked mannose, and GNA from Galanthus nivalis for α1,3-mannose residues; PHA-L from Phaseolus vulgaris L for large complex-type N-glycans; RCA-I from Ricinus communis I for β-galactose; SNA from Sambucus nigra and MAA from Maackia amurensis for α2,6- and α2,3-linked sialic acid, LTA from Lotus tetragonolobus and UEA-I from Ulex europaeus I for α-fucose; EY Laboratories, Inc. San Mateo, Calif., USA, www.eylabs.com; Vector Laboratories, Burlingame, Calif., USA, www.vectorlabs.com). Flow cytometry was performed on Becton Dickinson FACSCalibur™ and fluorescence was measured using 530/30 nm and 575/25 nm bandpass filters. The labeling results of MNCs show the overall frequency of specific glycosylation events. The double-labeled cell fraction specifies the glycans on the cell surface of stem cells.

Results

Structural Analysis

For the structural analysis, neutral and sialylated N-glycan fractions from total leucocytes were subjected to NMR. The NMR analyses yielded detailed data about the most abundant N-glycan structures present in leucocytes (unseparated mononuclear cells). High-mannose type N-glycans were detected in neutral N-glycan fraction, whereas the N-glycan backbone with α2,6- and α2,3-sialylated biantennary complex-type N-glycans were the major structures in the sialylated N-glycan fraction. Moreover, quantitative analysis of the spectrum showed that α2,6-sialylation was more abundant than α2,3-sialylation, and type 2 N-acetyllactosamine (Galβ4GlcNAc, 100%) dominated over type 1 N-acetyllactosamine (Galβ3GlcNAc, not detected) in the N-glycan antennae. (β1,4-branched triantennary N-glycans and α1,6-fucosylated N-glycan core were also detected.

To compare the quality and quantity of N-glycans on stem cells and mature leucocytes, CD133+ and CD133− cells were separately analyzed by MALDI-TOF mass spectrometry. The data from NMR was used to qualify structures presented in the mass spectrometric analysis. Over 80 signals containing some multiple isomeric structures were detected in both cell types (FIGS. 42 and 43A).

The profile of sialylated N-glycans was more divergent between CD133+ and CD133− cells (17% difference) than the neutral N-glycan profiles (9% difference). Major N-glycans in CD133+ and CD133− cells were high-mannose and biantennary complex-type structures (FIG. 21). CD133+ and CD133− cells also had monoantennary, hybrid, low-mannose and large complex-type N-glycans (FIGS. 42 and 43). To analyze the differences between CD133+ and CD133− cells, the proposed monosaccharide compositions assigned to each detected glycan signal (FIGS. 42 and 43; A and B) were quantitatively analyzed by grouping them into the major N-glycan classes (FIG. 42C and 43C) and by comparing the proportion of different major N-glycan classes between CD133+ and CD133− cells. The CD133+ cell N-glycome showed polarization towards high-mannose type N-glycans (FIG. 42C), biantennary complex-type N-glycans with core composition 5-hexose 4-N-acetyhexosamine and sialylated monoantennary N-glycans (FIG. 43C). In contrast, CD133− cells had increased amounts of large complex-type N-glycans with core composition 6-hexose 5-N-acetylhexosamine or larger, sialylated hybrid-type N-glycans and low-mannose type N-glycans.

The CD133− cell population presents an average of the phenotypes of multiple cell types. To compare the results with an independently isolated differentiated cell population, the CD8+ and CD8− cells were analyzed. CD8+ cells showed an N-glycosylation phenotype similar to CD133− cells. Especially the proportion of large complex-type N-glycans was elevated in these cells (data not shown). This indicates that demonstrated N-glycome in CD133+ cells is typical for the cell type.

To characterize terminal epitope profile on CD133+ and CD133− cells, specific exoglycosidase digestions was combined with mass spectrometric analysis. α-mannose, β1,4-galactose, and β-N-acetylglucosamine residues were found abundant in both cell types, whereas β1,3-linked galactose residues were not detected in significant amounts. The majority of both CD133+ and CD133+ cells carried α2,6-linked sialic acids, as demonstrated in α2,3-sialidase treatment. Neutral that is completely desialylated glycan components were produced from all sialylated N-glycan species from CD133+ cells, whereas CD133− cells contained minor components completely resistant to the α2,3-sialidase treatment. Further, the acidic glycan profile change during the specific sialidase treatment was quantitatively larger in CD133+ cells compared CD133− cells (FIG. 44). Taken together, the proportions of the N-glycan signals affected to α2,3-sialidase in CD133+ and CD133− cells were different showing enrichment in CD133+ cell α2,3-sialylated N-glycans (FIG. 44).

Biosynthetic Pathways of N-glycosylation

After glycan profiling, expression of genes encoding enzymes that modify N-glycan structures were studied. N-glycan biosynthesis is controlled with several glycosyltransferase and glycosidase enzyme families that act on different regions of the N-glycan chain; N-glycan core, backbone and terminal regions (FIG. 45). Biosynthesis of other important glycan classes such as O-glycans and glycolipids partly overlap with N-glycan biosynthesis, but different members of enzyme families are often specialized to synthesize certain glycan types. The target glycan classes for the gene products and the expression results of N-glycan structure-associated genes are shown in table 20.

N-Glycan Core Sequence

N-glycan core structures are formed by specialized mannosidase (MAN) and N-acetylglucosaminyltransfrerase (GlcNAcT) enzymes (16) (FIG. 44). MANs shape high-mannose and low-mannose type N-glycan structures and form the starting points for the other N-glycan types (8). MAN1 enzymes control the conversion from high-mannose to hybrid-type and monoantennary N-glycans, and MAN 2 enzymes control the further conversion to complex-type structures. GlcNAcTs determine the branching modes of hybrid, monoantennary, and complex-type N-glycans (17).

High-mannose type N-glycans were the prevalent neutral N-glycan group. The relative amounts of neutral α-mannosylated N-glycans were similar in CD133+ and CD133− cells (FIG. 44). However, terminal α-mannose was enriched in high-mannose type glycans in CD133+ cells, whereas terminal α-mannose was broadly found in low-mannose, hybrid, and monoantennary-type N-glycans in CD133− cells. The presence of α-mannose on the cell surface was further demonstrated by lectin labeling (Table 21). α-mannose and N-glycan core sequence-binding lectins PSA and HHA labeled 96-99% of mature leucocytes and the stem cell population. GNA labeled 73% of the mature leucocytes but only few stem cells. GNA has highest affinity towards low-mannose type N-glycans with terminal α1,3-mannose residues. Lectin labeling result suggests differential α-mannosylation for stem cells like the observations from structural analysis.

High-mannose type N-glycans are processed into other N-glycan types by glycosidase families MAN1 and MAN2 (8,16) (Table 20). Three of the four known MAN1 family genes MAN1 A1, MAN1A2 and MAN1B1 and all five known MAN2 family genes MAN2A1, MAN2A2, MAN2B1, MAN2B2 and MAN2C1 were similarly expressed in CD133+ and CD133− cells. The fourth member of MAN1 gene family, MAN1C1, was expressed in CD133− cells only. Its specific role within the MAN1 family is not known. However, In vitro the MAN1C1 encoded enzyme prefers removal of the GlcNAcT blocking mannose residues in the α1,3 branch (21).

The amount of N-glycan structures larger than biantennary complex-type N-glycans was decreased in CD133+ cells according to structural analysis. PHA-L that binds to branched complex-type N-glycans labeled 98% leucocytes and most of the stem cells (Table 21). The labeling result shows that dispute the quantitative difference in the large complex-type N-glycans between mature leucocytes and stem cells, these structures are typical for both cell types.

The biosynthesis of hybrid-type and complex-type N-glycans is controlled by a family of N-glycan core GlcNAcTs encoded by MGAT genes (Table 20). MGAT1, MGAT2 and MGAT4A/MGAT4B encode GlcNAcT1, GlcNAcT2 and GlcNAcT4, respectively. These genes were expressed in CD133+ and CD133− cells, but differences in their expression levels were demonstrated. In CD133+ cells MGAT2 was overexpressed by 1.9-fold and MGAT4A was underexpressed by 2.8-fold.

Together, both MAN1C1 and MGAT2 expression patterns in CD133+ cells indicates increased biosynthesis of high-mannose type and complex-type N-glycans, and decreased biosynthesis of hybrid-type and monoantennary N-glycans. In addition, underexpression of MGAT4A may result in the reduction of triantennary and larger N-glycans in stem cells.

N-Glycan Backbone

Glycan backbone structures include short antennae and extended poly-N-acetyllactosamine (poly-LacNAc) chains formed by the concerted action of galactosyltransferases (GalT; antennae and poly-LacNAc) and GlcNAcTs (poly-LacNAc) (FIG. 45). The present study focused on GalTs, because the short antennae-type structures dominated over poly-LacNAc in leucocytes. The terminal galactose residues were shown to be β1,4-linked, whereas β1,3-linked galactose was not detected. Lectin RCA-I that is specific for type 2 LacNAc labelled 91% of the leucocytes as well as the stem cells.

Genes encoding the β1,4-GalTs synthesizing type 2 LacNAc epitopes, such as B4GALT1, B4GALT3 and B4GATL4 were expressed in both CD133+ and CD133− cells (Table 20). However, the expression of B4GALT3 was decreased in CD133+ cells by 2.3-fold. Further, the expression of B4GALT2 was only seen in CD133+ cells. Type 1 LacNAc synthesizing β1,3-GalTs, encoded by B3GALT2 and B3GALT5 were absent in CD133+ and CD133− cells, as were the potential glycan products.

N-Glycan Terminal Epitopes

The terminal epitopes are added on the N-glycan structures during the final phase of the synthesis (FIG. 45). Common glycan moieties in terminal modifications of N-glycans include sialic acid and fucose residues. Sialyltransferase families α2,3SATs and α2,6SATs transfer sialic acids to terminal galactose residues. Such epitopes were found in CD133+ and CD133− cells. In addition, all known human fucosyltransferase synthetic pathways were analysed.

The α2,3-sialidase profiling revealed that α2,3-sialylated N-glycans were more common in CD133 + cells than in CD133− cells (FIG. 44), whereas α2,6-sialyl-LacNAc was common for both cell types. Lectin SNA was used to detect α2,6-sialylation, the product of ST6GAL1 on cell surface. SNA ligands were detected on 98% of the leucocytes including the stem cells. Labeling with MAA showed that α2,3-sialyl-LacNAc structures were present on only 62% of the leucocytes, and similarly in stem cells. This suggests that enriched α2,3-sialylation of CD133+ cells may be related to N-glycans only. ST6GAL1 encoding α2,6-SAT and ST3GAL6 encoding α2,3-SAT were expressed in CD133+ and CD133− cells (Table 20). 3.9-fold overexpression of ST3GAL6 was detected in CD133+ cells.

N-glycan core structures of CD133+ and CD133− cells were often α1,6-fucosylated as shown by mass spectrometric analysis. In addition, presence of two or more fucose residues on each N-glycan chain was observed in CD133+ and CD133− cells (FIGS. 42 and 3). Since type 1 LacNAc was prevalent neither in CD133+ or CD133− cells, the fucosylated epitopes were expected to carry α1,3- or α1,2-linked fucose residues. Lectin LTA has specificity towards α1,3-linked fucose, that is part of the Lex antigen. It labeled only 6% of the leucocytes. No labeling of stem cell population was shown. Lectin UEA-I with α1,2-linked fucose specificity recognized 53% of the leucocytes and the stem cells.

The expression of FUT4 that encodes the myeloid type α1,3-FucT4 (18,19) was found in both CD133+ and CD133− cells. FucT4 synthesizes the Lex (CD15) or sLex antigens by fucosylation of type 2 LacNAc or α3-sialyl LacNAc, respectively. FUT1 encoding a1,2-FucT was not expressed in CD133+ or CD133− cells. Moreover, only CD133+ cells expressed detectable levels of FUT8 encoding the N-glycan core α1,6-FucT a glycosylation abundantly detected in the structural analysis of CD133+ and CD133− cells. FUT8 is the only known gene encoding a glycosyltransferase promoting α1,6-fucosylation, yet previous reports show that an increase in a1,6-fucosylation can not be explained by the up-regulation of α1,6-FucT alone (20).

Discussion

The present work uses a new approach to characterize CD133+ cells. CD133+ cell-specific N-glycosylation and the transcriptional regulation of the glycosylation events were linked together to gather the expressed genes producing key N-glycan entities different between stem cells and mature leucocytes. In addition, lectin binding assay revealed divergences on the cell surface glycosylation between stem cells and mature leucocytes.

Although rare N-glycan structures may not be detected by MALDI-TOF and NMR analysis, the method allows quantitative analysis of glycan compositions between different cell types. Enrichment of high-mannose type glycans were representative of stem cells, also on the cell surface as shown with lectin labeling. Mature leucocytes contained more large complex-type N-glycans, whereas complex N-glycans were often biantennary in CD133+ cells. The gene expression seems to support the core glycosylation typical for the cell type. Putative role for the absence of MAN1C1 is suggested as slowing the conversion from high-mannose type to hybrid-type and monoantennary glycans.

The structures present in CD133+ cells, such as high-mannose and complex type N-glycans, are found on CD164 epitope (24). The function of the CD164 molecule is indeed N-glycan-dependent and modulates the CXCL12-mediated migration of cord blood-derived CD133+ cells (24,25). It also negatively regulates stem cell proliferation (26,27). Complex N-glycan determinants are also part of other adhesion molecules common to hematopoietic stem cells, such as the CD34+ cell-specific glycoform of CD44 molecule.

Different β1,4-galactosylation-related genes were involved in the β1,4-galactosylation of CD133+ and CD133− cells. No change in their glycan profiles was detected. However, these genes might galactosylate N-glycan backbones of single glycoproteins.

B4GALT2 expressed only in CD133+ cells has restricted expression pattern to fetal brain, adult heart, muscle and pancreas (28), whereas B4GALT3 is widely expressed in most tissues (28). B4GALT2 and B4GALT3 encoded enzymes have almost identical substrate specificity and they may substitute each other (29). Both B4GALT2 and B4GALT3 galactosylate biantennary and larger complex-type N-glycans. The expression of B4GALT2 in CD133+ cells may be compensated with the underexpression of B4GALT3. However, changes in glycoproteins present on lower abundances might not be detected by present methods therefore it is possible that differential glycosylation exist on single glycoproteins. B4GALTs synthesize the glycan backbones of selectin ligands, although selectin adhesion is regulated trough terminal glycosylation. Galactosylation has an important role in the proliferation and differentiation of epithelial cells in mice (30). If the differential biosynthetic pathways of CD133+ and CD133− cells have an influence on β1,4-galactosylation of certain glycoproteins, the significance of β1,4-galactosylated structures could participate in controlling the proliferation and differentiation of CD133+ cells. This interesting hypothesis requires closer examination.

α2,6-sialylation dominates the cell surface glycans of human bone marrow and peripheral blood-derived CD34+ and CD34− cells (31) similarly as in cord blood-derived CD133+ and CD133− cells. Moreover, granulocyte colony-stimulating factor mobilized CD34+ cells in peripheral blood and bone marrow-derived CD34+ cells have higher expression of ST6GAL1 with elevated α2,6-sialylation on the cell surface than noninduced peripheral blood-derived CD34+ cells indicating that α2,6-sialylation of CD34+ cells is dependent of granulocyte colony-stimulating factor in their environment (12). α2,6-sialylation of CD34+ cells might participate regulating their cellular adherence. α2,6-linked sialic acid, product of ST6GAL 1 is crucial for homing process of CD22+ B-cells (32). Expression of ST6GAL 1 reduces galectin-1 binding to cells (33). Galectin-1 stimulates stem cell expansion (34). Galectin-1 is abundantly secreted by mesenchymal stem cells (35), but its expression is not detected in CD133+ cells (gene expression profile in (14)). Hematopoietic stem cells expand and remain their long-term reconstruction capacity longer when they are co-cultured with mesenchymal stem cells (36). Mesenchymal and hematopoietic stem cell interaction in co-cultures could be assisted by galectin-1 binding.

In sialylated glycan biosynthesis, α2,3- and α2,6-SATs can compete for the same N-glycan substrates. In the present study we show enriched α2,3-sialylation in CD133+ cells, accompanied with overexpression of ST3GAL6. Previously lower proportion of α2,6-SAT1 together with lower α2,6-sialylation of N-glycans was demonstrated in murine T cell activation (11). The authors suggested that this may be due to α1,3-GalT expression competing from the same substrate with α2,6-SAT1. However, α1,3-GalT is not present in human and therefore, the similar substrate competition is not relevant. The present results show that in human CD133+ cells lower relative abundance of α2,6-sialylation is instead caused by increased α2,3-sialylation. Gene expression data strongly suggests that ST3GAL6 overexpression is responsible for the increased α2,3-sialylation in these cells. ST3GAL6 has got restricted substrate specificity which lead to suggest it is involvement to synthesis of sialyl-paragloboside, a precursor structure of sialyl-Lewis X determinant (37). However, the expression of ST3GAL6 was not shown to correlate with expression of sialyl-Lewis X.

CD34+ cells (also CD133+ cells), but not mature leucocytes, display a hematopoietic cell L and E-selectin ligand, a glycoform of the CD44 antigen, critically dependent on N-glycan sialylation(38-40). Selectin-ligand interactions promote homing of stem cells and may also control their proliferation. L-selectins present on CD34+ cells have been associated with faster hematopoietic recovery after stem cell transplantation (38). The α2,3-sialylation of N-glycans negatively regulates the ability of CD44 molecule to bind extracellular matrix (41). The main role of CD44 is binding to hyalyronic acid (42), yet only small amount of CD34+ cells carrying CD44 epitope are bound to hyaluronic acid in bone marrow (43). Therefore, α2,3-sialylation is probably at least needed to assist both the homing and proliferation of stem cells.

In addition to N-glycan core α1,6-fucosylation, small amounts of α1,2- or α1,3-linked fucose residues were present. The expression of FUT genes indicate the synthesis of myeloid type α1,3-linked fucose. However, the presence of α1,3-fucosylation was detected very low on cord blood-derived leucocytes, including stem cells. On the other hand, α1,2-linked fucose was detected on cell surface even expression of FUT1 processing α1,2-fucosylation was absent. FUT7 product is a key enzyme responsible for the synthesis of sLex that binds to selectins (44). In addition, FUT1 expression has been shown to inhibit sLex expression (45). cord blood-derived stem cells have been shown to have impaired α1,3-fucosylation trough reduced α1,3-fucosyltransferase expression which contribute to lower selectin binding and may delay engraftment of cord blood-derived cells in transplantation (5,7). During embryogenesis, only FUT4 and FUT9 are expressed. FUT4 expression has been shown to compensate low or absent FUT7 expression and production of such as sLe x required in selecting binding in adults with deficient FUT7 expression (46). At least two attempts to enforce fycosylation of stem cells have been performed (5,7), in both cases fucosylation was successful, and in one of them could show improved homing to bone marrow of noneobese diabetic/severe combined immune deficient mice (7). If defect in FUT7 expression in cord blood-derived cells cause delay in stem cell engraftment to human bone marrow, cell engineering techniques could be used to enhance stem cell fucosylation.

Taken together, the critical genes associated to characteristic N-glycosylation of CD133+ cells were, overexpression of MGAT2 and ST3GAL6, underexpression of MGAT4A and the absence of MAN1C1. In addition, β1,4-galactosylation was on molecular level regulated differently between CD133+ and CD133− cells with unknown function that is a matter of further investigation. CD34+ and CD133+ cells have highly similar genome-wide gene expression profile (47). It was expected that if the genes-related to N-glycosylation in CD133+ cells are pivotal to stem cell N-glycome, the genes should be similarly expressed in CD34+ cells as well. Expression of N-glycosylation-related genes in CD34+ cells was proved to be similar with CD133+ cells (gene expression results collected from published CD34+ expression profile (47)). In addition, the same change in the expression pattern was noticed between CD34+ and CD34− cells than between CD133+ and CD133− cells suggesting that N-glycome of cord blood-derived CD34+ cells is very similar to CD133+ cell N-glycome and differing from mature leucocytes.

The characterized N-glycan features in CD133+ cells have crucial role in known glycoproteins such as CD164, hematopoietic stem cell and progenitor specific CD44 glycoform, and binding of E-selectin, P-selectin and galectin ligands that are required for cell migration, proliferation, cell recognition and homing to BM. The N-glycome of CD 133+ cells may also be involved in many yet unknown functions. Combined information from changes in gene expression and glycan structures between CD133+ and CD133− cells allowed identification of novel genes regulating CD133+ cell-specific N-glycan biosynthesis. The new knowledge of hematopoietic stem cell-specific N-glycosylation helps to engineer novel therapeutic applications or to improve current protocols. Changing the glycosylation in vitro or in vivo can be used to enhance the natural properties of stem cells or to modify N-glycome that would target stem cells to specific tissues.

References of Example 18 and Table 20.

### REFERENCES

1. Apweiler R, Hermjakob H and Sharon N. (1999). On the frequency of protein glycosylation, as deduced from analysis of the SWISS-PROT database. Biochim Biophys Acta 1473:4-8.

2. Varki A. (1993). Biological roles of oligosaccharides: all of the theories are correct. Glycobiology 3:97-130.

3. Varki A. (2006). Nothing in glycobiology makes sense, except in the light of evolution. Cell 126:841-845.

4. Gallacher L, Murdoch B, Wu D M, Karanu F N, Keeney M and Bhatia M. (2000). Isolation and characterization of human CD34(−)Lin(−) and CD34(+)Lin(−) hematopoietic stem cells using cell surface markers AC133 and CD7. Blood 95:2813-2820.

5. Hidalgo A, Frenette P S. (2005). Enforced fucosylation of neonatal CD34+ cells generates selectin ligands that enhance the initial interactions with microvessels but not homing to bone marrow. Blood 105:567-575.

6. Sampathkumar S G, Li A V, Jones M B, Sun Z and Yarema K J. (2006). Metabolic installation of thiols into sialic acid modulates adhesion and stem cell biology. Nat Chem Biol 2:149-152.

7. Xia L, McDaniel J M, Yago T, Doeden A and McEver R P. (2004). Surface fucosylation of human cord blood cells augments binding to P-selectin and E-selectin and enhances engraftment in bone marrow. Blood 104:3091-3096.

8. Herscovics A. (1999). Importance of glycosidases in mammalian glycoprotein biosynthesis. Biochim Biophys Acta 1473:96-107.

9. Lowe J B. (2002). Glycosylation in the control of selectin counter-receptor structure and function. Immunol Rev 186:19-36.:19-36.

10. Le Marer N, Skacel P O. (1999). Up-regulation of alpha2,6 sialylation during myeloid maturation: a potential role in myeloid cell release from the bone marrow. J Cell Physiol 179:315-324.

11. Comelli E M, Sutton-Smith M, Yan Q, Amado M, Panico M, Gilmartin T, Whisenant T, Lanigan C M, Head S R, Goldberg D, Morris H R, Dell A and Paulson J C. (2006). Activation of murine CD4+ and CD8+ T lymphocytes leads to dramatic remodeling of N-linked glycans. J Immunol 177:2431-2440.

12. Hui N, Le Marer N. (2001). Alpha-2,6-sialylation regulation in CD34+ progenitor cells in the human bone marrow and granulocyte colony-stimulating factor mobilization. J Hematother Stem Cell Res 10:661-668.

13. Marino J H, Hoffman M, Meyer M and Miller K S. (2004). Sialyltransferase mRNA abundances in B cells are strictly controlled, correlated with cognate lectin binding, and differentially responsive to immune signaling in vitro. Glycobiology 14:1265-1274.

14. Jaatinen T, Hemmoranta H, Hautaniemi S, Niemi J, Nicorici D, Laine J, Yli-Harja O and Partanen J. (2006). Global gene expression profile of human cord blood-derived CD133+ cells. Stem Cells 24:631-641.

15. Kekarainen T, Mannelin S, Laine J and Jaatinen T. (2006). Optimization of immunomagnetic separation for cord blood-derived hematopoietic stem cells. BMC Cell Biol 7:30.:30.

16. Kornfeld R, Kornfeld S. (1985). Assembly of asparagine-linked oligosaccharides. Annu Rev Biochem 54:631-664.

17. Schachter H. (1991). The ‘yellow brick road’ to branched complex N-glycans. Glycobiology 1:453-461.

18. Mollicone R, Gibaud A, Francois A, Ratcliffe M and Oriol R. (1990). Acceptor specificity and tissue distribution of three human alpha-3-fucosyltransferases. Eur J Biochem 191:169-176.

19. Mollicone R, Candelier J J, Mennesson B, Couillin P, Venot A P and Oriol R. (1992). Five specificity patterns of (1-3)-alpha-L-fucosyltransferase activity defined by use of synthetic oligosaccharide acceptors. Differential expression of the enzymes during human embryonic development and in adult tissues. Carbohydr Res 228:265-276.

20. Miyoshi E, Noda K, Yamaguchi Y, Inoue S, Ikeda Y, Wang W, Ko J H, Uozumi N, Li W and Taniguchi N. (1999). The alpha1-6-fucosyltransferase gene and its biological significance. Biochim Biophys Acta 1473:9-20.

21. Herscovics A. (2001). Structure and function of Class I alpha 1,2-mannosidases involved in glycoprotein synthesis and endoplasmic reticulum quality control. Biochimie 83:757-762.

22. Lal A, Pang P, Kalelkar S, Romero P A, Herscovics A and Moremen K W. (1998). Substrate specificities of recombinant murine Golgi alpha1,2-mannosidases IA and IB and comparison with endoplasmic reticulum and Golgi processing alpha1,2-mannosidases. Glycobiology 8:981-995.

23. Yoshida A, Minowa M T, Takamatsu S, Hara T, Oguri S, Ikenaga H and Takeuchi M. (1999). Tissue specific expression and chromosomal mapping of a human UDP-N-acetylglucosamine: alpha1,3-d-mannoside beta1,4-N-acetylglucosaminyltransferase. Glycobiology 9:303-310.

24. Forde S P, Jorgensen T B, Newey S E, Roubelakis M, Smythe J, McGuckin C P, Pettengell R and Watt S M. (2006). Endolyn (CD164) Modulates the CXCL12-Mediated Migration of Umbilical Cord Blood CD133+Cells. Blood.:

25. Doyonnas R, Yi-Hsin C J, Butler L H, Rappold I, Lee-Prudhoe J E, Zannettino A C, Simmons P J, Buhring H J, Levesque J P and Watt S M. (2000). CD164 monoclonal antibodies that block hemopoietic progenitor cell adhesion and proliferation interact with the first mucin domain of the CD164 receptor. J Immunol 165:840-851.

26. Watt S M, Buhring H J, Rappold I, Chan J Y, Lee-Prudhoe J, Jones T, Zannettino A C, Simmons P J, Doyonnas R, Sheer D and Butler L H. (1998). CD164, a novel sialomucin on CD34(+) and erythroid subsets, is located on human chromosome 6q21. Blood 92:849-866.

27. Zannettino A C, Buhring H J, Niutta S, Watt S M, Benton M A and Simmons P J. (1998). The sialomucin CD164 (MGC-24v) is an adhesive glycoprotein expressed by human hematopoietic progenitors and bone marrow stromal cells that serves as a potent negative regulator of hematopoiesis. Blood 92:2613-2628.

28. Lo N W, Shaper J H, Pevsner J and Shaper N L. (1998). The expanding beta 4-galactosyltransferase gene family: messages from the databanks. Glycobiology 8:517-526.

29. Almeida R, Amado M, David L, Levery S B, Holmes E H, Merkx G, van Kessel A G, Rygaard E, Hassan H, Bennett E and Clausen H. (1997). A family of human beta4-galactosyltransferases. Cloning and expression of two novel UDP-galactose:beta-n-acetylglucosamine beta1,4-galactosyltransferases, beta4Gal-T2 and beta4Gal-T3. J Biol Chem 272:31979-31991.

30. Asano M, Furukawa K, Kido M, Matsumoto S, Umesaki Y, Kochibe N and Iwakura Y. (1997). Growth retardation and early death of beta-1,4-galactosyltransferase knockout mice with augmented proliferation and abnormal differentiation of epithelial cells. EMBO J 16:1850-1857.

31. Schwartz-Albiez R, Merling A, Martin S, Haas R and Gross H J. (2004). Cell surface sialylation and ecto-sialyltransferase activity of human CD34 progenitors from peripheral blood and bone marrow. Glycoconj J 21:451-459.

32. Ghosh S, Bandulet C and Nitschke L. (2006). Regulation of B cell development and B cell signalling by CD22 and its ligands alpha2,6-linked sialic acids. Int Immunol 18:603-611.

33. Amano M, Galvan M, He J and Baum L G. (2003). The ST6Gal I sialyltransferase selectively modifies N-glycans on CD45 to negatively regulate galectin-1-induced CD45 clustering, phosphatase modulation, and T cell death. J Biol Chem 278:7469-7475.

34. Kiss J, Kunstar A, Fajka-Boja R, Dudics V, Tovari J, Legradi A, Monostori E and Uher F. (2007). A novel anti-inflammatory function of human galectin-1: inhibition of hematopoietic progenitor cell mobilization. Exp Hematol 35:305-313.

35. Kadri T, Lataillade J J, Doucet C, Marie A, Ernou I, Bourin P, Joubert-Caron R, Caron M and Lutomski D. (2005). Proteomic study of Galectin-1 expression in human mesenchymal stem cells. Stem Cells Dev 14:204-212.

36. Robinson S N, Ng J, Niu T, Yang H, McMannis J D, Karandish S, Kaur I, Fu P, Del Angel M, Messinger R, Flagge F, de Lima M, Decker W, Xing D, Champlin R and Shpall E J. (2006). Superior ex vivo cord blood expansion following co-culture with bone marrow-derived mesenchymal stem cells. Bone Marrow Transplant 37:359-366.

37. Okajima T, Fukumoto S, Miyazaki H, Ishida H, Kiso M, Furukawa K, Urano T and Furukawa K. (1999). Molecular cloning of a novel alpha 2,3-sialyltransferase (ST3Gal VI) that sialylates type II lactosamine structures on glycoproteins and glycolipids. J Biol Chem 274:11479-11486.

38. Dercksen M W, Gerritsen W R, Rodenhuis S, Dirkson M K, Slaper-Cortenbach I C, Schaasberg W P, Pinedo H M, dem Borne A E and van der Schoot C E. (1995). Expression of adhesion molecules on CD34+ cells: CD34+ L-selectin+ cells predict a rapid platelet recovery after peripheral blood stem cell transplantation. Blood 85:3313-3319.

39. Dimitroff C J, Lee J Y, Fuhlbrigge R C and Sackstein R. (2000). A distinct glycoform of CD44 is an L-selectin ligand on human hematopoietic cells. Proc Natl Acad Sci USA 97:13841-13846.

40. Dimitroff C J, Lee J Y, Rafii S, Fuhlbrigge R C and Sackstein R. (2001). CD44 is a major E-selectin ligand on human hematopoietic progenitor cells. J Cell Biol 153:1277-1286.

41. Skelton T P, Zeng C, Nocks A and Stamenkovic I. (1998). Glycosylation provides both stimulatory and inhibitory effects on cell surface and soluble CD44 binding to hyaluronan. J Cell Biol 140:431-446.

42. Aruffo A, Stamenkovic I, Melnick M, Underhill C B and Seed B. (1990). CD44 is the principal cell surface receptor for hyaluronate. Cell 61:1303-1313.

43. Peled A, Grabovsky V, Habler L, Sandbank J, Arenzana-Seisdedos F, Petit I, Ben Hur H, Lapidot T and Alon R. (1999). The chemokine SDF-1 stimulates integrin-mediated arrest of CD34(+) cells on vascular endothelium under shear flow. J Clin Invest 104:1199-1211.

44. Sasaki K, Kurata K, Funayama K, Nagata M, Watanabe E, Ohta S, Hanai N and Nishi T. (1994). Expression cloning of a novel alpha 1,3-fucosyltransferase that is involved in biosynthesis of the sialyl Lewis x carbohydrate determinants in leukocytes. J Biol Chem % 20;269:14730-14737.

45. Zerfaoui M, Fukuda M, Sbarra V, Lombardo D and El Battari A. (2000). alpha(1,2)-facosylation prevents sialyl Lewis x expression and E-selectin-mediated adhesion of fucosyltransferase VII-transfected cells. Eur J Biochem 267:53-61.

46. Bengtson P, Lundblad A, Larson G and Pahlsson P. (2002). Polymorphonuclear leukocytes from individuals carrying the G329A mutation in the alpha 1,3-fucosyltransferase VII gene (FUT7) roll on E- and P-selectins. J Immunol 169:3940-3946.

47. Hemmoranta H, Hautaniemi S, Niemi J, Nicorici D, Laine J, Yli-Harja O, Partanen J and Jaatinen T. (2006). Transcriptional profiling reflects shared and unique characters for CD34+ and CD133+ cells. Stem Cells Dev 15:839-851.

48. Bause E, Bieberich E, Rolfs A, Volker C and Schmidt B. (1993). Molecular cloning and primary structure of Man9-mannosidase from human kidney. Eur J Biochem 217:535-540.

49. Misago M, Liao Y F, Kudo S, Eto S, Mattei M G, Moremen K W and Fukuda M N. (1995). Molecular cloning and expression of cDNAs encoding human alpha-mannosidase II and a previously unrecognized alpha-mannosidase IIx isozyme. Proc Natl Acad Sci USA 92:11766-11770.

50. Moremen K W, Robbins P W. (1991). Isolation, characterization, and expression of cDNAs encoding murine alpha-mannosidase II, a Golgi enzyme that controls conversion of high mannose to complex N-glycans. J Cell Biol 115:1521-1534.

51. Nebes V L, Schmidt M C. (1994). Human lysosomal alpha-mannosidase: isolation and nucleotide sequence of the full-length cDNA. Biochem Biophys Res Commun 200:239-245.

52. Saito S, Aoki H, Ito A, Ueno S, Wada T, Mitsuzuka K, Satoh M, Arai Y and Miyagi T. (2003). Human alpha 2,3-sialyltransferase (ST3Gal II) is a stage-specific embryonic antigen-4 synthase. J Biol Chem 278:26474-26479.

53. Tremblay L O, Campbell D N and Herscovics A. (1998). Molecular cloning, chromosomal mapping and tissue-specific expression of a novel human alpha1,2-mannosidase gene involved in N-glycan maturation. Glycobiology 8:585-595.

54. Tremblay L O, Herscovics A. (1999). Cloning and expression of a specific human alpha 1,2-mannosidase that trims Man9GlcNAc2 to Man8GlcNAc2 isomer B during N-glycan biosynthesis. Glycobiology 9:1073-1078.

55. Amado M, Almeida R, Carneiro F, Levery S B, Holmes E H, Nomoto M, Hollingsworth M A, Hassan H, Schwientek T, Nielsen P A, Bennett E P and Clausen H. (1998). A family of human beta3-galactosyltransferases. Characterization of four members of a UDP-galactose:beta-N-acetyl-glucosamine/beta-nacetyl-galactosamine beta-1,3-galactosyltransferase family. J Biol Chem 273:12770-12778.

56. Bai X, Zhou D, Brown J R, Crawford B E, Hennet T and Esko J D. (2001). Biosynthesis of the linkage region of glycosaminoglycans: cloning and activity of galactosyltransferase II, the sixth member of the beta 1,3-galactosyltransferase family (beta 3GalT6). J Biol Chem 276:48189-48195.

57. Isshiki S, Togayachi A, Kudo T, Nishihara S, Watanabe M, Kubota T, Kitajima M, Shiraishi N, Sasaki K, Andoh T and Narimatsu H. (1999). Cloning, expression, and characterization of a novel UDP-galactose:beta-N-acetylglucosamine beta1,3-galactosyltransferase (beta3Gal-T5) responsible for synthesis of type 1 chain in colorectal and pancreatic epithelia and tumor cells derived therefrom. J Biol Chem 274:12499-12507.

58. Kolbinger F, Streiff M B and Katopodis A G. (1998). Cloning of a human UDP-galactose:2-acetamido-2-deoxy-D-glucose 3beta-galactosyltransferase catalyzing the formation of type 1 chains. J Biol Chem 273:433-440.

59. Okajima T, Nakamura Y, Uchikawa M, Haslam D B, Numata S I, Furukawa K, Urano T and Furukawa K. (2000). Expression cloning of human globoside synthase cDNAs. Identification of beta 3Gal-T3 as UDP-N-acetylgalactosamine:globotriaosylceramide beta 1,3-N-acetylgalactosaminyltransferase. J Biol Chem 275:40498-40503.

60. Zhou D, Henion T R, Jungalwala F B, Berger E G and Hennet T. (2000). The beta 1,3-galactosyltransferase beta 3GalT-V is a stage-specific embryonic antigen-3 (SSEA-3) synthase. J Biol Chem 275:22631-22634.

61. Almeida R, Levery S B, Mandel U, Kresse H, Schwientek T, Bennett E P and Clausen H. (1999). Cloning and expression of a proteoglycan UDP-galactose:beta-xylose beta1,4-galactosyltransferase 1. A seventh member of the human beta4-galactosyltransferase gene family. J Biol Chem 274:26165-26171.

62. Amado M, Almeida R, Schwientek T and Clausen H. (1999). Identification and characterization of large galactosyltransferase gene families: galactosyltransferases for all functions. Biochim Biophys Acta 1473:35-53.

63. Okajima T, Yoshida K, Kondo T and Furukawa K. (1999). Human homolog of Caenorhabditis elegans sqv-3 gene is galactosyltransferase I involved in the biosynthesis of the glycosaminoglycan-protein linkage region of proteoglycans. J Biol Chem 274:22915-22918.

64. Schwientek T, Almeida R, Levery S B, Holmes E H, Bennett E and Clausen H. (1998). Cloning of a novel member of the UDP-galactose:beta-N-acetylglucosamine beta1,4-galactosyltransferase family, beta4Gal-T4, involved in glycosphingolipid biosynthesis. J Biol Chem 273:29331-29340.

65. van D, I, van Tetering A, Schiphorst W E, Sato T, Furukawa K and van den Eijnden D H. (1999). The acceptor substrate specificity of human beta4-galactosyltransferase V indicates its potential function in O-glycosylation. FEBS Lett 450:52-56.

66. Grundmann U, Nerlich C, Rein T and Zettlmeissl G. (1990). Complete cDNA sequence encoding human beta-galactoside alpha-2,6-sialyltransferase. Nucleic Acids Res 18:667.

67. Takashima S, Tsuji S and Tsujimoto M. (2002). Characterization of the second type of human beta-galactoside alpha 2,6-sialyltransferase (ST6Gal II), which sialylates Galbeta 1,4GlcNAc structures on oligosaccharides preferentially. Genomic analysis of human sialyltransferase genes. J Biol Chem 277:45719-45728.

68. Gillespie W, Kelm S and Paulson J C. (1992). Cloning and expression of the Gal beta 1, 3GalNAc alpha 2,3-sialyltransferase. J Biol Chem 267:21004-21010.

69. Kitagawa H, Paulson J C. (1994). Cloning of a novel alpha 2,3-sialyltransferase that sialylates glycoprotein and glycolipid carbohydrate groups. J Biol Chem 269:1394-1401.

70. Kitagawa H, Mattei M G and Paulson J C. (1996). Genomic organization and chromosomal mapping of the Gal beta 1,3GalNAc/Gal beta 1,4GlcNAc alpha 2,3-sialyltransferase. J Biol Chem 271:931-938.

71. Kono M, Takashima S, Liu H, Inoue M, Kojima N, Lee Y C, Hamamoto T and Tsuji S. (1998). Molecular cloning and functional expression of a fifth-type alpha 2,3-sialyltransferase (mST3Gal V: GM3 synthase). Biochem Biophys Res Commun 253:170-175.

72. Larsen R D, Ernst L K, Nair R P and Lowe J B. (1990). Molecular cloning, sequence, and expression of a human GDP-L-fucose:beta-D-galactoside 2-alpha-L-fucosyltransferase cDNA that can form the H blood group antigen. Proc Natl Acad Sci USA 87:6674-6678.

73. Koda Y, Kimura H and Mekada E. (1993). Analysis of Lewis fucosyltransferase genes from the human gastric mucosa of Lewis-positive and -negative individuals. Blood 82:2915-2919.

74. Kelly R J, Rouquier S, Giorgi D, Lennon G G and Lowe J B. (1995). Sequence and expression of a candidate for the human Secretor blood group alpha(1,2)fucosyltransferase gene (FUT2). Homozygosity for an enzyme-inactivating nonsense mutation commonly correlates with the non-secretor phenotype. J Biol Chem 270:4640-4649.

75. Couillin P, Mollicone R, Grisard M C, Gibaud A, Ravise N, Feingold J and Oriol R. (1991). Chromosome 11 q localization of one of the three expected genes for the human alpha-3-fucosyltransferases, by somatic hybridization. Cytogenet Cell Genet 56:108-111.

76. Weston B W, Nair R P, Larsen R D and Lowe J B. (1992). Isolation of a novel human alpha (1,3)fucosyltransferase gene and molecular comparison to the human Lewis blood group alpha (1,3/1,4)fucosyltransferase gene. Syntenic, homologous, nonallelic genes encoding enzymes with distinct acceptor substrate specificities. J Biol Chem 267:4152-4160.

77. Koszdin K L, Bowen B R. (1992). The cloning and expression of a human alpha-1,3 fucosyltransferase capable of forming the E-selectin ligand. Biochem Biophys Res Commun 187:152-157.

78. Natsuka S, Gersten K M, Zenita K, Kannagi R and Lowe J B. (1994). Molecular cloning of a cDNA encoding a novel human leukocyte alpha-1,3-fucosyltransferase capable of synthesizing the sialyl Lewis x determinant. J Biol Chem 269:16789-16794.

79. Yamaguchi Y, Fujii J, Inoue S, Uozumi N, Yanagidani S, Ikeda Y, Egashira M, Miyoshi O, Niikawa N and Taniguchi N. (1999). Mapping of the alpha-1,6-fucosyltransferase gene, FUT8, to human chromosome 14q24.3. Cytogenet Cell Genet 84:58-60.

80. Kaneko M, Kudo T, Iwasaki H, Ikehara Y, Nishihara S, Nakagawa S, Sasaki K, Shiina T, Inoko H, Saitou N and Narimatsu H. (1999). Alpha1,3-fucosyltransferase IX (Fuc-TIX) is very highly conserved between human and mouse; molecular cloning, characterization and tissue distribution of human Fuc-TIX. FEBS Lett 452:237-242.

### Example 19

### Evaluation of Cord Blood CD133+ and CD133− Cell Associated N-Glycans

N-glycan profile data was characterized from human cord blood hematopoietic CD 133+ and CD133− cells as described in Example 45. The data was evaluated according to the relative association of each glycan signal to either cell type as described in the legends of Tables 22 and 23, and sorted accordingly into CD133+ and CD133− associated glycan signals in Tables 22 and 23 for neutral and sialylated N-glycan signals, respectively. In this calculation, three groups of glycan signals were obtained for each cell type: over 2-fold difference (significant association), between 2 and 1.5-fold difference (substantial association), and below 1.5-fold difference (small but detected association). The data demonstrated that in addition to glycan signal groups identified in Example 45, also the other glycan signals were associated with either CD133+ or CD133− cells.

### Example 20

### Examples of Cell Sample Production

Cord Blood Derived Mesenchymal Stem Cell Lines

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400g/40 min) The mononuclear cell fragment was collected from the gradient and washed twice with PBS.

Umbilical cord blood cell isolation and culture. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http://www.invitrogen.com) 40% MCDB-201 (Sigma-Aldrich) 2% fetal calf serum (FCS), 1× penicillin-streptomycin (both form Gibco), 1× ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/cm². In later passages the cells were detached with trypsin/EDTA from defined area at defined time points, counted with hematocytometer and replated at density of 2000-3000 cells/cm².

Bone Marrow Derived Mesenchymal Stem Cell Lines

Isolation and culture of bone marrow derived stem cells. Bone marrow (BM)—derived MSCs were obtained as described by Leskelä et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

Experimental Procedures

Flow cytometric analysis of mesenchymal stem cell phenotype. Both UBC and BM derived mesenchymal stem cells were phenotyped by flow cytometry (FACSCalibur, Becton Dickinson). Fluorescein isothicyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abcam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD14, CD34, CD45 and HLA-DR and positive for CD13, CD29, CD44, CD90, CD105 and HLA-ABC.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/m1 insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Osteogenic differentiation. To induce the osteogenic differentiation of the BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

Cell harvesting for glycome analysis. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times.

The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

### Example 21

Experimental Procedures

Extraction of mononuclear cells (MNCs) from umbilical cord blood. Human term umbilical cord blood (CB) units were collected after delivery with informed consent of the mothers and the CB was processed within 24 hours of the collection. The mononuclear cells (MNCs) were isolated from each CB unit diluting the CB 1:1 with phosphate-buffered saline (PBS) followed by Ficoll-Paque Plus (Amersham Biosciences, Uppsala, Sweden) density gradient centrifugation (400×g/40 min). The mononuclear cell fragment was collected from the gradient and washed twice with PBS.

Depletion of red blood cell precursors by magnetic microbeads conjugated with anti-Glycophorin A (anti-CD235a). MNCs (10⁷) were suspended in 80 μl of 0.5% ultra pure BSA, 2 mM EDTA-PBS buffer. Red blood cell precursors were depleted with magnetic microbeads conjugated with anti-CD235a (Glycophorin a, Miltenyi Biotec) by adding 20 μl of magnetic microbead suspension/10⁷ cells and by incubating for 15 min at 4° C. Cell suspension was washed with 1-2 ml of buffer/10⁷ cells followed by centrifugation at 300×g for 10 min. Cells were resuspended 1,25×10⁸ cells/500 μl of buffer. MACS LD column (Miltenyi Biotec) was placed in a magnetic field and rinsed with 2 ml of buffer. Cell suspension was applied to the column and cells passing through were collected. Column was washed two times with 1 ml of buffer and total effluent was collected. Cells were centrifuged for 10 min at 300×g and resuspended in 10 ml of buffer. All together eight CB units were used for following antibody staining

Staining with anti-glycan antibodies. MNCs were aliquoted to FACS tubes in a small volume, i.e. 0.5×10⁶ cells/500 μl of 0,3% ultra pure BSA (Sigma), 2mM EDTA-PBS buffer. Ten microliters of primary antibody (list of primary antibodies is presented in Table 25) was added to cell suspension, vortexed and cells were incubated for 30 min at room temperature. Cells were washed with 2 ml of buffer and centrifuged at 500×g for 5 min. AlexaFluor 488-conjugated anti-mouse (1:500, Invitrogen) and anti-rabbit (1:500, Molecular Probes) and FITC-conjugated anti-rat (1:320, Sigma) secondary antibodies were used for appropriated primary antibodies. Secondary antibodies were diluted in 0.3% ultra pure BSA, 2mM EDTA-PBS buffer and 200 μl of dilution was added to the cell suspension. Samples were incubated for 30 min at room temperature in the dark. Cells were washed with 2 ml of buffer and centrifuged at 500×g for 5 min. As a negative control cells were incubated without primary antibody and otherwise treated similarly to labelled cells.

Double staining with PE-conjugated anti-CD34-antibody. After staining with anti-glycan antibodies, a double staining with PE-conjugated anti-CD34 antibody (BD Biosciences) was performed. Cells were suspended in 500 μl of buffer and 10 μl of anti-CD34 antibody was added and incubated for 30 min at +4° C. in dark. After incubation cells were washed with 2 ml of buffer and centrifugation at 500×g for 5 min. Supernatant was removed and cells were resuspended in 300 μl of buffer and stored at 4° C. overnight in the dark.

Flow cytometric analysis. The next day cells were analysed with flow cytometer BD FACSAria (BD Biosciences) using FITC and PE detectors. Approximately 250 000-300 000 cells were counted for each anti-glycan antibody. Data was analysed with BD FACSDiva Software version 5.0.2 (BD Biosciences).

Results and Discussion

Results from CB-HSC FACS analysis are shown in FIG. 47 and Table 24 and antibodies are indicated in Table 25. Some glycan structures, e.g. Tn, TF, Lewis x and sialyl Lewis x, are enriched in HSCs (CD34+) when compared to mature blood cells (CD34−). This was shown with several anti-glycan antibodies against same epitope and even between different CB units. The highest variations were observed with anti-Lex antibodies between distinct CB units. The glycan structures enriched with mature blood cells (CD34−) were asialo GM1, asialo GM2, Globoside GL4 and Lewis a.

### Example 22

### Antibody Profiling of Bone Marrow Derived and Cord Blood Derived Mesenchymal Stem Cell Lines

Experimental Procedures

Bone marrow derived mesenchymal stem cell lines (BM-MSC). Isolation and culture of BM-MSCs, as well as osteogenic differentiation of BM-MSCs, were performed as described in previous examples.

Umbilical cord blood mesenchymal stem cell (CB-MSC) isolation and culture. The isolation and culture of CB-MSCs was performed as described in Example 1 with some modifications. Osteogenic differentiation of CB-MSCs was induced as described for BM-MSCs for 16 days.

Adipogenic differentiation of CB-MSCs. Cells were grown in proliferation medium to almost confluence after which the adipogenic induction medium including α-MEM Glutamax supplemented with 10% FCS, 20 mM Hepes, 1× penicillin-streptomycin, 0.1 mM Indomethasin (all from Sigma), 0.5 mM IBMX-22, 0.4 μg/ml dexamethasone and 0.5 μg/ml Insulin (all three from Promocell) was added. After 3 days, terminal adipogenic differentiation medium including α-MEM Glutamax supplemented with 10% FCS, 20 mM Hepes, 1× penicillin-streptomycin, 0.1 mM Indomethasin (all from Sigma), 0.5 μg/ml Insulin and 3.0 μg/ml Ciglitazone (both two from Promocell) was added and cells were grown for 14 days (altogether 17 days) in 5% CO₂ at 37° C. Differentiation medium was refreshed twice a week throughout the differentiation period.

Flow cytometric analysis of mesenchymal stem cell phenotype. Both BM and CB derived MSCs were phenotyped by flow cytometry (BD FACSAria, Becton Dickinson). FITC, APC or PE conjugated antibodies against CD13, CD14, CD29, CD34, CD44, CD45, CD49e, CD73, CD90, HLA-DR and HLA-ABC (all from BD Biosciences) and CD105 (Abeam Ltd.) were used for direct labelling. For staining, cells in a small volume, i.e. 5×10⁴ cells/100 μl 0.3% ultra pure BSA, 2 mM EDTA-PBS buffer, were aliquoted to FACS-tubes. One microliter of each antibody was added to cells and incubated for 30 min at +4° C. Cells were washed with 2 ml of buffer and centrifuged at 300×g for 4 min. Cells were suspended in 200 μl of buffer for flow cytometric analysis.

Cell harvesting for antibody staining. Both BM and CB-MSCs were detached from cell culture plates with 2 mM EDTA-PBS solution (Versene), pH 7.4, for approximately 30 minutes at 37° C. Both osteogenic and adipogenic cells were detached with 10 mM EDTA-PBS solution, pH 7.4, for 30 minutes and 5 minutes at 37° C., respectively. Since the differentiated cells detached from culture plates as clusters, they were suspended by pipetting with Pasteur-pipette or by vortexing and by suspending through an 18 gauge needle to get a single cell suspension. Finally, the cell suspension was filtered through a 50 μm filter to get rid of unsuspended cell aggregates. Harvested cells were centrifuged at 300×g for 4 minutes and suspended for small volume of 0.3% ultra pure BSA (Sigma), 2 mM EDTA-PBS buffer.

Primary antibody staining. BM and CB derived cells were aliquoted to FACS-tubes in a small volume, i.e. 5-7×10⁴ cells/100 μl 0.3% ultra pure BSA, 2mM EDTA-PBS buffer. Four microliters of anti-glycan primary antibody was added to cell suspension, vortexed and incubated for 30 min at room temperature. Cells were washed with 2 ml of buffer and centrifuged for 4 min at 300×g, after which the supernatant was removed. Primary antibodies used for staining are listed in Table 26.

Secondary antibody staining. AlexaFluor 488-conjugated anti-mouse (1:500, Invitrogen) and anti-rabbit (1:500, Molecular Probes), as well as FITC-conjugated anti-rat (1:320, Sigma) and anti-human λ, (1:1000, Southern Biotech) secondary antibodies were used for appropriate primary antibodies. Secondary antibodies were diluted in 0.3% ultra pure BSA, 2mM EDTA-PBS buffer and 100 μl of dilution was added to the cell suspension. Samples were incubated for 30 min at room temperature in the dark. Cells were washed with 2 ml of buffer and centrifuged for 4 min at 300×g. Supernatant was removed and cells were suspended in 200 μl of buffer for flow cytometric analysis. As a negative control cells were incubated without primary antibody and otherwise treated similarly to labelled cells.

Flow cytometric analysis. Cells with fluorescently labelled antibodies were analysed with BD FACSAria (Becton Dickinson) using FITC detector at wavelength 488. Results were analysed with BD FACSDiva software version 5.0.1 (Becton Dickinson).

Results and Discussion

Flow cytometric analysis of mesenchymal stem cell phenotype. Both BM and CB-MSCs were negative for hematopoietic markers CD34, CD45 and CD14. The cells stained positively for the CD13 (aminopeptidase N), CD29 (β1-integrin), CD44 (hyaluronan receptor), CD73 (SH3), CD90 (Thy-1), CD105 (SH2/endoglin) and CD49e. The cells stained also positively for HLA-ABC, but negatively for HLA-DR.

Anti-glycan antibody profiling of BM-MSCs. BM-MSCs and osteogenic cells (BM-OG) differentiated thereof were analyzed with up to 60 anti-glycan antibodies by flow cytometry and also with 29 antibodies by immunohistochemistry (IHC). The results of BM-MSC staining are presented in Table 26 and in Figures.

The most prominent enrichment in stem cells is SSEA-4 and in osteogenic cells some glycolipid epitopes such as ganglioseries asialo GM1 and asialo GM2; globoseries structures globotriasyl ceramide Gb3 and globotetraose also known as globoside (GL4 or Gb4); as well as Lewis a and sialylated Cal5-3.

Lewis x structures seems not to be present in quantity over detection level under FACS analysis conditions in a major part of the MSCs in the preparations of MSCs or in differentiated cells based on staining with 5 different anti-Lex antibodies. There is however specific Lewis x expression recognizable by specific anti-Lewis x clones.

On the other hand, sialyl Lewis x structures are present on both stem cells and in osteogenic cells and the proportions differ between different anti-sLex antibodies, which is most probably due to the different carriers for sLex epitopes. For example GF526 anti-sLex antibody recognizes only sLex epitope carried by a specific O-glycan core II structure. The binding of GF526 has been determined to be related to P-selectin ligand glycoprotein PSGL-1, which represents the O-glycan effectively in large quantities on certain non-stem cell materials. It is however realised that core II O-glycans have been reported on several mucin type O-glycans and the present invention is not limited to analysis of the Core II sLex on PSGL-1 on the mesenchymal stem cells. The carrier and the exact binding epitope of sLex recognized by two other anti-sLex antibodies (GF516 and GF307) appears to include structures other than core II with optimal fine specificity different from the core two including polylactosamines with β3Gal elongation The antibodies with different fine and core/carrier glycan specifiy cell populations of different sizes.

Anti-glycan antibody profiling of CB-MSCs. CB-MSCs and both osteogenic and adipocytic cells differentiated thereof were analysed with up to 61 different anti-glycan antibodies by flow cytometry. The results of CB-MSC staining are presented in Tables and in Figures. Likewise in BM derived antibody profiling, there seems not to be a single specific glycan epitope determining either CB-MSCs or cells differentiated into osteogenic or adipocytic lineages. Some glycans, e.g. H disaccharide (GF394), TF (GF281), Glycodelin (GF375), Lewis x (GF517) and Galα3Gal (GF413), are highly enriched in CB derived MSCs, but their proportion in the whole stem cell population is rather low (10% or below). Interestingly, there seems to be also glycans, e.g. SSEA-4 (GF354), Lewis c (GF295), SSEA-3 (VPU009), GD2 (GF406), sialyl Lewis x (GF307) and Tra-1-60 (GF415), enriched in stem cells and in adipocytic cells, but not in osteogenic cells. BM-derived cells have not been differentiated into adipocytic direction, so we can not compare the data between different adipocytes from different sources. Osteogenic differentiation induces similar enrichment of glycans both in BM and CB derived cells. Only Gb3, increasing in BM derived osteogenic cells is not increased in CB derived osteogenic cells. Furthermore, gangliosides GT1b, GD2, GD3 and A2B5, not tested in BM-derived cells, are highly enriched in CB derived osteogenic cells. Most of the glycan epitopes revealed by specific antibodies of the example enriched in CB-derived osteoblasts are also enriched (even with higher percentage) in CB-derived adipocytes, but the invention reveals even for these targets that there are differences in expression levels between the cell types allowing characterization of both differentiation lineages. An interesting group of glycan epitopes after differentiation is glycan epitopes recognizable by known antibodies against gangliosides, in general increasing from stem cells (<10%) into osteoblasts and adipocytes (50-100%). Unlike in BM-derived MSCs, there seems to be some positivity with anti-Lewis x antibodies GF517 and GF525 in CB derived cells. The results with anti-sialyl-Lewis x antibodies are parallel with both cell types.

TABLE 1

| Reagent | Target | FES 22 | FES 30 | mEF | % stain |
|---|---|---|---|---|---|
| FITC-PSA | α-Man | − | − | + | |
| FITC-RCA | β-Gal (Galβ4GlcNAc) | + | − | +/− | |
| FITC-PNA | β-Gal (Galβ3GalNAc) | + | + | − | |
| FITC-MAA | α2,3-sialyl-LN | + | + | − | |
| FITC-SNA | α2,6-sialyl-LN | + | n.d. | + | |
| FITC-PWA | I-antigen | + | + | n.d. | |
| FITC-STA | i-antigen | + | − | + | |
| FITC-WFA | β-GalNAc | + | + | − | |
| NeuGc-PAA-biotin | NeuGc-lectin | + | + | + | |
| anti-GM3(Gc) mAb | NeuGcα3Galβ4Glc | + | + | + | |
| FITC-LTA | α-Fuc | + | − | + | |
| FITC-UEA | α-Fuc | + | − | + | |
| mAb Lex | Lewis^{x} | + | n.d. | − | |
| mAb sLex | sialyl-Lewis^{x} | + | n.d. | − | |
| GF 279 Le c | Galβ3GlcNAc | | + | − | 95-100 |
| GF 283 Le b | | | + | − | 20-35 |
| GF 284 H Type 2 | | | + | − | 15-20 |
| GF 285 H Type 2 | | | − | + | 95-100 |
| GF 286 H Type 2 | | | + | − | 10-20 |
| GF 287 H Type 1 | | | + | − | 90-100 |
| GF 288 Globo-H | | | + | − | 20-35 |
| GF 289 Ley | | | − | + | 95-100 |
| GF 290 H Type 2 | | | + | − | 20-35 |

+, specific binding.
−, no specific binding.
n.d., not determined.
% of stain means approximate percentage of cell stained with a binder.

| | | BM- | | |
|---|---|---|---|---|
| Code | Antigen | MSC | Osteog. | Change |
| GF275 | CA15-3 (Cancer antigen 15-3; sialylated carbohydrate epitope of the MUC-1 | +* | + | |
| | glycoprotein) | | | |
| GF276 | oncofetal antigen, tumor associated glycoprotein (TAG-72) or CA 72-4 | −* | + | ↑↑ |
| GF277 | human sialosyl-Tn antigen (STn, sCD175) | (+)* | + | ↑ |
| GF278 | human Tn antigen (Tn, CD175 B1.1) | (+)* | + | ↑ |
| GF295 | Blood group antigen precursor (BG1), Lewis c Gb3GN (pLN) | − | − | |
| GF280 | TF-antigen isoform (Nemod TF2) | −* | − | |
| GF281 | TF-antigen isoform (A68-E/E3) | −* | − | |
| GF296 | asialoganglioside GM1 | − | − | |
| GF297 | Globoside GL4 | + | + | |
| GF298 | Human CD77 (=blood group substance pk), GB3 | + | + | |
| GF299 | Forssman antigen, glycosphingolipid (FO GSL) differentiation ag | − | − | |
| GF300 | Asialo GM2 | − | − | |
| GF301 | Lewis b blood group antigen | −* | − | |
| GF302 | H type 2 blood group antigen | +* | + | |
| GF303 | Blood group H1(O) antigen (BG4) | −* | + | ↑↑ |
| GF288 | Globo-H | −* | − | |
| GF304 | Lewis a | − | − | |
| GF305 | Lewis x, CD15, 3-FAL, SSEA-1,3-fucosyl-N-acetyllactosamine | (+/−) | − | ↓ |
| GF306 | Sialyl Lewis a | − | − | |
| GF307 | Sialyl Lewis x | + | (+/−) | ↓ |
| GF353 | SSEA-3 (stage-specific embryonic antigen-3) | + | (+/−) | ↓↓ |
| GF354 | SSEA-4 (stage-specific embryonic antigen-4) | +* | − | ↓↓ |
| GF355 | Galactose-alpha(1,3)galactose | NT | NT | |
| GF365 | Nemod TF1, DC176, Galbeta1-3GalNAc | − | − | |

+ = positive,
(+) = weak positive,
(+/−) = single positive cells,
− = negative;
NT = not tested;
*= result has been confirmed by FACS analysis

See also Example 8.

TABLE 2

| Lectins | Target | % of positive cells |
|---|---|---|
| FITC-GNA | α-Man | 27.8 |
| FITC-HHA | α-Man | 95.3 |
| FITC-PSA | α-Man | 95.5 |
| FITC-RCA | β-Gal (Galβ4GlcNAc) | 94.8 |
| FITC-PNA | β-Gal (Galβ3GalNAc) | 31.1 |
| FITC-MAA | α2,3-sialylation | 89.9 |
| FITC-SNA | α2,6-sialylation | 14.3 |
| FITC-PWA | I-antigen | 1.9 |
| FITC-STA | i-antigen | 11.9 |
| FITC-LTA | α-Fuc | 2.8 |
| FITC-UEA | α-Fuc | 8.0 |

TABLE 3

| | | BM MSC | | | | | | | |
|---|---|---|---|---|---|---|---|---|---|
| | | lectin concentration, μg/ml | | | | | | | |
| Lectin | Target | 0.25 | 0.5 | 1 | 2.5 | 5 | 10 | 20 | 40 |
| FITC-GNA | α-Man | −¹⁾ | − | ++ | ++ | ++ | ++ | ++ | ++ |
| FITC-HHA | α-Man | ++ | ++ | +++ | +++ | +++ | +++ | +++ | +++ |
| FITC-PSA | α-Man | ++ | ++ | ++ | +++ | +++ | +++ | +++ | +++ |
| FITC-RCA | β-Gal (Galβ4GlcNAc) | − | − | +/− | +/− | + | + | ++ | ++ |
| FITC-PNA | β-Gal (Galβ3GalNAc) | − | − | − | − | +/− | +/− | +/− | + |
| FITC-MAA | α2,3-sialylation | − | − | − | +/− | + | ++ | ++ | ++ |
| FITC-SNA | α2,6-sialylation | − | − | − | − | +/− | +/− | + | + |
| FITC-PWA | l-antigen | − | − | − | − | − | − | +/− | +/− |
| FITC-STA | i-antigen | − | − | − | − | − | +/− | +/− | +/− |
| FITC-LTA | α-Fuc | − | − | − | − | − | − | − | − |
| FITC-UEA | α-Fuc | − | − | − | +/− | +/− | + | ++ | ++ |
| FITC-MBL | α-Man/β-GlcNAc | − | − | − | − | − | − | +/− | + |

¹⁾Grading of staining/labelling:
+++ very intense,
++ intense,
+ low,
+/− barely detectable,
− not labelled.

TABLE 4
Comparison of lectin ligand profile in hESCs and MEFs

| Lectin | hESC | MEF |
|---|---|---|
| PSA | − | + |
| MAA | + | − |
| PNA | + | − |
| RCA | + | + |

+ present in cell surface
− not present in cell surface

TABLE 5
Sialylated N-glycan difference analysis.

| | | | |
|---|---|---|---|
| composition¹⁾ | m/z²⁾ | class³⁾ | fold⁴⁾ |

+++ hESC⁵⁾

| | | | |
|---|---|---|---|
| S1H7N6F2 | 2953 | CE | ∞ |
| S1H8N7F1 | 3172 | CF | ∞ |
| S1H7N6F3 | 3099 | CE | 15.67 |
| S2H4N5F1 | 2408 | CF | 5.07 |
| G2H5N4 | 2253 | C | 4.56 |
| G1H5N4 | 1946 | C | 4.50 |
| S1H5N4F2 | 2222 | CE | 3.81 |
| S2H6N4 | 2383 | C | 3.51 |
| G1H5N4F1 | 2092 | CF | 3.13 |
| S1H6N5F2 | 2587 | CE | 2.94 |
| S1G1H5N4 | 2237 | C | 2.68 |
| S1H6N4F2 | 2384 | CE | 2.42 |
| S1H5N4F3 | 2368 | CE | 2.02 |

++ hESC

| | | | |
|---|---|---|---|
| S2H5N4F1 | 2367 | CF | 1.83 |
| S3H6N5 | 2878 | C | 1.82 |
| S2H6N5F1 | 2732 | CF | 1.80 |
| S1H4N5F2 | 2263 | CE | 1.59 |

+ hESC

| | | | |
|---|---|---|---|
| S2H6N5F2 | 2879 | CE | 1.49 |
| S1H7N6F1 | 2807 | CF | 1.39 |
| S1H6N5F1 | 2441 | CF | 1.20 |
| S1H5N4 | 1930 | C | 1.17 |
| S1H5N4F1 | 2076 | CF | 1.14 |
| S1H6N5F3 | 2733 | CE | 1.11 |
| S1H6N5 | 2295 | C | 1.06 |
| S1H6N4F1 | 2238 | CF | 1.03 |

| | | | |
|---|---|---|---|
| composition | m/z | class | fold |

+ Differentiated

| | | | |
|---|---|---|---|
| S2H7N6F1 | 3098 | CF | 0.75 |
| S1H5N5F2 | 2425 | CE | 0.71 |
| S2H5N4 | 2221 | C | 0.70 |
| S1H4N3F1 | 1711 | HF | 0.69 |
| S1H4N3 | 1565 | H | 0.68 |

++ Diff

| | | | |
|---|---|---|---|
| S1H4N5F1 | 2117 | CF | 0.66 |
| S2H5N3F1 | 2164 | HF | 0.56 |
| S1H5N3 | 1727 | H | 0.52 |

+++ Diff

| | | | |
|---|---|---|---|
| S1H6N3 | 1889 | H | 0.47 |
| S2H3N3F1 | 1840 | OF | 0.30 |
| S1H4N4F1 | 1914 | CF | 0.29 |
| S1H5N3F1 | 1873 | HF | 0.28 |
| S2H2N3F1 | 1678 | OF | 0.27 |
| S2H4N3F1 | 2002 | OF | 0.20 |
| S2H5N5F1 | 2570 | CF | 0.19 |
| S1H5N5F1 | 2279 | CF | 0.17 |
| S1H5N5 | 2133 | C | 0.15 |
| S1H6N4F1Ac | 2280 | CF | 0.13 |
| S1H6N3F1 | 2035 | HF | 0 |
| S1H6N6F1 | 2644 | CF | 0 |
| S1H5N6F2 | 2482 | CE | 0 |
| S1H7N5F1Ac | 2645 | CF | 0 |
| S1H5N5F3 | 2571 | CE | 0 |

¹⁾Proposed composition wherein the monosaccharide symbols are: S, NeuAc; G, NeuGc, H, Hex; N, HexNAc; F, dHex; Ac, acetyl ester.
²⁾Calculated m/z for [M − H]− ion rounded down to next integer.
³⁾N-glycan class symbols are: H, hybrid-type or monoantennary; C, complex-type; O, other type; F, fucosylated; E, complex-fucosylated, wherein at least one fucose residue is α1,2-, α1,3- or α1,4-linked.
⁴⁾‘fold’ is calculated as the relation of glycan signal intensities in hESC compared to differentiated cell types (hESC and St.3); ∞, not detected in differentiated cells; 0, not detected in hESC.
⁵⁾Association with differentiation type based on fold calculation: + low association, ++ substantial association, +++ high association.

TABLE 6
Characteristic N-glycan signals of hESC.
Neutral N-glycans:

| | m/z | Proposed | |
|---|---|---|---|
| No. | [M + Na]⁺ | composition | Proposed classification |
| 1. | 1905.6 | H9N2 | high-mannose |
| 2. | 1419.5 | H6N2 | high-mannose |
| 3. | 1743.6 | H8N2 | high-mannose |
| 4. | 1257.4 | H5N2 | high-mannose |
| 5. | 1581.5 | H7N2 | high-mannose |
| 6. | 1079.4 | H3N2F1 | low-mannose |
| 7. | 2067.7 | H10N2 | other types (glucosylated) |
| 8. | 1095.4 | H4N2 | low-mannose |
| 9. | 933.3 | H3N2 | low-mannose |
| 10. | 1663.6 | H5N4 | complex-type |
| 11. | 1622.6 | H6N3 | hybrid/monoantennary |
| 12. | 1809.6 | H5N4F1 | complex-type |
| 13. | 1460.5 | H5N3 | hybrid/monoantennary |
| 14. | 1485.5 | H3N4F1 | complex-type; terminal |
| | | | N-acetylhexosamine (N > H) |
| 15. | 1444.5 | H4N3F1 | hybrid/monoantennary |

Sialylated N-glycans:

| | m/z | Proposed | |
|---|---|---|---|
| No. | [M − H]⁻ | composition | Proposed classification |
| 1. | 2076.7 | S1H5N4F1 | complex-type |
| 2. | 2222.8 | S1H5N4F2 | complex-type; complex fucosylation |
| 3. | 2367.8 | S2H5N4F1 | complex-type |
| 4. | 1930.7 | S1H5N4 | complex-type |
| 5. | 2441.9 | S1H6N5F1 | complex-type |
| 6. | 2092.7 | G1H5N4F1 | complex-type |
| 7. | 2117.8 | S1H4N5F1 | complex-type; terminal |
| | | | N-acetylhexosamine (N > H) |
| 8. | 2587.9 | S1H6N5F2 | complex-type; complex fucosylation |
| 9. | 2368.9 | S1H5N4F3 | complex-type; complex fucosylation |
| 10. | 2263.8 | S1H4N5F2 | complex-type; complex fucosylation; |
| | | | terminal N-acetylhexosamine(N > H) |
| 11. | 1711.6 | S1H4N3F1 | hybrid/monoantennary |
| 12. | 2279.8 | S1H5N5F1 | complex-type; terminal |
| | | | N-acetylhexosamine (N═H ≧ 5) |
| 13. | 2238.8 | G1H5N4F2 | complex-type; complex fucosylation |
| 14. | 2733.0 | S2H6N5F1 | complex-type |
| 15. | 2807.0 | S1H7N6F1 | complex-type |

The 15 characteristic neutral (upper panel) and sialylated (lower panel) N-glycan signals of the hESC N-glycome. The signals are expressed in all the analyzed hESC samples and they are listed in order of relative abundance (No) in each N-glycan fraction.
H: hexose,
N: N-acetylhexosamine,
F: deoxyhexose,
S: N-acetylneuraminic acid,
G: N-glycolylneuraminic acid.
The proposed structural classification is according to FIG. 3A and as described in the text.

TABLE 7
NMR analysis of the major neutral N-glycans of hESC. The identified signals were
consistent with high-mannose type N-glycan structures such as the structures A-D that have
monosaccharide compositions H₇₋₉N₂. The significant signals in the NMR spectrum can be
explained by the following glycan structure combinations: A + B + C + D, A + B + D,
A + C + D, B + C + D, A + D, or B + C. Reference data is after Fu et al. (Fu, D., et al.,
1994, Carbohydr. Res. 261, 173-186) and Hård et al. (Hård, K., et al., 1991, Glycoconj.
J. 8, 17-28). Monosaccharide symbols are as in Supplementary Figure S1.

| | | | |
|---|---|---|---|
| A 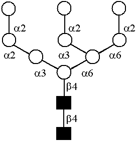 | B 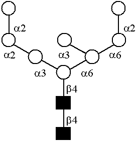 | C 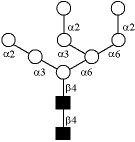 | D 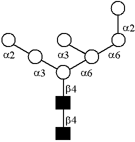 |

| Glycan residue | | | ¹H-NMR chemical shift (ppm) | | | | |
|---|---|---|---|---|---|---|---|
| Residue | Linkage | Proton | A | B | C | D | hESC¹⁾ |
| D-GlcNAc | | H-1α | 5.191 | 5.187 | 5.187 | 5.188 | 5.188 |
| | | H-1β | 4.690 | 4.693 | 4.693 | 4.695 | 4.694 |
| | | NAc | 2.042 | 2.037 | 2.037 | 2.038 | 2.038 |
| β-D-GlcNAc | 4 | H-1 | 4.596 | 4.586 | 4.586 | 4.600 | 4.596 |
| | | NAc | 2.072 | 2.063 | 2.063 | 2.064 | 2.061 |
| β-D-Man | 4,4 | H-1 | 4.775 | 4.771 | 4.771 | 4.780 | ²⁾ |
| | | H-2 | 4.238 | 4.234 | 4.234 | 4.240 | 4.234 |
| α-D-Man | 6,4,4 | H-1 | 4.869 | 4.870 | 4.870 | 4.870 | 4.869 |
| | | H-2 | 4.149 | 4.149 | 4.149 | 4.150 | 4.153 |
| α-D-Man | 6,6,4,4 | H-1 | 5.153 | 5.151 | 5.151 | 5.143 | 5.148 |
| | | H-2 | 4.025 | 4.021 | 4.021 | 4.020 | 4.023 |
| α-D-Man | 2,6,6,4,4 | H-1 | 5.047 | 5.042 | 5.042 | 5.041 | 5.042 |
| | | H-2 | 4.074 | 4.069 | 4.069 | 4.070 | 4.069 |
| α-D-Man | 3,6,4,4 | H-1 | 5.414 | 5.085 | 5.415 | 5.092 | 5.408/5.085 |
| | | H-2 | 4.108 | 4.069 | 4.099 | 4.070 | 4.102/4.069 |
| α-D-Man | 2,3,6,4,4 | H-1 | 5.047 | — | 5.042 | — | 5.042 |
| | | H-2 | 4.074 | — | 4.069 | — | 4.069 |
| α-D-Man | 3,4,4 | H-1 | 5.343 | 5.341 | 5.341 | 5.345 | 5.346/5.338 |
| | | H-2 | 4.108 | 4.099 | 4.099 | 4.120 | 4.102 |
| α-D-Man | 2,3,4,4 | H-1 | 5.317 | 5.309 | 5.050 | 5.055 | 5.310/5.057 |
| | | H-2 | 4.108 | 4.099 | 4.069 | 4.070 | 4.102/4.069 |
| α-D-Man | 2,2,3,4,4 | H-1 | 5.047 | 5.042 | — | — | 5.042 |
| | | H-2 | 4.074 | 4.069 | — | — | 4.069 |

¹⁾Chemical shifts determined from the center of the signal.
²⁾Signal under HDO.

TABLE 8
NMR analysis of the major sialylated N-glycan core structures of hESC. The identified
signals were consistent with sialylated biantennary complex-type N-glycan structures
such as the structures A-D that have monosaccharide compositions S₁₋₂H₅N₄F₀₋₁.
Reference data is after Hård et at (Hård, K., et at, 1992, Eur. J. Biochem. 209, 895-915)
and Helin et al. (Helin, J., et at, 1995, Carbohydr. Res. 266, 191-209). The significant
signals in the NMR spectrum can be explained by the structural components of these
reference structures (not shown). Monosaccharide symbols are as in Supplementary
Figure S1.

| | | | |
|---|---|---|---|
| A 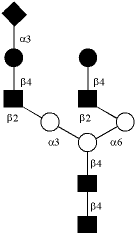 | B 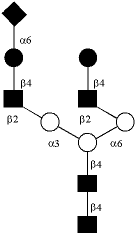 | C 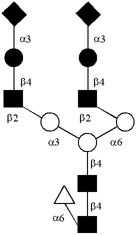 | D 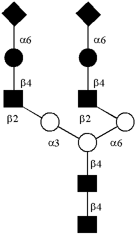 |

| Glycan residue | | | ¹H-NMR chemical shift (ppm) | | | | |
|---|---|---|---|---|---|---|---|
| Residue | Linkage | Proton | A | B | C | D | hESC¹⁾ |
| D-GlcNAc | | H-1α | 5.188 | 5.189 | 5.181 | 5.189 | 5.182/5.188 |
| | | NAc | 2.038 | 2.038 | 2.039 | 2.038 | 2.038 |
| α-L-Fuc | 6 | H-1α | — | — | 4.892 | — | 4.893 |
| | | H-1β | — | — | 4.900 | — | 4.893 |
| | | CH₃α | — | — | 1.211 | — | 1.210 |
| | | CH₃β | — | — | 1.223 | — | 1.219 |
| β-D-GlcNAc | 4 | H-1β | 4.604 | 4.606 | na. | 4.604 | 4.605 |
| | | NAc | 2.081 | 2.081 | 2.096 | 2.084 | 2.081/2.095 |
| β-D-Man | 4,4 | H-1 | n.a. | n.a. | n.a. | n.a. | n.a. |
| | | H-2 | 4.246 | 4.253 | 4.248 | 4.258 | 4.256 |
| α-D-Man | 6,4,4 | H-1 | 4.928 | 4.930 | 4.922 | 4.948 | 4.927 |
| | | H-2 | 4.11 | 4.112 | 4.11 | 4.117 | n.a. |
| β-D-GlcNAc | 2,6,4,4 | H-1 | 4.581 | 4.582 | 4.573 | 4.604 | 4.579/4.605 |
| | | NAc | 2.047 | 2.047 | 2.043 | 2.066 | 2.047/2.069 |
| β-D-Gal | 4,2,6,4,4 | H-1 | 4.473 | 4.473 | 4.550 | 4.447 | 4.447/4.472/4.545 |
| | | H-4 | n.a. | n.a. | n.a. | n.a. | 4.185 |
| α-D-Man | 3,4,4 | H-1 | 5.118 | 5.135 | 5.116 | 5.133 | 5.118/5.134 |
| | | H-2 | 4.190 | 4.196 | 4.189 | 4.197 | 4.195 |
| β-D-GlcNAc | 2,3,4,4 | H-1 | 4.573 | 4.606 | 4.573 | 4.604 | 4.579/4.605 |
| | | NAc | 2.047 | 2.069 | 2.048 | 2.070 | 2.047/2.069 |
| β-D-Gal | 4,2,3,4,4 | H-1 | 4.545 | 4.445 | 4.544 | 4.443 | 4.445/4.545 |
| | | H-3 | 4.113 | n.a. | 4.113 | n.a. | n.a. |

¹⁾Chemical shifts determined from the center of the signal.
n.a.: Not assigned.

TABLE 9
Proposed structures for acidic N-glycan signals in hESC or
differentiated cells, symbols Table 13.

| m/z | structure |
|---|---|
| 1151 | 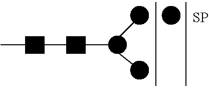 |
| 1338 | 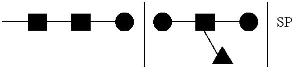 |
| 1354 | 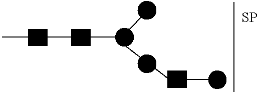 |
| 1362 | |
| 1403 | 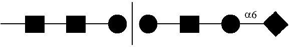 |
| 1475 | |
| 1500 | 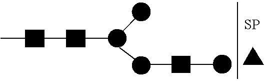 |
| 1516 | |
| 1541 | 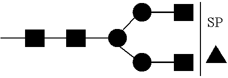 |
| 1549 | 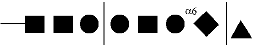 |
| 1557 | 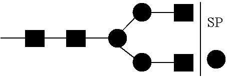 |
| 1565 | 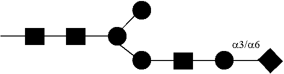 |
| 1637 | |
| 1678 | 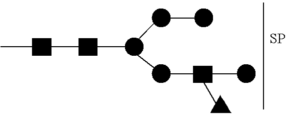 |
| 1703 | 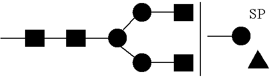 |
| 1711 | 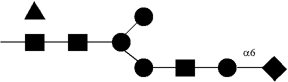 |
| 1719 | 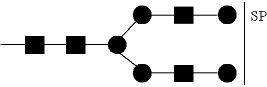 |
| 1727 | 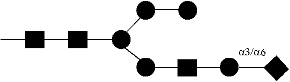 |
| 1744 | |
| 1752 | |
| 1760 | |
| 1768 | 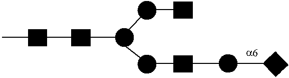 |
| 1791 | 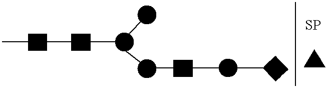 |
| 1799 | |
| 1808 | 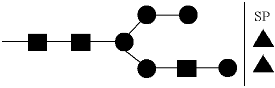 |
| 1824 | 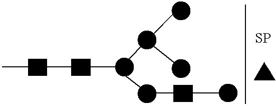 |
| 1831 | |
| 1840 | |
| 1849 | 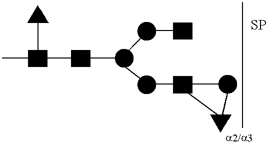 |
| 1865 | 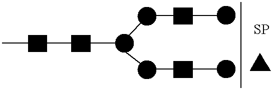 |
| 1873 | 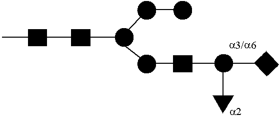 |
| 1889 | 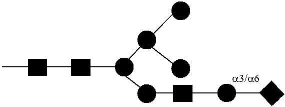 |
| 1906 | |
| 1914 | 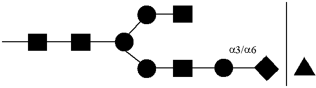 |
| 1930 | 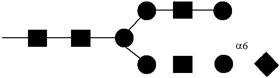 |
| 1946 | 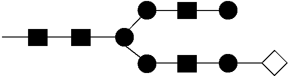 |
| 1947 | |
| 1971 | |
| 2002 | |
| 2003 | |
| 2010 | 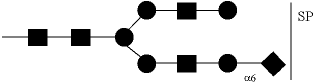 |
| 2011 | 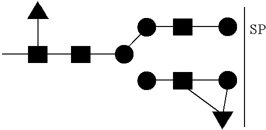 |
| 2018 | |
| 2027 | 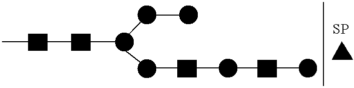 |
| 2035 | 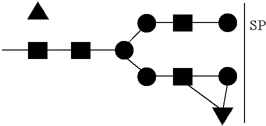 |
| 2051 | 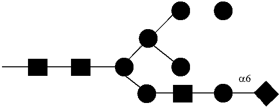 |
| 2052 | 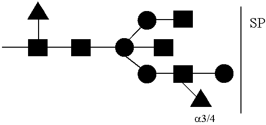 |
| 2068 | 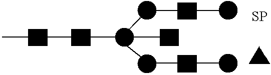 |
| 2076 | 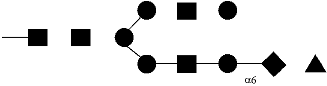 |
| 2082 | |
| 2092 | 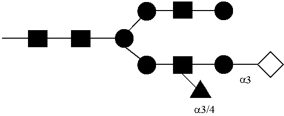 |
| 2117 | 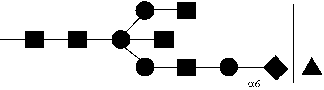 |
| 2133 | 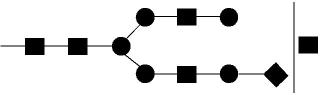 |
| 2156 | 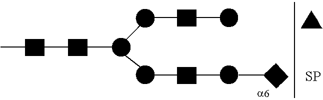 |
| 2157 | 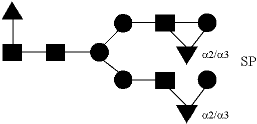 |
| 2164 | |
| 2174 | |
| 2178 | |
| 2214 | |
| 2221 | 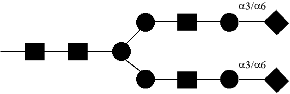 |
| 2222 | 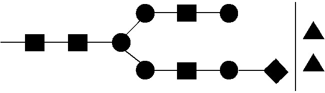 |
| 2230 | |
| 2237 | 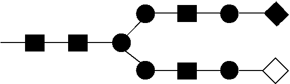 |
| 2238 | |
| 2239 | |
| 2246 | |
| 2253 | 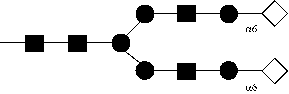 |
| 2254 | |
| 2263 | 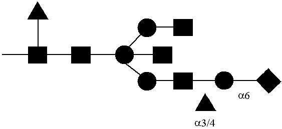 |
| 2279 | 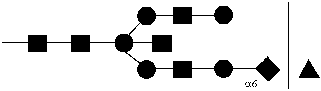 |
| 2280 | |
| 2295 | 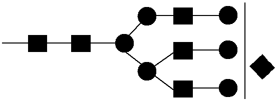 |
| 2302 | 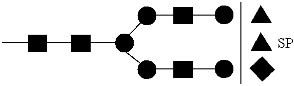 |
| 2319 | 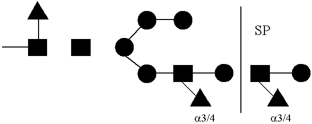 |
| 2320 | |
| 2321 | |
| 2367 | 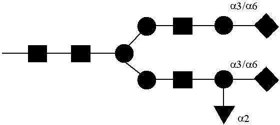 |
| 2368 | 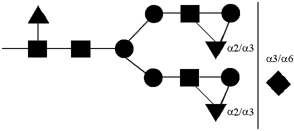 |
| 2376 | 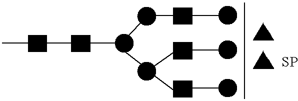 |
| 2383 | |
| 2384 | 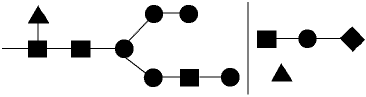 |
| 2390 | |
| 2391 | |
| 2400 | 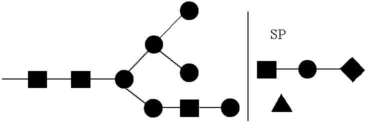 |
| 2408 | 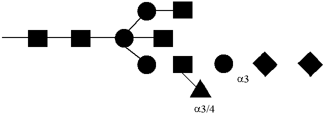 |
| 2425 | 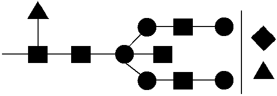 |
| 2433 | 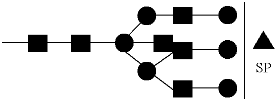 |
| 2441 | 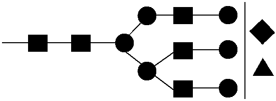 |
| 2447 | 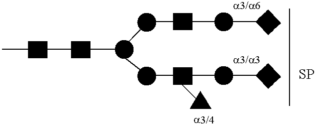 |
| 2448 | 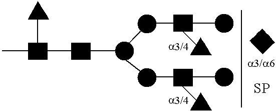 |
| 2456 | |
| 2457 | 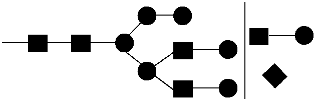 |
| 2482 | 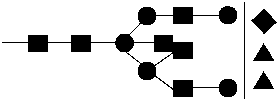 |
| 2483 | 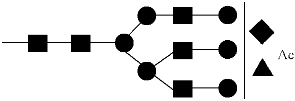 |
| 2512 | |
| 2521 | 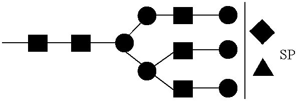 |
| 2522 | 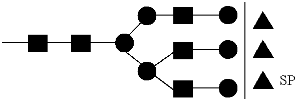 |
| 2528 | 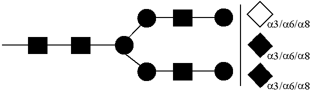 |
| 2529 | 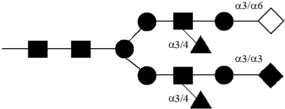 |
| 2544 | 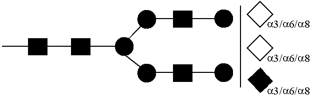 |
| 2570 | 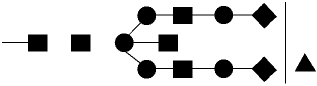 |
| 2571 | 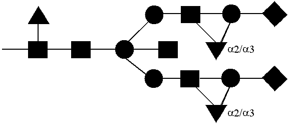 |
| 2579 | 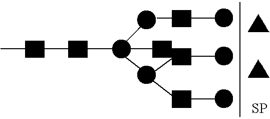 |
| 2586 | 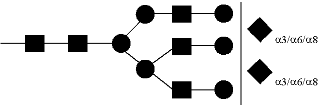 |
| 2587 | 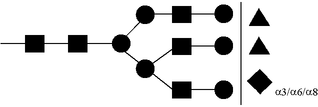 |
| 2603 | 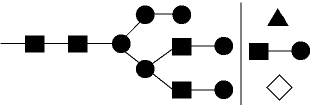 |
| 2627 | 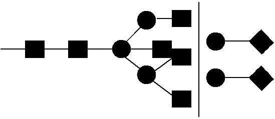 |
| 2644 | 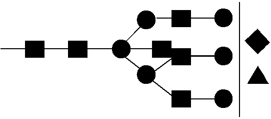 |
| 2645 | 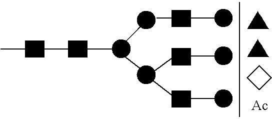 |
| 2660 | 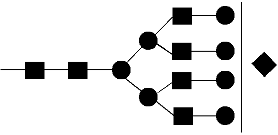 |
| 2668 | 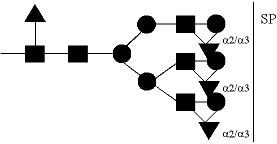 |
| 2683 | 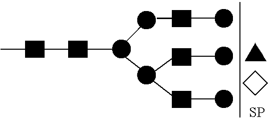 |
| 2714 | 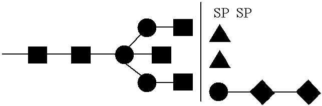 |
| 2725 | 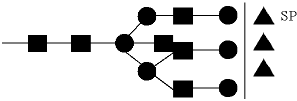 |
| 2732 | 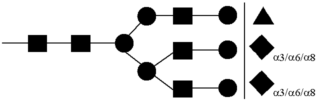 |
| 2733 | 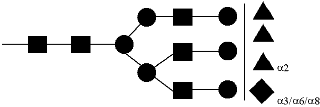 |
| 2791 | 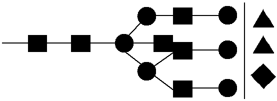 |
| 2806 | |
| 2807 | 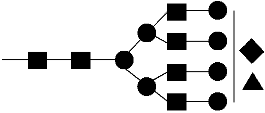 |
| 2813 | 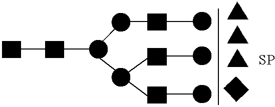 |
| 2848 | 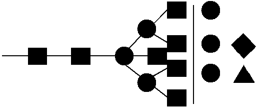 |
| 2864 | 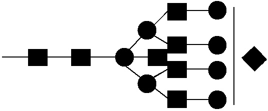 |
| 2878 | 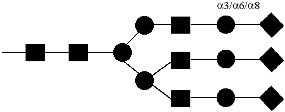 |
| 2879 | 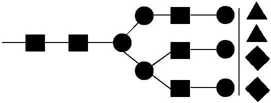 |
| 2880 | 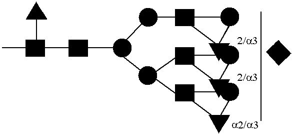 |
| 2886 | 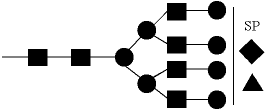 |
| 2887 | 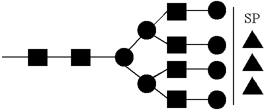 |
| 2936 | 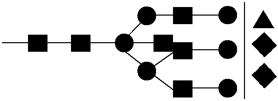 |
| 2953 | 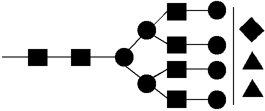 |
| 3024 | 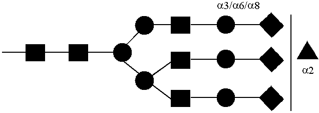 |
| 3025 | 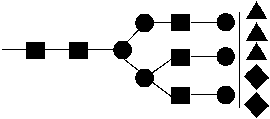 |
| 3026 | 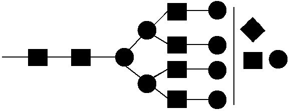 |
| 3098 | 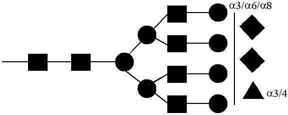 |
| 3099 | 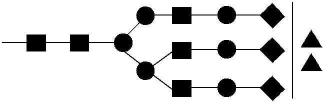 |
| 3170 | |
| 3172 | 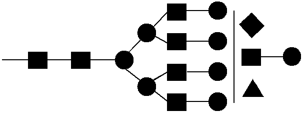 |
| 3245 | 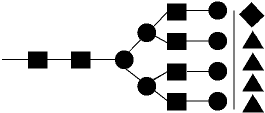 |
| 3317 | 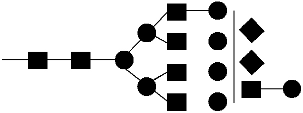 |
| 3390 | 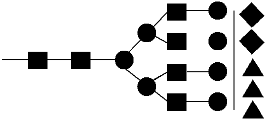 |
| 3463 | 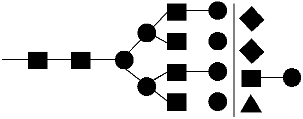 |
| 3608 | 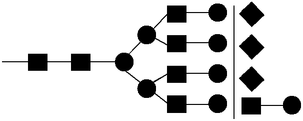 |
| 3610 | 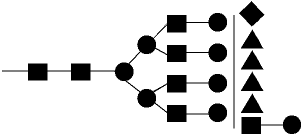 |
| 3682 | 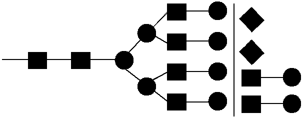 |
| 3756 | 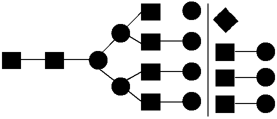 |

TABLE 10a
Proposed structures for neutral N-glycan signals detected in
hESC or differentiated cells. Symbols Table 14.

| m/z | Structure |
|---|---|
| 568.19 | 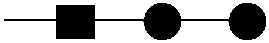 |
| 609.21 | 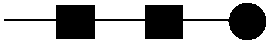 |
| 714.24 | |
| 730.24 | 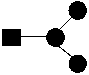 |
| 755.27 | 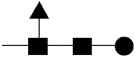 |
| 771.26 | 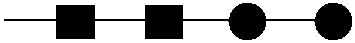 |
| 892.29 | 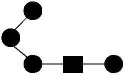 |
| 901.33 | |
| 917.32 | 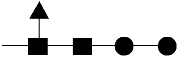 |
| 933.31 | 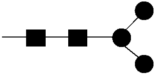 |
| 1031.33 | |
| 1054.34 | 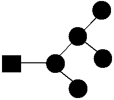 |
| 1079.38 | 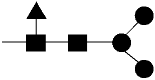 |
| 1095.37 | 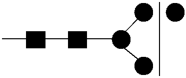 |
| 1120.4 | 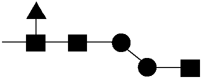 |
| 1136.4 | 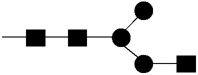 |
| 1209.44 | |
| 1216.4 | 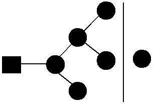 |
| 1225.43 | |
| 1241.43 | 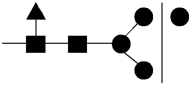 |
| 1257.42 | 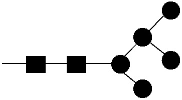 |
| 1266.46 | |
| 1282.45 | 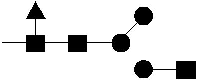 |
| 1298.45 | 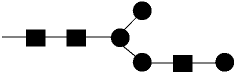 |
| 1323.48 | 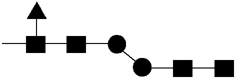 |
| 1339.48 | 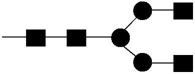 |
| 1378.45 | 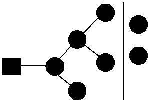 |
| 1393 | |
| 1403.48 | 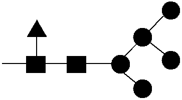 |
| 1419.48 | 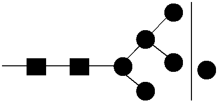 |
| 1444.51 | 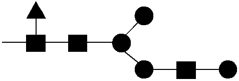 |
| 1460.5 | 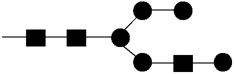 |
| 1485.53 | 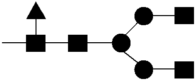 |
| 1501.53 | 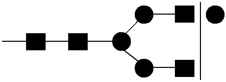 |
| 1517.55 | |
| 1540.5 | 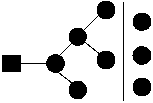 |
| 1542.56 | 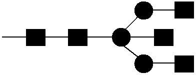 |
| 1555 | |
| 1565.53 | 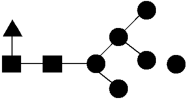 |
| 1581.53 | 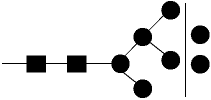 |
| 1590.57 | 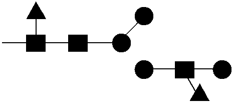 |
| 1606.56 | 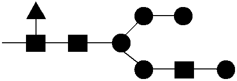 |
| 1622.56 | 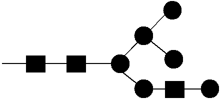 |
| 1631.59 | 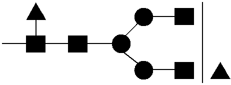 |
| 1647.59 | 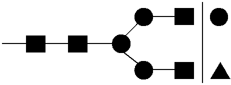 |
| 1663.58 | 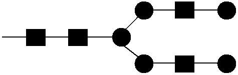 |
| 1688.61 | 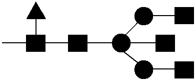 |
| 1702.56 | 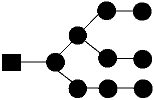 |
| 1704.61 | 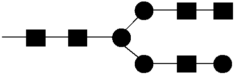 |
| 1717 | |
| 1720.63 | |
| 1743.58 | 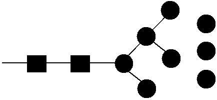 |
| 1752.62 | 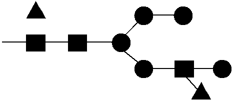 |
| 1768.61 | 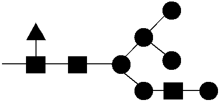 |
| 1784.61 | |
| 1793.64 | 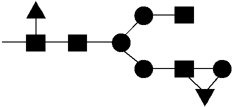 |
| 1809.64 | 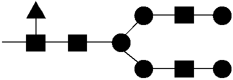 |
| 1825.63 | 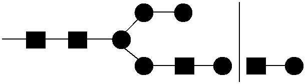 |
| 1850.67 | 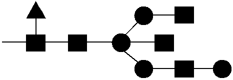 |
| 1864.61 | 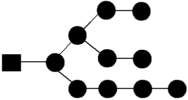 |
| 1866.66 | 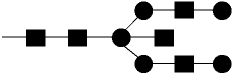 |
| 1882.68 | |
| 1905.63 | 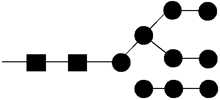 |
| 1914.67 | 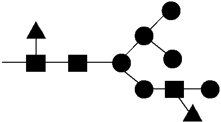 |
| 1955.7 | 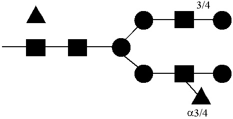 |
| 1971.69 | 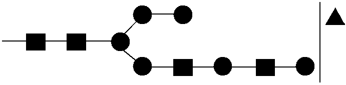 |
| 1980.73 | 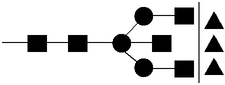 |
| 1987.69 | 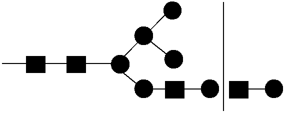 |
| 1996.72 | 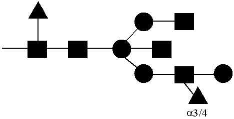 |
| 2012.72 | 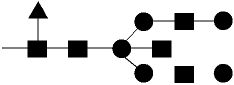 |
| 2019.7 | |
| 2021.76 | |
| 2028.71 | 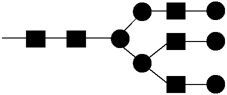 |
| 2037.75 | |
| 2041 | |
| 2053.75 | |
| 2067.69 | 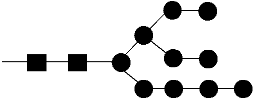 |
| 2101.76 | 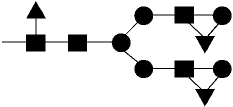 |
| 2117.75 | 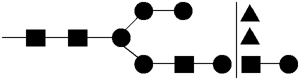 |
| 2126.79 | |
| 2133.75 | 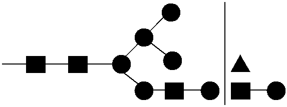 |
| 2142.78 | |
| 2149.74 | 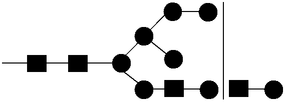 |
| 2158.78 | |
| 2174.77 | 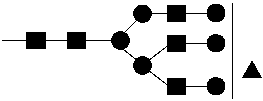 |
| 2183.81 | |
| 2190.77 | 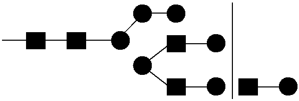 |
| 2199.8 | 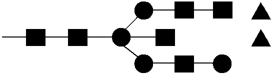 |
| 2215.8 | 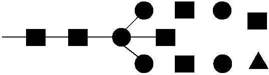 |
| 2229.74 | 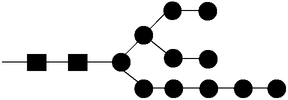 |
| 2231.79 | 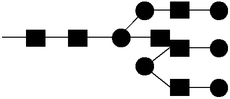 |
| 2304.84 | 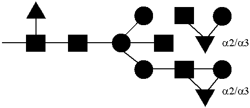 |
| 2320.83 | 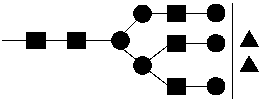 |
| 2361.87 | 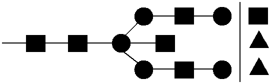 |
| 2391.79 | 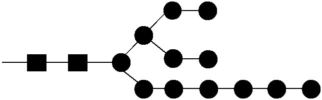 |
| 2393.85 | |
| 2466.89 | 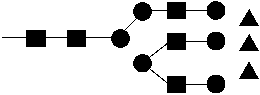 |

TABLE 10b
Lectin staining of human embryonic stem cells. The glycan structures are presented in
colour symbols, given at the end of Table 19. The reducing end of the N-glycans is on
left for N-glycans in Tables 12 and 13, and on right in Tables 14-19 (mirror images to
ones in 12 and 13). The linkages of N-glycans are indicated in NMR Tables 8 and 9, and
in Tables 12-19 based on the Consortium for Functional Glycomics, USA recommendations,
1-4 linkages (Manβ4, GlcNAcβ4, Galβ4, Galα4 on Lactosylresidue in globostructres,
GalNAcβ4 on on Lactosylresidue in ganliostructures) are horizontal-, 1-6 linkages
(Manα6, NeuAc/sialic acidα6, GlcNAcβ6) are\in Tables 14-19, except Fucα6 above above
reducing end GlcNAc in , and/in Tables 12 and 13, 1-3 linkages (Manα3, Fucα3,
Neu5Ac/Neu5Gc/sialic acidα3, Galβ3, GlcNAcβ3, GalNAcα3GalNAcβ3 and GalNAcβ3
on Galα4 at non-reducing end of Forsman and Globoside(Gb4) and elongated globoseries
glycolipid structures, respectively) are/in Tables 14-19, and\in Tables 12 and 13 (for N-glycan
compatible structures. Fucα2 is indicated by vertical line below Galβ3/Galβ4-residue.
SP in Tables 12 and 13 indicates sulphated or fosfate and is preferably sulfate on compelx
type N-aglycans comprising N-acetyllactosamine residues and fosfate in High/Low Mannose
glycans. In tables 14-19 S is sialic acid (preferably Neu5Ac and/or Neu5Gc), LN is
N-cetyl-lactosamine, preferably Galβ4GlcNAc, LN type 1 is Galβ3GlcNAc, Lex is
Lewis x, Ley is Lewis y, Leb is Lewis b. Regular abbreviations of plant leactins are
used, these are available e.g. from catalog of EY Labs USA. MEF is mouse embryonic
fibroblast feeder cell, FES indicates embryonic stem cell line and number specifies
the line, EB is embryonic body.

| | | | | | EB | |
|---|---|---|---|---|---|---|
| Lectin | epitope | | FES22 | FES30 | (29 + 30 | MEF |
| PSA | Manα | 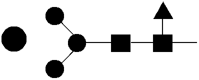 | − | − | | + |
| LTA | Lex | 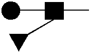 | −/+ | − | − | + |
| UEA | H type 2 | 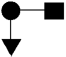 | 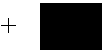 | 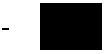 | ^{22+,29-} | +/− |
| MAA | Sα2-3 | 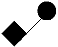 | + | + | + | − |
| SNA | Sα2-6 | 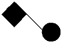 | (+/−) | (+/−) | | + |
| RCA | LN | 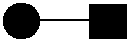 | 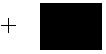 | + | + | + |
| PNA | Galβ1- | 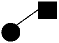 | + | 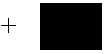 | + | − |
| PWA | polyLN (I) | 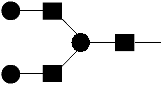 | + | 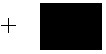 | + | + |
| STA | polyLN (i) | 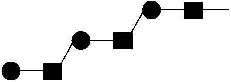 | (+/−) | − | | + |
| WFA | GalNAcβ | 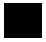 | + | 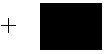 | + | − |

TABLE 11
TLC blot of human embryonic stem cells. Experiments with low amounts of
Sample, + indicates potential reactivity, − not done or need experiments,
2 columns on right for comparision. Monosacharide symbols below and with
Table 14, reducing end on the right.

| | | | | | | Cell |
|---|---|---|---|---|---|---|
| | | | | | FACS | Staining |
| epitope | | FES29 | FES30 | FES61 | (FES29) | FES22,29,30 |
| LN type 1 | 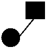 | − | − | − | + | + |
| asialo GM1 | 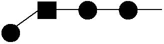 | + | − | − | | |
| SSEA-3 | 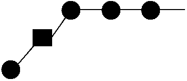 | − | − | − | + | + |
| SSEA-4 | 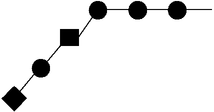 | − | − | + | + | + |
| Galβ1- 3-GalNAc | 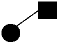 | − | − | − | | |
| asialo GM2 | 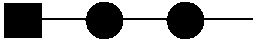 | + | − | − | | |
| globoside | 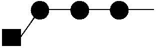 | − | − | − | | +/− |
| Forssman | 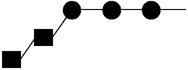 | + | + | + | | − |
| H(1) | 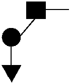 | − | − | − | | + |
| globo H | 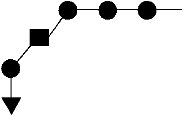 | − | − | − | | +/− |
| H(2) | 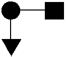 | − | − | − | | + |
| Ley | 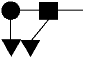 | − | − | − | | ? |
| Leb | 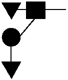 | − | − | − | | +/− |
| Lea | 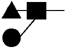 | − | − | − | | − |

TABLE 12

| Code | Producer code | Clone | Specificity | host/isotype |
|---|---|---|---|---|
| GF 279 | Abcam ab3352 | K21 | Lewis c, LacNAc (LN) Type 1 | mouse/IgM |
| GF 280 | Glycotope | MAB-S301 | TF-antigen (Galβ3GalNAc) | |
| | | (Nemod TF2) | | |
| GF 281 | Glycotope | MAB-S305 | TF-antigen (Galβ3GalNAc) | Mouse IgG1 |
| | | (A68-E/E3) | | |
| GF 283 | Acris DM3122 | 2-25LE | Lewis b (Leb) | mouse/IgG |
| GF 284 | Acris DM3015 | B393 | H Type 2 H (2) | mouse/IgM |
| GF 285 | Acris DM3014 | B389 | H Type 2, Lewis b, Lewis y | mouse/IgG1 |
| GF 286 | Acris BM258P | BRIC 231 | H Type 2, H (2) | mouse/IgG1 |
| GF 287 | Abcam ab3355 | 17-206 | H Type 1, H (1) | mouse/IgG3 |
| GF 288 | Glycotope MAB-S206 | A69-A/E8 | Globo H | mouse/IgM |
| GF403 | | | | |
| GF 289 | Glycotope MAB-S201 | A70-C/C8 | Lewis y (Ley) | mouse/IgM |
| GF 290 | Glycotope MAB-S204 | A51-B/A6 | H type 2, H (2) | mouse/IgA |
| GF 304 | Chemicon CBL205 | PR5C5 | Lewis a | |
| GF 305 | Chemicon CBL144 | 28 | Lewis x (Lex) | |
| GF 307 | Chemicon MAB2096 | KM93 | Sialyl Lewis x (Slex) | |
| GF 353 | Chemicon MAB4303 | MC-631 | SSEA-3 | |
| GF 366 | Abcam ab23949 | polyclonal | Gb4, globoside | rabbit |
| GF 367 | Acris | SM1160P | Gb3 globotriose | |
| GF 368 | Leiden University | 259-2A1 | LacdiNAc | mouse/IgG3 |
| GF 369 | Leiden University | 273-3F2 | LacdiNAc | mouse/IgM |
| GF 370 | Leiden University | 290-2E6 | α3-fucosyl-LacdiNAc | mouse/IgM |
| GF 371 | Leiden University | 291-3E9 | α3-fucosyl-LacdiNAc | |
| GF 372 | Acris | B35.1 | Sialyl-Tn | |
| GF 373 | Acris | DM3184P | PN-15 | |
| GF 305 | Chemicon CBL144 | 28 | Lewis x (Lex) | |
| GF 307 | Chemicon MAB2096 | KM93 | Sialyl Lewis x (Slex) | |
| GF 401 | Acris BM4091 | FOM-1 | Forssman antigen | rat/IgM |
| GF 402 | Leiden University | 100-4G11 | low-mannose N-glycan (low | mouse/IgG |
| GF 418 | Alexis | MBr1 | man) | |
| | | | Globo-H | |

TABLE 13
Comparison of lectin ligand profile in hESCs and MEFs

| Lectin | hESC | MEF |
|---|---|---|
| PSA | − | + |
| MAA | + | − |
| PNA | + | − |
| RCA | + | + |

+ present in cell surface
− not present in cell surface

TABLE 44
Lectins

| | FES29 | FES30 |
|---|---|---|
| PSA | − | − |
| LTA | +/− | − |
| UEA | + | − |
| MAA | + | + |
| SNA | (+/−) | (+/−) |
| RCA | + | + |
| PNA | + | + |
| PWA | + | + |
| STA | (+/−) | − |
| WFA | + | + |
| PHA-L | (+/−) | (+/−) |

TABLE 14
FACS

| | FES30 | FES61 |
|---|---|---|
| PSA | + | + |
| LTA | +/− | |
| UEA | + | + |
| MAA | + | + |
| SNA | + | |
| RCA | + | |
| PNA | + | + |
| PWA | +/− | − |
| STA | +/− | +/− |
| WFA | − | (+/−) |
| PHA-L | | |
| NPA | + | +/− |
| MBL | − | − |

TABLE 15
Antibodies

| | Immuno | FACS |
|---|---|---|
| GF281 | | − |
| GF285 | − | − |
| GF286 | +/− | + |
| GF287 | + | + |
| GF372 | | − |
| GF373 | | − |
| anti-Le a | | − |
| GF368 | +/− | − |
| GF279 | + | + |
| GF280 | | − |
| GF284 | +/− | − |
| GF288 | +/− | − |
| GF289 | (+/−) | − |

TABLE 16
Antibodies

| | Immuno | FACS |
|---|---|---|
| GF403 | | − |
| GF418 | | − |
| anti-Le x | | − |
| anti-sialyl | | − |
| Le x | | |
| GF369 | +/− | − |
| GF370 | +/− | − |
| GF371 | | − |
| GF367 | +/− | + |
| GF401 | − | − |
| GF283 | +/− | |
| GF290 | (+/−) | |
| GF402 | +/− | |
| GF366 | − | |

TABLE 17

| Reagent | Target | FES 22 | FES 30 | mEF | % stain |
|---|---|---|---|---|---|
| FITC-PSA | α-Man | − | − | + | |
| FITC-RCA | β-Gal (Galβ4GlcNAc) | + | − | +/− | |
| FITC-PNA | β-Gal (Galβ3GalNAc) | + | + | − | |
| FITC-MAA | α2,3-sialyl-LN | + | + | − | |
| FITC-SNA | α2,6-sialyl-LN | + | n.d. | + | |
| FITC-PWA | I-antigen | + | + | n.d. | |
| FITC-STA | i-antigen | + | − | + | |
| FITC-WFA | β-GalNAc | + | + | − | |
| NeuGc- | NeuGc-lectin | + | + | + | |
| PAA-biotin | | | | | |
| anti- | NeuGcα3Galβ4Glc | + | + | + | |
| GM3(Gc) | | | | | |
| mAb | | | | | |
| FITC-LTA | α-Fuc | + | − | + | |
| FITC-UEA | α-Fuc | + | − | + | |
| mAb Lex | Lewis^{x} | + | n.d. | − | |
| mAb sLex | sialyl-Lewis^{x} | + | n.d. | − | |
| GF 279 Le c | Galβ3GlcNAc | | + | − | 95-100 |
| GF 283 Le b | | | + | − | 20-35 |
| GF 284 H | | | + | − | 15-20 |
| Type 2 | | | | | |
| GF 285 H | | | − | + | 95-100 |
| Type 2 | | | | | |
| GF 286 H | | | + | − | 10-20 |
| Type 2 | | | | | |
| GF 287 H | | | + | − | 90-100 |
| Type 1 | | | | | |
| GF 288 | | | + | − | 20-35 |
| Globo-H | | | | | |
| GF 289 Ley | | | − | + | 95-100 |
| GF 290 H | | | + | − | 20-35 |
| Type 2 | | | | | |

+, specific binding.
−, no specific binding.
n.d., not determined.
% of stain means approximate percentage of cell stained with a binder.

TABLE 18
Summary of immunohistochemical stainings (IHC) and FACS analysis of bone
marrow derived mesenchymal stem cells (BM-MSC) and osteogenic cells derived thereof
(osteogenic). FACS results are shown as an average percentage of positive cells in a cell
population (n = 1-3 individual experiment(s)). Trypsin FACS results are from single
Experiment.

| | | BM- | | Tryps. | | Osteog. | Tryps. |
|---|---|---|---|---|---|---|---|
| | | MSC | BM-MSC | FACS | Osteog. | FACS | FACS |
| Code | Antigen | IHC | FACS (%) | (%) | IHC | (%) | (%) |
| GF274 | PNAd (peripheral lymph node addressin; CD62L ligand) closely | − | 0.9 | 0.4 | − | 1.8 | 0.5 |
| | associated with L-selectin (CD34, GlyCAM-1, MAdCAM-1), | | | | | | |
| | sulfo-mucin | | | | | | |
| GF275 | CA15-3 (Cancer antigen 15-3; sialylated carbohydrate epitope of | + | 46.5 | 57.9 | + | 79.1 | 14.1 |
| | the MUC-1 glycoprotein) | | | | | | |
| GF276 | oncofetal antigen, tumor associated glycoprotein (TAG-72) or CA | − | 0.8 | 0.5 | + | 0.8 | |
| | 72-4 | | | | | | |
| GF277 | human sialosyl-Tn antigen (STn, sCD175) | (+) | 7.3 | 0.4 | + | 1.0 | 0.7 |
| GF278 | human Tn antigen (Tn, CD175 B1.1) | (+) | 5.9 | 0.5 | + | 3.0 | 0.9 |
| GF295 | Blood group antigen precursor (BG1), Lewis c Gb3GN (pLN) | − | 9.6 | 0.7 | − | 2.7 | 1.0 |
| GF280 | TF-antigen isoform (Nemod TF2) | − | NT | | − | NT | |
| GF281 | TF-antigen isoform (A68-E/E3) | − | NT | | − | NT | |
| GF296 | asialoganglioside GM1 | − | 22 | 1.1 | − | 48.2 | 1.1 |
| GF297 | Globoside GL4 | + | 16.9 | 14.2 | + | 28.4 | 4.9 |
| GF298 | Human CD77 (=blood group substance pk), GB3 | + | 21.8 | 27.2 | + | 52.7 | 4.9 |
| GF299 | Forssman antigen, glycosphingolipid (FO GSL) differentiation ag | − | 4.1 | 0.4 | − | 5.5 | 0.4 |
| GF300 | Asialo GM2 | − | 17.1 | 0.9 | − | 53.8 | 1.7 |
| GF301 | Lewis b blood group antigen | − | 1.2 | | − | 1.3 | 0.7 |
| GF302 | H type 2 blood group antigen | + | 14.7 | 0.7 | + | 26.2 | 2.4 |
| GF303 | Blood group H1(O) antigen (BG4) | − | 1.4 | 0.3 | + | 0.7 | 0.6 |
| GF288 | Globo-H | − | NT | | NT | NT | |
| GF304 | Lewis a | − | 13 | 1.7 | − | 23.4 | 1.4 |
| GF305 | Lewis x, CD15, 3-FAL, SSEA-1, 3-fucosyl-N-acetyllactosamine | (+/−) | 1 | 0.5 | − | 1.1 | 0.7 |
| GF306 | Sialyl Lewis a | − | 4.9 | 0.8 | − | 2.7 | 0.7 |
| GF307 | Sialyl Lewis x | + | 82.1 | 70.4 | (+/−) | 55.7 | 33 |
| GF353 | SSEA-3 (stage-specific embryonic antigen-3) | + | 33.8 | 6.8 | (+/−) | 6.2 | 0.8 |
| GF354 | SSEA-4 (stage-specific embryonic antigen-4) | + | 77.2 | 53.7 | − | 34.0 | 2.4 |
| GF365 | Nemod TF1, DC176, GalB1-3GalNAc | − | 3.8 | | − | 1.1 | 0.8 |
| GF374 | Glycodelin A, GdA, PP14 (A87-D/F4) | (+/−) | 0.9 | | − | 0.3 | 0.6 |
| GF375 | Glycodelin A, GdA, PP14 (A87-D/C5) | − | 2.4 | | − | 0.6 | 0.8 |
| GF376 | Glycodelin A, GdA, PP14 (A87-B/D2) | − | 3.4 | | − | 0.6 | 0.6 |
| GF393 | Lewis y | − | NT | | − | 0.6 | 0.5 |
| GF394 | H disaccharide | − | NT | | − | 0.5 | 1.2 |

+ = positive,
(+) = weak positive,
(+/−) = single positive cells,
− = negative;
NT = not tested

TABLE 19
Protease sensitive glycan epitopes on the cell surface of BM-MSC and osteogenic
cells derived thereof. Results are shown as a percentage of positive cells in FACS analysis.
Codes for antibodies are as described in Example 15 (ab stainings).

| | | BM-MSC | BM-MSC | Osteog | Osteog |
|---|---|---|---|---|---|
| Code | Antigen | Versene (%) | Trypsin (%) | Versene (%) | Trypsin (%) |
| GF275 | CA15-3 (Cancer antigen 15-3; sialylated | | | 96.9 | 14.1 |
| | carbohydrate epitope of the MUC-1 | | | | |
| | glycoprotein) | | | | |
| GF277 | human sialosyl-Tn antigen (STn, sCD175) | 4.0 | 0.4 | | |
| GF278 | human Tn antigen (Tn, CD175 B1.1) | 4.7 | 0.5 | | |
| GF295 | Blood group antigen precursor (BG1), Lewis | 4.4 | 0.7 | | |
| | c Gβ3GN (pLN) | | | | |
| GF296 | asialoganglioside GM1 | 34.3 | 1.1 | 35.5 | 1.1 |
| GF299 | Forssman antigen, glycosphingolipid (FO | 4.1 | 0.4 | 6.7 | 0.4 |
| | GSL) differentiation ag | | | | |
| GF300 | asialoganglioside GM2 | 19.4 | 0.9 | 55.3 | 1.7 |
| GF302 | H type 2 blood group antigen | 6.0 | 0.7 | 23.3 | 2.4 |
| GF304 | Lewis a | 14.3 | 1.7 | 10.4 | 1.4 |
| GF306 | Sialyl Lewis a | 5.9 | 0.8 | 1.3 | 0.7 |
| GF307 | Sialyl Lewis x | 82.1 | 70.4 | 62.3 | 33.0 |
| GF354 | SSEA-4 (stage-specific embryonic antigen- | 77.2 | 53.7 | 21.4 | 2.4 |
| | 4) | | | | |

TABLE 20
Expression of the genes encoding glycosyltransferases and glycosidases involved in
the biosynthesis of N-glycans in CD133+ and CD133− cells. In addition,
gene name encoding glycosyltransferases and glycosidases of the same family
along with their glycan class and structure specifity is represented.

| Gene | | | | |
|---|---|---|---|---|
| expression | | Gene | Glycan | |
| CD133+ | CD133− | name | class | Structure specificity |

α-mannosidase (MAN) families I and II (21, 48-54)

| | | | | |
|---|---|---|---|---|
| P | P | MAN1A1 | N | α2MAN belonging to the MAN |
| P | P | MAN1A2 | N | I family |
| P | P | MAN1B1 | N | |
| A | P | MAN1C1 | N | |
| P | P | MAN2A1 | N | α3/6MAN belonging to the MAN II |
| P | P | MAN2A2 | N | family |
| P | P | MAN2B1 | N | |
| P | P | MAN2B2 | N | |
| P | P | MAN2C1 | N | |

N-glycan branching β-N-acetylglucosaminyltransferases (MGAT) (17)

| | | | | |
|---|---|---|---|---|
| P | P | MGAT1 | N | N-glycan branching enzymes; |
| P, I | P | MGAT2 | N | see also FIG. 4. |
| A | A | MGAT3 | N | |
| P, D | P | MGAT4A | N | |
| P | P | MGAT4B | N | |
| A | A | MGAT5 | N | |
| *NP | *NP | MGAT6 | N | |

β1,3-galactosyltransferases (β3GalT) (55-60)

| | | | | |
|---|---|---|---|---|
| A | P | B3GALT1 | N, O, L | |
| A | A | B3GALT2 | N, O, L | |
| | | B3GALT3 | L | globoside synthase |
| | | B3GALT4 | L | GM1 synthase |
| A | A | B3GALT5 | N, O, L | O-glycan Core 3 elongation |
| | | B3GALT6 | G | GAG GalT2 |
| | | B3GALT7 | (1 | |

β1,4-galactosyltransferases (β4GalT) (29, 61-65)

| | | | | |
|---|---|---|---|---|
| P, I | P | B4GALT1 | N, O, L | lactose/N-acetyllactosamine synthase |
| P | A | B4GALT2 | N, O, L | Lactose/N-acetyllactosamine synthase |
| P, D | P | B4GALT3 | N, O, L | |
| P | P | B4GALT4 | N, O, L | 6-sulfo-GlcNAc GalT |
| A | A | B4GALT5 | O > N, L | O-glycan Core 2 elongation |
| | | B4GALT6 | L | lactosylceramide synthase |
| | | B4GALT7 | G | GAG GalT1 |

α2,3-sialyltransferases (α3SAT) (33, 66, 67)

| | | | | |
|---|---|---|---|---|
| | | ST3GAL1 | O | O-glycan Core 1 sialylation |
| A | A | ST3GAL2 | N, O, L | |
| A | A | ST3GAL3 | N, O, L | type 1 LacNAc sialylation |
| A | A | ST3GAL4 | N, O, L | type 2 LacNAc sialylation |
| | | ST3GAL5 | L | GM3 synthase |
| P, I | P | ST3GAL6 | N, O, L | type 2 LacNAc sialylation |

α2,6-sialyltransferases (α6SAT) 37, 52, 68-71

| | | | | |
|---|---|---|---|---|
| P | P | ST6GAL1 | N, O, L | type 2 LacNAc sialylation |
| A | A | ST6GAL2 | N, O, L | type 2 LacNAc sialylation |

α1,2-/α1,3-/α1,4-/α1,6-fucosyltransferases (FucT) (18, 19, 44, 72, 73, 73-80)

| | | | | |
|---|---|---|---|---|
| A | A | FUT1 | N, O, L | α1,2-FucT (H-2 synthesis) |
| *NP | *NP | FUT2 | N, O, L | α1,2-FucT (Secretor, H-1 synthesis) |
| A | A | FUT3 | N, O, L | α1,3/4-FucT |
| P | P | FUT4 | N, O, L | α1,3-FucT (Lex/sLex synthesis) |
| A | A | FUT5 | N, O, L | α1,3-FucT (Lex/sLex synthesis) |
| A | A | FUT6 | N, O, L | α1,3-FucT (Lex/sLex synthesis) |
| A | A | FUT7 | N, O, L | α1,3-FucT (Lex/sLex synthesis) |
| P | A | FUT8 | N | α1,6-FucT (N-glycan core fucosylation) |
| A | A | FUT9 | N, O, L | α1,3-FucT (Lex/sLex synthesis) |

1) May be a false annotation, should be B3GNT1
Abreviations:
A; gene not expressed,
P; gene expression;
I; increased expression in CD133+ cells,
D; decreased gene expression in CD133+ cells,
*NP; no probe available,
N; N-glycan,
O; O-glycan,
L; glycosphingolipids;
G, glycosaminoglycans.

TABLE 21
Cell surface glycan epitope assay with lectins.

| | | | stem |
|---|---|---|---|
| Lectin | Specificity | leucocytes | cells |
| PSA | α-mannose, N-glycan core structure | 96% | +++ |
| HHA | α-mannose | 99% | +++ |
| GNA | α-mannose, less to α1,3-linked mannose | 73% | + |
| | residues | | |
| PHA-L | large complex-type N-glycans with β1,6- | 98% | ++ |
| | branch | | |
| RCA-I | β1,4-linked galactose, type 2 LacNAc | 91% | +++ |
| SNA | α2,6-linked sialic acid | 98% | +++ |
| MAA | α2,3-linked sialic acid in type 2 LacNAc | 62% | ++ |
| LTA | α1,3-linked fucose (Lex) | 6% | − |
| UEA-I | α1,2-linked fucose in type 2 LacNAc (H-2) | 53% | +++ |

TABLE 22
Neutral N-glycan difference analysis.

| | | | |
|---|---|---|---|
| composition¹⁾ | m/z²⁾ | class³⁾ | fold⁴⁾ |

+++ CD133+⁵⁾

| | | | |
|---|---|---|---|
| H2N3 | 974 | H | +∞ |
| H4N5 | 1704 | CT | +∞ |

++ CD133+

| | | | |
|---|---|---|---|
| H3N5 | 1542 | CT | 1.91 |
| H5N4F3 | 2101 | CE | 1.91 |
| H4N4F1 | 1647 | CF | 1.76 |
| H3N5F1 | 1688 | CFT | 1.55 |
| H1N2F1 | 755 | LF | 1.50 |

+CD133+

| H3N3F2 | 1428 | HE | 1.49 |
|---|---|---|---|
| H2N3F1 | 1120 | HF | 1.46 |
| H5N4F2 | 1955 | CE | 1.36 |
| H4N4F2 | 1793 | CE | 1.34 |
| H5N3F2 | 1752 | HE | 1.33 |
| H5N2 | 1257 | M | 1.30 |
| H4N3F2 | 1590 | HE | 1.27 |
| H5N4F1 | 1809 | CF | 1.22 |
| H5N3F1 | 1606 | HF | 1.21 |
| H4N3F1 | 1444 | HF | 1.21 |
| H6N2F1 | 1565 | MF | 1.19 |
| H9N2 | 1905 | M | 1.18 |
| H8N2 | 1743 | M | 1.12 |
| H3N3F1 | 1282 | HF | 1.08 |
| H6N3F1 | 1768 | HF | 1.05 |
| H5N2F1 | 1403 | MF | 1.03 |
| H4N5F3 | 2142 | CET | 1.03 |
| H6N5 | 2028 | CR | 1.02 |
| H6N5F1 | 2174 | CFR | 1.01 |
| composition | m/z | class | fold |

+CD133−

| | | | |
|---|---|---|---|
| H3N4F1 | 1485 | CFT | −1.02 |
| H4N2 | 1095 | L | −1.03 |
| H10N2 | 2067 | MG | −1.03 |
| H7N2 | 1581 | M | −1.05 |
| H6N2 | 1419 | M | −1.07 |
| H2N2F1 | 917 | LF | −1.10 |
| H6N3 | 1622 | H | −1.18 |
| H4N2F1 | 1241 | LF | −1.19 |
| H5N4 | 1663 | C | −1.40 |
| H5N3 | 1460 | H | −1.41 |

++CD133−

| | | | |
|---|---|---|---|
| H3N2F1 | 1079 | LF | −1.53 |
| H2N2 | 771 | L | −1.54 |
| H3N2 | 933 | L | −1.56 |
| H3N3 | 1136 | H | −1.63 |
| H4N3 | 1298 | H | −1.67 |
| H1N2 | 609 | L | −1.77 |

+++CD133−

| | | | |
|---|---|---|---|
| H5N5 | 1866 | CT | −∞ |

¹⁾Proposed composition wherein the monosaccharide symbols are:
H, Hex; N, HexNAc; F, dHex.
²⁾Calculated m/z for [M + Na]+ ion rounded down to next integer.
³⁾N-glycan class symbols are: M, high-mannose type; L, low-mannose
type; H, hybrid-type or monoantennary; C, complex-type; O, other type;
F, fucosylated; E, complex-fucosylated, wherein at least one fucose
residue is α1,2-, α1,3- or α1,4-linked; R, large complex-type;
G, glucosylated; T, non-reducing terminal HexNAc.
${ }^{4 )} ‘ fold ’ is calculated according to the equation \text{:} fold = {x ( \frac{{P}_{a}}{{P}_{b}} )}^{x} , wherein$
P is the relative abundancy (%) of the glycan signal in profile a or b,
x is 1 when Pₐ ≧ P_{b}, and x is −1 when a < b; +∞, detected only in
CD133+ cells; −∞, not detected in CD133+ cells.
⁵⁾Association with human cord blood mononuclear cell type based
on fold calculation: + low association, ++ substantial association,
+++ high association.

TABLE 23
Sialylated N-glycan difference analysis.

| | | | |
|---|---|---|---|
| composition¹⁾ | m/z²⁾ | class³⁾ | fold⁴⁾ |

+++ CD133+⁵⁾

| | | | |
|---|---|---|---|
| S1H3N3 | 1403 | H | +∞ |
| S1H4N3F1P | 1791 | HFP | +∞ |
| S4H3N3 | 1856 | H | +∞ |
| S3H4N3F1 | 2293 | HF | +∞ |
| S1H7N6F2 | 2953 | CER | +∞ |

++ CD133+

| | | | |
|---|---|---|---|
| S2H5N4 | 2221 | C | 1.55 |
| S2H5N4F1 | 2367 | CF | 1.53 |
| S1H3N3F1 | 1549 | HF | 1.51 |

+ CD133+

| S1H3N2 | 1200 | | 1.39 |
|---|---|---|---|
| S1H5N4F3 | 2368 | CE | 1.35 |
| S1H5N4F2 | 2222 | CE | 1.26 |
| S1H5N4 | 1930 | C | 1.20 |
| S1H4N4F1 | 1914 | CF | 1.13 |
| S1H4N4 | 1768 | C | 1.08 |
| composition | m/z | class | fold |

+CD133−

| | | | |
|---|---|---|---|
| S1H5N4F1 | 2076 | CF | −1.02 |
| S1H4N3F1 | 1711 | HF | −1.02 |
| S1H5N3F1 | 1873 | HF | −1.11 |
| S1H4N3 | 1565 | H | −1.20 |
| S2H6N5F1 | 2732 | CFR | −1.22 |
| S2H5N4F4 | 2806 | CE | −1.32 |
| S1H7N6F3 | 3099 | CER | −1.36 |
| S1H5N3 | 1727 | H | −1.43 |

++ CD133−

| | | | |
|---|---|---|---|
| S1H5N5F1 | 2279 | CFT | −1.60 |
| S1H6N3 | 1889 | H | −1.61 |
| S1H6N5F1 | 2441 | CFR | −1.82 |

+++ CD133−

| | | | |
|---|---|---|---|
| S1H7N6F1 | 2807 | CFR | −7.60 |
| S1H5N5 | 2133 | CT | −∞ |
| S1H6N5 | 2295 | CR | −∞ |
| S1H6N5F2 | 2587 | CER | −∞ |
| S1H6N5F3 | 2733 | CER | −∞ |
| S3H6N5F1 | 3024 | CFR | −∞ |
| S2H7N6F1 | 3098 | CFR | −∞ |
| S2H7N6F3 | 3390 | CER | −∞ |

¹⁾Proposed composition wherein the monosaccharide symbols are:
S, NeuAc; H, Hex; N, HexNAc; F, dHex; P, SP = sulphate or
phosphate ester.
²⁾Calculated m/z for [M − H]− ion rounded down to next integer.
³⁾N-glycan class symbols are: H, hybrid-type or monoantennary;
C, complex-type; O, other type; F, fucosylated; E, complex-fucosylated,
wherein at least one fucose residue is α1,2-, α1,3- or α1,4-linked;
R, large complex-type; non-reducing terminal
HexNAc.
${ }^{4 )} ‘ fold ’ is calculated according to the equation \text{:} fold = {x ( \frac{{P}_{a}}{{P}_{b}} )}^{x} , wherein$
P is the relative abundancy (%) of the glycan signal in profile a or b,
x is 1 when Pₐ ≧ P_{b}, and x is −1 when a < b; +∞, detected only in
CD133+ cells; −∞, not detected in CD133+ cells.
⁵⁾Association with human cord blood mononuclear cell type based
on fold calculation: + low association, ++ substantial association,
+++ high association.

TABLE 24
Flow cytometric (FACS) analysis of cord blood hematopoietic stem cells
(CB-HSCs, CD34+) and mature blood cells (CD34−).

| | | | | CB-HSC | | CB-HSC | |
|---|---|---|---|---|---|---|---|
| Code | Trivial name | Structure | Terminal epitope | CD34+ | SD | CD34− | SD |
| GF 416 | Mannose | 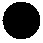 | Man | 6.0 | 1.1 | 7.7 | 2.3 |
| GF 278 | Tn | 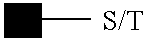 | GalNAcαS/T | 36.6 | 11.0 | 12.8 | 0.5 |
| VPU 006 | Tn antigen, CD175 | 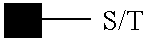 | GalNAcαS/T | 36.5 | | 12.6 | |
| VPU 007 | sialyl Tn, sCD175 | 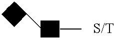 | SA(α6)GalNAcαS/T | 3.8 | | 3.2 | |
| GF 277 | Sialosyl-Tn | 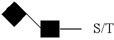 | SA(α6)GalNAcαS/T | 4.7 | 1.9 | 10.7 | 1.8 |
| GF 276 | TAG-72, CA 72-4 | 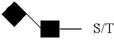 | | 11.7 | 4.4 | 7.6 | 2.8 |
| GF 280 | TF-antigen | 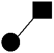 | Gal(β3)GalNAc(α/β) | 19.1 | 12.1 | 7.0 | 1.0 |
| GF 281 | TF-antigen | 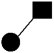 | Gal(β3)GalNAc(α/β) | 40.2 | 6.8 | 11.1 | 1.9 |
| GF 365 | TF-antigen | 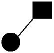 | Gal(β3)GalNAc(α/β) | 18.6 | 12.4 | 7.2 | 0.5 |
| GF 274 | MECA-79, Sulfo- mucin, PNAD | 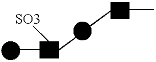 | Sulfo-mucin | 14.6 | 14.0 | 18.4 | 0.1 |
| GF 374 | Glycodelin A | 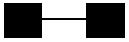 | LacdiNAc | 9.0 | 3.9 | 14.2 | 1.6 |
| GF 375 | Glycodelin A | 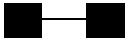 | LacdiNAc | 18.3 | 15.5 | 15.4 | 3.2 |
| GF 376 | Glycodelin A | 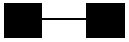 | LacdiNAc | 18.5 | 11.0 | 11.5 | 0.8 |
| GF 413 | Gal(α3)Gal | 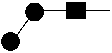 | Gal(α3)Gal | 7.3 | 2.8 | 4.1 | 1.5 |
| GF 295 | Lewis c | 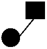 | Gal(β3)GlcNAcβ(3Lac) | 13.2 | 1.4 | 19.6 | 8.2 |
| GF 300 GF 428 | asialo GM2 | 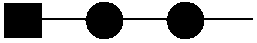 | GalNAc(β4)Gal(β4)GlcβCer | 10.0 | 10.1 | 32.4 | 11.2 |
| GF 296 GF 427 | asialo GM1 | 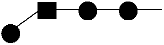 | Gal(β3)GalNAc(β4)Gal(β4)GlcβCer | 11.1 | 12.5 | 30.8 | 7.7 |
| GF 406 | GD2 | 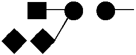 | | 4.5 | | 5.0 | |
| GF 298 | Gb3 | 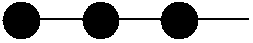 | Gal(α4)Gal(β4)GlcβCer | 11.3 | 4.7 | 18.5 | 0.7 |
| GF 297 VPU 001 | Globoside GL4 | 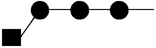 | GalNAc(β3)Gal(α4)Gal(β4)GlcβCer | 6.0 | 2.4 | 23.2 | 6.5 |
| GF 353 | SSEA-3 | 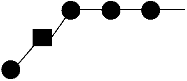 | Gal(β3)GalNAc(β3)Gal | 5.9 | 1.2 | 20.1 | 0.7 |
| GF 354 | SSEA-4 | 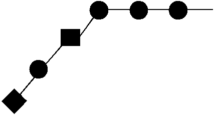 | SA(α3)Gal(β3)GalNAc(β3)Gal | 4.9 | 1.6 | 15.1 | 7.0 |
| GF 299 | Forssman ag | 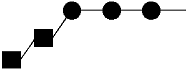 | GalNAc(α3)GalNAc(β4)Gal(α4)Gal (β4)GlcβCer, GalNAc(α3)GalNAcβ-R | 9.1 | 6.4 | 20.2 | 3.2 |
| GF 288 | Globo-H | 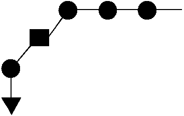 | Fuc(α2)Gal(β3)GalNAc(β3)Gal(α4) Gal(β4)GlcβCer | 10.6 | 2.8 | 5.4 | 1.5 |
| GF 394 | H disaccharide | 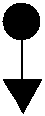 | Fuc(α2)Galβ | 10.1 | 4.8 | 5.7 | 1.6 |
| GF 303 | H Type 1 | 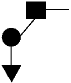 | Fuc(α2)Gal(β3)GlcNAc | 4.7 | 1.3 | 13.7 | 2.0 |
| GF 304 | Lewis a | 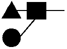 | Gal(β3)[Fuc(α4)]GlcNAc | 11.2 | 3.8 | 18.1 | 4.3 |
| GF 306 | sialyl Lewis a | 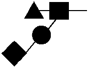 | SA(α3)Gal(β3)[Fuc(α4)]GlcNAc | 6.4 | 2.9 | 18.1 | 7.3 |
| GF 301 | Lewis b | 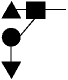 | Fuc(α2)Gal(β3)[Fuc(α4)]GlcNAc | 7.3 | | 19.3 | |
| GF 302 | H Type 2 | 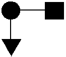 | Fuc(α2)Gal(β4)GlcNAc | 6.2 | 3.5 | 19.1 | 2.6 |
| GF 410 | blood group ABH | 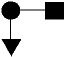 | | 8.9 | 5.4 | 7.8 | 1.1 |
| GF 305 | Lewis x | 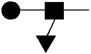 | Gal(β4)[Fuc(α3)]GlcNAc | 26.9 | 21.7 | 6.9 | 3.9 |
| GF 515 | Lex, CD15 | 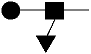 | Gal(β4)[Fuc(α3)]GlcNAc | 8.8 | 11.4 | 13.2 | 7.6 |
| GF 517 | Lex, CD15 | 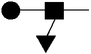 | Gal(β4)[Fuc(α3)]GlcNAc | 28.7 | 31.5 | 4.2 | 2.1 |
| GF 518 | SSEA-1 (CD15, Lex) | 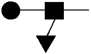 | Gal(β4)[Fuc(α3)]GlcNAc | 22.7 | 29.2 | 6.7 | 3.0 |
| GF 525 | CD15 (Lex) | 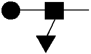 | Gal(β4)[Fuc(α3)]GlcNAc | 14.3 | 16.1 | 13.8 | 3.1 |
| GF 516 | sLex, sCD15 | 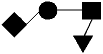 | SA(α3)Gal(β4)[Fuc(α3)]GlcNAc | 43.4 | 15.1 | 5.9 | 3.6 |
| GF 307 | sialyl Lewis x | 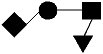 | SA(α3)Gal(β4)[Fuc(α3)]GlcNAc | 85.5 | 6.3 | 13.7 | 3.4 |
| GF 526 | PSGL-1 (sLex on core 2 O-glycans) | 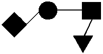 | SA(α3)Gal(β4)[Fuc(α3)]GlcNAc | 97.6 | 0.4 | 33.7 | 11.1 |
| GF 393 | Lewis y | 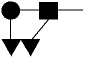 | Fuc(α2)Gal(β4)[Fuc(α3)]GlcNAcβ | 5.5 | 1.3 | 7.1 | 1.9 |
| GF 408 | blood group Ag A-b45.1 | 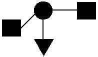 | GalNAc(α3)Fuc(α2)Galβ | 5.1 | 2.3 | 17.1 | 4.6 |
| GF 409 | blood group A | 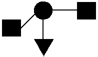 | | 6.8 | 3.0 | 8.0 | 1.0 |
| GF 411 | blood group B (secretor) | 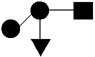 | | 5.9 | 1.9 | 8.7 | 2.3 |
| GF 412 | blood group B (general) | 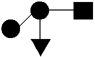 | | 8.0 | 5.8 | 6.9 | 1.0 |
| GF 414 | TRA-1-81 Ag | | | 6.1 | | 9.7 | |
| GF 415 | TRA-1-60 Ag | | | 11.2 | | 5.6 | |

TABLE 25
Detailed information of the primary anti-glycan antibodies used in these examples.
Alternative antibody clones in italics.

| Code | Epitope | Terminal structure |
|---|---|---|
| GF 274 | Sulfo-mucin, PNAD, MECA-79, CD62L, | Sulfo-mucin |
| | extended core 1 | |
| GF 275 | Ca15-3 sialyted epitope | SAα-mucin |
| GF 553 | | |
| GF 276 | TAG-72, CA 72-4, cancer glycoprotein | |
| GF 277 | Sialosyl-Tn, sCD175 | SA(α6)GalNAcαS/T |
| GF 372 | | |
| GF 278 | Tn, CD175 | GalNAcαS/T |
| VPU008 | | |
| GF 280 | TF-antigen isoform, CD176 | Gal(β3)GalNAc(α/β) (α 40x > β) |
| GF 281 | TF-antigen isoform, CD176 | Gal(β3)GalNAcβ |
| GF 285 | H Type 2, Lewis b, Lewis y | Fuc(α2)Gal, Fuc(a2)Gal(β4)GlcNAc, |
| | | Fuc(α2)Gal(β4)[Fuc(α3)]GlcNAc |
| GF 286 | H Type 2, CD173 | Fuc(α2)Gal(β4)GlcNAc |
| GF 288 | Globo-H | Fuc(α2)Gal(β3)GalNAc(β3)Gal(α4)Gal(β4)GlcβCer |
| GF 403 | | |
| GF 295, | Lewis c, pLN, Gal(β3)GlcNAc | Gal(β3)GlcNAβ(3Lac) |
| GF 279 GF | | |
| 555 | | |
| GF 296, | asialo GM1 | Gal(β3)GalNAc(β4)Gal(β4)GlcβCer |
| GF 282 | | |
| GF 427 | | |
| GF 297, | Globoside Gb4, GL4, globotetraose | GalNAc(β3)Gal(α4)Gal(β4)GlcβCer |
| GF 366 | | |
| VPU001 | | |
| GF 298 | Globoside Gb3, globotriose, CD77, | Gal(α4)Gal(β4)GlcβCer |
| GF 367 | blood group pk | |
| GF 299, | Forssman ag, glycosphingolipid | GalNAc(α3)GalNAc(β4)Gal(α4)Gal(β4)GlcβCer, |
| GF 401 | | GalNAc(α3)GalNAcβ-R |
| GF 554 | | |
| GF 300 | asialo GM2 | GalNAc(β4)Gal(β4)GlcβCer |
| GF 428 | | |
| GF 301, | Lewis b | Fuc(α2)Gal(β3)[Fuc(α4)]GlcNAc |
| GF 283 | | |
| VPU004 | | |
| GF 302 | H Type 2 | Fuc(α2)Gal(β4)GlcNAc |
| GF 284 | | |
| GF 303 | H Type 1, blood group antigen H1 | Fuc(α2)Gal(β3)GlcNAc |
| GF 287 | | |
| GF 304 | Lewis a | Gal(β3)[Fuc(α4)]GlcNAc |
| GF 429 | | |
| GF 305 | Lewis x, CD15, SSEA-1 | Gal(β4)[Fuc(α3)]GlcNAc |
| GF 306, | sialyl Lewis a | SA(α3)Gal(β3)[Fuc(α4)]GlcNAc |
| GF 430 | | |
| VPU002 | | |
| | sialyl Lewis a, c | |
| GF 307 | sialyl Lewis x | SA(α3)Gal(β4)[Fuc(α3)]GlcNAc |
| GF 353 | SSEA-3, galactosylgloboside | Gal(β3)GalNAc(β3)Gal |
| GF 431 | | |
| GF 354, | SSEA-4, sialylgalactosylgloboside | SA(α3)Gal(β3)GalNAc(β3)Gal |
| GF 432 | | |
| VPU003 | | |
| GF 355 | Gal(α3)Gal | Gal(α3)Gal |
| GF 365 | TF-antigen isoform, CD176 | Gal(β3)GalNAc(α/β) (α 10x > β) |
| GF 368 | LacdiNAc | GalNAc(β4)GlcNAc |
| GF 369 | LacdiNAc | GalNAc(β4)GlcNAc |
| GF 370 | α3-Fuc-LacdiNAc | GalNAc(β4)[Fuc(α3)]GlcNAc |
| GF 371 | α3-Fuc-LacdiNAc | GalNAc(β4)[Fuc(α3)]GlcNAc |
| GF 374 | Glycodelin A, isoform | LacdiNAc |
| GF 375 | Glycodelin A, isoform | LacdiNAc |
| GF 376 | Glycodelin A, isoform | LacdiNAc |
| GF 377 | PN-15 renal gp200, cancer glycoprotein | |
| GF 373 | | |
| GF 393 | Lewis y, CD174 | Fuc(α2)Gal(β4)[Fuc(α3)]GlcNAcβ |
| GF 289 | | |
| GF 394 | H disaccharide | Fuc(α2)Galβ |
| GF 290 | | |
| GF 406 | GD2 | GalNAc(β4)(SA(α8)SA)(α3)Gal(β4)Glc |
| GF 558 | | |
| GF 407 | GD3 | SA(α8)SA(α3)Gal(β4)Glc |
| GF 559 | | |
| GF 408 | blood group Ag A-b45.1 (A1, A2) | GalNAc(α3)Fuc(α2)Galβ |
| GF 409 | blood group A (A3, Ax, A3B, AxB) | |
| GF 410 | blood group ABH | |
| GF 411 | blood group B (secretor) | |
| GF 412 | blood group Ag B (general) | |
| GF 413 | Gal(α3)Gal | Gal(α3)Gal(β4)GlcNAc-R |
| GF 414 | TRA-1-81 Ag | |
| GF 556 | | |
| GF 415 | TRA-1-60 Ag | |
| GF 557 | | |
| GF 416 | Mannose | Man |
| GF 418 | Globo-H | Fuc(α2)Gal(β3)GalNAc(β3)Gal(α4)Gal(β4)GlcβCer |
| GF 515 | CD15, Lewis x | Gal(β4)[Fuc(α3)]GlcNAc |
| GF 516 | sCD15, sialyl Lewis x | SA(α3)Gal(β4)[Fuc(α3)]GlcNAc |
| GF 517 | CD15, Lewis x | Gal(β4)[Fuc(α3)]GlcNAc |
| GF 518 | SSEA-1 | Gal(β4)[Fuc(α3)]GlcNAc |
| GF 525 | CD15, reacts with 220 kD protein | Gal(β4)[Fuc(α3)]GlcNAc |
| GF 526 | PSGL-1, sLex on core 2 O-glycans | SA(α3)Gal(β4)[Fuc(α3)]GlcNAc |
| GF 621 | GD3 | SA(α8)SA(α3)Gal(β4)Glc |
| GF 622 | GD2 | GalNAc(β4)(SA(α8)SA)(α3)Gal(β4)Glc |
| GF 623 | GT1b | |
| GF 624 | GD1b | |
| GF 625 | GD2 | GalNAc(β4)(SA(α8)SA)(α3)Gal(β4)Glc |
| GF 626 | GD3 | SA(α8)SA(α3)Gal(β4)Glc |
| GF 627 | OAcGD3 | |
| GF 628 | A2B5 | |
| VPU005 | GD3 | SA(α8)SA(α3)Gal |
| VPU006 | Tn antigen, CD175 | GalNAcαS/T |
| VPU007 | sialyl Tn, sCD175 | SA(α6)GalNAcαS/T |
| VPU009 | SSEA-3, galactosylgloboside | Gal(β3)GalNAc(β3)Gal |
| | GlcNAcβ1-6R | |
| | Galβ1-4GlcNAcβ1-3R | |
| | Galβ1-4GlcNAcβ1-6R | |

| Code | Company | Cat number | Clone | Host/Class |
|---|---|---|---|---|
| GF 274 | BD | 553863 | MECA-79 | rat/IgM |
| | Pharmingen | | | |
| GF 275 | Acris | BM3359 | 695 | mouse/IgG1 |
| GF 553 | | | | |
| GF 276 | Acris | DM288 | B72.3 | mouse/IgG1 |
| GF 277 | Acris | DM3197 | B35.1 | mouse/IgG1 |
| GF 372 | | | | |
| GF 278 | Acris | DM3218 | B1.1 | mouse/IgM |
| VPU008 | | | | |
| GF 280 | Glycotope | MAB-S301 | Nemod | mouse/IgM |
| | | | TF2 | |
| GF 281 | Glycotope | MAB-S305 | A68-E/E3 | mouse/IgG1 |
| GF 285 | Acris | DM3014 | B389 | mouse/IgG1 |
| GF 286 | Acris | BM258P | BRIC 231 | mouse/IgG1 |
| GF 288 | Glycotope | MAB-S206 | A69-A/E8 | mouse/IgM |
| GF 403 | | | | |
| GF 295, | Abcam | ab3352 | K21 | mouse/IgM |
| GF 279 GF | | | | |
| 555 | | | | |
| GF 296, | Acris | BP282 | polyclonal | rabbit |
| GF 282 | | | | |
| GF 427 | | | | |
| GF 297, | Abcam | ab23949 | polyclonal | rabbit/IgG |
| GF 366 | | | | |
| VPU001 | | | | |
| GF 298 | Acris | SM1160P | 38-13 | rat/IgM |
| GF 367 | | | | |
| GF 299, | Acris | BM4091 | FOM-1 | rat/IgM |
| GF 401 | | | | |
| GF 554 | | | | |
| GF 300 | Acris | BP283 | polyclonal | rabbit |
| GF 428 | | | | |
| GF 301, | Acris | SM3092P | 2-25LE | mouse/IgG1 |
| GF 283 | | DM3122 | | |
| VPU004 | | | | |
| GF 302 | Acris | DM3015 | B393 | mouse/IgM |
| GF 284 | | | | |
| GF 303 | Abcam | ab3355 | 17-206 | mouse/IgG3 |
| GF 287 | | | | |
| GF 304 | Chemicon | CBL205 | PR5C5 | mouse/IgG1 |
| GF 429 | Abcam | Ab3967 | 7LE | |
| | | Ab3356 | T174 | |
| | Genetex | GTX28602 | B369 | |
| GF 305 | Chemicon | CBL144 | 28 | mouse/IgM |
| GF 306, | Chemicon | MAB2095 | KM231 | mouse/IgG1 |
| GF 430 | Invitrogen | 18-7240 | 116-NS- | |
| VPU002 | | | 19-9 | |
| | BioGenex | MU424-UC | C241:5:1:4 | |
| | Seikagaku | 270443 | 2D3 | mouse/IgM |
| GF 307 | Chemicon | MAB2096 | KM93 | mouse/IgM |
| GF 353 | Chemicon | MAB4303 | MC-631 | rat/IgM |
| GF 431 | | | | |
| GF 354, | Chemicon | MAB4304 | MC-813- | mouse/IgG3 |
| GF 432 | | | 70 | |
| VPU003 | | | | |
| GF 355 | Chemicon | AB2052 | | baboon |
| GF 365 | Glycotope | MAB-S302 | Nemod | mouse/IgM |
| | | | TF1 | |
| GF 368 | LUMC | anti-LDN | 259-2A1 | IgG3 |
| | (Leiden Univ | mAb | | |
| | Medical | | | |
| | Center) | | | |
| GF 369 | LUMC | anti-LDN | 273-3F2 | IgM |
| | (Leiden Univ | mAb | | |
| | Medical | | | |
| | Center) | | | |
| GF 370 | LUMC | anti LDN-F | 290-2E6 | IgM |
| | (Leiden Univ | mAb | | |
| | Medical | | | |
| | Center) | | | |
| GF 371 | LUMC | anti LDN-F | 291-3E9 | IgM |
| | (Leiden Univ | mAb | | |
| | Medical | | | |
| | Center) | | | |
| GF 374 | Glycotope | MAB-S901 | A87-D/C5 | mouse/IgG1, |
| | | | | IgG2b, IgM |
| GF 375 | Glycotope | MAB-S902 | A87-D/F4 | mouse/IgG1 |
| GF 376 | Glycotope | MAB-S903 | A87-B/D2 | mouse/IgG1 |
| GF 377 | Acris | DM3184P | PN-15 | mouse/IgG1 |
| GF 373 | | | | |
| GF 393 | Glycotope | MAB-S201 | A70-C/C8 | mouse/IgM |
| GF 289 | | | | |
| GF 394 | Glycotope | MAB-S204 | A51-B/A6 | mouse/IgA |
| GF 290 | | | | |
| GF 406 | Chemicon | MAB4309 | VIN-2PB- | mouse/IgM |
| GF 558 | | | 22 | |
| GF 407 | Chemicon | MAB4308 | VIN-IS-56 | mouse/IgM |
| GF 559 | | | | |
| GF 408 | Acris | DM3108 | B480 | mouse/IgG1 |
| GF 409 | Acris | BM255 | HE-195 | mouse/IgM |
| GF 410 | Acris | SM3004 | HE-10 | mouse/IgM |
| GF 411 | Acris | BM256 | HEB-29 | mouse/IgM |
| GF 412 | Acris | DM3012 | B460 | mouse/IgM |
| GF 413 | Alexis | ALX-801-090 | M86 | mouse/IgM |
| | Biochemicals | | | |
| GF 414 | Chemicon | MAB4381 | TRA-1-81 | mouse/IgM |
| GF 556 | | | | |
| GF 415 | Chemicon | MAB4360 | TRA-1-60 | mouse/IgM |
| GF 557 | | | | |
| GF 416 | | | | mouse/IgM |
| GF 418 | Alexis | ALX-804- | MBr1 | mouse/IgM |
| | biochemicals | 550-C050 | | |
| GF 515 | BD | 557895 | W6D3 | mouse/IgG1, k |
| | Pharmingen | | | |
| GF 516 | BD | 551344 | CSLEX1 | mouse/IgM, k |
| | Pharmingen | | | |
| GF 517 | Abcam | ab34200 | TG-1 | mouse/IgM |
| GF 518 | Abcam | ab16285 | MC480 | mouse/IgM |
| GF 525 | Abcam | ab17080 | MMA | mouse/IgM |
| GF 526 | R&D | MAB996 | CHO131 | mouse/IgM |
| | Systems | | | |
| GF 621 | BD | 554274 | MB3.6 | mouse/IgG3 |
| | Pharmingen | | | |
| GF 622 | BD | 554272 | 14.G2a | mouse/IgG2 |
| | Pharmingen | | | |
| GF 623 | US Biological | G2006-90A | 3C96 | mouse/IgM |
| GF 624 | US Biological | G2004-90B | 2S1 | mouse/IgG3 |
| GF 625 | US Biological | G2205-02 | 2Q549 | mouse/IgG2 |
| GF 626 | Covalab | mab0014 | 4F6 | mouse/IgG3 |
| GF 627 | US Biological | G2005-67 | 4i283 | mouse/IgG3 |
| GF 628 | Chemicon | MAB312R | A2B5-105 | mouse/IgM |
| VPU005 | Seikagaku | 270554 | S2-566 | mouse/IgM |
| VPU006 | Abcam | ab31775 | 0.BG.12 | mouse/IgG |
| VPU007 | Abcam | ab24005 | BRIC111 | mouse/IgG |
| VPU009 | R&D | MAB1434 | MC-631 | rat/IgM |
| | Systems | | | |
| | Jeffersson | FE-J1 | | mouse/IgM |
| | medical | | | |
| | college | | | |
| | Jeffersson | FE-A5 | | mouse/IgM |
| | medical | | | |
| | college | | | |
| | Jeffersson | FE-A6 | | mouse/IgM |
| | medical | | | |
| | college | | | |

TABLE 26
Flow cytometric (FACS) and immunohistochemical (IHC) analysis of mesenchymal stem
cells (MSC) and cells differentiated into osteogenic (OG) and adipogenic (adipo) lineages.

| | | | | BM-MSC¹⁾ | BM-OG | | | |
|---|---|---|---|---|---|---|---|---|
| | Trivial | | Terminal | FACS(% ± SD) | FACS(% ± SD) | CB-MSC | CB-OG | CB-Adipo |
| Code | name | Structure | epitope | IHC²⁾ | IHC | FACS(% ± SD) | FACS(%) | FACS(%) |
| GF416 | Mannose | 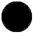 | Man | 0.8 ± 0.42 | 13.2 | 2.90 ± 2.8 | 8.60 | 34.9 |
| GF278 VPU008 | Tn | 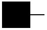 | GalNAcαS/T | 5.9 ± 1.7 + | 2.95 ± 2.6 ++ | 2.43 ± 2.75 | 0.70 | 1.8 |
| VPU006 | Tn antigen, CD175 | 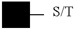 | GalNAcαS/T | 0.9 ± 0.35 | ND | 0.6 ± 0.17 | 0.5 | 0.6 |
| VPU007 | sialyl Tn, sCD175 | 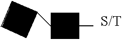 | SAα6GalNAcαS/T | 1.3 ± 0.28 | ND | 0.5 ± 0.17 | 0.8 | 1 |
| GF277 | Sialosyl-Tn | 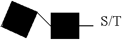 | SAα6GalNAcαS/T | 7.3 ± 4.67 + | 0.95 ± 0.21 ++ | 2.63 ± 1.6 | 0.8 | 5.7 |
| GF276 | TAG-72, CA 72-4 | 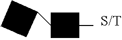 | TAG-72 carried sialyl-Tn, cancer glycoprotein | 0.75 ± 0.35 − | 0.75 ± 0.64 ++ | 0.90 ± 0.28 | 0.6 | 0.6 |
| GF280 | TF-antigen | 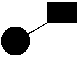 | Galβ3GalNAcα/β (α 40x > β) | 5 − | ND − | 1.97 ± 1.65 | 0.7 | 0.8 |
| GF281 | TF-antigen | 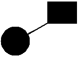 | Galβ3GalNAcα | 1.3 − | ND − | 6.2 ± 7.3 | 0.9 | 2.5 |
| GF365 | TF-antigen | 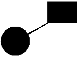 | Galβ3GalNAcα/β (α 10x > β) | 2.95 ± 1.2 − | 1.1 − | 4.25 ± 4.2 | 1.4 | 11.6 |
| GF274 | MECA-79, Sulfo-mucin, PNAD | 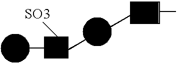 | Sulfo-mucin | 0.9 − | 1.8 ± 0.14 − | 2.4 ± 2.3 | 1.1 | 1.7 |
| GF275 GF553 | Cal 5-3 sialyted epitope | | SAα-mucin | 46.5 ± 38.0 ++ | 79.1 ± 25.2 +++ | 2.0 ± 0.0 | 6.9 | 30.8 |
| GF374 | Glycodelin A | 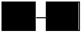 | N-glycan/LacdiNAc | 0.9 ± 0.0 +/− | 0.3 − | 1.80 ± 1.3 | 0.9 | 0.9 |
| GF375 | Glycodelin A | 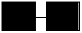 | N-glycan/LacdiNAc | 1.9 ± 0.71 − | 0.6 − | 5.85 ± 6.9 | 0.8 | 1.0 |
| GF376 | Glycodelin A | 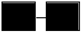 | N-glycan/LacdiNAc | 3.4 − | 0.6 − | 2.2 ± 0.85 | 1.8 | 1.4 |
| GF413 | Galα3Gal | 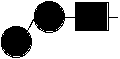 | Galα3Galβ4GlcNAc | 0.9 ± 0.42 | 0.8 | 7.45 ± 3.9 | 0.7 | 1.7 |
| GF295 GF555 | Lewis c | 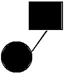 | pLN, Galβ3GlcNAc | 9.6 ± 7.4 − | 2.7 ± 2.5 − | 7.15 ± 2.8 | 1.9 | 17.2 |
| GF300 GF428 | asialo GM2 | 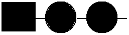 | GalNAcβ4Galβ4GlcβCer | 17.1 ± 3.3 − | 53.8 ± 2.1 − | 7.40 ± 3.4 | 47.9 | 63.4 |
| GF296 GF427 | asialo GM1 | 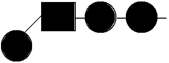 | Galβ3GalNAcβ4Galβ4GlcβCer | 22 ± 17.4 − | 48.2 ± 18.0 − | 10.30 ± 6.8 | 44.5 | 66.1 |
| GF624 | GD1b | 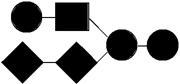 | | 3.5 ± 0.35 | ND | 7.4 ± 8.3 | 10.7 | 22.2 |
| GF623 | GT1b | 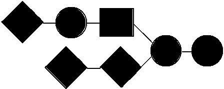 | | 30.7 ± 10.5 | ND | 20.85 ± 15.9 | 72.7 | 74.3 |
| GF406 GF558 | GD2 | 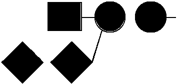 | GalNAcβ4(SAα8SAα3)Galβ4Glc | 0.9 ± 0.71 | 1.2 | 7.45 ± 7.6 | 1.4 | 20.6 |
| GF622 | GD2 | 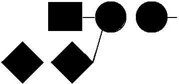 | GalNAcβ4(SAα8SAα3)Galβ4Glc | 50.8 ± 4.45 | ND | 5.25 ± 0.64 | 91.5 | 97.3 |
| GF625 | GD2 | 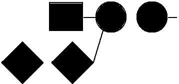 | GalNAcβ4(SAα8SAα3)Galβ4Glc | 44.2 ± 0.42 | ND | 7.2 ± 0.57 | 92.1 | 95.7 |
| GF407 GF559 | GD3 | 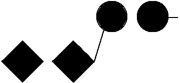 | SAα8SAα3Galβ4Glc | 0.8 | ND | 4.75 ± 0.92 | 1.4 | 58.3 |
| GF621 | GD3 | 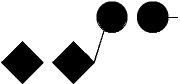 | SAα8SAα3Galβ4Glc | 18.4 ± 7.2 | ND | 2.8 ± 2.1 | 89.4 | 99 |
| GF626 | GD3 | 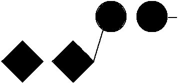 | SAα8SAα3Galβ4Glc | 2.9 ± 0.64 | ND | 1.95 ± 0.6 | 4.1 | 41.5 |
| VPU005 | GD3 | 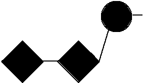 | SAα8SAα3Gal | 27.5 ± 4.45 | 29.9 | 10.1 ± 1.84 | 98.0 | 99.8 |
| GF627 | OAcGD3 | 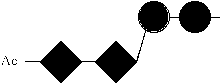 | Acetyl-SAα8SAα3Galβ4Glc | 0.6 ± 0.14 | ND | 1.35 ± 0.78 | 0.8 | 0.7 |
| GF628 | A2B5 | | | 27.6 ± 11.0 | ND | 37.2 ± 15.0 | 58 | 81 |
| GF298 | Gb3 | 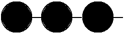 | Galα4Galβ4GlcβCer | 21.8 +++ | 52.7 ± 2.3 ++ | 6.15 ± 0.92 | 5.8 | 6.1 |
| GF297 VPU001 | Globoside GL4 | 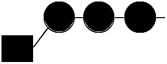 | GalNAcβ3Galα4Galβ4GlcβCer | 16.9 +++ | 28.4 ++ | 9.75 ± 4.2 | 30.1 | 61.2 |
| GF353 GF431 | SSEA-3 | 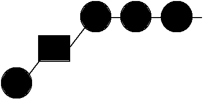 | Galβ3GalNAcβ3Gal | 3.4 ± 2.26 ++ | 6.2 ± 3.3 + | 1.95 ± 1.5 | 0.9 | 1.2 |
| VPU009 | SSEA-3 | 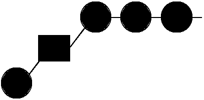 | Galβ3GalNAcβ3Gal | 11.9 ± 8.5 | ND | 75.75 ± 2.8 | 38.3 | 71.7 |
| GF354, GF432 VPU003 | SSEA-4 | 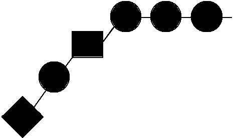 | SAα3Galβ3GalNAcβ3Galα4Galβ4Glc | 58.3 ± 23.6 +++ | 26.5 ± 18.0 +/− | 59.8 ± 0.57 | 32.6 | 80.5 |
| GF299 GF554 | Forssman ag | 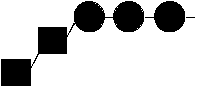 | GalNAcβ3GalNAcβ3Galα4Galβ4Glc | 4.1 − | 5.5 ± 1.7 − | 2.85 ± 2.1 | 0.4 | 2.4 |
| GF630 | Forssman ag | 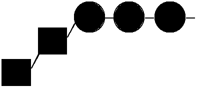 | GalNAcβ3GalNAcβ3Galα4Galβ4Glc | 0.3 | ND | 1.4 | 0.3 | 0.7 |
| GF288 | Globo-H | 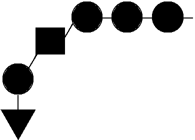 | Fucα2Galβ3GalNAcβ3Galα4Galβ4GlcβCer | 0.4 ± 0.07 − | 0.6 − | 1.35 ± 0.49 | 0.5 | 0.7 |
| GF394 | H disaccharide | 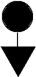 | Fucα2Galβ | 1.5 ± 0.42 − | 0.6 ± 0.14 − | 12.90 ± 8.9 | 0.6 | 0.5 |
| GF303 | H Type 1 | 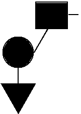 | Fucα2Galβ3GlcNAc | 1.4 ± 0.07 − | 0.7 ± 0.0 ++ | 1.2 ± 0.28 | 0.8 | 1.3 |
| GF304 GF429 | Lewis a | 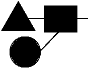 | Galβ3(Fucα4)GlcNAc | 13 ± 1.8 − | 23.4 ± 18.4 − | 11.3 ± 0.79 | 31.1 | 59.3 |
| GF306, GF430 VPU002 | sialyl Lewis a | 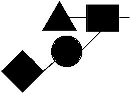 | SAα3Galβ3(Fucα4)GlcNAc | 3.0 ± 2.3 − | 5.1 ± 4.4 − | 7.6 ± 5.1 | 4.9 | 14.6 |
| GF629 | sialyl Lewis a | 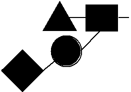 | SAα3Galβ3(Fucα4)GlcNAc | 0.5 | ND | 1.4 | 1.3 | 2.4 |
| GF301 VPU004 | Lewis b | 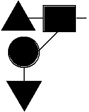 | Fucα2Galβ3(Fucα4)GlcNAc | 1.2 ± 0.0 − | 1.3 ± 0.49 − | 1.2 ± 0.85 | 0.7 | 1.4 |
| GF302 | H Type 2 | 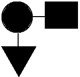 | Fucα2Galβ4GlcNAc | 14.7 ± 12.3 ++ | 26.2 ± 4.0 ++ | 9.4 ± 0.57 | 46.0 | 61.5 |
| GF410 | blood group ABH | 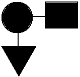 | Fucα2Galβ4GlcNAc | 0.4 ± 0.07 | 0.7 | 0.85 ± 0.21 | 0.7 | 0.7 |
| GF305 | Lewis x | 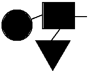 | Galβ4(Fucα3)GlcNAc | 1.0 +/− | 1.1 ± 0.49 − | 3.2 ± 2.5 | 0.8 | 3.0 |
| GF515 | Lewis x, CD15 | 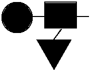 | Galβ4(Fucα3)GlcNAc | 0.3 ± 0.14 | 0.7 | 1.57 ± 0.49 | 0.7 | 2.9 |
| GF517 | Lewis x, CD15 | 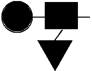 | Galβ4(Fucα3)GlcNAc | 0.3 ± 0.0 | 0.7 | 6.5 ± 8.7 | 0.5 | 2.4 |
| GF518 | SSEA-1 (CD15, Lex) | 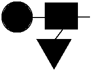 | Galβ4(Fucα3)GlcNAc | 0.3 ± 0.0 | 0.6 | 0.9 ± 0.14 | 1.0 | 1.8 |
| GF525 | CD15 (Lex), reacts with 220 kD protein | 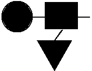 | Galβ4(Fucα3)GlcNAc | 1.1 ± 0.64 | 2.7 | 6.97 ± 2.4 | 2.5 | 48.3 |
| GF516 | sialyl Lewis x, sCD15 | 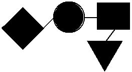 | SAα3Galβ4(Fucα3)GlcNAc | 8.5 ± 13.5 | 10.4 | 7.8 ± 5.9 | 19.0 | 13.5 |
| GF307 | sialyl Lewis x | 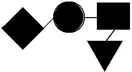 | SAα3Galβ4(Fucα3)GlcNAc | 82.1 ++ | 55.7 ± 9.4 + | 67.5 ± 4.6 | 12.6 | 49.1 |
| GF526 | PSGL-1, sLex on core 2 O-glycans | 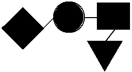 | SAα3Galβ4(Fucα3)GlcNAc | 90.8 ± 11.5 | 97.5 | 99.7 ± 0.12 | 98.6 | 99.9 |
| GF393 | Lewis y | 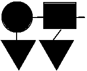 | Fucα2Galβ4(Fucα3)GlcNAcβ | 0.3 ± 0.0 − | 0.6 ± 0.0 − | 1.15 ± 0.92 | 1.0 | 0.8 |
| GF408 | blood group Ag A:(A1, A2) | 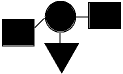 | GalNAcα3(Fucα2)GalβGlcNAc | 0.4 ± 0.21 | 0.6 | 1.40 ± 0.85 | 0.7 | 3.0 |
| GF409 | blood group A: (A3, Ax, A3B, AxB) | 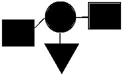 | GalNAcα3(Fucα2)GalβGlcNAc | 0.3 ± 0.0 | 0.5 | 0.95 ± 0.07 | 0.6 | 1.4 |
| GF411 | blood group B (secretor) | 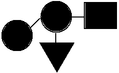 | Galα3(Fucα2)GalβGlcNAc | 0.8 ± 0.57 | 0.8 | 5.0 ± 2.7 | 2.1 | 13.5 |
| GF412 | blood group B (general) | 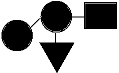 | Galα3(Fucα2)GalβGlcNAc | 3.3 ± 2.6 | 3.0 | 7.95 ± 0.07 | 18.2 | 58.9 |
| GF414 GF556 | TRA-1-81 Ag | | keratan sulphate in podocalyxin | 11.6 ± 13.8 | ND | 12.0 ± 0.71 | 10.4 | 69.7 |
| GF415 GF557 | TRA-1-60 Ag | | sialylated keratan sulphate in podocalyxin | 8.2 ± 10.6 | 2.6 | 10.9 ± 5.8 | 2.0 | 25.2 |
| GF377 | PN-15 renal gp200 | | | ND | ND | 5.35 ± 3.0 | 2.8 | 40.4 |

¹⁾Bone marrow/cord blood derived mesenchymal stem cells (BM/CB-MSC), ostegenic or adipocytic cells differentiated from MSC (OG/adipo);
²⁾Code for IHC: −, negative; +/−, occasional low expression; +, low expression; ++, common; +++, abundant.

TABLE 27
Stem cell and differentiated cell glycan binder target table based on structural analyses and binder specificities.
See explanations in footnotes 1) and 2).

| | | CB & BM | adipo/ | | | EB & st.3 | CD34+, | CD34−, |
|---|---|---|---|---|---|---|---|---|
| Trivial name | Terminal epitope | MSC | chondro diff. | osteo diff. | hESC | diff. | CD133+ | CD133− |
| LN type 1, Lec | Galβ3GlcNAcβ | + | + | +/− | ++ | + | +/− | +/− |
| | | L+ | Lq | L+ | O+ | L+ | L+ | L+ |
| | | | | | L++ | | | |
| | | | | | Nq | | | |
| | Lecβ3Galβ4Glc[NAc]β | +/− | q | +/− | ++ | + | +/− | +/− |
| Lea | Galβ3(Fucα4)GlcNAcβ | + | ++ | + | q | q | +/− | +/− |
| | | L+/− | | L+/− | | | L+ | L+ |
| | Leaβ3Galβ4Glc[NAc]β | +/− | | +/− | q | q | +/− | +/− |
| H type 1, H1 | Fucα2Galβ3GlcNAcβ | +/− | +/− | +/− | ++ | + | | |
| | | L+ | | L+ | L++ | | | |
| | H1β3Galβ4Glc[NAc]β | +/− | | +/− | ++ | +/− | | |
| Leb | Fucα2Galβ3(Fucα4)GlcNAcβ | +/− | +/− | +/− | + | ++ | q | q |
| sialyl Lea, sLea | SAα3Galβ3(Fucα4)GlcNAcβ | +/− | ++ | + | q | q | +/− | + |
| | | L+ | | L+ | | | Lq | Lq |
| | sLeaβ3Galβ4Glc[NAc]β | +/− | | +/− | | | q | q |
| α3′-sialyl Lec | SAα3Galβ3GlcNAcβ | +/− | ++ | + | + | +/− | q | q |
| | | Lq | Lq | Lq | Oq | | | |
| | | | | | Lq | | | |
| LN type 2, LN | Galβ4GlcNAcβ | ++ | + | ++ | ++ | + | + | + |
| | | N++ | N+ | N++ | N++ | | N+ | N+ |
| | | O+ | O+ | O+ | O+ | | O+ | O+ |
| | | Lq | Lq | Lq | Lq | | Lq | Lq |
| | LNβ2Manα3/6 | ++ | + | ++ | ++ | + | + | + |
| | LNβ4Manα3 | +/− | + | ++ | q | + | +/− | + |
| | LNβ2Manα3(LNβ2Manα6)Man | + | + | + | + | +/− | + | + |
| | LNβ2(LNβ4)Manα3(LNβ2Manα6)Man | q | q | ++ | q | q | q | +/− |
| | LNβ6(R-Galβ3)GalNAc | + | + | + | +/− | q | + | + |
| | LNβ3Galβ4Glc[NAc]β | q | q | q | +/− | q | q | q |
| | LNβ6(R-GlcNAcβ3)Galβ4Glc[NAc]β | q | | q | q | q | q | q |
| | LNβ3(R-GlcNAcβ6)Galβ4Glc[NAc]β | q | | q | q | q | q | q |
| | LNβ3(LNβ6)Galβ4Glc[NAc]β | q | | q | q | q | q | q |
| Lex | Galβ4(Fucα3)GlcNAcβ | +/− | + | +/− | ++ | +/− | + | + |
| | | L− | | L− | N++ | | Nq | Nq |
| | | | | | O+/− | | Oq | Oq |
| | | | | | Lq | | Lq | Lq |
| | Lexβ2Manα3/6 | q | q | q | + | q | +/− | +/− |
| | Lexβ6(R-Galβ3)GalNAc | q | q | q | +/− | q | + | q |
| | Lexβ3Galβ4Glc[NAc]β | q | ++ | q | +/− | q | q | q |
| | Lexβ2Manα3(Lexβ2Manα6)Man | q | q | q | +/− | − | q | q |
| H type 2, H2 | Fucα2Galβ4GlcNAcβ | + | ++ | + | ++ | ++ | +/− | + |
| | | L+ | Nq | L+ | N+ | | Nq | Nq |
| | | Nq | | Nq | Lq | | | |
| | H2β2Manα3/6 | q | q | q | + | q | q | q |
| | H2β3Galβ4Glc[NAc]β | + | | + | q | | q | q |
| Ley | Fucα2Galβ4(Fucα3)GlcNAcβ | +/− | +/− | +/− | q | q | +/− | +/− |
| | | L+ | | L+ | | | Lq | Lq |
| | Leyβ3Galβ4Glc[NAc]β | q | | q | q | | q | q |
| sialyl Lex, sLex | SAα3Galβ4(Fucα3)GlcNAcβ | ++ | ++ | ++ | ++ | +/− | ++ | + |
| | | O++ | O++ | O++ | Nq | | Nq | Nq |
| | | L− | | L− | O+ | | O++ | O+ |
| | | | | | Lq | | Lq | Lq |
| | sLexβ2Manα3/6 | Q | q | q | +/− | q | q | q |
| | sLexβ6(R-Galβ3)GalNAc | ++ | ++ | ++ | + | | ++ | + |
| | sLexβ3Galβ4Glc[NAc]β | + | + | +/− | q | | q | q |
| α3′-sialyl LN, | SAα3Galβ4GlcNAcβ | + | + | + | ++ | + | ++ | + |
| s3LN | | N+ | N+ | N+ | N+ | | N++ | N+ |
| | | O+ | O+ | O+ | O++ | | O+ | O+ |
| | | Lq | Lq | Lq | Lq | | Lq | Lq |
| | s3LNβ2Manα3/6 | + | + | + | ++ | + | ++ | + |
| | s3LNβ4Manα3 | +/− | + | ++ | q | q | +/− | + |
| | s3LNβ2Manα3(s3LNβ2Manα6)Man | + | + | + | + | q | ++ | + |
| | s3LNβ6(R-Galβ3)GalNAc | + | + | + | + | q | + | + |
| | s3LNβ3Galβ4Glc[NAc]β | + | + | + | q | q | q | q |
| | s3LNβ6(R-GlcNAcβ3)Galβ4Glc[NAc]β | q | | q | q | q | q | q |
| | s3LNβ3(R-GlcNAcβ6)Galβ4Glc[NAc]β | q | | q | q | q | q | q |
| α6′-sialyl LN, | SAα3Galβ4GlcNAcβ | q | q | q | + | + | q | q |
| s6LN | | Nq | Nq | Nq | N+ | | Nq | Nq |
| | s6LNβ2Manα3/6 | q | q | q | + | q | q | q |
| | s6LNβ4Manα3 | q | q | q | q | q | q | q |
| | s6LNβ2Manα3(s6LNβ2Manα6)Man | q | q | q | q | q | q | q |
| | s6LNβ3Galβ4Glc[NAc]β | − | − | − | q | q | q | q |
| Core 1 | Galβ3GalNAcα | +/− | +/− | +/− | q | q | + | +/− |
| H type 3 | Fucα2Galβ3GalNAcα | − | − | − | + | − | − | − |
| sialyl Core 1 | SAα3Galβ3GalNAcα | + | + | q | + | q | + | + |
| disialyl Core 1 | SAα3Galβ3Saα6GalNAcα | + | + | q | + | q | + | + |
| type 4 chain | Galβ3GalNAcβ | +/− | ++ | +/− | ++ | q | +/− | + |
| | | L+ | | L+ | L++ | | L+ | L+ |
| asialo-GM1 | Galβ3GalNAcβ4Galβ4Glc | + | ++ | ++ | | | +/− | + |
| Gb5, “SSEA-3” | Galβ3GalNAcβ3Galα4Galβ4Glc | + | + | +/− | ++ | q | +/− | + |
| H type4, “Globo | Fucα2Galβ3GalNAcβ | q | +/− | q | ++ | q | +/− | q |
| H” | | L+/− | | L+/− | | | | |
| α3′-sialyl type 4 | SAα3Galβ3GalNAcβ | ++ | q | + | + | q | q | q |
| | | L+ | | L+ | L++ | | L+ | L+ |
| “SSEA-4” | SAα3Galβ3GalNAcβ3Galα4Galβ4Glc | ++ | ++ | + | ++ | q | +/− | + |
| GalNAcβ | GalNAcβ | + | ++ | ++ | ++ | + | +/− | + |
| | | | | | L++ | | | |
| | | | | | N+ | | | |
| asialo-GM2 | GalNAcβ4Galβ4Glc | + | ++ | ++ | q | q | +/− | + |
| Gb4 | GalNAcβ3Galα4Galβ4Glc | + | | ++ | ++ | + | +/− | + |
| LacdiNAc | GalNAcβ4GlcNAcβ | | | | + | q | | |
| Galα | Galβ4Glc | +/− | +/− | +/− | q | q | +/− | + |
| Gb3 | Galα4Galβ4Glc | + | + | ++ | q | q | +/− | + |
| Lac | Galβ4Glc | q | q | q | q | q | q | q |
| GalNAcα, “Tn” | GalNAcα | +/− | + | q | q | | +/− | q |
| Forssman | GalNAcα3GalNAcβ | +/− | q | q | + | | +/− | + |
| sialyl Tn | SAα6GalNAcα | +/− | + | q | q | | q | +/− |
| oligosialic acid | NeuAcα8NeuAcα | + | ++ | ++ | +/− | + | q | q |
| | | L+ | L++ | L++ | | L+ | Lq | Lq |
| GD3 | NeuAcα8NeuAcα2Galβ4Glc | + | ++ | ++ | − | q | | |
| GD2 | NeuAcα8NeuAcα2(GalNAcβ4)Galβ4Glc | ++ | ++ | ++ | − | q | q | q |
| GD1b | NeuAcα8NeuAcα2(Galβ3GalNAcβ4)Galβ4 | +/− | ++ | +/− | − | q | | |
| | Glc | | | | | | | |
| GT1b | SAα8SAα2(Saα3Galβ3GalNAcβ4)Galβ4Glc | + | ++ | ++ | − | q | | |
| Manα | Manα | ++ | ++ | ++ | ++ | ++ | ++ | ++ |
| | Manα2Manα | ++ | + | + | ++ | + | ++ | + |
| | Manα3Manα6/β4 | + | ++ | + | + | ++ | + | ++ |
| | Manα6Manα6/β4 | + | ++ | + | + | ++ | + | ++ |
| | Manα3(Manα6)Manα6/β4 | + | ++ | + | + | ++ | + | ++ |
| | Manα3(Manα6)Manβ4GlcNAc[β4GlcNAc] | N+/− | N++ | N+ | N+/− | N++ | +/− | +/− |
| Manβ | Manβ | +/− | + | +/− | +/− | + | +/− | +/− |
| | Manβ4GlcNAc | +/− | + | +/− | +/− | + | +/− | +/− |
| Glcα | Glcα | + | +/− | +/ | + | +/− | + | +/− |
| | Glcα3Manα | + | +/ | +/ | + | +/ | + | +/− |
| | Glcα2Glcα3[Glcα3Manα] | +/− | +/ | +/ | +/− | +/ | +/− | +/− |
| core-Fuc | Fucα6GlcNAc | N+ | N+ | N+/− | N+ | N+/− | N+ | N+/− |
| | Fucα6(R-GlcNAcβ4)GlcNAc | + | + | +/− | + | +/− | + | +/− |
| GlcNAcβ, Gn | GlcNAcβ | + | + | +/− | + | +/− | + | +/− |
| | | N+ | N+ | Nq | N+ | Nq | N+ | Nq |
| | Gnβ2Manα3/6 | + | + | q | + | q | + | q |
| | Gnβ4Manα3 | + | q | | + | q | + | q |
| | Gnβ2Manα3(Gnβ2Manα6)Man | + | q | q | + | q | + | q |
| | Gnβ4Gn | q | q | q | q | q | q | q |
| | Gnβ4(Fucα6)Gn | q | q | q | q | q | q | q |
| | Gnβ6(R-Galβ3)GalNAc | − | − | − | − | − | q | q |
| | Gnβ3Galβ4Glc[NAc]β | q | q | q | q | q | q | q |
| | Gnβ6(R-GlcNAcβ3)Galβ4Glc[NAc]β | q | | q | q | | q | q |
| | Gnβ3(R-GlcNAcβ6)Galβ4Glc[NAc]β | q | | q | q | | q | q |

1) Stem cell and differentiated cell types are abbreviated as in other parts of the present document; CB/BM indicates MSC derived from cord blood or bone marrow; adipo/osteo/chondro diff. indicates cells differentiated into adipocyte, osteoblast, or chondrocyte direction from MSC; hESC indicates human embryonic stem cells, EB embryonic bodies, and st.3 diff. indicates stage 3 differentiated cells, preferentially to ectodermal/neuronal direction; and CD34+/CD133+ indicates HSC derived from cord blood, peripheral blood, or bone marrow; CD34−/CD133− indicates differentiated cells from the same source MNC fraction.
2) Occurrence of terminal epitopes in glycoconjugates and/or specifically in N-glycans (N), O-glycans (O), and/or glycosphingolipids (L). Code: q, qualitative data; +/−, low expression; +, common; ++, abundant.

1. A method of evaluating the status of a stern cell preparation comprising the step of detecting the presence of an elongated glycan structure or a group, at least two, of glycan structures in said preparation, wherein said glycan structure or a group of glycan structures is according to Formula T1 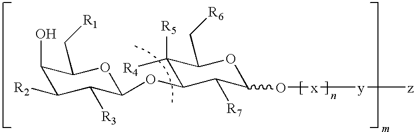
wherein X is linkage position
R₁, R₂, and R₆ are OH or glycosidically linked monosaccharide residue Sialic acid, preferably Neu5Acα2 or Neu5Gc α2, most preferably Neu5Acα2 or
R₃, is OH or glycosidically linked monosaccharide residue Fucα1 (L-fucose) or N-acetyl (N-acetamido, NCOCH₃);
R₄, is H, OH or glycosidically linked monosaccharide residue Fucα1 (L-fucose),
R₅ is OH, when R₄ is H, and R₅ is H, when R₄ is not H;
R7 is N-acetyl or OH
X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0;
Z is the carrier structure, preferably natural carrier produced by the cells, such as protein or lipid, which is preferably a ceramide or branched glycan core structure on the carrier or H;
The arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
With the provisions that one of R2 and R3 is OH or R3 is N-acetyl,
R6 is OH, when the first residue on left is linked to position 4 of the residue on right:
X is not Galα4Galβ4Glc, (the core structure of SSEA-3 or 4) or R3 is Fucosyl,
for the analysis of the status of stem cells and/or manipulation of the stem cells, and wherein said cell preparation is embryonic type stem cell preparation.
and when the glycan structure is an elongated structure, wherein the binder binds to the structure and additionally to at least one reducing end elongation epitope, preferably monosaccharide epitope, (replacing X and/or Y) according to the Formula E1:
AxHex(NAc)ₙ, wherein A is anomeric structure alfa or beta, X is linkage position 2, 3, or 6; and
Hex is hexopyranosyl residue Gal, or Man, and n is integer being 0 or 1, with the provisions that
when n is 1 then AxHexNAc is β4GalNAc or β6GalNAc,
when Hex is Man, then AxHex is β2Man, and
when Hex is Gal, then AxHex is β3Gal or β36Gal or α3Gal or α4Gal;
or
the binder epitope binds additionally to reducing end elongation epitope
Ser/Thr linked to reducing end GalNAcα-comprising structures or
βCer linked to Galβ4Glc comprising structures, and the glycan structure is the stem cell population determined from associated or contaminating cell population.

2. A method for the analysis of the status of the stem cells and/or for manipulation of stem cells comprising a step of detecting an elongated glycan structure or at least two glycan structures from a sample of stem cells, wherein said glycan structure is selected from the group consisting of: a terminal lactosamine structure
(R1)ₙ₁Gal(NAc)ₙ₃β3/4(Fucα4/3)ₙ₂GlcNAcβR wherein R1 is Fucα2, or SAα3, or SAα6 linked to Galβ4GlcNAc, and
R is the reducing end core structure of N-glycan, O-glycan and/or glycolipid; a, or structure
(SAα3)ₙ₁Galβ3(SAα6)ₙ₂GalNAc; wherein
n1, n2 and n3 are 0 or 1 indicating presence or absence of a structure wherein SA is a sialic acid; or branched epitope
Galβ3(GlcNAcβ6)GalNAc or
R₁Galβ4(R₃)GlcNAcβ6(R₂Galβ3)GalNAc,
wherein R₁ and R₂ are independently either nothing or SAα3; and R₃ is independently either nothing or Fucα3 ; or
Manβ4GlcNAc structure in the core structure of N-linked glycan; or epitope Galβ4Glc,
or terminal mannose
or terminal SAα3/6Gal, wherein SA is a sialic acid, with the provisions that
i) the stem cells are not cells of a cancer cell line and

3. The method according to claim 1, wherein said binding agent recognizes structure according to the Formula T8Ebeta
[Mα]ₘGalβ1-3/4[Nα]ₙGlcNAcβxHex(NAc)ₚ
wherein
wherein x is linkage position 2, 3, or 6
wherein m, n and p are integers 0, or 1, independently
M and N are monosaccharide residues being
i) independently nothing (free hydroxyl groups at the positions) and/or
ii)SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc,
with the provision that m, n and p are 0 or 1, independently.
Hex is hexopyranosyl residue Gal, or Man,
with the provisions that when p is 1 then βxHexNAc is β6GalNAc,
when p is 0
then Hex is Man and βxHex is β2Man, or Hex is Gal and βxHex is β3Gal or β6Gal.

4. The method according to claim 1, wherein said binding agent recognizes type II Lactosmine based structures according to the Formula T10E
[Mα]ₘGalβ1-4[Nα]ₙGlcNAcβxHex(NAc)ₚ
with the provisions that when p is 1 then βxHexNAc is β6GalNAc,
when p is 0, then Hex is Man and βxHex is β2Man, or Hex is Gal and βxHex is β6Gal.

5. The method according to claim 4, wherein said binding agent recognizes type II Lactosmine based structures according to the Formula T10EMan:
[Mα]ₘGalβ1-4[Nα]ₙGlcNAcβ2Man,
wherein the variables are as described for Formula T8Ebeta.

6. The method according to claim 5, wherein the structures are selected from the group consisting of Galβ4GlcNAcβ2Man, Galβ4(Fucα3)GlcNAcβ2Man, Fucα2Galβ4GlcNAcβ2Man, SAα6Galβ4GlcNAcβ2Man, SAα3Galβ4GlcNAcβ2Man

7. The method according to claim 5, wherein the structure is H type II structure Fucα2Galβ4GlcNAcβ2Man

8. The method according to claim 5, wherein the structure is Lewis x structure Galβ4(Fucα3)GlcNAcβ2Man.

9. The method according to claim 4, wherein said binding agent recognizes type II Lactosmines according to the Formula T10EGal(NAc):
[Mα]ₘGalβ1-4[Nα]ₙGlcNAcβ6Gal(NAc)ₚ
wherein the variables are as described for Formula T8Ebeta.

10. The method according to claim 9, wherein the structures are selected from the group consisting of
Galβ4GlcNAcβ6Gal, Galβ4GlcNAcβ6GalNAc, Galβ4(Fucα3)GlcNAcβ6GalNAc, Fucα2Galβ4GlcNAcβ6GalNAc, SAα3/6Galβ4GlcNAcβ6GalNAc, and SAα3Galβ4GlcNAcβ6GalNAc, SAα3Galβ4(Fucα3)GlcNAcβ6GalNAc, SAα3Galβ4(Fuca3)GlcNAcβ6(RGalβ3)GalNAc, wherein R is SAα3 or nothing.

11. The method according to claim 1, wherein said binding agent recognizes type I Lactosmine based structures according to the Formula T9E
[Mα]ₘGalβ1-3[Nα]ₙGlcNAcβ3Gal

12. The method according to claim 11, wherein the structures are selected from the group consisting of
Galβ3GlcNAcβ3Gal, Galβ3(Fucα4)βGlcNAcβ3Gal, and Fucα2Galβ3GlcNAcβ3Gal, and Fucα2Galβ3(Fucα4)GlcNAcβ3Gal , and SAα3Galβ3(Fucα4)GlcNAcβ3Gal.

13. The method according to claim 11, wherein the structures is H type I structure Fucα2Galβ3GlcNAcβ3Gal or type I LAcNAc-structure Galβ3GlcNAcβ3Gal.

14. The method according to claim 1, wherein the detection is performed by analysing the amount or presence of at least one glycan structure in said preparation by a specific binding agent or a controlled binder.

15. The method according to claim 1, wherein said structure comprises at least one Fucα-residue.

16. The method according to claim 2, wherein the elongated oligosaccahride structures are selected from the group consisting of (SAα3)₀ₒᵣ₁Galβ3/4(Fucα4/3)GlcNAc, Fucα2Galβ3GalNAcα/β3 and Fucα2Galβ3(Fucα4)₀ₒᵣ₁GlcNAcβ.

17. The method according to claim 2, wherein the elongated oligosaccahride are selected from the group consisting of Galβ4Glc, Galβ3GlcNAc, Galβ3GalNAc, Galβ4GlcNAc, Galβ3GlcNAcβ, Galβ3GalNAcβ/α, Galβ4GlcNAcβ, GalNAcβ4GlcNAc, SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, SAα3Galβ4GlcNAcβ, SAα6Galβ4Glc, SAα6Galβ4Glcβ, SAα6Galβ4GlcNAc, SAα6Galβ4GlcNAcβ, Galβ3(Fucα4)GlcNAc (Lewis a), SAα3Galβ3(Fucα4)GlcNAc (sialyl-Lewis a), Fucα2Galβ3GlcNAc (H-type 1), Fucα2Galβ3(Fucα4)GlcNAc (Lewis b), Galβ4GlcNAc (type 2 lactosamine based), Galβ4(Fucα3)GlcNAc (Lewis x), SAα3Galβ3(Fucα4)GlcNAc (sialyl-Lewis x), Fucα2Galβ4GlcNAc (H-type 2) and Fucα2Galβ4(Fucα3)GlcNAc (Lewis y).

18. The method according to claim 1, when the structure is used together with at least one terminal ManαMan-structure.

19. The method according to claim 1, wherein the detection is performed by a binder being a recombinant protein selected from the group consisting of monoclonal antibody, glycosidase, glycosyl transferring enzyme, plant lectin, animal lectin and a peptide mimetic thereof.

20. The method according to claim 19, wherein the said binding agent binds to the same epitope than the antibodies selected from the group consisting of GF 287, GF 279, GF 288, GF 284, GF 283, GF 286, GF 290, GF 289, GF275, GF276, GF277, GF278, GF297, GF298, GF302, GF303, GF305, GF296, GF300, GF304, GF307, GF353, and GF354.

21. The method according to claim 19, wherein said binding agent is selected from the group consisting of GF 287, GF 279, GF 288, GF 284, GF 283, GF 286, GF 290, and GF 289, GF275, GF276, GF277, GF278, GF297, GF298, GF302, GF303, GF305, GF296, GF300, GF304, GF307, GF353, GF354, and GF 367.

22. The method according to the claim 19, wherein the recombinant protein is a high specificity binder recognizing at least partially two monosaccharide structures and bond structure between the monosaccharide residues.

23. The method according to the claim 19, wherein the binder is used for sorting or selecting human stem cells from biological materials or samples including cell materials comprising other cell types.

24. The method according to the claim 19, wherein the binder is used for sorting or selecting between different human stem cell types.

25. The method according to claim 19, wherein sorting or selecting is performed by FACS or any other means to enrich a cell population.

26. A cell population obtained by the method according to claim 25.

27. The method according to claim 24, wherein the cell preparation is selected from the group consisting of blood related cell population.

28. The method according to claim 1, wherein the amount of cells to be analysed is between 10³ and 10⁶ cells.

29. The method according to claim 1, wherein the glycan structure is present in a N-glycan subglycome comprising N-Glycans with N-glycan core structure and said N-Glycans being releasable from cells by N-glycosidase.

30. The method according to claim 29, wherein the N-glycan core structure is Manβ4GlcNAcβ4(Fucα6)ₙGlcNAc, wherein n is 0 or 1.

31. The method according to claim 1, wherein the glycan structure is present in a O-glycan subglycome comprising O-Glycans with O-glycan core structure, or the glycan structure is present in a glycolipid subglycome comprising glycolipidss with glycolipid core structure and the glycans are releasable by glycosylceramidase.

32. The method according to claim 1, wherein the group of glycan structures comprises oligosaccharides in specific amounts shown in Tables and Figures of the specification.

33. The method according to claim 1, wherein the presence or absence of cell surface glycomes of said cell preparation is detected.

34. The method according to claim 1, wherein said cell preparation is evaluated/detected with regard to a contaminating structure in a cell population of said cell preparation, time dependent changes or a change in the status of the cell population by glycosylation analysis using mass spectrometric analysis of glycans in said cell preparation.

35. The method according to claim 34, wherein the cell status is controlled during cell culture or during cell purification, in context with cell storage or handling at lower temperatures, or in context with cryopreservation of cells.

36. The method according to claim 34, wherein time dependent changes of cell status depend on the nutritional status of the cells, confluency of the cell culture, density of the cells, changes in genetic stability of the cells, integrity of the cell structures or cell age, or chemical, physical, or biochemical factors affecting the cells.

37. A method for identifying, characterizing, selecting or isolating stem cells in a population of mammalian cells which comprises using a binder or binding agent, said binder/binding agent binding to a glycan structure or glycan structures according to claim 1, wherein said structure
(i) exhibits expression on/in stem cells and an absence of expression or low expression in feeder cells, or differentiated cells;
(ii) exhibits absence of expression or low expression in stem cells and expression or high expression or mainly expressed in feeder cells or differentiated cells;
(iii) exhibits expression in subpopulations of stem cells; or (iv) exhibits expression in subpopulations of differentiated stem cells.

38. The method according to claim 37, wherein stem cells are totopotent, pluripotent, or multipotent.

39. The method of claim 38 wherein the embryonic stem cell binder is used for identifying the pluripotent or multipotent stem cells and the method further comprises selecting the identified pluripotent or multipotent stem cells for collection.

40. The method of claim 39 which further comprises separating the selected pluripotent or multipotent stem cells from the population of mammalian cells.

41. The method of claim 40 which further comprises isolating the separated pluripotent or multipotent stem cells.

42. The method of claim 40 wherein the cell population is selected from cord blood, embryonal body fluids, embryonal tissue samples, embryonal tissue cultures, cell lines and cell cultures of non mesenchymal adult origin.

43. The method of claim 40 wherein the stem cells are adult stem cells, embryonic stem cells or stem cells of fetal origin, preferably of human fetal origin within a maternal cell population.

44. The method of claim 40, wherein the stem cells are dedifferentiated somatic cells.

45. The method of claim 1, wherein the antibody is selected from the group consisting of a polyclonal antibody, a monoclonal antibody, and an antibody fragment.

46. The method of claim 1, wherein the binder is controlled binder.

47. The method of claim 1, wherein the binder comprises at least the glycan structure binding portion of an antibody, lectin, or glycosidase specific to at least one epitope of a glycan structure according to claim 1; and said glycan structure is attached to a stem cell and/or a differentiated cell.

48. A method for identification, selection or characterization of embryonic stem cells from mammalian fluids or tissues which comprises obtaining an antibody, lectin or glycosidase specific to at least one epitope of the glycan structure according to claim 1, and contacting the antibody, lectin or glycosidase with the stem cells to identify, select, isolate and/or characterize such cells.

49. Mammalian stem cells isolated by the method of claim 48.

50. A method for identifying a selective stem cell binder to a glycan structure of claim 1, which comprises:
selecting a glycan structure exhibiting specific expression in/on stem cells and absence of expression in/on feeder cells and/or differentiated somatic cells; and
confirming the binding of the binder to the glycan structure in/on stem cells.

51. A kit for enrichment and detection of stem cells within a specimen, comprising: at least one reagent comprising a binder to detect glycan structure according to claim 1; and instructions for performing stem cell enrichment using the reagent, optionally including means for performing stem cell enrichment.

52. The kit of claim 51, wherein the reagent is a labeled with a detectable tracer.

53. A composition comprising glycan structure according to claim 1, bearing stem cell and a binder that binds with a glycan structure to any the claims 14-8 on a stem cell.

54. A method of evaluating the status of a stem cell preparation comprising the step of detecting the presence of a glycan structure or a group of glycan structures in said preparation, wherein said glycan structure or a group of glycan structures is according to Formula T11:
[M]ₘGalβ1-x[Nα]ₙHex(NAc)ₚ, wherein m, n and p are integers 0, or 1, independently Hex is Gal or Glc, X is linkage position;
M and N are monosaccharide residues being independently nothing (free hydroxyl groups at the positions) and/or
SAα which is Sialic acid linked to 3-position of Gal or/and 6-position of HexNAc Galα linked to 3 or 4-position of Gal, or
GalNAcβ linked to 4-position of Gal and/or
Fuc (L-fucose) residue linked to 2-position of Gal and/or 3 or 4 position of HexNAc, when Gal is linked to the other position (4 or 3),
and HexNAc is GlcNAc, or 3-position of Glc when Gal is linked to the other position (3),
with the provision that sum of m and n is 2
preferably m and n are 0 or 1, independently, and
with the provision that when M is Gala then there is no sialic acid linked to Galη1, and
n is 0 and preferably x is 4.
with the provision that when M is GalNAcβ, then there is no sialic acid α6-linked to Galβ1, and
n is 0 and x is 4.

55. The method according to claim 54, wherein the structure is according to the Formula T12:
[M][SAα3]ₙGalβ1-4Glc(NAc)ₚ,
wherein n and p are integers 0, or 1, independently
M is Gala linked to 3 or 4-position of Gal, or GalNAcβ linked to 4-position of Gal
and/or SAα is Sialic acid branch linked to 3-position of Gal
with the provision that when M is Gala then there is no sialic acid linked to Galβ1 (n is 0).

56. The method according to claim 54, wherein the structure comprises globotriose (Gb3) non-reducing end terminal structure Galα4Gal.

57. A use of binder molecules as described in claim 1 for isolation of cellular components from stem cells comprising the novel target/marker structures.

58. The use according to the claim 57, wherein the isolated cellular components are free glycans or glycans conjugated to proteins or lipids or fragment thereof.

59. Method to isolate cellular component including following steps using the binder molecules according to claim 57 comprising steps
1) Providing a stem cell sample.
2) Contacting the binder molecule according to the invention to the corresponding target structures.
3) Isolating the complex of the binder and target structure at least from part of cellular materials.

60. A target structure composition produced by the method according to claim 59, comprising glycoproteins or glycopeptides comprising glycan structure corresponding to the binder structure and peptide or protein epitopes specifically expressed in stem cells or in proportions characteristic to stem cells.

61. A method of evaluating the status of a human blood related, preferably hematopietic, stem cell preparation and/or contaminating cell population comprising the step of detecting the presence of an elongated glycan structure or a group, at least two, of glycan structures in said preparation, wherein said glycan structure or a group of glycan Tn and sialyl-Tn structures is according to Formula MUC
(R)GalNAcαSer/Thr)ₘ
wherein n and m are 0 or 1, independently and R is SA1:16 or Galβ3, SA is sialic acid preferably Neu5Ac, and when R is Galβ3 n is 1, preferably Tn antiges:
(SAα6)ₙGalNAcα(Ser/Thr)ₘ,
wherein n and m are 0 or 1, independently and SA is sialic acid preferably Neu5Ac, or TF antigen
Galβ3GalNAcα(Ser/Thr)ₘ.
